(19) United States
(12) Patent Application Publication
(10) Pub. No.: US 2012/0225062 A1
(43) Pub. Date: Sep. 6, 2012

(54) NOVEL KINASE INHIBITORS

(76) Inventors: Matthew Burger, Albany, CA (US); Gisele Nishiguchi, Albany, CA (US); Timothy D. Machajewski, Martinez, CA (US); Alice Rico, Castro Valley, CA (US); Robert Lowell Simmons, San Francisco, CA (US); Aaron R. Smith, Fremont, CA (US); Victoriano Tamez, JR., Emeryville, CA (US); Huw Tanner, Alameda, CA (US); Lifeng Wan, Union City, CA (US)

(21) Appl. No.: 13/410,845

(22) Filed: Mar. 2, 2012

Related U.S. Application Data
(60) Provisional application No. 61/449,229, filed on Mar. 4, 2011.
(60) Provisional application No. 61/480,015, filed on Apr. 28, 2011.

(51) Int. Cl. C07D 401/12; A61K 31/444; A61K 31/4545; C07D 413/14; A61K 31/4375; A61K 31/7068; A61K 31/513; A61K 31/351; A61K 31/519; A61K 31/282; A61K 33/24; A61K 31/675; A61K 31/506; A61K 31/165; A61P 35/04; A61K 35/02; A61P 37/00; A61P 1/04; A61P 19/02; C07D 401/14

(52) U.S. Cl. 424/133.1; 546/262; 546/194; 514/332; 514/318; 546/256; 544/131; 514/235.5; 514/283; 514/49; 514/274; 514/459; 514/255.05; 514/492; 424/649; 514/90; 514/252.18; 514/154

(57) ABSTRACT

The present invention provides compounds of Formula I:

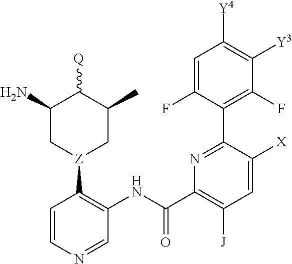

and related compounds as further described herein, and pharmaceutical compositions comprising these compounds. The invention further provides methods to use these compounds and compositions for treating disorders associated with undesired levels of Pim kinase activity, including cancers and autoimmune disorders.

### CROSS-REFERENCE TO RELATED APPLICATIONS

This application claims the benefit of priority under 35 U.S.C. §119(e) to U.S. provisional application Ser. No. 61/449,229 filed on Mar. 4, 2011, and U.S. provisional application Ser. No. 61/480,015 filed on Apr. 28, 2011, which are incorporated herein by reference in their entirety.

### FIELD OF THE INVENTION

The present invention relates to new compounds and their tautomers and stereoisomers, and pharmaceutically acceptable salts, esters, metabolites or prodrugs thereof, compositions of the new compounds together with pharmaceutically acceptable carriers, and uses of the new compounds, either alone or in combination with at least one additional therapeutic agent, in the prophylaxis or treatment of cancer and other cellular proliferation disorders.

### BACKGROUND

Infection with the Maloney retrovirus and genome integration in the host cell genome results in development of lymphomas in mice. Provirus Integration of Maloney Kinase (PIM-Kinase) was identified as one of the frequent proto-oncogenes capable of being transcriptionally activated by this retrovirus integration event (Cuypers H T et al., “Murine leukemia virus-induced T-cell lymphomagenesis: integration of proviruses in a distinct chromosomal region,” Cell 37(1):141-50 (1984); Selten G, et al., “Proviral activation of the putative oncogene Pim-1 in MuLV induced T-cell lymphomas” EMBO J 4(7):1793-8 (1985)), thus establishing a correlation between over-expression of this kinase and its oncogenic potential. Sequence homology analysis demonstrated that there are three highly homologous Pim-Kinases (Pim1, 2 & 3), Pim1 being the proto-oncogene originally identified by retrovirus integration. Furthermore, transgenic mice over-expressing Pim1 or Pim2 show increased incidence of T-cell lymphomas (Breuer M et al., “Very high frequency of lymphoma induction by a chemical carcinogen in pim-1 transgenic mice” Nature 340(6228):61-3 (1989)), while over-expression in conjunction with c-myc is associated with incidence of B-cell lymphomas (Verbeek S et al., “Mice bearing the E mu-myc and E mu-pim-1 transgenes develop pre-B-cell leukemia prenatally” Mol Cell Biol 11(2):1176-9 (1991)). Thus, these animal models establish a strong correlation between Pim over-expression and oncogenesis in hematopoietic malignancies.

In addition to these animal models, Pim over-expression has been reported in many human malignancies. Pim1, 2 & 3 over-expression is frequently observed in hematopoietic malignancies (Amson R et al., “The human protooncogene product p33pim is expressed during fetal hematopoiesis and in diverse leukemias,” PNAS USA 86(22):8857-61 (1989); Cohen A M et al., “Increased expression of the hPim-2 gene in human chronic lymphocytic leukemia and non-Hodgkin lymphoma,” Leuk Lymph 45(5):951-5 (2004), Huttmann A et al., “Gene expression signatures separate B-cell chronic lymphocytic leukeamia prognostic subgroups defined by ZAP-70 and CD38 expression status,” Leukemia 20:1774-1782 (2006)) and in prostate cancer (Dhanasekaran S M, et al., “Delineation of prognostic biomarkers in prostate cancer,” Nature 412(6849):822-6 (2001); Cibull T L, et al., “Overexpression of Pim-1 during progression of prostatic adenocarcinoma,” J Clin Pathol 59(3):285-8 (2006)), while over-expression of Pim3 is frequently observed in hepatocellular carcinoma (Fujii C, et al., “Aberrant expression of serine/threonine kinase Pim-3 in hepatocellular carcinoma development and its role in the proliferation of human hepatoma cell lines,” Int J Cancer 114:209-218 (2005)) and pancreatic cancer (Li Y Y et al., “Pim-3, a proto-oncogene with serine/threonine kinase activity, is aberrantly expressed in human pancreatic cancer and phosphorylates bad to block bad-mediated apoptosis in human pancreatic cancer cell lines,” Cancer Res 66(13):6741-7 (2006)).

Pim1, 2 & 3 are Serine/Threonine kinases that normally function in survival and proliferation of hematopoietic cells in response to growth factors and cytokines Cytokines signaling through the Jak/Stat pathway leads to activation of transcription of the Pim genes and synthesis of the proteins. No further post-translational modifications are required for the Kinase Pim activity. Thus, signaling downstream is primarily controlled at the transcriptional/translational and protein turnover level. Substrates for Pim kinases include regulators of apoptosis such as the Bcl-2 family member BAD (Aho T et al., “Pim-1 kinase promotes inactivation of the pro-apoptotic Bad protein by phosphorylating it on the Ser112 gatekeeper site,: FEBS Letters 571: 43-49 (2004)), cell cycle regulators such as p21^{WFA1/CIP1} (Wang Z, et al., “Phosphorylation of the cell cycle inhibitor p21Cip1/WAF1 by Pim-1 kinase,” Biochem Biophys Acta 1593:45-55 (2002)), CDC25A (1999), C-TAK (Bachmann M et al., “The Oncogenic Serine/Threonine Kinase Pim-1 Phosphorylates and Inhibits the Activity of Cdc25C-associated Kinase 1 (C-TAK1). A novel role for Pim-1 at the G2/M cell cycle checkpoint,” J Biol Chem 179:48319-48328 (2004)) and NuMA (Bhattacharya N, et al., “Pim-1 associates with protein complexes necessary for mitosis,” Chromosoma 111(2):80-95 (2002)) and the protein synthesis regulator 4EBP1 (Hammerman P S et al., “Pim and Akt oncogenes are independent regulators of hematopoietic cell growth and survival,” Blood 105(11):4477-83 (2005)). The effects of Pim(s) in these regulators are consistent with a role in protection from apoptosis and promotion of cell proliferation and growth. Thus, over-expression of Pim(s) in cancer is thought to play a role in promoting survival and proliferation of cancer cells and, therefore, their inhibitions should be an effective way of treating cancers in which they are over-expressed. In fact several reports indicate that knocking down expression of Pim(s) with siRNA results in inhibition of proliferation and cell death (Dai J M, et al., “Antisense oligodeoxynucleotides targeting the serine/threonine kinase Pim-2 inhibited proliferation of DU-145 cells,” Acta Pharmacol Sin 26(3):364-8 (2005); Fujii et al. 2005; Li et al. 2006).

Furthermore, mutational activation of several well known oncogenes in hematopoietic malignancies is thought to exert its effects at least in part through Pim(s). For example, targeted down-regulation of Pim expression impairs survival of hematopoietic cells transformed by Flt3 and BCR/ABL (Adam et al. 2006). Thus, inhibitors to Pim1, 2 and 3 would be useful in the treatment of these malignancies.

In addition to a potential role in cancer treatment and myeloproliferative diseases, such inhibitor could be useful to control expansion of immune cells in other pathologic condition such as autoimmune diseases, allergic reactions and in organ transplantation rejection syndromes. This notion is supported by the findings that differentiation of Th1 Helper T-cells by IL-12 and IFN-α results in induction of expression of both Pim1 and Pim2 (Aho T et al., “Expression of human Pim family genes is selectively up-regulated by cytokines promoting T helper type 1, but not T helper type 2, cell differentiation,” Immunology 116: 82-88 (2005)). Moreover, Pim(s) expression is inhibited in both cell types by the immunosuppressive TGF-β (Aho et al. 2005). These results suggest that Pim kinases are involved in the early differentiation process of Helper T-cells, which coordinate the immunological responses in autoimmune diseases, allergic reaction and tissue transplant rejection. Recent reports demonstrate that Pim kinase inhibitors show activity in animal models of inflammation and autoimmune diseases. See J E Robinson “Targeting the Pim Kinase Pathway for Treatment of Autoimmune and Inflammatory Diseases,” for the Second Annual Conference on Anti-Inflammatories: Small Molecule Approaches, San Diego, Calif. (Conf. April 2011; Abstract published earlier on-line).

A continuing need exists for compounds that inhibit the proliferation of capillaries, inhibit the growth of tumors, treat cancer, modulate cell cycle arrest, and/or inhibit molecules such as Pim1, Pim2 and Pim3, and pharmaceutical formulations and medicaments that contain such compounds. A need also exists for methods of administering such compounds, pharmaceutical formulations, and medicaments to patients or subjects in need thereof. The present invention addresses such needs.

Earlier patent applications have described compounds that inhibit Pims and function as anticancer therapeutics, see, e.g., WO 2008/106692 and PCT/EP2009/057606, and as treatment for inflammatory conditions such as Crohn's disease, inflammatory bowel disease, rheumatoid arthritis, and chronic inflammatory diseases, see e.g., WO 2008/022164. The present invention provides novel compounds that inhibit activity of one or more Pims and exhibit distinctive characteristics such as improved toxicological properties that are believed to provide improved therapeutic effects. Compounds of the invention contain novel substitution patterns on one or more rings, particularly the phenyl ring, that provide these distinctive properties.

### SUMMARY OF THE INVENTION

The invention provides compounds of Formula I:

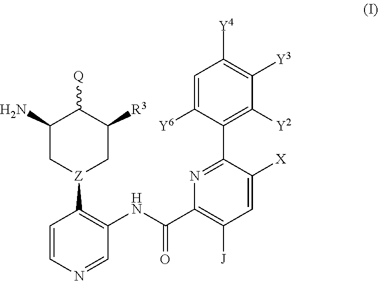

or a pharmaceutically acceptable salt thereof, wherein:

### Z is N or CH;

### Q is H, Me, or —OH;

R³ is H, Me, or C₂₋₄ alkyl;

### X is H or F;

### J is H or NH₂;

Y² and Y⁶ are each independently F or Cl, preferably F;
Y³ is H or is selected from the group consisting of CN, OEt, S(O)ₚR, —O(CH₂)_{q}—OH, —O(CH₂)_{q}—OR, —(CH₂)_{q}—OH, —C(CH₃)₂OH, —(CH₂)_{q}—OR, —(CR′₂)₁₋₃—OR′ or —O—(CR′₂)₁₋₃—OR′ where each R′ is independently H or Me, and an optionally substituted member selected from the group consisting of C₁₋₄ alkyl, C₂₋₄ alkyenyl, C₂₋₄ alkynyl, C₁₋₄alkoxy, C₂₋₄ alkyenyloxy, C₂₋₄ alkynyloxy, C₁₋₄ alkylthio, C₁₋₄ alkylsulfonyl, C₁₋₄hydroxyalkyl, C₁₋₄ hydroxyalkyloxy, C₃₋₇ cycloalkyl, C₃₋₇ heterocycloalkyl, C₅₋₁₀ heteroaryl, and C₆₋₁₀ aryl, each of which is optionally substituted with up to three groups independently selected from halo, hydroxy, amino, OMe, CN, oxo, R and OR; when Y³ is H, Y⁴ is selected from the group consisting of CN, R, vinyl, COOH, COOR, S(O)_{q}R, —O(CH₂)_{q}—OH, —O(CH₂)_{q}—OR, —(CH₂)_{q}—OH, —C(CH₃)₂OH, —(CH₂)ₚ—OR, —(CH₂)_{q}—R, —O—(CH₂)_{q}—R, —(CR′₂)₁₋₃—OR′ or —O—(CR′₂)₁₋₃—OR′ where each R′ is independently H or Me, and an optionally substituted member selected from the group consisting of C₁₋₄ alkyl, C₁₋₄ alkoxy, C₁₋₄ alkylthio, C₁₋₄ alkylsulfonyl, C₁₋₄hydroxyalkyl, C₁₋₄ hydroxyalkyloxy, C₃₋₇ cycloalkyl, C₃₋₇ heterocycloalkyl, C₅₋₁₀ heteroaryl, and C₆₋₁₀ aryl, each of which is optionally substituted with up to two groups independently selected from halo, hydroxy, amino, OMe, CN, oxo, R and OR; and
Y⁴ can be H when Y³ is not H;
or Y³ and Y⁴ taken together form a 5-6 membered ring selected from cycloalkyl, cycloalkenyl, heterocyclyl, heteroaryl and aryl, which ring is optionally substituted with up to two groups independently selected from R, halo, —OH, —OR, —(CH₂)₁₋₃—OR, —O—(CH₂)₁₋₃—OR, —(CH₂)_{q}—OH, and —(CH₂)_{q}—OH;
each R is independently an optionally substituted C₁₋₄ alkyl, C₃₋₇ cycloalkyl, C₅₋₆ cycloalkenyl, C₅₋₆ heterocyclyl, or 3-7 membered cyclic ether, wherein the optional substitutents for R are independently selected from OH, Me, —CH₂OH, COOH, COOMe, CONH₂, CONHMe, CONMe₂, CF₃, OMe, CN, NH₂, halo, oxo, and CN;
each q is independently 1 or 2; and
each p is independently 0, 1 or 2.

In the compounds of Formula (I), the phenyl ring shown has at least one substituent at the positions corresponding to Y³ or Y⁴ that is not H. Various embodiments of these compounds are described herein, and provide improved biological effects relative to compounds known in the art.

In certain embodiments, the compound of Formula (I) is a compound of Formula (Ia),

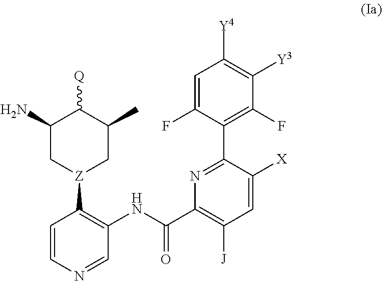

or a pharmaceutically acceptable salt thereof, wherein:

### Z is N or CH;

### Q is H, Me or —OH;

### X is H or F;

### J is H or NH₂;

Y³ is H or is selected from the group consisting of CN, OEt, S(O)ₚR, —O(CH₂)_{q}—OH, —O(CH₂)_{q}—OR, —(CH₂)_{q}—OH, —(CH₂)_{q}—OR, —(CR′₂)₁₋₃—OR′ or —O—(CR′₂)₁₋₃—OR′ where each R′ is independently H or Me, and an optionally substituted member selected from the group consisting of C₁₋₄ alkyl, C₁₋₄ alkoxy, C₁₋₄ alkylthio, C₁₋₄ alkylsulfonyl, C₁₋₄ hydroxyalkyl, C₁₋₄ hydroxyalkyloxy, C₃₋₇ cycloalkyl, C₃₋₇ heterocycloalkyl, C₅₋₁₀ heteroaryl, and C₆₋₁₀ aryl, each of which is optionally substituted with up to two groups independently selected from halo, hydroxy, amino, OMe, CN, oxo, R and OR;
when Y³ is H, Y⁴ is selected from the group consisting of CN, R, vinyl, COOH, COOR, S(O)_{q}R, —O(CH₂)_{q}—OH, —O(CH₂)_{q}—OR, —(CH₂)_{q}—OH, —(CH₂)ₚ—OR, —(CR′₂)₁₋₃—OH or —O—(CR′₂)₁₋₃—OH, where each R′ is independently H or Me, and an optionally substituted member selected from the group consisting of C₁₋₄ alkyl, C₁₋₄ alkoxy, C₁₋₄ alkylthio, C₁₋₄ alkylsulfonyl, C₁₋₄ hydroxyalkyl, C₁₋₄ hydroxyalkyloxy, C₃₋₇ cycloalkyl, C₃₋₇ heterocycloalkyl, C₅₋₁₀ heteroaryl, and C₆₋₁₀ aryl, each of which is optionally substituted with up to two groups independently selected from halo, hydroxy, amino, OMe, CN, oxo, R and OR;
and when Y³ is not H Y⁴ is H;
each R is independently an optionally substituted C₁₋₄ alkyl, C₃₋₇ cycloalkyl, or 3-7 membered cyclic ether, wherein the optional substituents are independently selected from OH, OMe, CN, NH₂, halo, oxo, and CN;
each q is 1 or 2; and
each p is independently 0, 1 or 2.

The invention also provides compounds of Formula IIa and IIb:

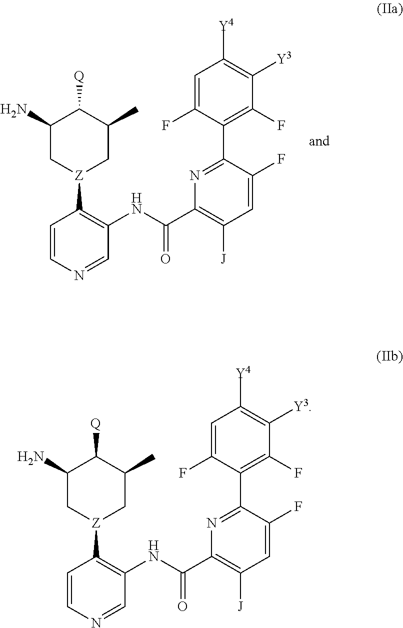

as further described herein.

In some embodiments of the compounds of Formula I or IIa or IIb, J is H. In other embodiments of these compounds, J is NH₂. Preferably, J is H. In some embodiments, Z is CH, and the stereochemistry of the ring containing Z is as shown in the Formula; in other embodiments of these compounds, Z is N. In many embodiments of Formula I, R³ is Me, and Y² and Y⁶ are each F.

In some embodiments of the compounds of Formula I or IIa or IIb, R is preferably an optionally substituted C₁₋₄ alkyl, such as cyclopropylmethyl, hydroxyalkyl, or haloalkyl, or an optionally substituted 3-7 membered cyclic ether such as an oxetanyl, tetrahydrofuranyl or tetrahydropyranyl group.

The invention also provides specific compounds including:
N-(4-((1R,3S,5S)-3-amino-5-methylcyclohexyl)pyridin-3-yl)-6-(2,6-difluoro-4-(methylsulfonyl)phenyl)-5-fluoropicolinamide
N-(4-((1R,3S,5S)-3-amino-5-methylcyclohexyl)pyridin-3-yl)-6-(4-((R)-2,3-dihydroxypropoxy)-2,6-difluorophenyl)-5-fluoropicolinamide
N-(4-((1R,3S,5S)-3-amino-5-methylcyclohexyl)pyridin-3-yl)-6-(4-((S)-2,3-dihydroxypropoxy)-2,6-difluorophenyl)-5-fluoropicolinamide
N-(4-((1R,3R,4R,5S)-3-amino-4-hydroxy-5-methylcyclohexyl)pyridin-3-yl)-6-(2,6-difluoro-4-methylphenyl)-5-fluoropicolinamide
N-(4-((1R,3R,4R,5S)-3-amino-4-hydroxy-5-methylcyclohexyl)pyridin-3-yl)-6-(2,6-difluoro-4-methoxyphenyl)-5-fluoropicolinamide
N-(4-((1R,3R,4S,5S)-3-amino-4-hydroxy-5-methylcyclohexyl)pyridin-3-yl)-6-(2,6-difluoro-4-methylphenyl)-5-fluoropicolinamide
N-(4-((1R,3R,4S,5S)-3-amino-4-hydroxy-5-methylcyclohexyl)pyridin-3-yl)-6-(2,6-difluoro-4-methoxyphenyl)-5-fluoropicolinamide
N-(4-((1R,3S,5S)-3-amino-5-methylcyclohexyl)pyridin-3-yl)-6-(2,6-difluoro-4-methylphenyl)-5-fluoropicolinamide
N-(4-((1R,3S,5S)-3-amino-5-methylcyclohexyl)pyridin-3-yl)-6-(3-((R)-2,3-dihydroxypropoxy)-2,6-difluorophenyl)-5-fluoropicolinamide
N-(4-((1R,3S,5S)-3-amino-5-methylcyclohexyl)pyridin-3-yl)-6-(3-((S)-2,3-dihydroxypropoxy)-2,6-difluorophenyl)-5-fluoropicolinamide
N-(4-((1R,3S,5S)-3-amino-5-methylcyclohexyl)pyridin-3-yl)-6-(2,6-difluoro-4-methoxyphenyl)-5-fluoropicolinamide
N-(4-((1R,3S,5S)-3-amino-5-methylcyclohexyl)pyridin-3-yl)-6-(2,6-difluoro-4-(2-methoxyethoxy)phenyl)-5-fluoropicolinamide
N-(4-((1R,3S,5S)-3-amino-5-methylcyclohexyl)pyridin-3-yl)-6-(2,6-difluoro-3-(2-methoxyethoxy)phenyl)-5-fluoropicolinamide
N-(4-((1R,3R,4R,5S)-3-amino-4-hydroxy-5-methylcyclohexyl)pyridin-3-yl)-6-(2,6-difluoro-4-(methylsulfonyl)phenyl)-5-fluoropicolinamide
N-(4-((3R,4R,5S)-3-amino-4-hydroxy-5-methylpiperidin-1-yl)pyridin-3-yl)-6-(2,6-difluoro-4-methoxyphenyl)-5-fluoropicolinamide
N-(4-((3R,4R,5S)-3-amino-4-hydroxy-5-methylpiperidin-1-yl)pyridin-3-yl)-6-(2,6-difluoro-4-(methylsulfonyl)phenyl)-5-fluoropicolinamide
N-(4-((3R,4R,5S)-3-amino-4-hydroxy-5-methylpiperidin-1-yl)pyridin-3-yl)-6-(2,6-difluoro-3-(2-methoxyethoxy)phenyl)-5-fluoropicolinamide
N-(4-((1R,3R,4R,5S)-3-amino-4-hydroxy-5-methylcyclohexyl)pyridin-3-yl)-6-(2,6-difluoro-3-(2-methoxyethoxy)phenyl)-5-fluoropicolinamide
N-(4-((1R,3R,4S,5S)-3-amino-4-hydroxy-5-methylcyclohexyl)pyridin-3-yl)-6-(2,6-difluoro-4-(methylthio)phenyl)-5-fluoropicolinamide
N-(4-((3R,4R,5S)-3-amino-4-hydroxy-5-methylpiperidin-1-yl)pyridin-3-yl)-6-(4-ethoxy-2,6-difluorophenyl)-5-fluoropicolinamide
N-(4-((3R,4R,5S)-3-amino-4-hydroxy-5-methylpiperidin-1-yl)pyridin-3-yl)-6-(2,6-difluoro-4-(2-hydroxyethoxy)phenyl)-5-fluoropicolinamide
N-(4-((3R,4R,5S)-3-amino-4-hydroxy-5-methylpiperidin-1-yl)pyridin-3-yl)-6-(2,6-difluoro-4-(2-methoxyethoxy)phenyl)-5-fluoropicolinamide
N-(4-((3R,4R,5S)-3-amino-4-hydroxy-5-methylpiperidin-1-yl)pyridin-3-yl)-6-(2,6-difluoro-4-(methylthio)phenyl)-5-fluoropicolinamide
N-(4-((1R,3R,4R,5S)-3-amino-4-hydroxy-5-methylcyclohexyl)pyridin-3-yl)-6-(2,6-difluoro-4-(2-methoxyethoxy)phenyl)-5-fluoropicolinamide
N-(4-((3R,4R,5S)-3-amino-4-hydroxy-5-methylpiperidin-1-yl)pyridin-3-yl)-6-(2,6-difluoro-4-((S)-methylsulfinyl)phenyl)-5-fluoropicolinamide
N-(4-((3R,4R,5S)-3-amino-4-hydroxy-5-methylpiperidin-1-yl)pyridin-3-yl)-6-(2,6-difluoro-4-((R)-methylsulfinyl)phenyl)-5-fluoropicolinamide
N-(4-((1R,3R,4R,5S)-3-amino-4-hydroxy-5-methylcyclohexyl)pyridin-3-yl)-6-(2,6-difluoro-4-((S)-methylsulfinyl)phenyl)-5-fluoropicolinamide
N-(4-((1R,3R,4R,5S)-3-amino-4-hydroxy-5-methylcyclohexyl)pyridin-3-yl)-6-(2,6-difluoro-4-((R)-methylsulfinyl)phenyl)-5-fluoropicolinamide
N-(4-((1R,3S,5S)-3-amino-5-methylcyclohexyl)pyridin-3-yl)-6-(2,6-difluoro-3-(2-hydroxyethoxy)phenyl)-5-fluoropicolinamide
N-(4-((1R,3R,4R,5S)-3-amino-4-hydroxy-5-methylcyclohexyl)pyridin-3-yl)-6-(2,6-difluoro-3-(2-hydroxyethoxy)phenyl)-5-fluoropicolinamide
N-(4-((3R,4R,5S)-3-amino-4-hydroxy-5-methylpiperidin-1-yl)pyridin-3-yl)-6-(2,6-difluoro-3-(2-hydroxyethoxy)phenyl)-5-fluoropicolinamide
N-(4-((1R,3R,4R,5S)-3-amino-4-hydroxy-5-methylcyclohexyl)pyridin-3-yl)-6-(2,6-difluoro-4-(2-hydroxyethoxy)phenyl)-5-fluoropicolinamide
N-(4-((1R,3S,5S)-3-amino-5-methylcyclohexyl)pyridin-3-yl)-6-(2,6-difluoro-4-(2-hydroxyethyl)phenyl)-5-fluoropicolinamide
N-(4-((1R,3R,4R,5S)-3-amino-4-hydroxy-5-methylcyclohexyl)pyridin-3-yl)-6-(2,6-difluoro-4-(2-hydroxyethyl)phenyl)-5-fluoropicolinamide
N-(4-((3R,4R,5S)-3-amino-4-hydroxy-5-methylpiperidin-1-yl)pyridin-3-yl)-6-(2,6-difluoro-4-(2-hydroxyethyl)phenyl)-5-fluoropicolinamide
N-(4-((1R,3R,4R,5S)-3-amino-4-hydroxy-5-methylcyclohexyl)pyridin-3-yl)-6-(2,6-difluoro-4-(methylthio)phenyl)-5-fluoropicolinamide
N-(4-((3R,4R,5S)-3-amino-4-hydroxy-5-methylpiperidin-1-yl)pyridin-3-yl)-6-(2,6-difluoro-4-methylphenyl)-5-fluoropicolinamide
N-(4-((3R,4R,5S)-3-amino-4-hydroxy-5-methylpiperidin-1-yl)pyridin-3-yl)-6-(2,6-difluoro-4-(methoxymethyl)phenyl)-5-fluoropicolinamide
N-(4-((1R,3R,4R,5S)-3-amino-4-hydroxy-5-methylcyclohexyl)pyridin-3-yl)-6-(4-ethyl-2,6-difluorophenyl)-5-fluoropicolinamide
N-(4-((1R,3R,4R,5S)-3-amino-4-hydroxy-5-methylcyclohexyl)pyridin-3-yl)-6-(2,6-difluoro-4-(methoxymethyl)phenyl)-5-fluoropicolinamide
N-(4-((1R,3S,5S)-3-amino-5-methylcyclohexyl)pyridin-3-yl)-6-(2,6-difluoro-4-(methoxymethyl)phenyl)-5-fluoropicolinamide
N-(4-((1R,3S,5S)-3-amino-5-methylcyclohexyl)pyridin-3-yl)-6-(4-chloro-2,6-difluorophenyl)-5-fluoropicolinamide
N-(4-((1R,3S,5S)-3-amino-5-methylcyclohexyl)pyridin-3-yl)-5-fluoro-6-(2,4,6-trifluorophenyl)picolinamide
4-(6-(4-((1R,3S,5S)-3-amino-5-methylcyclohexyl)pyridin-3-ylcarbamoyl)-3-fluoropyridin-2-yl)-3,5-difluorobenzoic acid methyl 4-(6-(4-((1R,3S,5S)-3-amino-5-methylcyclohexyl)pyridin-3-ylcarbamoyl)-3-fluoropyridin-2-yl)-3,5-difluorobenzoate
N-(4-((1R,3S,5S)-3-amino-5-methylcyclohexyl)pyridin-3-yl)-6-(2,6-difluoro-3-methoxyphenyl)-5-fluoropicolinamide
N-(4-((1R,3S,5S)-3-amino-5-methylcyclohexyl)pyridin-3-yl)-6-(3-ethoxy-2,6-difluorophenyl)-5-fluoropicolinamide
N-(4-((3R,4R,5S)-3-amino-4-hydroxy-5-methylpiperidin-1-yl)pyridin-3-yl)-6-(2,6-difluoro-3-methoxyphenyl)-5-fluoropicolinamide methyl 4-(6-((4-((3R,4R,5S)-3-amino-4-hydroxy-5-methylpiperidin-1-yl)pyridin-3-yl)carbamoyl)-3-fluoropyridin-2-yl)-3,5-difluorobenzoate
N-(4-((3R,4R,5S)-3-amino-4-hydroxy-5-methylpiperidin-1-yl)pyridin-3-yl)-6-(3-ethoxy-2,6-difluorophenyl)-5-fluoropicolinamide
N-(4-((3R,4R,5S)-3-amino-4-hydroxy-5-methylpiperidin-1-yl)pyridin-3-yl)-6-(4-chloro-2,6-difluorophenyl)-5-fluoropicolinamide
N-(4-((3R,4R,5S)-3-amino-4-hydroxy-5-methylpiperidin-1-yl)pyridin-3-yl)-5-fluoro-6-(2,4,6-trifluorophenyl)picolinamide
N-(4-((1R,3S,5S)-3-amino-5-methylcyclohexyl)pyridin-3-yl)-6-(2,6-difluoro-4-(methylthio)phenyl)-5-fluoropicolinamide
N-(4-((1R,3S,5S)-3-amino-5-methylcyclohexyl)pyridin-3-yl)-6-(2,6-difluoro-4-(hydroxymethyl)phenyl)-5-fluoropicolinamide
N-(4-((1R,3S,5S)-3-amino-5-methylcyclohexyl)pyridin-3-yl)-6-(4-ethoxy-2,6-difluorophenyl)-5-fluoropicolinamide
N-(4-((1R,3R,4R,5S)-3-amino-4-hydroxy-5-methylcyclohexyl)pyridin-3-yl)-6-(4-ethoxy-2,6-difluorophenyl)-5-fluoropicolinamide
N-(4-((1R,3S,5S)-3-amino-5-methylcyclohexyl)pyridin-3-yl)-6-(2,6-difluoro-4-((S)-methylsulfinyl)phenyl)-5-fluoropicolinamide
N-(4-((1R,3S,5S)-3-amino-5-methylcyclohexyl)pyridin-3-yl)-6-(2,6-difluoro-4-((R)-methylsulfinyl)phenyl)-5-fluoropicolinamide
N-(4-((1R,3S,5S)-3-amino-5-methylcyclohexyl)pyridin-3-yl)-6-(2,6-difluoro-4-(2-hydroxyethoxy)phenyl)-5-fluoropicolinamide
N-(4-((1R,3S,5S)-3-amino-5-methylcyclohexyl)pyridin-3-yl)-6-(4-ethyl-2,6-difluorophenyl)-5-fluoropicolinamide
N-(4-((1R,3S,5S)-3-amino-5-methylcyclohexyl)pyridin-3-yl)-6-(4-((S)-1,2-dihydroxyethyl)-2,6-difluorophenyl)-5-fluoropicolinamide
N-(4-((1R,3S,5S)-3-amino-5-methylcyclohexyl)pyridin-3-yl)-6-(4-((R)-1,2-dihydroxyethyl)-2,6-difluorophenyl)-5-fluoropicolinamide
N-(4-((3R,4R,5S)-3-amino-4-hydroxy-5-methylpiperidin-1-yl)pyridin-3-yl)-6-(4-((S)-1,2-dihydroxyethyl)-2,6-difluorophenyl)-5-fluoropicolinamide
N-(4-((3R,4R,5S)-3-amino-4-hydroxy-5-methylpiperidin-1-yl)pyridin-3-yl)-6-(4-((R)-1,2-dihydroxyethyl)-2,6-difluorophenyl)-5-fluoropicolinamide
N-(4-((1R,3S,5S)-3-amino-5-methylcyclohexyl)pyridin-3-yl)-6-(2,6-difluoro-4-(2-hydroxypropan-2-yl)phenyl)-5-fluoropicolinamide
N-(4-((1R,3R,4R,5S)-3-amino-4-hydroxy-5-methylcyclohexyl)pyridin-3-yl)-6-(4-(cyclopropylmethoxy)-2,6-difluorophenyl)-5-fluoropicolinamide
N-(4-((1R,3S,5S)-3-amino-5-methylcyclohexyl)pyridin-3-yl)-6-(2,6-difluoro-4-propionylphenyl)-5-fluoropicolinamide
N-(4-((1R,3S,5S)-3-amino-5-methylcyclohexyl)pyridin-3-yl)-6-(2,6-difluoro-4-(1-hydroxycyclopropyl)phenyl)-5-fluoropicolinamide
N-(4-((1R,3R,4R,5S)-3-amino-4-hydroxy-5-methylcyclohexyl)pyridin-3-yl)-6-(2,6-difluoro-4-(2-methoxy-2-methylpropoxy)phenyl)-5-fluoropicolinamide
N-(4-((1R,3S,5S)-3-amino-5-methylcyclohexyl)pyridin-3-yl)-6-(2,6-difluoro-4-(2-methoxy-2-methylpropoxy)phenyl)-5-fluoropicolinamide
N-(4-((1R,3R,4R,5S)-3-amino-4-hydroxy-5-methylcyclohexyl)pyridin-3-yl)-6-(2,6-difluoro-4-(oxetan-3-yloxy)phenyl)-5-fluoropicolinamide
N-(4-((1R,3S,5S)-3-amino-5-methylcyclohexyl)pyridin-3-yl)-6-(2,6-difluoro-4-(oxetan-3-yloxy)phenyl)-5-fluoropicolinamide
N-(4-((1R,3S,5S)-3-amino-5-methylcyclohexyl)pyridin-3-yl)-6-(2,6-difluoro-4-(2-methoxypropan-2-yl)phenyl)-5-fluoropicolinamide
N-(4-((1R,3S,5S)-3-amino-5-methylcyclohexyl)pyridin-3-yl)-6-(2,6-difluoro-4-(2-hydroxy-2-methylpropoxy)phenyl)-5-fluoropicolinamide
N-(4-((1R,3S,5S)-3-amino-5-methylcyclohexyl)pyridin-3-yl)-6-(2,6-difluoro-4-(2-hydroxy-2-methylpropoxy)phenyl)-5-fluoropicolinamide
N-(4-((1R,3R,4R,5S)-3-amino-4-hydroxy-5-methylcyclohexyl)pyridin-3-yl)-6-(2,6-difluoro-4-(2-hydroxy-2-methylpropoxy)phenyl)-5-fluoropicolinamide
N-(4-((1R,3R,4R,5S)-3-amino-4-hydroxy-5-methylcyclohexyl)pyridin-3-yl)-6-(2,6-difluoro-4-(3-methoxyoxetan-3-yl)phenyl)-5-fluoropicolinamide
N-(4-((1R,3S,5S)-3-amino-5-methylcyclohexyl)pyridin-3-yl)-6-(2,6-difluoro-4-(3-methoxyoxetan-3-yl)phenyl)-5-fluoropicolinamide
N-(4-((1R,3R,4R,5S)-3-amino-4-hydroxy-5-methylcyclohexyl)pyridin-3-yl)-6-(2,6-difluoro-4-(3-hydroxyoxetan-3-yl)phenyl)-5-fluoropicolinamide
N-(4-((1R,3S,5S)-3-amino-5-methylcyclohexyl)pyridin-3-yl)-6-(2,6-difluoro-4-(3-hydroxyoxetan-3-yl)phenyl)-5-fluoropicolinamide
N-(4-((1R,3R,4R,5S)-3-amino-4-hydroxy-5-methylcyclohexyl)pyridin-3-yl)-6-(2,6-difluoro-4-(oxetan-3-yl)phenyl)-5-fluoropicolinamide
N-(4-((1R,3S,5S)-3-amino-5-methylcyclohexyl)pyridin-3-yl)-6-(4-(difluoromethyl)-2,6-difluorophenyl)-5-fluoropicolinamide
N-(4-((1R,3R,4R,5S)-3-amino-4-hydroxy-5-methylcyclohexyl)pyridin-3-yl)-6-(4-(difluoromethyl)-2,6-difluorophenyl)-5-fluoropicolinamide
N-(4-((1R,3R,4R,5S)-3-amino-4-hydroxy-5-methylcyclohexyl)pyridin-3-yl)-6-(2,6-difluoro-4-(tetrahydro-2H-pyran-4-yloxy)phenyl)-5-fluoropicolinamide
N-(4-((1R,3R,4R,5S)-3-amino-4-hydroxy-5-methylcyclohexyl)pyridin-3-yl)-6-(2,6-difluoro-4-(1-hydroxycyclobutyl)phenyl)-5-fluoropicolinamide
N-(4-((1R,3S,5S)-3-amino-5-methylcyclohexyl)pyridin-3-yl)-6-(2,6-difluoro-4-(1-hydroxycyclobutyl)phenyl)-5-fluoropicolinamide
N-(4-((1R,3R,4R,5S)-3-amino-4-hydroxy-5-methylcyclohexyl)pyridin-3-yl)-6-(2,6-difluoro-4-isopropoxyphenyl)-5-fluoropicolinamide
N-(4-((1R,3S,5S)-3-amino-5-methylcyclohexyl)pyridin-3-yl)-6-(2,6-difluoro-4-((tetrahydro-2H-pyran-4-yl)oxy)phenyl)-5-fluoropicolinamide
N-(4-((1R,3S,5S)-3-amino-5-methylcyclohexyl)pyridin-3-yl)-6-(4-(cyclopropylmethoxy)-2,6-difluorophenyl)-5-fluoropicolinamide
N-(4-((1R,3S,5S)-3-amino-5-methylcyclohexyl)pyridin-3-yl)-6-(4-(difluoromethoxy)-2,6-difluorophenyl)-5-fluoropicolinamide
N-(4-((1R,3R,4R,5S)-3-amino-4-hydroxy-5-methylcyclohexyl)pyridin-3-yl)-6-(2,6-difluoro-4-(2-hydroxypropan-2-yl)phenyl)-5-fluoropicolinamide
N-(4-((1R,3S,5S)-3-amino-5-methylcyclohexyl)pyridin-3-yl)-6-(2,6-difluoro-4-(2-methoxyethyl)phenyl)-5-fluoropicolinamide
N-(4-((1R,3S,5S)-3-amino-5-methylcyclohexyl)pyridin-3-yl)-6-(2,6-difluoro-4-(oxetan-3-yl)phenyl)-5-fluoropicolinamide
N-(4-((1R,3R,4R,5S)-3-amino-4-hydroxy-5-methylcyclohexyl)pyridin-3-yl)-6-(2,6-difluoro-4-((R)-tetrahydrofuran-3-yloxy)phenyl)-5-fluoropicolinamide
N-(4-((1R,3R,4R,5S)-3-amino-4-hydroxy-5-methylcyclohexyl)pyridin-3-yl)-6-(2,6-difluoro-4-((S)-tetrahydrofuran-3-yloxy)phenyl)-5-fluoropicolinamide
N-(4-((1R,3S,5S)-3-amino-5-methylcyclohexyl)pyridin-3-yl)-6-(2,6-difluoro-4-((R)-tetrahydrofuran-3-yloxy)phenyl)-5-fluoropicolinamide
N-(4-((1R,3S,5S)-3-amino-5-methylcyclohexyl)pyridin-3-yl)-6-(2,6-difluoro-4-((S)-tetrahydrofuran-3-yloxy)phenyl)-5-fluoropicolinamide
N-(4-((1R,3R,4R,5S)-3-amino-4-hydroxy-5-methylcyclohexyl)pyridin-3-yl)-6-(4-cyclopropyl-2,6-difluorophenyl)-5-fluoropicolinamide
N-(4-((1R,3S,5S)-3-amino-5-methylcyclohexyl)pyridin-3-yl)-6-(2,6-difluoro-4-((S)-1-hydroxyethyl)phenyl)-5-fluoropicolinamide
N-(4-((1R,3S,5S)-3-amino-5-methylcyclohexyl)pyridin-3-yl)-6-(2,6-difluoro-4-((R)-1-hydroxyethyl)phenyl)-5-fluoropicolinamide
3-amino-N-(4-((1R,3R,4R,5S)-3-amino-4-hydroxy-5-methylcyclohexyl)pyridin-3-yl)-6-(2,6-difluoro-4-methylphenyl)-5-fluoropicolinamide
3-amino-N-(4-((1R,3S,5S)-3-amino-5-methylcyclohexyl)pyridin-3-yl)-6-(2,6-difluoro-4-(1-hydroxycyclobutyl)phenyl)-5-fluoropicolinamide and
3-amino-N-(4-((1R,3S,5S)-3-amino-5-methylcyclohexyl)pyridin-3-yl)-6-(2,6-difluoro-4-(3-hydroxyoxetan-3-yl)phenyl)-5-fluoropicolinamide;

and the pharmaceutically acceptable salts of these compounds.

In some embodiments, the compound is any compound selected from Table 1, Table 2 or Table 3.

The compounds described above are inhibitors of Pim kinases as further discussed herein. These compounds and their pharmaceutically acceptable salts, and pharmaceutical compositions containing these compounds and salts are useful for therapeutic methods such as treatment of cancers and autoimmune disorders that are caused by or exacerbated by excessive levels of Pim kinase activity.

### DETAILED DESCRIPTION OF EMBODIMENTS OF THE INVENTION

“PIM inhibitor” or “Pim inhibitor” is used herein to refer to a compound that exhibits an IC₅₀ with respect to PIM Kinase activity of no more than about 100 μM and more typically not more than about 50 μM, as measured in the PIM depletion assays described herein below for at least one of Pim1, Pim2 and Pim3. Preferred compounds have on IC₅₀ below about 1 micromolar on at least one Pim, and generally have an IC₅₀ below 100 nM on each of Pim1, Pim2 and Pim3.

The phrase “alkyl” refers to hydrocarbon groups that do not contain heteroatoms, i.e., they consist of carbon atoms and hydrogen atoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂ CH(CH₃)(CH₂CH₃), —CH₂CH(CH₂CH₃)₂, —CH₂C(CH₃)₃, —CH₂C(CH₂CH₃)₃, —CH(CH₃)—CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. Thus the term ‘alkyl’ includes primary alkyl groups, secondary alkyl groups, and tertiary alkyl groups. Typical alkyl groups include straight and branched chain alkyl groups having 1 to 12 carbon atoms, preferably 1-6 carbon atoms. The term ‘lower alkyl’ or “loweralkyl” and similar terms refer to alkyl groups containing up to 6 carbon atoms.

The term “alkenyl” refers to alkyl groups as defined above, wherein there is at least one carbon-carbon double bond, i.e., wherein two adjacent carbon atoms are attached by a double bond. The term “alkynyl” refers to alkyl groups wherein two adjacent carbon atoms are attached by a triple bond. Typical alkenyl and alkynyl groups contain 2-12 carbon atoms, preferably 2-6 carbon atoms. Lower alkenyl or lower alkynyl refers to groups having up to 6 carbon atoms. An alkenyl or alkynyl group may contain more than one unsaturated bond, and may include both double and triple bonds, but of course their bonding is consistent with well-known valence limitations.

The term ‘alkoxy” refers to —OR, wherein R is alkyl.

As used herein, the term “halogen” or “halo” refers to chloro, bromo, fluoro and iodo groups. Typical halo substituents are F and/or Cl. “Haloalkyl” refers to an alkyl radical substituted with one or more halogen atoms. The term “haloalkyl” thus includes monohalo alkyl, dihalo alkyl, trihalo alkyl, perhaloalkyl, and the like.

“Amino” refers herein to the group —NH₂. The term “alkylamino” refers herein to the group —NRR′ where R and R′ are each independently selected from hydrogen or a lower alkyl, provided —NRR′ is not —NH₂. The term “arylamino” refers herein to the group —NRR′ where R is aryl and R′ is hydrogen, a lower alkyl, or an aryl. The term “aralkylamino” refers herein to the group —NRR′ where R is a lower aralkyl and R′ is hydrogen, a loweralkyl, an aryl, or a loweraralkyl. The term cyano refers to the group —CN. The term nitro refers to the group —NO₂.

The term “alkoxyalkyl” refers to the group -alk₁-O-alk₂ where alk₁ is an alkyl or alkenyl linking group, and alk₂ is alkyl or alkenyl. The term “loweralkoxyalkyl” refers to an alkoxyalkyl where alk₁ is loweralkyl or loweralkenyl, and alk₂ is loweralkyl or loweralkenyl. The term “aryloxyalkyl” refers to the group -alkyl-O-aryl, where -alkyl- is a C₁₋₁₂ straight or branched chain alkyl linking group, preferably C₁₋₆. The term “aralkoxyalkyl” refers to the group -alkylenyl-O-aralkyl, where aralkyl is preferably a loweraralkyl.

The term “aminocarbonyl” refers herein to the group —C(O)—NH₂. “Substituted aminocarbonyl” refers herein to the group —C(O)—NRR′ where R is loweralkyl and R′ is hydrogen or a loweralkyl. In some embodiments, R and R′, together with the N atom attached to them may be taken together to form a “heterocycloalkylcarbonyl” group. The term “arylaminocarbonyl” refers herein to the group —C(O)—NRR′ where R is an aryl and R′ is hydrogen, loweralkyl or aryl.

“aralkylaminocarbonyl” refers herein to the group —C(O)—NRR′ where R is loweraralkyl and R′ is hydrogen, loweralkyl, aryl, or loweraralkyl.

“Aminosulfonyl” refers herein to the group —S(O)₂—NH₂. “Substituted aminosulfonyl” refers herein to the group —S(O)₂—NRR′ where R is loweralkyl and R′ is hydrogen or a loweralkyl. The term “aralkylaminosulfonlyaryl” refers herein to the group -aryl-S(O)₂—NH-aralkyl, where the aralkyl is loweraralkyl.

“Carbonyl” refers to the divalent group —C(O)—. “Carboxy” refers to —C(═O)—OH. “Alkoxycarbonyl” refers to ester —C(═O)—OR wherein R is optionally substituted lower alkyl. “Loweralkoxycarbonyl” refers to ester —C(═O)—OR wherein R is optionally substituted lower loweralkyl. “Cycloalkyloxycarbonyl” refers to —C(═O)—OR wherein R is optionally substituted C₃-C₈ cycloalkyl.

“Cycloalkyl” refers to a mono- or polycyclic, carbocyclic non-aromatic alkyl substituent. Carbocycloalkyl groups are cycloalkyl groups in which all ring atoms are carbon. Typical cycloalkyl substituents have from 3 to 8 backbone (i.e., ring) atoms. When used in connection with cycloalkyl substituents, the term “polycyclic” refers herein to fused and non-fused alkyl cyclic structures. The term “partially unsaturated cycloalkyl”, “partially saturated cycloalkyl”, and “cycloalkenyl” all refer to a cycloalkyl group wherein there is at least one point of unsaturation, i.e., wherein to adjacent ring atoms are connected by a double bond or a triple bond. Such rings typically contain 1-2 double bonds for 5-6 membered rings, and 1-2 double bonds or one triple bond for 7-8 membered rings. Illustrative examples include cyclohexenyl, cyclooctynyl, cyclopropenyl, cyclobutenyl, cyclohexadienyl, and the like.

The term “heterocycloalkyl” refers herein to cycloalkyl substituents that have from 1 to 5, and more typically from 1 to 4 heteroatoms as ring members in place of carbon atoms. Preferably, heterocycloalkyl or “heterocyclyl” groups contain one or two heteroatoms as ring members, typically only one heteroatom for 3-5 membered rings and 1-2 heteroatoms for 6-8 membered rings. Suitable heteroatoms employed in heterocyclic groups of the present invention are nitrogen, oxygen, and sulfur. Representative heterocycloalkyl moieties include, for example, pyrrolidinyl, tetrahydrofuranyl, oxirane, oxetane, oxepane, thiirane, thietane, azetidine, morpholino, piperazinyl, piperidinyl and the like.

The terms “substituted heterocycle”, “heterocyclic group” or “heterocycle” as used herein refers to any 3- or 4-membered ring containing a heteroatom selected from nitrogen, oxygen, and sulfur or a 5- or 6-membered ring containing from one to three heteroatoms, preferably 1-2 heteroatoms, selected from the group consisting of nitrogen, oxygen, or sulfur;

wherein the 5-membered ring has 0-2 double bonds and the 6-membered ring has 0-3 double bonds; wherein the nitrogen and sulfur atom may be optionally oxidized; wherein the nitrogen and sulfur heteroatoms may be optionally quarternized; and including any bicyclic group in which any of the above heterocyclic rings is fused to a benzene ring or another 5- or 6-membered heterocyclic ring as described herein. Preferred heterocycles include, for example: diazapinyl, pyrrolinyl, pyrrolidinyl, pyrazolinyl, pyrazolidinyl, imidazolinyl, imidazolidinyl, piperidinyl, piperazinyl, N-methyl piperazinyl, azetidinyl, N-methylazetidinyl, oxazolidinyl, isoazolidinyl, morpholinyl, thiazolidinyl, isothiazolidinyl, and oxiranyl. The heterocyclic groups may be attached at ring various positions as will be apparent to those having skill in the organic and medicinal chemistry arts in conjunction with the disclosure herein.

Heterocyclic moieties can be unsubstituted or they can be substituted with one or more substituents independently selected from hydroxy, halo, oxo (C═O), alkylimino (RN═, wherein R is a loweralkyl or loweralkoxy group), amino, alkylamino, dialkylamino, acylaminoalkyl, alkoxy, thioalkoxy, lower alkoxyalkoxy, loweralkyl, cycloalkyl or haloalkyl. Typically, substituted heterocyclic groups will have up to four substituent groups.

The term “cyclic ether” as used herein refers to a 3-7 membered ring containing one oxygen atom (O) as a ring member. Where the cyclic ether is “optionally substituted” it can be substituted at any carbon atom with a group suitable as a substituent for a heterocyclic group, typically up to three substituents selected from lower alkyl, lower alkoxy, oxo, halo, hydroxy, —C(O)-lower alkyl, and —C(O)-lower alkoxy unless otherwise specified. In preferred embodiments, halo, hydroxy and lower alkoxy are not attached to the carbon atoms of the ring that are bonded directly to the oxygen atom in the cyclic ether ring. Specific examples include oxirane, oxetane (e.g., 3-oxetane), tetrahydrofuran (including 2-tetrahydrofuranyl and 3-tetrahydrofuranyl), tetrahydropyran (e.g., 4-tetrahydropyranyl), and oxepane.

“Aryl” refers to monocyclic and polycyclic aromatic groups having from 5 to 14 backbone carbon or hetero atoms, and includes both carbocyclic aryl groups and heteroaromatic aryl groups. Carbocyclic aryl groups are aryl groups in which all ring atoms in the aromatic ring are carbon, typically including phenyl and naphthyl. Exemplary aryl moieties employed as substituents in compounds of the present invention include phenyl, pyridyl, pyrimidinyl, thiazolyl, indolyl, imidazolyl, oxadiazolyl, tetrazolyl, pyrazinyl, triazolyl, thiophenyl, furanyl, quinolinyl, purinyl, naphthyl, benzothiazolyl, benzopyridyl, and benzimidazolyl, and the like. When used in connection with aryl substituents, the term “polycyclic aryl” refers herein to fused and non-fused cyclic structures in which at least one cyclic structure is aromatic, such as, for example, benzodioxozolo (which has a heterocyclic structure fused to a phenyl group, naphthyl, and the like. Where “aryl” is used, the group is preferably a carbocyclic group; the term “heteroaryl” is used for aryl groups when ones containing one or more heteroatoms are preferred.

The term “heteroaryl” refers herein to aryl groups having from 1 to 4 heteroatoms as ring atoms in an aromatic ring with the remainder of the ring atoms being carbon atoms, in a 5-14 atom aromatic ring system that can be monocyclic or polycyclic. Monocyclic heteroaryl rings are typically 5-6 atoms in size. Exemplary heteroaryl moieties employed as substituents in compounds of the present invention include pyridyl, pyrimidinyl, thiazolyl, indolyl, imidazolyl, oxadiazolyl, tetrazolyl, pyrazinyl, triazolyl, thiophenyl, furanyl, quinolinyl, purinyl, benzothiazolyl, benzopyridyl, and benzimidazolyl, and the like.

“Aralkyl” or “arylalkyl” refers to an aryl group connected to a structure through an alkylene linking group, e.g., a structure such as —(CH₂)₁₋₄—Ar, where Ar represents an aryl group. “Lower aralkyl” or similar terms indicate that the alkyl linking group has up to 6 carbon atoms.

“Optionally substituted” or “substituted” refers to the replacement of one or more hydrogen atoms with a monovalent or divalent radical. Alkyl, alkenyl, alkynyl, cycloalkyl, heterocycloalkyl, aryl, and heteroaryl groups described herein may be substituted or unsubstituted. Suitable substitution groups include, for example, hydroxy, nitro, amino, imino, cyano, halo, thio, sulfonyl, thioamido, amidino, imidino, oxo, oxamidino, methoxamidino, imidino, guanidino, sulfonamido, carboxyl, formyl, loweralkyl, haloloweralkyl, loweralkylamino, haloloweralkylamino, loweralkoxy, haloloweralkoxy, loweralkoxyalkyl, alkylcarbonyl, aminocarbonyl, arylcarbonyl, aralkylcarbonyl, heteroarylcarbonyl, heteroaralkyl-carbonyl, alkylthio, aminoalkyl, cyanoalkyl, aryl and the like, provided that oxo, imidino or other divalent substitution groups are not placed on aryl or heteroaryl rings due to the well known valence limitations of such rings.

The substitution group can itself be substituted where valence permits, i.e., where the substitution group contains at least one CH, NH or OH having a hydrogen atom that can be replaced. The group substituted onto the substitution group can be carboxyl, halo (on carbon only); nitro, amino, cyano, hydroxy, loweralkyl, loweralkoxy, C(O)R, —OC(O)R, —OC(O)OR, —NRCOR, —CONR₂, —NRCOOR, —C(S)NR₂, —NRC(S)R, —OC(O)NR₂—SR, —SO₃H, —SO₂R or C3-8 cycloalkyl or 3-8 membered heterocycloalkyl, where each R is independently selected from hydrogen, lower haloalkyl, lower alkoxyalkyl, and loweralkyl, and where two R on the same atom or on directly connected atoms can be linked together to form a 5-6 membered heterocyclic ring.

When a substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon or heteroatoms.

It is understood that the above definitions are not intended to include impermissible substitution patterns (e.g., methyl substituted with five fluoro groups or a halogen atom substituted with another halogen atom). Such impermissible substitution patterns are well known to the skilled artisan.

It will also be apparent to those skilled in the art that the compounds of the invention, or their stereoisomers, as well as the pharmaceutically acceptable salts, esters, metabolites and prodrugs of any of them, may be subject to tautomerization and may therefore exist in various tautomeric forms wherein a proton of one atom of a molecule shifts to another atom and the chemical bonds between the atoms of the molecules are consequently rearranged. See, e.g., March, Advanced Organic Chemistry: Reactions, Mechanisms and Structures, Fourth Edition, John Wiley & Sons, pages 69-74 (1992). As used herein, the term “tautomer” refers to the compounds produced by the proton shift, and it should be understood that all tautomeric forms, insofar as they may exist, are included within the invention.

The compounds of the invention may comprise one or more asymmetrically substituted carbon atoms. Such asymmetrically substituted carbon atoms can result in the compounds of the invention existing in enantiomers, diastereomers, and other stereoisomeric forms that may be defined, in terms of absolute stereochemistry, such as in (R)- or (S)-forms. The compounds of the invention are sometimes depicted herein as single enantiomers, and are intended to encompass the specific configuration depicted and the enantiomer of that specific configuration (the mirror image isomer of the depicted configuration), unless otherwise specified. The depicted structures herein describe the relative stereochemistry of the compounds where two or more chiral centers, but the invention is not limited to the depicted enantiomer's absolute stereochemistry unless otherwise stated. The invention includes both enantiomers, each of which will exhibit PIM inhibition, even though one will be more potent than the other. In some instances, compounds of the invention have been synthesized in racemic form and separated into individual isomers by chiral chromatography or similar conventional methods, which do not provide definitive information about absolute stereochemical configuration. In such cases, the absolute stereochemistry of the most active enantiomer has been identified based on correlation with similar compounds of known absolute stereochemistry, rather than by a definitive physical method such as X-ray crystallography. In other instances, the chiral centers are derived from starting materials or reactions that provide a specific, known enantiomer, so the absolute configuration of the chiral centers is known. Therefore, in certain embodiments, the preferred enantiomer of a compound described herein is the specific isomer depicted or its opposite enantiomer, whichever has the lower IC-50 for PIM kinase inhibition using the assay methods described herein, i.e., the enantiomer that is more potent as a PIM inhibitor.

The terms “S” and “R” configuration, as used herein, are as defined by the IUPAC 1974 RECOMMENDATIONS FOR SECTION E, FUNDAMENTAL STEREOCHEMISTRY, Pure Appl. Chem. 45:13-30 (1976). The terms α and β are employed for ring positions of cyclic compounds. The α-side of the reference plane is that side on which the preferred substituent lies at the lower numbered position. Those substituents lying on the opposite side of the reference plane are assigned β descriptor. It should be noted that this usage differs from that for cyclic stereoparents, in which “α” means “below the plane” and denotes absolute configuration. The terms α and β configuration, as used herein, are as defined by the CHEMICAL ABSTRACTS INDEX GUIDE-APPENDIX IV (1987) paragraph 203.

As used herein, the term “pharmaceutically acceptable salts” refers to the nontoxic acid or base addition salts of the compounds of Formula I or II, wherein the compound acquires a positive or negative charge as a result of adding or removing a proton; the salt then includes a counterion of opposite charge from the compound itself, and the counterion is preferably one suitable for pharmaceutical administration under the conditions where the compound would be used. These salts can be prepared in situ during the final isolation and purification of the compounds of Formula I or II, or by separately reacting the base or acid functions with a suitable organic or inorganic acid or base, respectively. Representative salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, pamoate, pectinate, persulfate, 3-phenylproionate, picrate, pivalate, propionate, succinate, sulfate, tartrate, thiocyanate, p-toluenesulfonate and undecanoate.

Also, a basic nitrogen-containing group in compounds of the invention can be quaternized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained. These quaternized ammonium salts when paired with a pharmaceutically acceptable anion can also serve as pharmaceutically acceptable salts.

Examples of acids which may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of the compounds of formula (I), or separately by reacting carboxylic acid moieties with a suitable base such as the hydroxide, carbonate or bicarbonate of a pharmaceutically acceptable metal cation or with ammonia, or an organic primary, secondary or tertiary amine. Counterions for pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

As used herein, the term “pharmaceutically acceptable ester” refers to esters, which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular pharmaceutically acceptable esters include formates, acetates, propionates, maleates, lactates, hydroxyacetates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, PRO-DRUGS AS NOVEL DELIVERY SYSTEMS, Vol. 14 of the A.C.S. Symposium Series, and in Edward B. Roche, ed., BIOREVERSIBLE CARRIERS IN DRUG DESIGN, American Pharmaceutical Association and Pergamon Press, 1987, both of which are incorporated herein by reference.

It will be apparent to those skilled in the art that the compounds of the invention, or their tautomers, prodrugs and stereoisomers, as well as the pharmaceutically acceptable salts, esters and prodrugs of any of them, may be processed in vivo through metabolism in a human or animal body or cell to produce metabolites. The term “metabolite” as used herein refers to the formula of any derivative produced in a subject after administration of a parent compound. The derivatives may be produced from the parent compound by various biochemical transformations in the subject such as, for example, oxidation, reduction, hydrolysis, or conjugation and include, for example, oxides and demethylated derivatives. The metabolites of a compound of the invention may be identified using routine techniques known in the art. See, e.g., Bertolini, G. et al., J. Med. Chem. 40:2011-2016 (1997); Shan, D. et al., J. Pharm. Sci. 86(7):765-767; Bagshawe K., Drug Dev. Res. 34:220-230 (1995); Bodor, N., Advances in Drug Res. 13:224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991). It should be understood that individual chemical compounds that are metabolites of the compounds of formula (I) or their tautomers, prodrugs and stereoisomers, as well as the pharmaceutically acceptable salts, esters and prodrugs of any of them, are included within the invention.

The following enumerated aspects and embodiments of the invention illustrate its scope.

1. In one aspect, the invention provides compounds of Formula I as described above, including compounds of Formula (Ia):

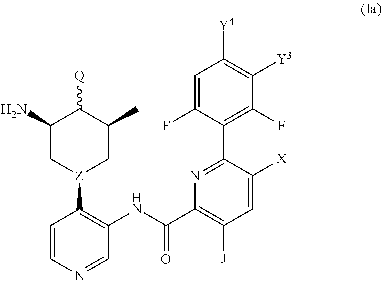

or a pharmaceutically acceptable salt thereof, wherein:

### Z is N or CH;

### Q is H, Me or —OH;

### X is H or F;

### J is H or NH₂;

Y³ is H or is selected from the group consisting of CN, OEt, S(O)ₚR, —O(CH₂)_{q}—OH, —O(CH₂)_{q}—OR, —(CH₂)_{q}—OH, —C(CH₃)₂OH, —(CH₂)_{q}—OR, —(CR′₂)₁₋₃—OR′ or —O—(CR′₂)₁₋₃—OR′ where each R′ is independently H or Me, and an optionally substituted member selected from the group consisting of C₁₋₄ alkyl, C₁₋₄ alkoxy, C₁₋₄ alkylthio, C₁₋₄ alkylsulfonyl, C₁₋₄ hydroxyalkyl, C₁₋₄ hydroxyalkyloxy, C₃₋₇ cycloalkyl, C₃₋₇ heterocycloalkyl, C₅₋₁₀ heteroaryl, and C₆₋₁₀ aryl, each of which is optionally substituted with up to two groups independently selected from halo, hydroxy, amino, OMe, CN, oxo, R and OR;
when Y³ is H, Y⁴ is selected from the group consisting of CN, R, vinyl, COOH, COOR, S(O)_{q}R, —O(CH₂)_{q}—OH, —O(CH₂)_{q}—OR, —(CH₂)_{q}—OH, —C(CH₃)₂OH, —(CH₂)ₚ—OR, —(CR′₂)₁₋₃—OH or —O—(CR′₂)₁₋₃—OH where each R′ is independently H or Me, and an optionally substituted member selected from the group consisting of C₁₋₄ alkyl, C₁₋₄ alkoxy, C₁₋₄ alkylthio, C₁₋₄ alkylsulfonyl, C₁₋₄ hydroxyalkyl, C₁₋₄ hydroxyalkyloxy, C₃₋₇ cycloalkyl, C₃₋₇ heterocycloalkyl, C₅₋₁₀ heteroaryl, and C₆₋₁₀ aryl, each of which is optionally substituted with up to two groups independently selected from halo, hydroxy, amino, OMe, CN, oxo, R and OR;
and when Y³ is not H Y⁴ is H;
each R is independently an optionally substituted C₁₋₄ alkyl or 3-7 membered cyclic ether, wherein the optional substitutents are independently selected from OH, OMe, CN, NH₂, halo, oxo, and CN;
each q is 1 or 2; and
each p is independently 0, 1 or 2.

In some preferred embodiments, J is H.

In some embodiments, R is selected from an optionally substituted C₁₋₄ alkyl, such as cyclopropylmethyl, hydroxyalkyl, or haloalkyl, and an optionally substituted 3-7 membered cyclic ether such as an oxetanyl, tetrahydrofuranyl or tetrahydropyranyl group.

The relative stereochemistry of the groups on the cyclohexyl or piperidine ring in compounds of Formula I and Formulas Ia, Ha, and IIb has an important impact on activity, and Formula I depicts the preferred relative stereochemistry for groups on the cyclohexyl (Z═CH) or piperidine (Z═N) rings of these compounds. While Formula I is depicted as a single enantiomer for convenience, though, both enantiomers can exhibit Pim kinase inhibition, and the invention includes each enantiomer and mixtures of the two enantiomers of the depicted cyclohexyl and piperidine ring of Formula I. In preferred embodiments, the compounds have the absolute stereochemistry shown in the Formulas herein.

In some embodiments, the compounds are used in optically active form, where one enantiomer is present in excess over the other; in other embodiments, a racemic mixture can be used. The invention thus includes the specific isomer depicted above as well as its enantiomer, and mixtures of the two enantiomers in various proportions, including racemic mixtures. In some embodiments, the enantiomer that is the more potent inhibitor of Pim kinases is used in substantially pure isomeric form, e.g., it can be used as an enriched isomeric mixture having an enantiomeric excess of over 80%, typically over 90% and preferably more than about 95%. It may be substantially free of the opposite enantiomer. In many embodiments, the specific isomer (enantiomer) depicted as Formula I is preferred.

Where compounds of Formula I have substituents such as Y³ and/or Y⁴ that contain one or more additional chiral centers, the chirality of those substituents is less important. Such compounds may thus be obtained and used as single diastereomers, or as mixtures of diastereomers with regard to the chirality of Y³ or Y⁴; i.e., the substituent groups may comprise single enantiomers of any chiral centers they contain, or mixtures of enantiomers of such chiral centers. Thus the compounds of Formula I include mixtures of diastereomers with respect to chiral centers not depicted in Formula I itself, e.g., those related to the Y³ and/or Y⁴ groups, and the invention contemplates making and using all such diastereomers, while retaining the specific relative stereochemistry of the chiral centers depicted in Formula I.

2. In some embodiments of the compounds of Formula I, Z is N.

3. In other embodiments of the compounds of Formula I, Z is CH, and in these embodiments Z is a chiral center having the relative stereochemistry depicted in Formula I. Where Z is N, the center does not behave as a chiral center.

4. In some embodiments, the invention provides a compound of any of the preceding embodiments wherein Q is H.

5. In alternative embodiments to embodiment 4, the invention provides a compound of any of embodiments 1-3, wherein Q is OH. When Q is OH, it is attached to a chiral center, which may be of either configuration, or the compound can be used as a mixture of isomers at this stereocenter. Where Q is other than H, the compound is often of Formula Ib:

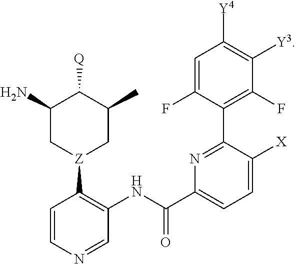

6. In certain embodiments, the compound of embodiment 5 is a compound of Formula (IIa):

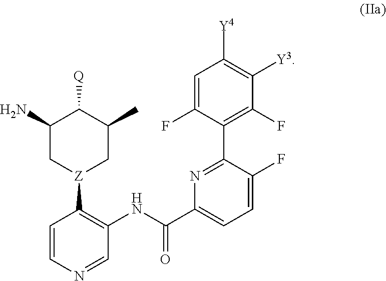

7. In certain embodiments, the compound of embodiment 5 is a compound of Formula (IIa):

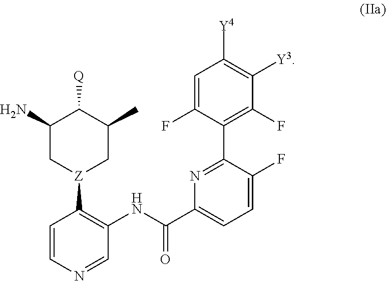

In other embodiments, it is of Formula IIb:

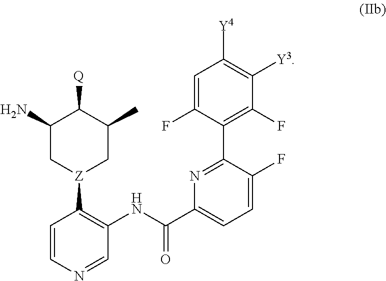

8. In certain embodiments, X is F in the compound of any of embodiments 1-6.

9. In certain embodiments, X is H in the compound of any of embodiments 1-6.

10. In certain embodiments of the compound of any of the preceding embodiments, one of Y³ and Y⁴ is selected from the group consisting of OMe, Me, Et, —CH₂—OEt, —CH₂OMe, COOH, COOMe, S(O)ₚMe, —O(CH₂)₂—OH, —(CH₂)₂—OH, —O(CH₂)₂—OMe, —OCH₂—CH(OH)—CH₂OH, —CH(OH)—CH₂OH, —(CH₂)_{q}—OH, —C(CH₃)₂OH, 4-tetrahydropyranyl, and —(CH₂)_{q}—OR; where p is 0, 1 or 2, and each q is 1 or 2. Other groups that can be used as Y³ or Y⁴ are of general formula —(CR'₂)₁₋₃—OR′ or —O—(CR'₂)₁₋₃—OR′, where each R′ is independently H or Me; in some embodiments of these compounds at least one R′ is Me.

Some specific embodiments of Y³ or Y⁴ for compounds of Formula I or IIa or IIb can include: methyl, ethyl, isopropyl, cyclopropyl, cyclobutyl, fluoromethyl, difluoromethyl, trifluoromethyl, trifluoroethyl, fluoromethoxy, difluoromethoxy, trifluoromethoxy, trifluoroethoxy, methoxy, ethoxy, isopropoxy, 1-hydroxyethyl, 2-hydroxyethyl, 1-hydroxypropyl, 2-hydroxypropyl, 3-hydroxypropyl, 2-methoxyethyl, 1-methoxypropyl, 2-methoxypropyl, 3-methoxypropyl, 2-hydroxyethoxy, 2-methoxyethoxy, 2-methoxyethyl, methoxymethyl, 1-methoxyethoxy, 1,2-dihydroxyethyl, 1,2-dimethoxyethyl, cyclopropoxy, 1-hydroxycyclopropyl, cyclopropoxymethyl, cyclopropylmethyl, cyclopropylmethoxy, cyclobutoxy, cyclobutylmethyl, cyclobutylmethoxy, 1-hydroxycyclobutyl, 2-hydroxycyclobutyl, 3-hydroxycyclobutyl, 2-hydroxycyclobutoxy, 3-hydroxycyclobutoxy, 3-methoxycyclobutoxy, 1-ethanone, 1-propanone, 2-propanone, 2-methyl-2-methoxypropyl, 2-hydroxy-2-methylethyl, 2-methoxy-2-methylethoxy, 2-hydroxy-2-methylpropoxy, 2-methoxy-2-methylpropoxy, 2-methoxy-2-propyl, 2-hydroxy-2-propyl, 2-methoxypropyl, 2-methoxypropoxy, 3-oxetanyl, 3-oxetanyloxy, 3-hydroxy-3-oxetanyl, 3-methoxy-3-oxetanyl, 2-tetrahydrofuranyl, 3-tetrahydrofuranyl, 3-tetrahydrofuranyloxy, 3-tetrahydropyranyl, 3-tetrahydropyranyloxy, 2-tetrahydropyranyl, 4-tetrahydropyranyl, 4-tetrahydropyranyloxy, 4-tetrahydrothiopyranyl, 4-tetrahydrothiopyranyl dioxide, methylthio, methylsulfinyl, methylsulfonyl; 2,3-dihydroxypropyl, 2,3-dihydroxypropoxy, F, Cl, COOH, COOMe, COOEt, and CN.

11. In certain compounds of any of the preceding embodiments, Y³ is H and Y⁴ is selected from the group consisting of CN, OMe, OEt, Me, Et, COOH, COOMe, S(O)_{q}Me, —O(CH₂)₂—OH, —O(CH₂)₂—OMe, —OCH₂—CH(OH)—CH₂OH, —CH(OH)—CH₂OH, —(CH₂)₂—OH, —C(CH₃)₂OH, —CH₂OH, methoxymethyl, ethoxymethyl, 3-hydroxy-3-oxetanyl, 3-oxetanyloxy, cyclopropyl, 1-hydroxycyclopropyl, 2-hydroxy-2-methylpropoxy, 1-hydroxycyclobutyl, 2-methoxy-2-methylpropoxy, difluoromethyl, isopropoxy, 2-hydroxy-2-methylethyl, 3-tetrahydrofuranyloxy, 1-hydroxyethyl, cyclopropylmethoxy, 4-tetrahydropyranyl, 4-tetrahydropyranyloxy, 4-tetrahydrothiopyranyl, 4-tetrahydrothiopyranyl dioxide, difluoromethoxy, and —CH₂OMe. Preferred embodiments include CN, OMe, OEt, Me, Et, COOH, COOMe, S(O)_{q}Me, —O(CH₂)₂—OH, —O(CH₂)₂—OMe, —OCH₂—CH(OH)—CH₂OH, —CH(OH)—CH₂OH, —(CH₂)₂—OH, —C(CH₃)₂OH, —CH₂OH, and —CH₂OMe.

12. In certain compounds of any of embodiments 1-10, Y⁴ is H and Y³ is selected from the group consisting of CN, Et, COOH, COOMe, S(O)_{q}Me, —O(CH₂)₂—OH, —O(CH₂)₂—OMe, —(CH₂)₂—OH, —OCH₂—CH(OH)—CH₂OH, —CH(OH)—CH₂OH, —C(CH₃)₂OH, —CH₂OH and —CH₂OMe.

13. In some embodiments of the compounds described above, Y³ is H. In alternative embodiments, Y⁴ is H. In some preferred embodiments, Y³ is H and Y⁴ is as described in any of the preceding embodiments and is preferably selected from —OCH₂CH₂OMe, 4-tetrahydropyranyl, methoxymethyl, 3-oxetanyl, carboxymethyl, methylsulfonyl, difluoromethoxy, and ethoxymethyl; or when Y⁴ is H, Y³ is —OCH₂CH₂OH or —OCH₂CH₂OMe.

14. In some preferred compounds of any of embodiments 1-10, Y⁴ is selected from Me, OMe, —CH₂OMe, —CH₂OEt, COOMe, S(O)ₚMe, —O(CH₂)₂—OH, 4-tetrayhydropyranyl, 4-tetrahydropyranyloxy, —O(CH₂)₂—OMe, —OCH₂—CH(OH)—CH₂OH, —CH(OH)—CH₂OH, —(CH₂)₁₋₂—OH, —C(CH₃)₂OH, and —(CH₂)₁₋₂—OMe, where p is 0, 1 or 2.

In some embodiments of any of the compounds of embodiments 1-14, J is H. In other embodiments of these compounds, J is NH₂.

15. Specific embodiments of compounds of the invention are listed in Table 1;

additional embodiments are listed in Table 2; and more embodiments are listed in Table 3. Many, but not all, of these compounds are compounds of Formula I. The invention includes each of the compounds in these tables, as well as subsets of two or more of these compounds, as preferred embodiments. Some preferred embodiments are any compound selected from the compounds of Examples 16, 22, 38, 99, and 102; or from the compounds of Examples 86, 87, 100, 101, 113, 118, and 120; or from the compounds of Examples 12, 14, 40, 41, 63, 65, 66, 67, 71, 72, 77, 81, 82, 83, 84, 85, 94, 124, 138, 140, 141, 151, 152, 156, 164, 165, 170, 171, 188, 192, 211, 215, and 236.

In many of the foregoing embodiments, the compounds described have at least one amine group and are accordingly often used as acid addition salts. Thus the pharmaceutically acceptable acid addition salts of any of these compounds are preferred embodiments.

16. In another aspect, the invention provides a pharmaceutical composition comprising any of the compounds described specifically or generically in the preceding embodiments 1-15. The pharmaceutical composition also comprises one or more, sometimes two or more, pharmaceutically acceptable excipients or carriers. In some embodiments, the pharmaceutical composition also comprises an additional therapeutic agent, such as those known to be useful for treating a condition for which the compound is to be administered. In some embodiments, the additional therapeutic agent is selected from irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib, anthracyclines, rituximab, and trastuzumab.

17. In another aspect, the invention provides a compound as described in any of embodiments 1-15 for use in therapy, or for use in the preparation of a medicament. The therapy or medicament may be for treatment of a condition characterized by excessive or undesired levels of Pim kinase activity. Typically, it is for treatment of a mammal, often a human, diagnosed as being in need of such treatment. In certain embodiments, the therapy or the medicament is one for treatment of a cancer, or of an autoimmune disorder. In some embodiments, the cancer is selected from carcinoma of the lungs, pancreas, thyroid, ovary, bladder, breast, prostate, or colon, melanoma, myeloid leukemia, multiple myeloma, erythroleukemia, villous colon adenoma, and osteosarcoma.

18. In another aspect, the invention thus provides a method to treat a condition associated with excessive levels of PIM Kinase activity, in a subject in need of such treatment. The subject is often a human. The method comprises administering to a subject having such a condition, typically a human subject, an effective amount of a compound or a pharmaceutical composition according to any the above-described embodiments 1-16.

19. In some embodiments of embodiment 17 or 18, the method or compound is for treatment of cancer or an autoimmune disorder. In specific embodiments, the cancer is a cancer selected from carcinoma of the lungs, pancreas, thyroid, ovary, bladder, breast, prostate, or colon, melanoma, myeloid leukemia, multiple myeloma, erythroleukemia, villous colon adenoma, and osteosarcoma; or the autoimmune disorder is selected from Crohn's disease, inflammatory bowel disease, rheumatoid arthritis, and chronic inflammatory diseases.

### Synthetic Methods

The compounds of the invention can be obtained through procedures known to those skilled in the art. For example, as shown in Scheme 1, cyclohexanediones can be converted via monotriflates to the corresponding cyclohexenoneboronate esters which can undergo palladium mediated carbon bond formation with 4-chloro, 3-nitro pyridine to yield nitropyridine substituted cyclohexenones I. Reduction of the enone functionality can yield a cyclohexenol II, which upon alcohol protection, nitro and alkene reduction, amide coupling and deprotection can yield cyclohexanol amides III. Cyclohexenol II can also undergo Mitsunobu reaction with phthalimide to yield a protected aminocyclohexene IV. Following nitro and alkene reduction, phthalimide protected aminocyclohexyl pyridyl aniline Va can undergo amide coupling and deprotection, to yield aminocyclohexane amides VI. The corresponding Boc protected aminocyclohexane pyridyl aniline Vb can also be prepared from cyclohexenol II in the following manner: alcohol protection, alkene and nitro reduction, pyridyl amine Cbz protection, silyl ether deprotection, Dess-Martin oxidation to the cyclohexanone, reductive amination with benzylamine, Cbz and Bn deprotection and primary aliphatic amine Boc protection. In the amide products III and VI, if R₂ is halo or triflate, the amides III and VI can be further modified by standard modifications to introduce substituted aryls, alkyls and heteroaryls on place of R₂. For example, if R₂ is Br, by reaction with boronic acids or organometallic reagents, or conversion to the corresponding boronate ester and reaction with aryl/heteroaryl halides or triflates, a variety of R₂ replacements are possible.

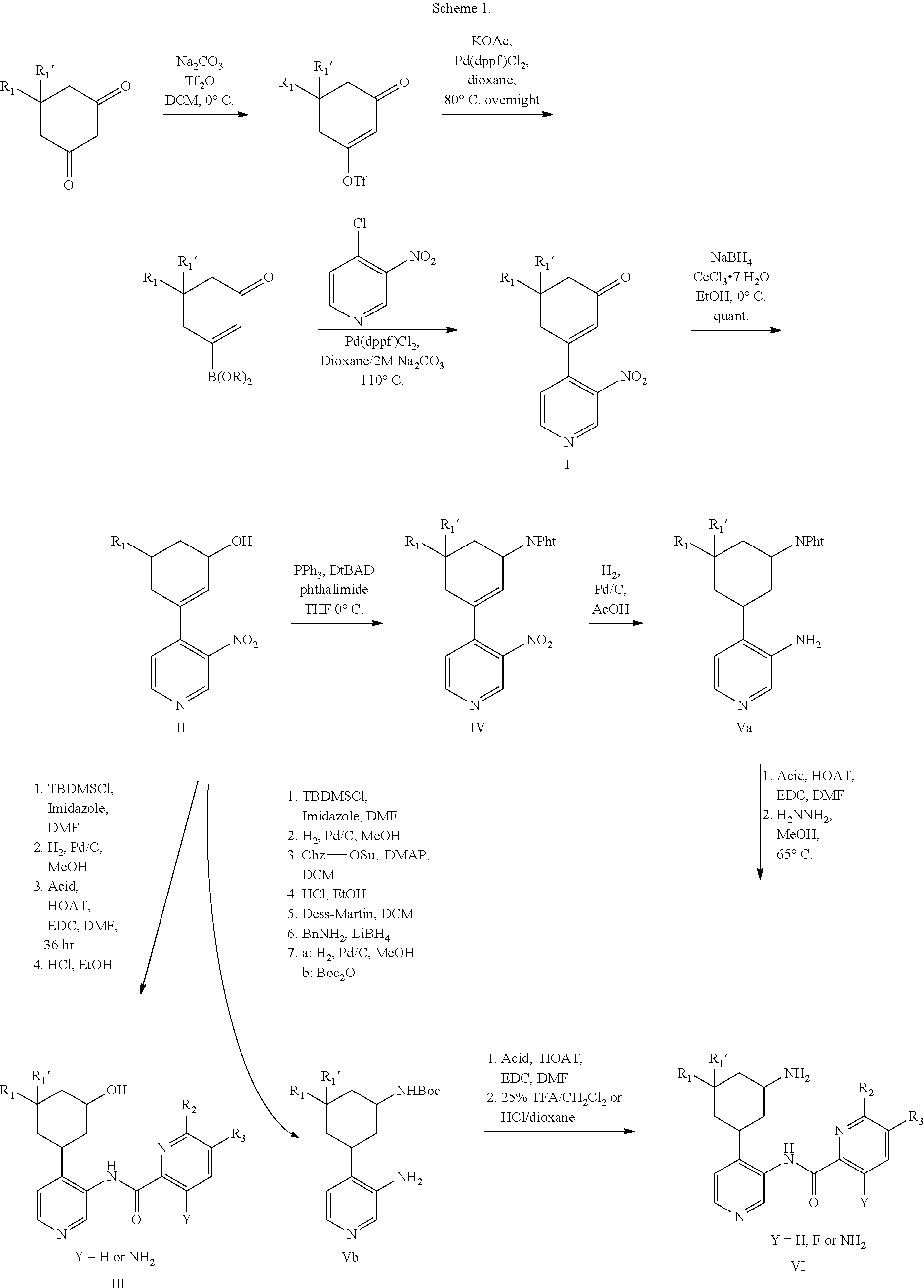

Alternatively, as shown in Scheme 2, cyclohexenol II can be dehydrated yielding a cyclohexadiene which upon epoxidation (via bromohydrin formation and HBr elimination or from mCPBA directly) and azide epoxide opening yields cyclohexenyl azido alcohol VI. Cyclohexenyl azido alcohol VI can be converted to the trans protected amino hydroxy aniline VIIa by azide reduction, alcohol protection and alkene and nitro reduction. Alternatively, the cyclohexenyl azido alcohol VI can be converted to the protected cis amino hydroxy aniline VIIb by azide reduction and Boc protection, alcohol mesylation and intramolecular cyclization to the cis cyclic carbamate, followed by Boc protection and alkene and nitro reduction. The resulting cyclohexylpyridyl anilines VIIa and VIIb can be converted to the corresponding pyridine amides VIIIa and VIIIb by amide coupling, acetate or cyclic carbamate cleavage and Boc deprotection. If R2 is halo or triflate, the amides VIIIa and VIIIb can be further modified by standard modifications to introduce substituted aryls, alkyls and heteroaryls at R2 after amide bond formation and prior to full deprotection. For example, if R2 is Br, by reaction with boronic acids or organometallic reagents, or conversion to the corresponding boronate ester and reaction with aryl/heteroaryl halides or triflates, a variety of R2 modifications are possible. Additionally, the cyclohexenol epoxide can be opened up with water to yield a diol which can lead to dihydroxycyclohexyl containing compounds of the invention.

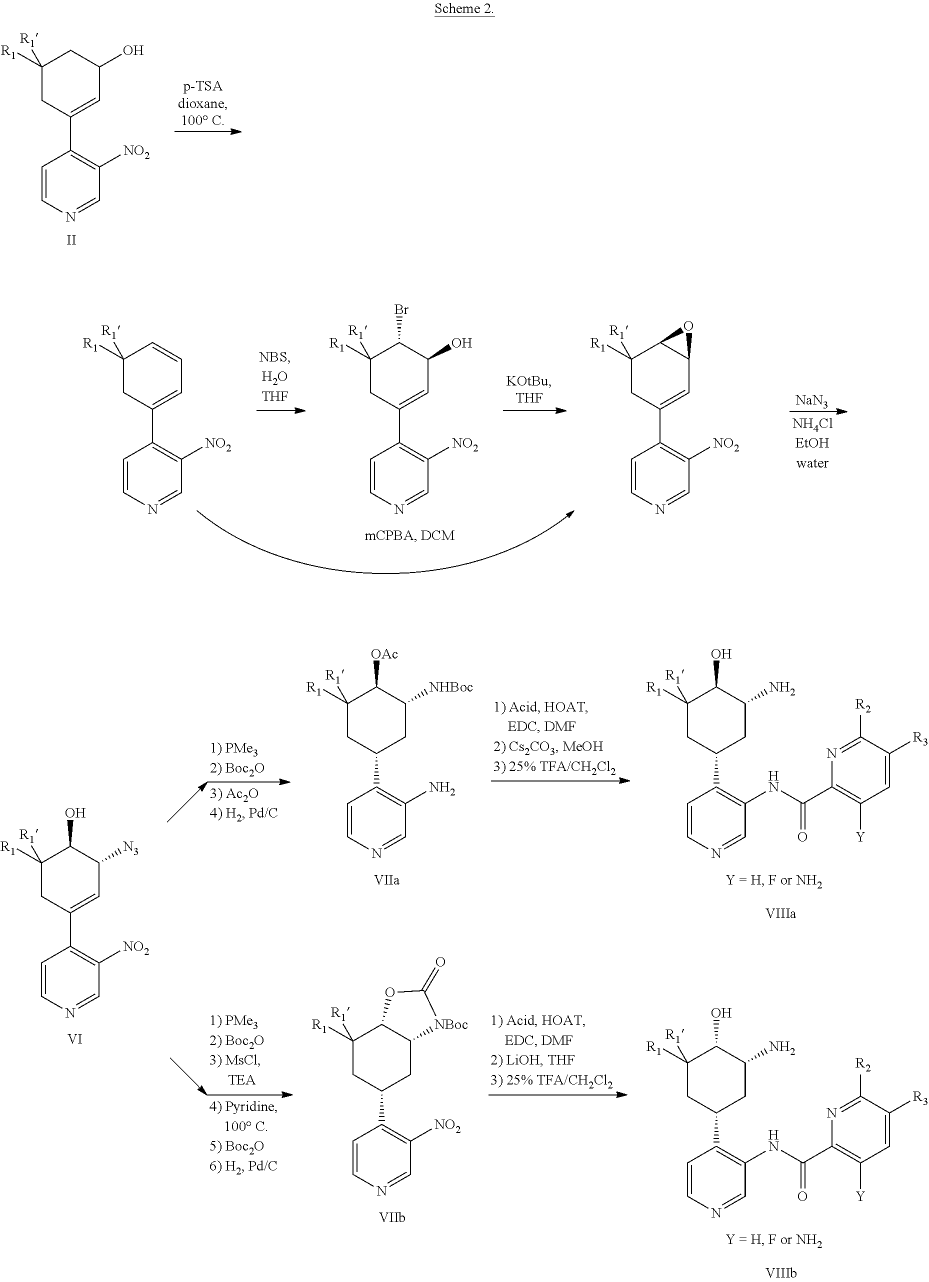

Alternatively, as shown in Scheme 3, trisubstituted 5-alkyl, 4-hydroxy, 3-aminopiperidines can be prepared and modified to yield trisubstituted 5-alkyl, 4-hydroxy, 3-aminopiperidinyl pyridine amides IX as follows. Reaction of Garner's aldehyde with (R)-4-benzyl-3-propionyloxazolidin-2-one followed by TBS protection of the resulting alcohol affords compound X. Reduction of the oxazolidinone followed by introduction of the azide group yields intermediate XI. Deprotection under acidic conditions reveals the corresponding amino alcohol, which upon protection with the Boc group followed by mesylation of the primary alcohol yields intermediate XII. Reduction of the azide affords formation of the piperidine which is subsequently reacted with 4-chloro-3-nitropyridine, reduced to the amine, coupled with the corresponding carboxylic acid and deprotected to provide trisubstituted 5-methyl,4-hydroxy-3-aminopiperidinyl pyridine amides IX. If R₁ is halo or triflate, the amide IX can be further modified by standard modifications to introduce substituted aryls, alkyls and heteroaryls at R₁ after amide bond formation and prior to full deprotection. For example, if R₁ is Br, by reaction with boronic acids or organometallic reagents, or conversion to the corresponding boronate ester and reaction with aryl/heteroaryl halides or triflates, a variety of R₁ modifications are possible. If the starting aldehyde used is gylceraldehyde acetonide, dihydroxypiperidine compounds can be obtained following the methods of Scheme 3.

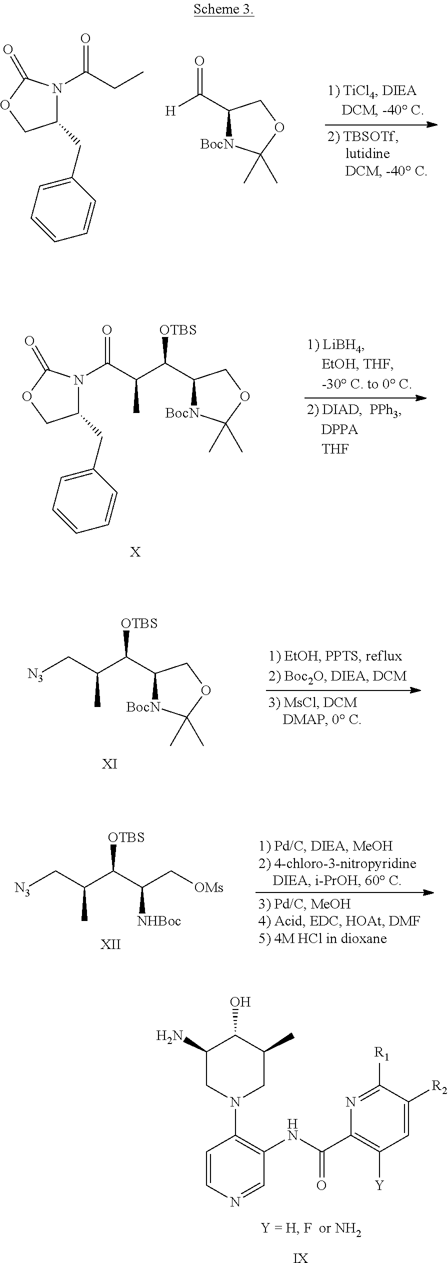

Note that this sequence produces compounds of known absolute stereochemistry, while other methods may produce racemic compounds that require chiral separation and thus produce both enantiomers of the product.

The compounds of the invention are useful in vitro and/or in vivo in inhibiting the growth of cancer cells and are accordingly useful to treat cancer. The compounds may be used alone or in compositions together with a pharmaceutically acceptable carrier or excipient. Suitable pharmaceutically acceptable carriers or excipients include, for example, processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinyl-pyrrolidinone, low melting waxes, ion exchange resins, and the like, as well as combinations of any two or more thereof. Preferred pharmaceutical compositions include one or more sterile carriers or excipients. Other suitable pharmaceutically acceptable excipients are described in REMINGTON'S PHARMACEUTICAL SCIENCES, Mack Pub. Co., New Jersey (1991), incorporated herein by reference.

Effective amounts of the compounds of the invention generally include any amount sufficient to detectably inhibit Pim activity by any of the assays described herein, by other Pim kinase activity assays known to those having ordinary skill in the art or by detecting an inhibition or alleviation of symptoms of cancer.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

For purposes of the present invention, a therapeutically effective dose will generally be a total daily dose administered to a host in single or divided doses may be in amounts, for example, of from 0.001 to 1000 mg/kg body weight daily and more preferred from 1.0 to 30 mg/kg body weight daily. Typical daily dosages for a human subject would be 10 to 2000 mg/day, more commonly 20 to 1500 mg/day, and frequently 50 to 1000 mg/day. Dosage unit compositions may contain such amounts of submultiples thereof to make up the daily dose. The compounds of the present invention may be administered orally, parenterally, sublingually, by aerosolization or inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq. (1976).

While the compounds of the invention can be administered as the sole active pharmaceutical agent, they can also be used in combination with one or more other agents used in the treatment of cancer. The compounds of the present invention are also useful in combination with known therapeutic agents and anti-cancer agents, and combinations of the presently disclosed compounds with other anti-cancer or chemotherapeutic agents are within the scope of the invention. Examples of such agents can be found in Cancer Principles and Practice of Oncology, V. T. Devita and S. Hellman (editors), 6th edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The compounds of the invention are also useful when co-administered with radiation therapy.

Therefore, in one embodiment of the invention, the compounds of the invention are also used in combination with known therapeutic or anticancer agents including, for example, estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

In certain presently preferred embodiments of the invention, representative therapeutic agents useful in combination with the compounds of the invention for the treatment of cancer include, for example, irinotecan, topotecan, gemcitabine, 5-fluorouracil, cytarabine, daunorubicin, PI3 Kinase inhibitors, mTOR inhibitors, DNA synthesis inhibitors, leucovorin carboplatin, cisplatin, taxanes, tezacitabine, cyclophosphamide, vinca alkaloids, imatinib (Gleevec), anthracyclines, rituximab, trastuzumab, Revlimid, Velcade, dexamethasone, daunorubicin, cytaribine, clofarabine, Mylotarg, as well as other cancer chemotherapeutic agents including targeted therapeutics.

The above compounds to be employed in combination with the compounds of the invention will be used in therapeutic amounts as indicated in the Physicians' Desk Reference (PDR) 47th Edition (1993), which is incorporated herein by reference, or such therapeutically useful amounts as would be known to one of ordinary skill in the art, or provided in prescribing materials such as a drug label for the additional therapeutic agent.

The compounds of the invention and the other anticancer agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions, which are given at the same time or different times, or the therapeutic agents, can be given as a single composition.

In one embodiment, the invention provides a method of inhibiting Pim1, Pim2 or Pim3 in a human or animal subject. The method includes administering an effective amount of a compound, or a pharmaceutically acceptable salt thereof, of any of the embodiments of compounds of Formula I or II to a subject in need thereof.

The present invention will be understood more readily by reference to the following examples, which are provided by way of illustration and are not intended to be limiting of the present invention.

### EXAMPLES

Referring to the examples that follow, compounds of the preferred embodiments were synthesized using the methods described herein, or other methods, which are known in the art.

The compounds and/or intermediates were characterized by high performance liquid chromatography (HPLC) using a Waters Millenium chromatography system with a 2695 Separation Module (Milford, Mass.). The analytical columns were reversed phase Phenomenex Luna C18-5μ, 4.6×50 mm, from Alltech (Deerfield, Ill.). A gradient elution was used (flow 2.5 mL/min), typically starting with 5% acetonitrile/95% water and progressing to 100% acetonitrile over a period of 10 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well-known iodine vapor and other various staining techniques.

Mass spectrometric analysis was performed on one of three LCMS instruments: a Waters System (Alliance HT HPLC and a Micromass ZQ mass spectrometer; Column: Eclipse XDB-C18, 2.1×50 mm; gradient: 5-95% (or 35-95%, or 65-95% or 95-95%) acetonitrile in water with 0.05% TFA over a 4 min period; flow rate 0.8 mL/min; molecular weight range 200-1500; cone Voltage 20 V; column temperature 40° C.), another Waters System (ACQUITY HPLC system and a ZQ 2000 system; Column: ACQUITY HPLC HSS-C18, 1.8 um, 2.1×50 mm; gradient: 5-95% (or 35-95%, or 65-95% or 95-95%) acetonitrile in water with 0.05% TFA over a 1.3 min period; flow rate 1.2 mL/min; molecular weight range 150-850; cone Voltage 20 V; column temperature 50° C.) or a Hewlett Packard System (Series 1100 HPLC; Column: Eclipse XDB-C18, 2.1×50 mm; gradient: 5-95% acetonitrile in water with 0.05% TFA over a 4 min period; flow rate 0.8 mL/min; molecular weight range 150-850; cone Voltage 50 V; column temperature 30° C.). All masses were reported as those of the protonated parent ions.

Nuclear magnetic resonance (NMR) analysis was performed on some of the compounds with a Varian 400 MHz NMR (Palo Alto, Calif.). The spectral reference was either TMS or the known chemical shift of the solvent.

Preparative separations are carried out using a Flash 40 chromatography system and KP-Sil, 60A (Biotage, Charlottesville, Va.), or by flash column chromatography using silica gel (230-400 mesh) packing material on ISCO or Analogix purification systems, or by HPLC using a Waters 2767 Sample Manager, C-18 reversed phase column, 30×50 mm, flow 75 mL/min. Typical solvents employed for the Flash 40 Biotage, ISCO or Analogixsystem for silica gel column chromatography are dichloromethane, methanol, ethyl acetate, hexane, n-heptanes, acetone, aqueous ammonia (or ammonium hydroxide), and triethyl amine. Typical solvents employed for the reverse phase HPLC are varying concentrations of acetonitrile and water with 0.1% trifluoroacetic acid.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

It is understood that the invention is not limited to the embodiments set forth herein for illustration, but embraces all such forms thereof as come within the scope of the above disclosure.

The examples below as well as throughout the application, the following abbreviations have the following meanings If not defined, the terms have their generally accepted meanings

| | ABBREVIATIONS |
|---|---|
| DAST | (diethylamino)sulfurtrifluoride |
| DCM | Dichloromethane |
| DIAD | diisopropylazodicarboxylate |
| DIEA | diisopropylethylamine |
| DMA | Dimethylacetamide |
| DMAP | 4-dimethylaminopyridine |
| DME | 1,2-dimethoxyethane |
| DMF | N,N-dimethylformamide |
| DPPF | 1,1′-bis(diphenylphosphino)ferrocene |
| EDC | 1-(3-Dimethylaminopropyl)-3-ethylcarbodiimide |
| | hydrochloride |
| EtOAc | ethyl acetate |
| EtOH | Ethanol |
| HOAT | Hydroxyazabenzotriazole |
| K₂CO₃ | Potassium carbonate |
| MeCN | Acetonitrile |
| MgSO₄ | Magnesium sulfate |
| MeOH | Methanol |
| Na₂CO₃ | sodium carbonate |
| NaCl | Sodium chloride |
| NaHCO₃ | sodium bicarbonate |
| NBS | N-bromosuccinimide |
| NMP | N-methyl-2-pyrrolidone |
| Pd₂(dba)₃ | Tris(dibenzylideneacetone)dipalladium(0) |
| Pd(PPh₃)₄ | Tetrakis(triphenylphospine)palladium(0) |
| Pd(dppf)Cl₂- | Dichloro-(1,2-bis(diphenylphosphino)ethan)- |
| DCM | Palladium(II)-dichloromothethane adduct |
| RT or rt | room temperature |
| TBDMSCl | tert-butyldimethylsilylchloride |
| TEA | Triethylamine |
| THF | tetrahydrofuran |

### Examples

### Synthesis of 5-methyl-3-oxocyclohex-1-enyltrifluoromethanesulfonate

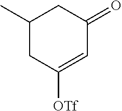

To a solution of 5-methylcyclohexane-1,3-dione (1.0 equiv.) in DCM (0.5M) was added Na₂CO₃ (1.1 equiv.) and cooled to 0° C. Added Tf₂O (1.0 equiv.) in DCM (5.0 M) dropwise over 1 hr at 0° C. under a nitrogen atmosphere. Upon addition, the reaction was stirred for 1 hr at room temperature (dark red solution). The solution was filtered and the filtrate was quenched by careful addition of saturated NaHCO₃ with vigorous stirring until pH=7. The solution was transferred to a separatory funnel and the layers were separated. The organic layer was washed with brine, dried with Na₂SO₄, filtered, concentrated under vacuo and dried under high vacuum for 15 min to yield 5-methyl-3-oxocyclohex-1-enyl trifluoromethanesulfonate as light yellow oil in 78% yield. The triflate decomposes upon storage and should be used immediately for the next reaction. LC/MS=259.1/300.1 (M+H and M+CH₃CN); Rt=0.86 min, LC=3.84 min. ¹H-NMR (400 MHz, CDCl₃) δ ppm: 6.05 (s, 1H), 2.70 (dd, J=17.2, 4.3, 1H), 2.53 (dd, J=16.6, 3.7, 1H), 2.48-2.31 (m, 2H), 2.16 (dd, J=16.4, 11.7, 1H), 1.16 (d, J=5.9, 3H).

### Synthesis of 5-methyl-3-(4,4,5,5-tetramethyl-1,3,2-dioxaborolan-2-yl)cyclohex-2-enone

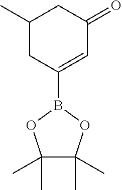

To a solution of 5-methyl-3-oxocyclohex-1-enyl trifluoromethanesulfonate (1.0 equiv.) in degassed dioxane (0.7 M) was added bis(pinacolato)diboron (2.0 equiv.), KOAc (3.0 equiv.), and Pd(dppf)Cl₂-DCM (0.03 equiv.). The reaction was heated to 80° C. for 10 h (initial heating at large scale results in exothermic formation of an orange foam on top of the solution, the heating bath should be removed until the foam retracts, reheating to 80° C. at this point appears to be fine), then cooled to room temperature and filtered through a coarse frit glass funnel. The cake was rinsed with more dioxane and the filtrate solution was used for the next step without further purification. LC/MS=155.1 (M+H of boronic acid); Rt=0.41 min, LC=1.37 min.

### Synthesis of 5-methyl-3-(3-nitropyridin-4-yl)cyclohex-2-enone

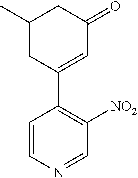

To a solution of 5-methyl-3-(4,4,5,5-tetramethyl-1,3,2-dioxaborolan-2-yl)cyclohex-2-enone (1.0 equiv.) in degassed dioxane (0.5 M) and 2M Na₂CO₃ (2 equiv.) was added 4-chloro-3-nitropyridine (1.3 equiv.) and Pd(dppf)Cl₂-DCM (0.05 equiv.). The reaction was placed under a reflux condenser and heated in an oil bath to 110° C. for 1 h. Cooled to room temperature, filtered through a pad of Celite, washed the pad with ethyl acetate and concentrated the filtrate under vacuo. The residue was further pumped at 80° C. on a rotary evaporator for one hour to remove boronate by-products (M+H=101) via sublimation. The residue was partitioned between brine and ethyl acetate, and the layers were separated, the aqueous phase was further extracted with ethyl acetate (4×), the organics were combined, dried over sodium sulfate, filtered, and concentrated. The crude was purified via silica gel chromatography loading in DCM and eluting with 2-50% ethyl acetate and hexanes. The pure fractions were concentrated in vacuo to yield an orange oil. The oil was placed under high vacuum (˜500 mtorr) with seed crystals overnight to yield an orange solid. The solid was further purified via trituration in hexanes to yield 5-methyl-3-(3-nitropyridin-4-yl)cyclohex-2-enone (48% 2 steps). LC/MS=233.2 (M+H); Rt=0.69 min, LC=2.70 min. ¹H-NMR (400 MHz, CdCl₃) δ ppm: 9.31 (s, 1H), 8.88 (d, J=5.1, 1H), 7.30 (d, J=5.1, 1H), 6.00 (d, J=2.4, 1H), 2.62 (dd, J=16.4, 3.5, 1H), 2.53-2.34 (m, 3H), 2.23 (dd, J=16.1, 11.7, 1H), 1.16 (d, J=6.3, 3H).

### Synthesis of cis-(+/−)-5-methyl-3-(3-nitropyridin-4-yl)cyclohex-2-enol

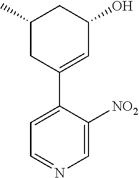

To a solution of 5-methyl-3-(3-nitropyridin-4-yl)cyclohex-2-enone (1.0 equiv.) in EtOH (0.3 M) was added CeCl₃-7H₂O (1.2 equiv.). The reaction was cooled to 0° C., then NaBH₄ (1.2 equiv.) was added in portions. Stirred for 1 h at 0° C., then quenched by adding water, concentrated to remove the EtOH, added EtOAc, extracted the organics, washed with brine, then dried with Na₂SO₄, filtered and concentrated to yield cis-(+/−)-5-methyl-3-(3-nitropyridin-4-yl)cyclohex-2-enol (94%). LC/MS=235.2 (M+H), LC=2.62 min.

### Synthesis of 4-(3-(tert-butyldimethylsilyloxy)-5-methylcyclohex-1-enyl)-3-nitropyridine

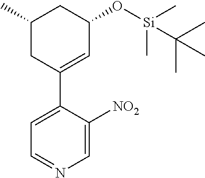

To a solution of 5-methyl-3-(3-nitropyridin-4-yl)cyclohex-2-enol (1.0 equiv.) in DMF (0.5 M) was added imidazole (4.0 equiv.) and TBDMSCl (2.5 equiv.). After stirring for 18 hours the solution was portioned between EtOAc and H₂O and separated. After washing further with H₂O (3×) and NaCl (sat.), drying over MgSO₄, filtering and removal of solvents, 4-(3-(tert-butyldimethylsilyloxy)-5-methylcyclohex-1-enyl)-3-nitropyridine was obtained (85%). LC/MS=349.2 (M+H), LC=5.99 min.

### Synthesis of 4-(3-(tert-butyldimethylsilyloxy)-5-methylcyclohex-1-enyl)pyridin-3-amine

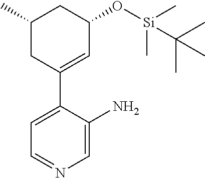

A heterogeneous solution of 4-(3-(tert-butyldimethylsilyloxy)-5-methylcyclohex-1-enyl)-3-nitropyridine (1.0 eq.) and iron (6.0 eq) in acetic acid, at a concentration of 0.4 M, was stirred vigorously for 2 hours. The mixture was then passed through a celite pad, eluting with MeOH. Upon removal of the volatiles in vacuo, the residue was dissolved in EtOAc, washed with Na₂CO_{3(sat.)}, NaCl_{(sat.)}, was dried over MgSO₄, was filtered and the volatiles were removed in vacuo yielding 4-(3-(tert-butyldimethylsilyloxy)-5-methylcyclohex-1-enyl)pyridin-3-amine (78%). LCMS (m/z): 319.3 (MH⁺); LC Rₜ=3.77 min.

### Synthesis of 4-(3-(tert-butyldimethylsilyloxy)-5-methylcyclohexyl)pyridin-3-amine

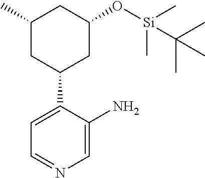

To a solution of 4-(3-(tert-butyldimethylsilyloxy)-5-methylcyclohex-1-enyl)-3-nitropyridine (1.0 equiv.) in methanol, at a concentration of 0.1 M, was added 10% palladium on carbon (0.1 eq.). The resultant heterogeneous solution was put under an atmosphere of hydrogen and was stirred for 15 hours. At this time the mixture was filtered through a pad of celite eluting with methanol. The volatiles were removed in vacuo yielding 4-(3-(tert-butyldimethylsilyloxy)-5-methylcyclohexyl)pyridin-3-amine (90%). LCMS (m/z): 321.3 (MH⁺); LC Rₜ=3.85 min.

### Synthesis of cis (+/−) benzyl 4-3-(tert-butyldimethylsilyloxy)-5-methylcyclohexyl)pyridin-3-ylcarbamate

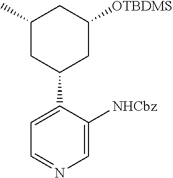

To a solution of cis-(+/−)-4-(3-(tert-butyldimethylsilyloxy)-5-methylcyclohexyl)pyridin-3-amine in dichloromethane at a concentration of 0.5 M was added benzyl 2,5-dioxopyrrolidin-1-yl carbonate (1.1 equiv.) and DMAP (0.05 equiv.). After stirring for 16 hours at rt, additional benzyl 2,5-dioxopyrrolidin-1-yl carbonate (0.55 equiv.) and DMAP (0.03 equiv.) were added. After stirring for an additional 24 hours at rt, additional benzyl 2,5-dioxopyrrolidin-1-yl carbonate (0.1 equiv.) and DMAP (0.03 equiv.) were added. After stirring for 18 more hours the solution was partitioned between EtOAc and Na₂CO_{3(sat.)} and separated. Upon further washing with Na₂CO_{3(sat.)} (2×) and NaCl_{(sat.)}, drying over MgSO₄, filtering and removal of solvents, cis (+/−) benzyl 4-3-(tert-butyldimethylsilyloxy)-5-methylcyclohexyl)pyridin-3-ylcarbamate was obtained. The crude material was used as is. LC/MS=455.3 (M+H), LC=4.39 min.

### Synthesis of cis-(+/−)benzyl 4-(3-hydroxy-5-methylcyclohexyl)pyridin-3-ylcarbamate

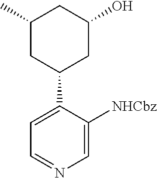

A solution of cis (+/−) benzyl 4-3-(tert-butyldimethylsilyloxy)-5-methylcyclohexyl)pyridin-3-ylcarbamate in 1:2:1 6N HCl/THF/MeOH at a concentration of 0.1 M was stirred at rt for 6 hours. The pH was than adjusted to pH=7 by addition of 6N NaOH and the volatiles were removed in vacuo. The aqueous layer was extracted with EtOAc and the organic was washed with NaCl_{(sat.)}, dried over MgSO₄, filtered and upon removal of the volatiles in vacuo, cis-(+/−)benzyl 4-(3-hydroxy-5-methylcyclohexyl)pyridin-3-ylcarbamate was obtained. The crude material was used as is. LC/MS=341.2 (M+H), LC=2.38 min.

### Synthesis of cis (+/−)-benzyl 4-(3-methyl-5-oxocyclohexyl)pyridin-3-ylcarbamate

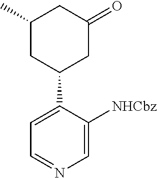

To a 0° C. solution of cis-(+/−)-benzyl 4-(3-hydroxy-5-methylcyclohexyl)pyridin-3-ylcarbamate in wet CH₂Cl₂ at a concentration of 0.16 M was added Dess-Martin Periodinane (1.5 equiv.) and the solution was stirred for 18 hours as it warmed to rt. The solution was partitioned between EtOAc and 1:1 10% Na₂S₂O₃/NaHCO_{3(sat.)} and separated. Upon further washing with 1:1 10% Na₂S₂O₃/NaHCO_{3(sat.)} (2×) and NaCl_{(sat.)}, drying over MgSO₄, filtering, removal of solvents and purification by silica gel chromatography (75-100% EtOAc/hexanes), cis-(+/−)-benzyl-4-(3-methyl-5-oxocyclohexyl)pyridin-3-ylcarbamate was obtained as a white solid (53%, 5 steps). LC/MS=339.2 (M+H).

### Synthesis of cis-(+/−)-benzyl 4-(−3-(benzylamino)-5-methylcyclohexyl)pyridin-3-ylcarbamate

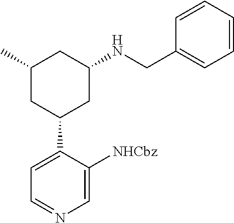

A solution of cis-(+/−)-benzyl-4-(3-methyl-5-oxocyclohexyl)pyridin-3-ylcarbamate (1.0 equiv) and benzylamine (3.0 equiv) in MeOH, at a concentration of 0.25 M, was stirred at rt for 2 hours. Upon cooling in a −78° C. bath, LiBH₄ (1.1 equiv, 2.0 M in THF) was added and the solution was allowed to warm to rt with stirring over 16 hours. The solution was partitioned between EtOAc and NaHCO_{3(sat.)}, separated, washed further with NaHCO_{3(sat.)} and NaCl_{(sat.)}, dried over MgSO₄, filtered and after removal of volatiles in vacuo, cis-(+/−)-benzyl 4-(−3-(benzylamino)-5-methylcyclohexyl)pyridin-3-ylcarbamate was obtained as a 4:1 mixture of isomers, with the all cis as predominant LC/MS=430.3 (M+H), LC=0.62 min.

### Synthesis of cis (+/−)-tert-butyl (−3-(3-aminopyridin-4-yl)-5-methylcyclohexylcarbamate

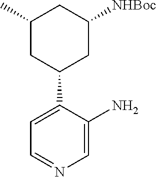

To a solution of cis-(+/−)-benzyl 4-(−3-(benzylamino)-5-methylcyclohexyl)pyridin-3-ylcarbamate was (1.0 equiv.) in methanol, at a concentration of 0.07 M, was added 20% palladium hydroxide on carbon (0.2 eq.). The resultant heterogeneous solution was put under an atmosphere of hydrogen and was stirred for 14 hours. At this time the reaction was purged with Ar, Boc₂O (1.0 equiv.) was added and the solution was stirred for 8 hours. Additional Boc₂O (1.0 equiv.) was added and the solution was stirred for 16 more hours. At this time the mixture was filtered through a pad of celite eluting with methanol. Upon removal of volatiles in vacuo, purification by silica gel chromatography (2.5-2.5 MeOH/CH₂Cl₂ with 0.1% DIEA) and recrystallization from 10% EtOAc/hexanes yielded cis (+/−)-tert-butyl (−3-(3-aminopyridin-4-yl)-5-methylcyclohexylcarbamate (49%). LCMS (m/z): 306.3 (MH⁺), LC Rₜ=2.59 min. Pure enantiomers could be obtained by chiral chromatography.

### Synthesis of (+/−)-4-(5-methylcyclohexa-1,3-dienyl)-3-nitropyridine

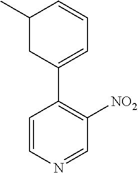

To a solution of (+/−)-5-methyl-3-(3-nitropyridin-4-yl)cyclohex-2-enol (1.0 equiv.) in dioxane (0.1M) was added p-TSA (1.0 equiv.), and the reaction was stirred at 100° C. for 3 h. The solution was cooled to room temperature, then passed through a pad of neutral alumina eluting with EtOAc to yield (+/−)-4-(5-methylcyclohexa-1,3-dienyl)-3-nitropyridine as a yellow oil in 68% yield. LC/MS=217.1 (M+H), LC=3.908 min.

### Synthesis of (+/−)-6-bromo-5-methyl-3-(3-nitropyridin-4-yl)cyclohex-2-enol

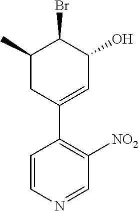

To a solution of 4-(5-methylcyclohexa-1,3-dienyl)-3-nitropyridine (1.0 equiv.) in THF and water (1:1, 0.13 M) was added NBS (1.5 equiv.) and the reaction was stirred at room temperature for 30 min. Upon completion, ethyl acetate and water were added to the reaction, the organic phase was dried with brine, then sodium sulfate, filtered, and concentrated. The crude material was purified via silica gel column chromatography eluting with ethyl acetate and hexanes (1:1) to give (+/−)-6-bromo-5-methyl-3-(3-nitropyridin-4-yl)cyclohex-2-enol as a yellow oil in 80% yield. LC/MS=315.0/313.0 (M+H), LC=2.966 min.

### Synthesis of (+/−)-2-azido-6-methyl-4-(3-nitropyridin-4-yl)cyclohex-3-enol

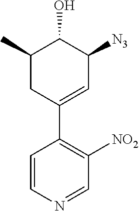

To a solution of (+/−)-6-bromo-5-methyl-3-(3-nitropyridin-4-yl)cyclohex-2-enol (1.0 equiv.) in THF (0.1 M) was added potassium tert-butoxide (1.5 equiv.). The reaction turned from orange to black almost immediately. By TLC, the formation of product is clean in 30 min. Quenched by adding saturated ammonium chloride and ethyl acetate. The organic phase was dried with brine, then sodium sulfate, filtered, and concentrated. The crude product was dissolved in ethanol and water (3:1, 0.1 M), and ammonium chloride (2.0 equiv) and sodium azide (2.0 equiv.) were added. The dark orange reaction was stirred at room temperature overnight. The conversion to product is clean as indicated by LC/MS. The reaction was concentrated to remove the ethanol, ethyl acetate and water were added, and the organic phase was dried with sodium sulfate, filtered, and concentrated. The crude material was purified via silica gel column chromatography eluting with ethyl acetate and hexanes (1:1) to give (+/−)-2-azido-6-methyl-4-(3-nitropyridin-4-yl)cyclohex-3-enol in 55% yield. LC/MS=276.0 (M+H), LC=2.803 min.

### Synthesis of (+/−)-tert-butyl 6-hydroxy-5-methyl-3-(3-nitropyridin-4-yl)cyclohex-2-enylcarbamate

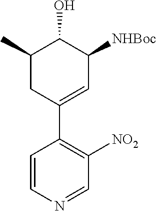

To a solution of (+/−)-2-azido-6-methyl-4-(3-nitropyridin-4-yl)cyclohex-3-enol (1.0 equiv.) in pyridine and ammonium hydroxide (8:1, 0.08 M) was added trimethylphosphine (3.0 equiv.) and the brown solution was stirred at room temperature for 2 h. Upon completion, EtOH was added and the solution was concentrated in vacuo. More ethanol was added and the reaction was concentrated again. Dioxane and sat. NaHCO₃ (1:1, 0.08 M) were added to the crude, followed by Boc₂O (1.0 equiv.). Stirred the reaction mixture at room temperature for 2 h, then added water and ethyl acetate. The organic phase was dried with MgSO₄, and concentrated. The crude product was purified via silica gel column chromatography eluting with ethyl acetate and hexanes (1:1) to afford (+/−)-tert-butyl 6-hydroxy-5-methyl-3-(3-nitropyridin-4-yl)cyclohex-2-enylcarbamate (59%). LC/MS=350.1 (M+H), Rt: 0.76 min.

### Synthesis of (+/−)-2-(tert-butoxycarbonylamino)-6-methyl-4-(3-nitropyridin-4-yl)cyclohex-3-enyl acetate

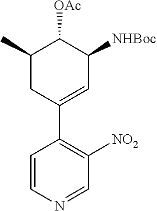

To a solution of (+/−)-tert-butyl 6-hydroxy-5-methyl-3-(3-nitropyridin-4-yl)cyclohex-2-enylcarbamate (1.0 equiv.) in pyridine (0.1 M) was added Ac₂O (2.0 equiv.) and the reaction was stirred at room temperature overnight. Upon completion, the reaction was concentrated to dryness, then worked-up with ethyl acetate and water. The organic phase was dried with brine, then sodium sulfate, filtered, and concentrated to give (+/−)-2-(tert-butoxycarbonylamino)-6-methyl-4-(3-nitropyridin-4-yl)cyclohex-3-enyl acetate in 94% yield. LC/MS=392.2 (M+H), Rt=0.94 min.

### Synthesis of (+/−)-4-(3-aminopyridin-4-yl)-2-(tert-butoxycarbonylamino)-6-methylcyclohexyl acetate

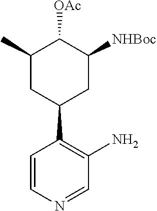

To a degassed solution of (+/−)-2-(tert-butoxycarbonylamino)-6-methyl-4-(3-nitropyridin-4-yl)cyclohex-3-enyl acetate (1.0 equiv.) in MeOH and EtOAc (1:1, 0.1 M) was added 10% Pd/C (0.1 equiv.) and the reaction was stirred at room temperature under a hydrogen balloon for 3 days. Upon completion, the solution was filtered through a pad of Celite, the pad was washed with ethyl acetate and the filtrate was concentrated. The crude material contained about 10% of the undesired isomer. The crude was dissolved in ethyl acetate (˜20%) and hexanes and heated until all dissolved. The solution was allowed to sit at room temperature for 2 days. The precipitate was then collected to give (+/−)-4-(3-aminopyridin-4-yl)-2-(tert-butoxycarbonylamino)-6-methylcyclohexyl acetate as the pure product in 59% yield. LC/MS=364.3 (M+H), Rt=0.63 min.

### Synthesis of 2-(tert-butoxycarbonylamino)-6-methyl-4-(3-nitropyridin-4-yl)cyclohex-3-enyl methanesulfonate

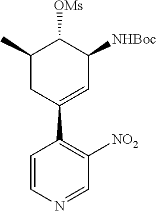

To a solution of tert-butyl 6-hydroxy-5-methyl-3-(3-nitropyridin-4-yl)cyclohex-2-enylcarbamate (1.0 equiv.) in DCM (0.09 M) was added triethylamine (1.5 equiv.) and the reaction was cooled to 0° C. MsCl (1.2 equiv.) was added to the reaction and stirred for 3 h. Another 1.0 equiv. of MsCl was added to the reaction and stirred for another 2 h. Worked up the reaction by adding water, the organic phase was dried with brine, sodium sulfate, and concentrated. The crude product was purified via silica gel column chromatography eluting with ethyl acetate and hexanes (1:1) to afford 2-(tert-butoxycarbonylamino)-6-methyl-4-(3-nitropyridin-4-yl)cyclohex-3-enyl methanesulfonate as a white foam in 65% yield. LC/MS=428.2 (M+H), LC: 3.542 min.

### Synthesis of (+/−)-tert-butyl 7-methyl-5-(3-nitropyridin-4-yl)-2-oxo-3a,6,7,7a-tetrahydrobenzo[d]oxazole-3(2H)-carboxylate

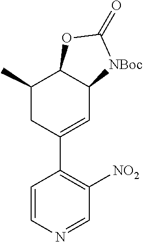

A solution of (+/−)-2-(tert-butoxycarbonylamino)-6-methyl-4-(3-nitropyridin-4-yl)cyclohex-3-enyl methanesulfonate (1.0 equiv.) in pyridine (0.2 M) was heated in the microwave at 110° C. for 10 min. The orange reaction was then concentrated under vacuo, the crude was dissolved in ethyl acetate and water, the organic phase was dried with sodium sulfate and concentrated under vacuo. The crude material was dissolved in DCM (0.2 M), triethylamine (1.8 equiv.) was added, followed by Boc₂O (1.2 equiv.). The reaction was stirred for 40 min, then concentrated to dryness. The crude material was purified via silica gel column chromatography eluting with hexane and ethyl acetate (1:1) to afford (+/−)-tert-butyl 7-methyl-5-(3-nitropyridin-4-yl)-2-oxo-3a,6,7,7a-tetrahydrobenzo[d]oxazole-3(2H)-carboxylate as a white foam in 66% yield. LC/MS=376.0 (M+H), LC: 3.424 min.

### Synthesis of (+/−)-tert-butyl 5-(3-aminopyridin-4-yl)-7-methyl-2-oxohexahydrobenzo[d]oxazole-3(2H)-carboxylate

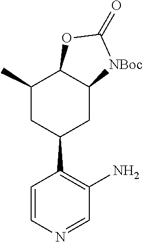

To a degassed solution of (+/−)-tert-butyl 7-methyl-5-(3-nitropyridin-4-yl)-2-oxo-3a,6,7,7a-tetrahydrobenzo[d]oxazole-3(2H)-carboxylate (1.0 equiv.) in MeOH and EtOAc (1:1, 0.1 M) was added 10% Pd/C (0.1 equiv.). The reaction was stirred under a hydrogen balloon overnight. Upon completion, the solution was filtered through a pad of Celite and the pad was washed with ethyl acetate. The filtrate was concentrated under vacuo to give (+/−)-tert-butyl 5-(3-aminopyridin-4-yl)-7-methyl-2-oxohexahydrobenzo[d]oxazole-3(2H)-carboxylate as the desired product as a yellow foam in 93% yield. LC/MS=348.1 (M+H), Rt=055 min.

### Synthesis of (R)-tert-butyl 4-((1R,2R)-3-((R)-4-benzyl-2-oxooxazolidin-3-yl)-1-hydroxy-2-methyl-3-oxopropyl)-2,2-dimethyloxazolidine-3-carboxylate

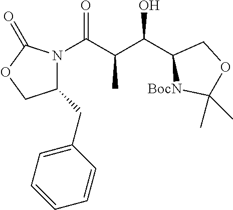

To a solution of (R)-4-benzyl-3-propionyloxazolidin-2-one (1.0 equiv.) in DCM (0.13 M) was added TiCl₄ (1.0 equiv.) at −40° C. The mixture was stirred at −40° C. for 10 min (yellow suspension), then DIPEA (2.5 equiv.) was added (dark red solution) and stirred at 0° C. for 20 min. (R)-tert-butyl 4-formyl-2,2-dimethyloxazolidine-3-carboxylate (1.0 equiv.) in DCM (0.5 M) was then added dropwise and the resulting mixture was stirred for 1.5 hours. The reaction was quenched by the addition of aqueous ammonium chloride and the mixture was extracted with ethyl acetate. The organic phase was separated, washed with brine, dried with magnesium sulfate, filtered, and concentrated. The residue was purified via column chromatography eluting with ethyl acetate and hexanes (1:4) to give (R)-tert-butyl 4-((1R,2R)-3-((R)-4-benzyl-2-oxooxazolidin-3-yl)-1-hydroxy-2-methyl-3-oxopropyl)-2,2-dimethyloxazolidine-3-carboxylate as the major product (5:2) in 58% yield. LC/MS=363.3 (M+H-Boc), Rt=1.09 min.

### Synthesis of (R)-tert-butyl 4-((1R,2R)-3-((R)-4-benzyl-2-oxooxazolidin-3-yl)-1-(tert-butyldimethylsilyloxy)-2-methyl-3-oxopropyl)-2,2-dimethyloxazolidine-3-carboxylate

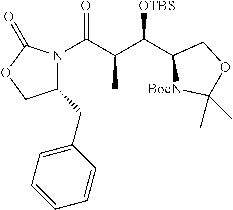

To a solution of (R)-tert-butyl 4-((1R,2R)-3-(R)-4-benzyl-2-oxooxazolidin-3-yl)-1-hydroxy-2-methyl-3-oxopropyl)-2,2-dimethyloxazolidine-3-carboxylate (1.0 equiv.) and lutidine (1.8 equiv.) in DCM (0.1M) was added TBSOTf (1.4 equiv.) at −40° C. The reaction mixture was stirred at −40° C. for 2 hours. The solution was diluted with ethyl acetate and washed with sat. NaHCO₃, sat. NaCl, dried with magnesium sulfate, filtered, and concentrated. The residue was purified by silica gel column chromatography eluting with ethyl acetate and hexanes (1:4) to give (R)-tert-butyl 4-((1R,2R)-3-(R)-4-benzyl-2-oxooxazolidin-3-yl)-1-(tert-butyldimethylsilyloxy)-2-methyl-3-oxopropyl)-2,2-dimethyloxazolidine-3-carboxylate as the major product (5:2) in 83% yield. LC/MS=577.3 (M+H), Rt=1.33 min (Frac 65%-95% method).

### Synthesis of (R)-tert-butyl 4-((1R,2S)-1-(tert-butyldimethylsilyloxy)-3-hydroxy-2-methylpropyl)-2,2-dimethyloxazolidine-3-carboxylate

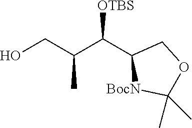

To a solution of (R)-tert-butyl 4-((1R,2R)-3-(R)-4-benzyl-2-oxooxazolidin-3-yl)-1-(tert-butyldimethylsilyloxy)-2-methyl-3-oxopropyl)-2,2-dimethyloxazolidine-3-carboxylate (1.0 equiv.) and ethanol (3.0 equiv.) in THF (0.09 M) was added LiBH₄ (3.0 equiv.) at −30° C. The reaction mixture was allowed to warm up to 0° C. and stirred at that temperature for 3 hours. The solution was then diluted with diethyl ether and 1N NaOH was added. The resulting mixture was extracted with ethyl acetate, the organic layer was separated, washed with sat. NaCl, dried over magnesium sulfate, filtered, and concentrated. The residue was purified via silica gel column chromatography eluting with ethyl acetate and hexanes (1:4) to give (R)-tert-butyl 4-((1R,2S)-1-(tert-butyldimethylsilyloxy)-3-hydroxy-2-methylpropyl)-2,2-dimethyloxazolidine-3-carboxylate as the major product (5:2 ratio) in 71% yield. LC/MS=304.3 (M+H-Boc), Rt=0.95 min (Frac 65%-95% method).

### Synthesis of (R)-tert-butyl 4-((1R,2S)-3-azido-1-(tert-butyldimethylsilyloxy)-2-methylpropyl)-2,2-dimethyloxazolidine-3-carboxylate

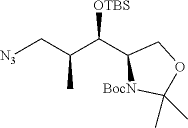

To a solution of (R)-tert-butyl 4-((1R,2S)-1-(tert-butyldimethylsilyloxy)-3-hydroxy-2-methylpropyl)-2,2-dimethyloxazolidine-3-carboxylate (1.0 equiv.), DIAD (2.0 equiv.), and PPh₃ (2.0 equiv.) in THF (0.18 M) was added DPPA (2.0 equiv., 1M solution in THF). The reaction mixture was stirred at room temperature overnight. Upon removal of the volatiles under vacuo, the residue was purified by silica gel column chromatography eluting with ethyl acetate and hexanes (1:6) to give (R)-tert-butyl 4-((1R,2S)-3-azido-1-(tert-butyldimethylsilyloxy)-2-methylpropyl)-2,2-dimethyloxazolidine-3-carboxylate as the major product (5:2) in 86% yield. LC/MS=329.3 (M+H-Boc), Rt=1.40 min (Frac 65%-95% method).

### Synthesis of tert-butyl (2R,3R,4S)-5-azido-3-(tert-butyldimethylsilyloxy)-1-hydroxy-4-methylpentan-2-ylcarbamate

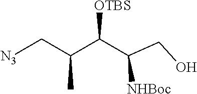

To a solution of (R)-tert-butyl 4-((1R,2S)-3-azido-1-(tert-butyldimethylsilyloxy)-2-methylpropyl)-2,2-dimethyloxazolidine-3-carboxylate (1.0 equiv.) in EtOH (0.1 M) was added PPTS (1.3 equiv.) and the mixture was refluxed for 2 days. The volatiles were removed under vacuo, the residue was dissolved in DCM (0.1 M) and DIEA (1.5 equiv.) and Boc₂O (1.0 equiv.) were added to the reaction mixture. The solution was stirred for 3 hours at room temperature. The solvents were removed under reduced pressure and the residue was diluted with ethyl acetate, washed with water, aqueous NaHSO₄, aqueous NaHCO₃, sat. NaCl, the organic phase was dried with magnesium sulfate, filtered, and concentrated. The residue was purified via silica gel column chromatography eluting with ethyl acetate and hexanes (1:3) to give tert-butyl (2R,3R,4S)-5-azido-3-(tert-butyldimethylsilyloxy)-1-hydroxy-4-methylpentan-2-ylcarbamate as the major isomer (5:2) in 70% yield. LC/MS=289.3 (M+H-Boc), Rt=0.76 min (Frac 65%-95% method).

### Synthesis of (2R,3R,4S)-5-azido-2-(tert-butoxycarbonylamino)-3-(tert-butyldimethylsilyloxy)-4-methylpentyl methanesulfonate

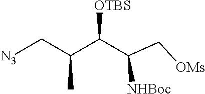

To a solution of tert-butyl (2R,3R,4S)-5-azido-3-(tert-butyldimethylsilyloxy)-1-hydroxy-4-methylpentan-2-ylcarbamate (1.0 equiv.) in pyridine (0.2 M) was added MsCl (1.3 equiv.) followed by DMAP (catalytic amount) at 0° C. The mixture was stirred at that temperature for 1 hour. The solution was diluted with ether and ethyl acetate (4:1), washed with aq. NaHSO₄, sat. NaHCO₃, brine, dried over magnesium sulfate, filtered, and concentrated. The residue was purified by silica gel column chromatography eluting with ethyl acetate and hexanes (1:3) to give (2R,3R,4S)-5-azido-2-(tert-butoxycarbonylamino)-3-(tert-butyldimethylsilyloxy)-4-methylpentyl methanesulfonate as the major isomer (5:2) in 90% yield. LC/MS=367.3 (M+H-Boc), Rt=0.81 min (Frac 65%-95% method).

### Synthesis of tert-butyl (3R,4R,5S)-4-(tert-butyldimethylsilyloxy)-5-methylpiperidin-3-ylcarbamate

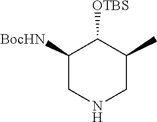

A solution of (2R,3R,4S)-5-azido-2-(tert-butoxycarbonylamino)-3-(tert-butyldimethylsilyloxy)-4-methylpentyl methanesulfonate in MeOH (0.09 M) was degassed with nitrogen for 20 min. DIEA (2.5 equiv.) was added, followed by 10% Pd/C (0.1 equiv.). The reaction mixture was stirred under a hydrogen balloon for 2 hours. The solution was filtered and the filtrate was concentrated under vacuo to afford tert-butyl (3R,4R,5S)-4-(tert-butyldimethylsilyloxy)-5-methylpiperidin-3-ylcarbamate as the major isomer (5:2) in >99% yield. LC/MS=345.2 (M+H-Boc), Rt=0.95 and 0.99 min.

### Synthesis of tert-butyl (3R,4R,5S)-4-(tert-butyldimethylsilyloxy)-5-methyl-1-(3-nitropyridin-4-yl)piperidin-3-ylcarbamate

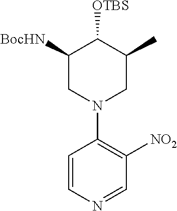

To a solution of tert-butyl (3R,4R,5S)-4-(tert-butyldimethylsilyloxy)-5-methylpiperidin-3-ylcarbamate (1.0 equiv.) in i-PrOH (0.09 M) was added DIEA (2.5 equiv.) and 4-chloro-3-nitropyridine (1.5 equiv.). The reaction mixture was stirred at 60° C. for 2 hours. The volatiles were removed under vacuo, the residue was diluted with ethyl acetate and washed with sat. NaCl. The organic phase was dried with magnesium sulfate, filtered, and concentrated. The crude material was purified by silica gel column chromatography eluting with ethyl acetate and hexanes (1:2) to give tert-butyl (3R,4R,5S)-4-(tert-butyldimethylsilyloxy)-5-methyl-1-(3-nitropyridin-4-yl)piperidin-3-ylcarbamate in 76% yield. LC/MS=467.3 (M+H), Rt=1.09 min.

### Synthesis of tert-butyl (3R,4R,5S)-1-(3-aminopyridin-4-yl)-4-(tert-butyldimethylsilyloxy)-5-methylpiperidin-3-ylcarbamate

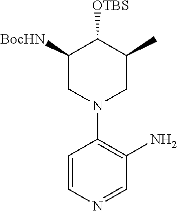

A solution of tert-butyl (3R,4R,5S)-4-(tert-butyldimethylsilyloxy)-5-methyl-1-(3-nitropyridin-4-yl)piperidin-3-ylcarbamate (1.0 equiv.) in MeOH (0.05 M) was degassed with nitrogen for 20 min. 10% Pd/C (0.2 equiv.) was added to the mixture and the solution was stirred under a hydrogen balloon for 3 hours. The reaction was filtered and the filtrate was concentrated under reduced pressure to give tert-butyl (3R,4R,5S)-1-(3-aminopyridin-4-yl)-4-(tert-butyldimethylsilyloxy)-5-methylpiperidin-3-ylcarbamate as the desired product in 94% yield. LC/MS=437.4 (M+H), Rt=1.08 min. ¹H-NMR (300 MHz, CDCl₃): δ 8.01 (s, 1H), 7.95 (d, J=6.0 Hz, 1H), 6.76 (d, J=6.0 Hz, 1H), 4.44 (br s, 1H), 3.74 (br s, 2H), 3.59-3.55 (m, 1H), 3.25-3.13 (m, 2H), 2.47-2.35 (m, 2H), 1.89 (br s, 2H), 1.44 (s, 9H), 1.04 (d, J=6.0, 3H), 0.92 (s, 9H), 0.13 (d, J=9.0, 6H).

### Synthesis of (+/−)-5-isopropyl-3-oxocyclohex-1-en-1-yl trifluoromethanesulfonate

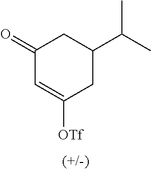

To a 0.39 M solution of (+/−)-5-isopropylcyclohexane-1,3-dione (1.0 equiv.) in DCM under an atmosphere of nitrogen and cooled in an ice water bath was added sodium carbonate (1.1 equiv.). To the solution was added trifluoromethanesulfonic anhydride (1.05 equiv.) in DCM dropwise via an addition funnel at 0° C. over 20 min. After the complete addition, the reaction was stirred at 0° C. for 20 min, then allowed to warm to room temperature and stirred for 1 hour. The solution was then quenched by the addition of saturated aqueous sodium bicarbonate. The organic phase was filtered through a pad of Celite (due to emulsions), then it was dried with sodium sulfate, filtered and concentrated under vacuo. The crude material was used for the next step without further purification. Isolated (+/−)-5-isopropyl-3-oxocyclohex-1-en-1-yltrifluoromethanesulfonate in 73% yield as an orange oil. ¹H NMR (400 MHz, <cdcl3>) δ ppm 0.96 (s, 3H), 0.98 (s, 3H), 1.68 (dq, J=13.3, 6.7, 1H), 1.92-2.10 (m, 1H), 2.11-2.25 (m, 1H), 2.45-2.67 (m, 3H), 6.06 (d, J=2, 1H)

### Synthesis of (+/−)-5-isopropyl-3-(3-nitropyridin-4-yl)cyclohex-2-enone

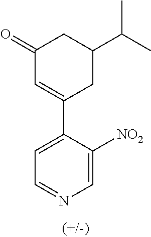

To a 0.29 M solution of (+/−)-5-isopropyl-3-oxocyclohex-1-en-1-yl trifluoromethanesulfonate (1.0 equiv.) in dioxane was added potassium acetate (3.0 equiv.) and bis(pinacolato)diboron (2.0 equiv.). The solution was degassed with nitrogen and PdCl₂(dppf)-DCM (0.03 equiv.) was added. The reaction was heated to 80° C. overnight. LC/MS of the reaction upon cooling indicated complete conversion to product (MH+=183 for the boronic acid, Rt=0.56 min). The reaction was filtered through a coarse frit glass funnel and further washed with dioxane. The filtrate solution was used for the next step without further purification. To this dioxane solution was added 4-chloro-3-nitropyridine (1.3 equiv.), 2M sodium carbonate solution (4.0 equiv.) and PdCl₂(dppf)-DCM (0.05 equiv.). The reaction was heated to 110° C. for one hour. Upon cooling to room temperature, the reaction was complete as indicated by LC/MS. Partitioned between water and ethyl acetate, the aqueous phase was extracted three more times with ethyl acetate. The organics were combined, dried with sodium sulfate, filtered and concentrated under vacuo. The crude material was purified via silica gel chromatography eluting with ethyl acetate and heptanes (0-50% ethyl acetate). The pure fractions were concentrated to give (+/−)-5-isopropyl-3-(3-nitropyridin-4-yl)cyclohex-2-enone as the desired product in 83% yield. LC/MS (m/z): 261.0 (MH⁺), Rₜ=0.83 min. ¹H NMR (400 MHz, <cdcl3>) δ ppm 0.96 (dd, J=6.65, 3.52 Hz, 6H) 1.67 (dt, J=13.01, 6.60 Hz, 1H) 2.13-2.31 (m, 2H) 2.43 (d, J=6.26 Hz, 2H) 2.64 (d, J=13.30 Hz, 1H) 6.00 (s, 1H) 7.27 (br. s., 1H) 8.87 (d, J=5.09 Hz, 1H) 9.32 (s, 1H).

### Synthesis of (+/−)—N-benzyl-5-isopropyl-3-(3-nitropyridin-4-yl)cyclohex-2-enamine

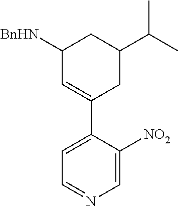

To a solution of (+/−)-5-isopropyl-3-(3-nitropyridin-4-yl)cyclohex-2-enone (1.0 equiv.) in methanol (0.19 M) was added benzyl amine (1.5 equiv.) and the reaction was stirred at rt for 4 h. Cooled to −78° C., then lithium borohydride (2M solution in THF, 1.1 equiv.) was added dropwise. Allowed the reaction to warm to rt over 30 min and stirred at rt for 30 min. The solution was partitioned between water and ethyl acetate. The organic phase was dried with sodium sulfate, filtered and concentrated under vacuo. The crude material was purified via silica gel chromatography eluting with ethyl acetate and heptanes (0-100% ethyl acetate). The pure fractions were concentrated under vacuo to give (+/−)—N-benzyl-5-isopropyl-3-(3-nitropyridin-4-yl)cyclohex-2-enamine in 30% yield. LC/MS (m/z): 352.1 (MH⁺), Rₜ=0.77 min.

### Synthesis of (+/−)-tert-butyl benzyl(5-isopropyl-3-(3-nitropyridin-4-yl)cyclohex-2-en-1-yl)carbamate

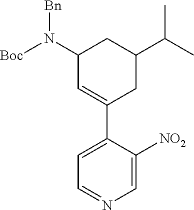

To a solution of (+/−)-N-benzyl-5-isopropyl-3-(3-nitropyridin-4-yl)cyclohex-2-enamine (1.0 equiv.) in DCM (0.1 M) was added triethylamine (2.0 equiv.) followed by Boc₂O (1.1 equiv.). The solution was stirred at rt overnight. The reaction was partitioned between water and DCM, the organic phase was dried with sodium sulfate, filtered and concentrated under vacuo. The crude material was purified via silica gel column chromatography eluting with ethyl acetate and heptanes (0-50% ethyl acetate). The pure fractions were concentrated to give (+/−)-tert-butyl benzyl(5-isopropyl-3-(3-nitropyridin-4-yl)cyclohex-2-en-1-yl)carbamate as a yellow oil in 70% yield. LC/MS (m/z): 452.2 (MH⁺), Rₜ=1.30 min.

### Synthesis of tert-butyl ((1R,3S,5R)-3-(3-aminopyridin-4-yl)-5-isopropylcyclohexyl)carbamate and tert-butyl ((1S,3R,5S)-3-(3-aminopyridin-4-yl)-5-isopropylcyclohexyl)carbamate

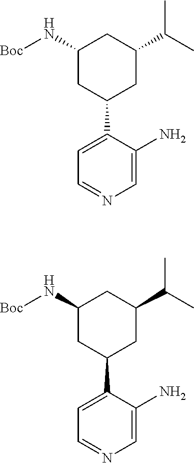

To a degassed solution of (+/−)-tert-butyl benzyl(5-isopropyl-3-(3-nitropyridin-4-yl)cyclohex-2-en-1-yl)carbamate (1.0 equiv.) in ethanol (0.1 M) was added palladium hydroxide (0.2 equiv) and the reaction was stirred at rt under a hydrogen balloon for 4 hours. After 4 h, LC/MS indicated double bond and nitro reduction. The solution was filtered through a pad of Celite and washed with ethanol. To this filtrate solution was added HCl (12 M, 5 equiv.) and a fresh batch of palladium hydroxide (0.2 equiv.) and degassed. The reaction was stirred under a hydrogen balloon for another 4 hours until completion of the benzyl deprotection. It was then filtered through a pad of Celite and washed with ethyl acetate. The filtrate was further neutralized with sodium bicarbonate then concentrated under vacuo. The crude was partitioned between water and ethyl acetate, the organic phase was dried with sodium sulfate, filtered and concentrated under vacuo. The crude material was purified via silica gel chromatography eluting with DCM/MeOH/NH₄OH (95:5:0.5). The pure fractions were concentrated to yield (+/−)-tert-butyl (3-(3-aminopyridin-4-yl)-5-isopropylcyclohexyl)carbamate in 73% yield. LC/MS (m/z): 334.2 (MH⁺), Rₜ=0.76 min. This compound was further purified via chiral HPLC (IC column, heptanes:ethanol, 95:5) to yield Peak 1: tert-butyl ((1R,3S,5R)-3-(3-aminopyridin-4-yl)-5-isopropylcyclohexyl)carbamate (15.626 min, >99% ee) and Peak 2: tert-butyl ((1S,3R,5S)-3-(3-aminopyridin-4-yl)-5-isopropylcyclohexyl)carbamate (18.635 min, >99% ee) LC/MS (m/z): 334.2 (MH⁺), Rₜ=0.76 min.

### Synthesis of 6-bromo-5-fluoropicolinic acid

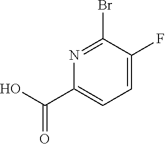

To 2-bromo-3-fluoro-6-methylpyridine (1.0 equiv.) in H₂O (30 mL) was added potassium permanganate (1.0 equiv.). The solution was heated at 100° C. for 5 hours at which time more potassium permanganate (1.0 equiv.) was added. After heating for an additional 48 hours the material was filtered through celite (4 cm×2 inches) and rinsed with H₂O (150 mL). The combined aqueous was acidified with 1N HCl to pH=4, extracted with ethyl acetate (200 mL), washed with NaCl(sat.), dried over MgSO₄, filtered and concentrated to yield 6-bromo-5-fluoropicolinic acid (17%) as a white solid. LCMS (m/z): 221.9 (MH+); LC Rt=2.05 min.

### Synthesis of methyl 6-bromo-5-fluoropicolinate

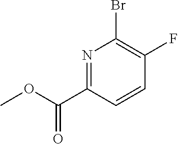

To a solution of 6-bromo-5-fluoropicolinic acid (1.0 equiv.) in methanol (0.2 M) was added H₂SO₄ (4.2 equiv.) and the reaction was stirred at room temperature for two hours. Upon completion of the reaction as monitored by LC/MS, the reaction was diluted with ethyl acetate and quenched slowly with saturated aqueous NaHCO₃. The reaction was poured into a separatory funnel and extracted with ethyl acetate. The organic phase was dried with magnesium sulfate, filtered, and concentrated in vacuo to provide methyl 6-bromo-5-fluoropicolinate as a white solid (>99%). LC/MS=233.9/235.9 (M+H), Rt=0.69 min.

### Method 1

### Synthesis of methyl 6-(3-(benzyloxy)-2,6-difluorophenyl)-5-fluoropicolinate

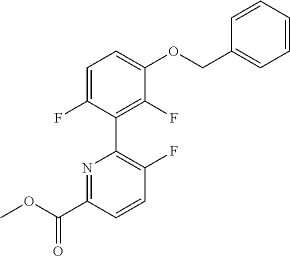

To a solution of methyl 6-bromo-5-fluoropicolinate (1.0 equiv.) in THF and water (10:1, 0.1 M) was added 3-(benzyloxy)-2,6-difluorophenylboronic acid (2.5 equiv.) and potassium fluoride (3.3 equiv.). The reaction was degassed with nitrogen, then Pd₂(dba)₃ (0.25 equiv.) and tri-tert-butylphosphine (0.5 equiv.) were added and the reaction was heated to 80° C. for one hour. LC/MS analysis indicated complete conversion of the starting material to product. The reaction was cooled to room temperature, then concentrated in vacuo and fused to silica gel. The crude product was purified by ISCO flash chromatography eluting with ethyl acetate and hexanes (0% to 30% ethyl acetate) to provide methyl 6-(3-(benzyloxy)-2,6-difluorophenyl)-5-fluoropicolinate as the desired product as a light yellow oil in 96% yield. LC/MS=374.0 (M+H), Rt=1.07 min.

### Synthesis of methyl 6-(2,6-difluoro-4-methoxyphenyl)-5-fluoropicolinate

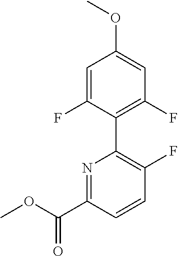

Method 1 was followed using 6-bromo-5-fluoropicolinate (1.0 equiv.) and 2,6-difluoro-4-methoxyphenylboronic acid (2.5 equiv.) to give methyl 6-(2,6-difluoro-4-methoxyphenyl)-5-fluoropicolinate as a white solid in 85% yield. LC/MS=298.0 (M+H), Rt=0.89 min.

### Method 2

### Synthesis of 6-(2,6-difluoro-4-methoxyphenyl)-5-fluoropicolinic acid

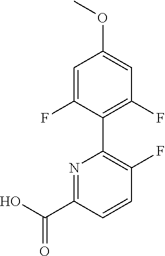

To a solution of methyl 6-(2,6-difluoro-4-methoxyphenyl)-5-fluoropicolinate (1.0 equiv.) in THF/MeOH (2:1, 0.09 M) was added LiOH (1.5 equiv.) and the reaction was stirred at room temperature for 1 hour. The solution was quenched with 1N HCl, extracted with ethyl acetate, washed with brine, dried with sodium sulfate, filtered and concentrated to give 6-(2,6-difluoro-4-methoxyphenyl)-5-fluoropicolinic acid in 84% yield. LC/MS=284.1 (M+H), Rt=0.76 min.

### Method 3

### Synthesis of 2-(2,6-difluoro-4-methylphenyl)-4,4,5,5-tetramethyl-1,3,2-dioxaboroane

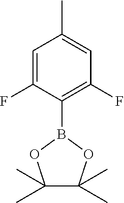

To a solution of 1,3-difluoro-5-methylbenzene (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield 2-(2,6-difluoro-4-methylphenyl)-4,4,5,5-tetramethyl-1,3,2-dioxaboroane as a white solid in 92%. 1H NMR (400 MHz, <cdcl3>) δ ppm 6.67 (dd, J=9.39, 0.78 Hz, 2H), 2.34 (s, 3H), 1.38 (s, 12H).

### Synthesis of 6-(2,6-difluoro-4-methylphenyl)-5-fluoropicolinate

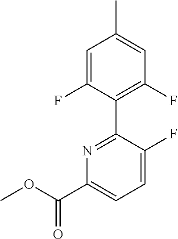

Method 1 was followed using 6-bromo-5-fluoropicolinate (1.0 equiv.) and 2-(2,6-difluoro-4-methylphenyl)-4,4,5,5-tetramethyl-1,3,2-dioxaboroane (1.75 equiv.) to give methyl 6-(2,6-difluoro-4-methylphenyl)-5-fluoropicolinate as a solid in 85% yield. LC/MS=282.0 (M+H), Rt=0.87 min.

### Synthesis of 6-(2,6-difluoro-4-methylphenyl)-5-fluoropicolinic acid

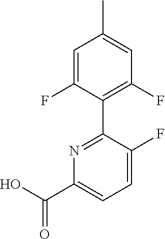

To a solution of 6-(2,6-difluoro-4-methylphenyl)-5-fluoropicolinate (1.0 eq) in THF (0.1M) was added LiOH (5.5 eq, 2M) and allowed to stir at room temperature for 4 hrs. The volatiles were removed in vacuo, and the residual aqueous was acidified with 2M HCl to pH 4. The precipitate was filtered and dried to yield 6-(2,6-difluoro-4-methylphenyl)-5-fluoropicolinic acid as al light yellow solid in 73.5%. LCMS (m/z): 268.0 (MH⁺), Rₜ=0.76 min.

### Synthesis of methyl 6-(2,6-difluoro-4-formylphenyl)-5-fluoropicolinate

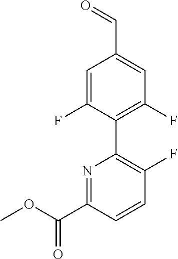

Method 1 was followed using 6-bromo-5-fluoropicolinate (1.0 equiv.) and 3,5-difluoro-4-(4,4,5,5-tetramethyl-1,3,2-dioxaborolan-2-yl)benzaldehyde (1.8 equiv.) to give methyl 6-(2,6-difluoro-4-formylphenyl)-5-fluoropicolinate as an off-white solid in 66% yield. LC/MS=295.9 (M+H), Rt=0.73 min.

### Synthesis of methyl 6-(2,6-difluoro-4-vinylphenyl)-5-fluoropicolinate

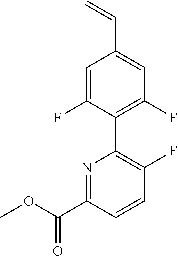

To a solution of Methyltriphenylphosphonium bromide (1.5 equiv) in THF (0.1 M) was added potassium tert-butoxide (1.45 eq.) After stirring at rt for 2 hours the solution was cooled to −78° C. and a solution of methyl 6-(2,6-difluoro-4-formylphenyl)-5-fluoropicolinate (1.0 eq.) in THF was added dropwise. The solution was stirred for 16 hours as the temperature gradually warmed to rt. The solution was partitioned between EtOAc and water, washed with NaHCO_{3(sat.)}, NaCl_{(sat.)}, dried over MgSO₄, filtered, concentrated and purified by ISCO SiO₂ chromatography to yield methyl 6-(2,6-difluoro-4-vinylphenyl)-5-fluoropicolinate as a white solid in 63% yield. LC/MS=293.9 (M+H), Rₜ=0.90 min.

### Synthesis of 6-(2,6-difluoro-4-vinylphenyl)-5-fluoropicolinic acid

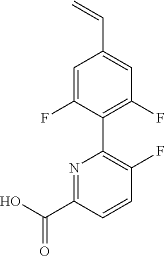

Method 2 was followed using methyl 6-(2,6-difluoro-4-vinylphenyl)-5-fluoropicolinate to give 6-(2,6-difluoro-4-vinylphenyl)-5-fluoropicolinic acid in 94% yield. LC/MS=279.9 (M+H), Rₜ=0.78 min.

### Synthesis of methyl 6-(2,6-difluoro-4-(hydroxymethyl)phenyl)-5-fluoropicolinate

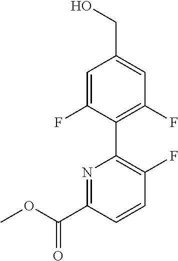

To a solution of methyl 6-(2,6-difluoro-4-formylphenyl)-5-fluoropicolinate (1.0 eq.) in THF (0.24 M) at 0° C. was added sodium borohydride. After stirring for 10 minutes, water was added and the solution was extracted with EtOAc, washed with NaCl(sat.), dried over Na₂SO₄, filtered and concentrated to yield methyl 6-(2,6-difluoro-4-(hydroxymethyl)phenyl)-5-fluoropicolinate in 87% yield. LC/MS=297.9 (M+H), Rₜ=0.66 min.

### Synthesis of methyl 6-(2,6-difluoro-4-(methoxymethyl)phenyl)-5-fluoropicolinate

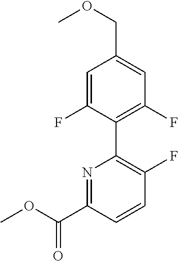

To a solution of methyl 6-(2,6-difluoro-4-(hydroxymethyl)phenyl)-5-fluoropicolinate (1.0 eq.) in DMF (0.03 M) at 0° C. was added sodium hydride (1.5 eq). After stirring for 2 minutes, methyl iodide (1.5 eq.) was added. After stirring for 1 hour, water was added and the solution was extracted with EtOAc (3×), the combined organics were dried over Na₂SO₄, filtered, concentrated and purified by ISCO SiO₂ chromatography (eluting with 0-20% EtOAc/n-heptanes) to yield methyl 6-(2,6-difluoro-4-(methoxymethyl)phenyl)-5-fluoropicolinate in 29% yield. LC/MS=312.0 (M+H), Rₜ=0.80 min.

### Synthesis of 6-(2,6-difluoro-4-(methoxymethyl)phenyl)-5-fluoropicolinic acid

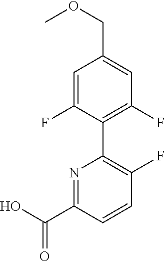

Method 2 was followed using methyl 6-(2,6-difluoro-4-(methoxymethyl)phenyl)-5-fluoropicolinate to give 6-(2,6-difluoro-4-(methoxymethyl)phenyl)-5-fluoropicolinic acid in 84% yield. LC/MS=297.9 (M+H), Rₜ=0.70 min.

### Synthesis of 2-(2,6-difluoro-4-(methylthio)phenyl)-4,4,5,5-tetramethyl-1,3,2-dioxaborolane

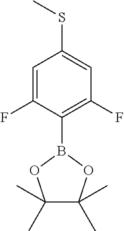

To a solution of (3,5-difluorophenyl)(methyl)sulfane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield a 2-(2,6-difluoro-4-(methylthio)phenyl)-4,4,5,5-tetramethyl-1,3,2-dioxaborolane in 91%. 1H NMR (400 MHz, <cdcl3>) δ ppm 6.71 (dd, 2H), 2.48 (s, 3H), 1.37 (s, 12H).

### Synthesis of methyl 6-(2,6-difluoro-4-(methylthio)phenyl)-5-fluoropicolinate

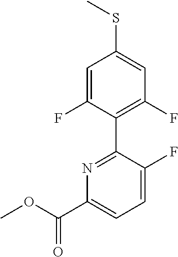

Method 1 was followed using 6-bromo-5-fluoropicolinate (1.0 equiv.) and 2-(2,6-difluoro-4-(methylthio)phenyl)-4,4,5,5-tetramethyl-1,3,2-dioxaborolane (1.75 equiv.) to give methyl 6-(2,6-difluoro-4-(methylthio)phenyl)-5-fluoropicolinate in 73% yield. LC/MS=313.9 (M+H), Rt=0.90 min.

### Synthesis of 6-(2,6-difluoro-4-(methylthio)phenyl)-5-fluoropicolinic acid

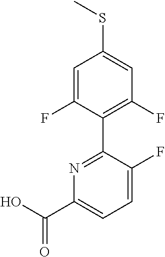

To a solution of 6-(2,6-difluoro-4-(methylthio)phenyl)-5-fluoropicolinate (1.0 eq) in THF (0.2 M) was added LiOH (5.5 eq, 2M) and allowed to stir at rt for 3 hrs. The volatiles were removed in vacuo, and the residual aqueous was acidified with 2M HCl to pH 4. The precipitate was filtered and dried to yield 6-(2,6-difluoro-4-(methylthio)phenyl)-5-fluoropicolinic acid as a solid in 92% yield. LCMS (m/z): 299.9 (MH⁺), Rₜ=0.78 min.

### Synthesis of methyl 6-(2,6-difluoro-4-(methylsulfinyl)phenyl)-5-fluoropicolinate

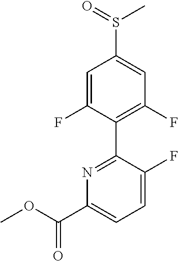

To a solution of 6-(2,6-difluoro-4-(methylthio)phenyl)-5-fluoropicolinate (1.0 eq) in CH₂Cl₂ (0.03 M) was added oxone (2.0 eq). After stirring for 96 hours at rt, the solution was partitioned between EtOAc and water, washed further with NaCl_{(sat.)}, dried over MgSO₄, filtered, concentrated and purified by ISCO SiO₂ chromatography to yield methyl 6-(2,6-difluoro-4-(methylsulfinyl)phenyl)-5-fluoropicolinate as a solid in 60% yield. LCMS (m/z): 329.9 (MH⁺) Rₜ=0.62 min. Additionally, methyl 6-(2,6-difluoro-4-(methylsulfonyl)phenyl)-5-fluoropicolinate was obtained as a solid in 18%. LCMS (m/z): 345.9 (MH⁺) Rₜ=0.69 min.

### Synthesis of 6-(2,6-difluoro-4-(methylsulfinyl)phenyl)-5-fluoropicolinic acid

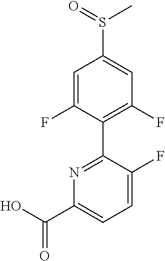

To a solution of methyl 6-(2,6-difluoro-4-(methylsulfinyl)phenyl)-5-fluoropicolinate (1.0 eq) in 2:1 THF/MeOH (0.13 M) was added LiOH (1.2 eq, 1M). After stirring for 16 hours at rt, the solution was neutralized by addition of 1N HCl (1.2 eq.) and the volatiles were removed in vacuo. The residue was partitioned between EtOAc and NaCl_{(sat.)}, mixed, separated, dried over MgSO₄ filtered and concentrated to yield 6-(2,6-difluoro-4-(methylsulfinyl)phenyl)-5-fluoropicolinic acid as a solid in 94% yield. LCMS (m/z): 315.9 (MH⁺) Rₜ=0.53 min.

### Synthesis of methyl 6-(2,6-difluoro-4-(methylsulfonyl)phenyl)-5-fluoropicolinate

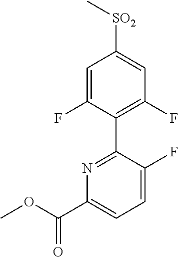

To a solution of methyl 6-(2,6-difluoro-4-(methylthio)phenyl)-5-fluoropicolinate (1.0 equiv) in CH₂Cl₂ (0.2 M) at 0° C. was added MCPBA (3.2 equiv.). After stirring for 40 minutes, the reaction was quenched with Na₂S₂O_{3(aq.)}, diluted with EtOAc, washed with NaHCO_{3(sat.)}, brine, dried over MgSO₄, filtered, concentrate, purified by SiO₂ chromatography to yield methyl 6-(2,6-difluoro-4-(methylsulfonyl)phenyl)-5-fluoropicolinate in 56% yield. LC/MS=345.9 (M+H), Rt=0.69 min.

### Synthesis of 6-(2,6-difluoro-4-(methylsulfonyl)phenyl)-5-fluoropicolinic acid

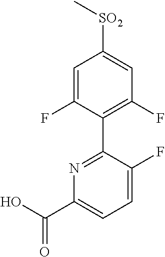

To a solution of 6-(2,6-difluoro-4-(methylsulfonyl)phenyl)-5-fluoropicolinate (1.0 eq) in THF (0.1M) was added LiOH (5.5 eq, 2M) and allowed to stir at 37° C. for 2 hrs. The volatiles were removed in vacuo, and the residual aqueous was acidified with 2M HCl to pH 4. The precipitate was filtered and dried to yield 6-(2,6-difluoro-4-(methylsulfonyl)phenyl)-5-fluoropicolinic acid as a solid in 91% yield. LCMS (m/z): 331.8 (MH⁺) Rₜ=0.59 min.

### Synthesis of (2-(3,5-difluorophenyl)propan-2-yloxy)triisopropylsilane

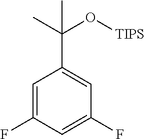

To a solution of 1-(3,5-difluorophenyl)ethanone (1.0 equiv) in THF (0.2 M) at 0° C. was added methylmagnesium bromide (1.0 M in THF, 1.15 equiv). After stirring for 4 hours the reaction was quenched by addition of NH₄Cl_{(sat.)}, diluted with EtOAc, washed with NaCl_{(sat.)}, dried over MgSO₄, filtered, concentrated and purified by ISCO SiO₂ chromatography to yield 2-(3,5-difluorophenyl)propan-2-ol. To a solution of 2-(3,5-difluorophenyl)propan-2-ol in CH₂Cl₂ (0.1 M) at 0° C. was added 2,6 lutidine (6 equiv.) and than triisopropylsilyl trifluoromethanesulfonate (3.0 equiv.). After stirring for 3 hours at 0° C. and six hours at rt the solution was partitioned between EtOAc and NaHCO_{3(sat.)}, separated, washed with NaCl_{(sat.)}, dried over MgSO₄, filtered, concentrated and purified by ISCO SiO₂ chromatography to yield (2-(3,5-difluorophenyl)propan-2-yloxy)triisopropylsilane. (400 MHz, <cdcl3>) δ ppm 1.05-1.08 (m, 21H) 1.57 (s, 6H) 6.63 (s, 1H) 7.00 (dd, J=9.39, 2.35 Hz, 2H). Synthesis of (2-(3,5-difluoro-4-(4,4,5,5-tetramethyl-1,3,2-dioxaborolan-2-yl)phenyl)propan-2-yloxy)triisopropylsilane

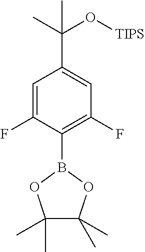

To a solution of (2-(3,5-difluorophenyl)propan-2-yloxy)triisopropylsilane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield (2-(3,5-difluoro-4-(4,4,5,5-tetramethyl-1,3,2-dioxaborolan-2-yl)phenyl)propan-2-yloxy)triisopropylsilane in 99%. ¹H NMR (400 MHz, <cdcl3>) δ ppm 1.03-1.08 (m, 21H) 1.24 (s, 12H) 1.38 (s, 3H) 1.57 (s, 3H) 6.92-7.03 (m, 2H).

### Synthesis of tert-butyl(3,5-difluorophenoxy)dimethylsilane

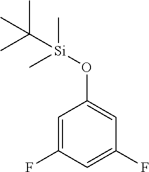

To a solution of 3,5-difluorophenol (1.0 equiv.) and imidazole (2.2 equiv.) in DMF (0.8 M) at 0° C. was added TBDMSCl (1.1 equiv.). The ice bath was removed and after stirring for 3 hours the solution was diluted with EtOAc, washed with water, brine, dried over MgSO₄, filtered, concentrated and purified by SiO₂ chromatography to yield tert-butyl(3,5-difluorophenoxy)dimethylsilane in 73% yield. ¹H NMR (400 MHz, <cdcl3>) δ ppm 0.23 (s, 6 H) 0.99 (s, 9H) 6.33-6.40 (m, 2H) 6.44 (tt 1H).

### Synthesis of tert-butyl(3,5-difluoro-4-(4,4,5,5-tetramethyl-1,3,2-dioxaborolan-2-yl)phenoxy)dimethylsilane

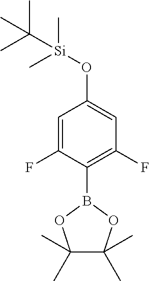

To a solution of tert-butyl(3,5-difluorophenoxy)dimethylsilane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 1 hr at −78° C., followed by the addition of 2-isopropoxy-4,4,5,5-tetramethyl-1,3,2-dioxaborolane (2.1 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield tert-butyl(3,5-difluoro-4-(4,4,5,5-tetramethyl-1,3,2-dioxaborolan-2-yl)phenoxy)dimethylsilane in 91% yield. ¹H NMR (400 MHz, <cdcl3>) δ ppm 0.21 (s, 6H) 0.97 (s, 9H) 1.37 (s, 12H) 6.33 (d, J=9.39 Hz, 2H).

### Synthesis of methyl 6-(2,6-difluoro-4-hydroxyphenyl)-5-fluoropicolinate

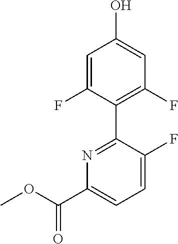

Method 1 was followed using 6-bromo-5-fluoropicolinate (1.0 equiv.) and tert-butyl(3,5-difluoro-4-(4,4,5,5-tetramethyl-1,3,2-dioxaborolan-2-yl)phenoxy)dimethylsilane (1.75 equiv.) to give methyl 6-(2,6-difluoro-4-hydroxyphenyl)-5-fluoropicolinate in 65% yield. The reaction was heated for an additional 30 minutes at 100° C. in the microwave to drive to completion the deprotection of the TBDMS group. LC/MS=283.9 (M+H), Rt=0.69 min.

### Synthesis of methyl 6-(4-(2-(tert-butyldimethylsilyloxy)ethoxy)-2,6-difluorophenyl)-5-fluoropicolinate

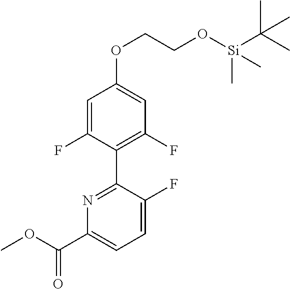

To a solution of methyl 6-(2,6-difluoro-4-hydroxyphenyl)-5-fluoropicolinate (1.0 equiv.) and potassium carbonate (4.0 equiv.) in DMF (0.4 M) was added (2-bromoethoxy)(tert-butyl)dimethylsilane (2 equiv.). After stirring for 72 hours at rt the heterogeneous solution was diluted with water, extracted with EtOAc, dried over MgSO₄, filtered, concentrated and purified by ISCO SiO₂ chromatography to yield methyl 6-(4-(2-(tert-butyldimethylsilyloxy)ethoxy)-2,6-difluorophenyl)-5-fluoropicolinate in 74% yield. LC/MS=442.1 (M+H), Rₜ=1.22 min.

### Synthesis of 6-(4-(2-(tert-butyldimethylsilyloxy)ethoxy)-2,6-difluorophenyl)-5-fluoropicolinic acid

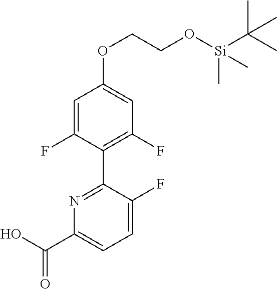

Method 2 was followed using methyl 6-(4-(2-(tert-butyldimethylsilyloxy)ethoxy)-2,6-difluorophenyl)-5-fluoropicolinate to give 6-(4-(2-(tert-butyldimethylsilyloxy)ethoxy)-2,6-difluorophenyl)-5-fluoropicolinic acid in 94% yield. LC/MS=428.1 (M+H), Rₜ=1.13 min.

### Synthesis of methyl 6-(4-ethoxy-2,6-difluorophenyl)-5-fluoropicolinate

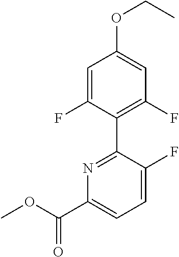

To a solution of methyl 6-(2,6-difluoro-4-hydroxyphenyl)-5-fluoropicolinate (1.0 equiv.), ethanol (3.0 eq.) and triphenylphosphine (3.0 eq.) in THF (0.18 M) at 0° C. was added diisopropyl azodicarboxylate (3.0 eq.) After stirring for 16 hours at rt as the solution slowly warmed to rt, the volatiles were removed in vacuo and the residue was purified by ISCO SiO₂ chromatography to yield methyl 6-(4-ethoxy-2,6-difluorophenyl)-5-fluoropicolinate in 99% yield. LC/MS=311.9 (M+H), Rₜ=0.91 min.

### Synthesis of 6-(4-ethoxy-2,6-difluorophenyl)-5-fluoropicolinic acid

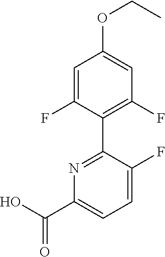

Method 2 was followed using methyl 6-(4-ethoxy-2,6-difluorophenyl)-5-fluoropicolinate to give 6-(4-ethoxy-2,6-difluorophenyl)-5-fluoropicolinic acid in 38% yield. LC/MS=297.9 (M+H), Rₜ=0.80 min.

### Synthesis of 1,3-difluoro-5-(2-methoxyethoxy)benzene

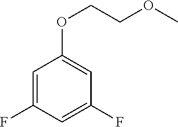

To a solution of 3,5-difluorophenol (1.0 equiv.), 2-methoxyethanol (3.0 equiv.) and triphenylphosphine (3.0 equiv) in THF (0.1 M) was added DIAD (3.0 equiv.). After stirring at rt for 18 hours, the volatiles were removed in vacuo and the residue was purified by SiO₂ chromatography to yield 1,3-difluoro-5-(2-methoxyethoxy)benzene in 95% yield. ¹H NMR (400 MHz, <cdcl3>) δ ppm 6.41-6.47 m (3H), 4.08 (t, 2H), 3.74 (t, 2H), 3.45 (s, 3H).

### Synthesis of 2-(2,6-difluoro-4-(2-methoxyethoxy)phenyl)-4,4,5,5-tetramethyl-1,3,2-dioxaborolane

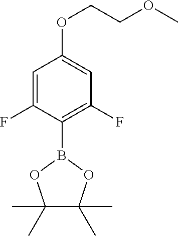

To a solution of 1,3-difluoro-5-(2-methoxyethoxy)benzene (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 1 hr at −78° C., followed by the addition of 2-isopropoxy-4,4,5,5-tetramethyl-1,3,2-dioxaborolane (2.1 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield 2-(2,6-difluoro-4-(2-methoxyethoxy)phenyl)-4,4,5,5-tetramethyl-1,3,2-dioxaborolane. ¹H NMR (400 MHz, <cdcl3>) δ ppm 6.42 (d, 2H), 4.10 (m, 2H), 3.74 (m, 2H), 3.44 (s, 3H), 1.37 (s, 12H).

### Synthesis of methyl 6-(2,6-difluoro-4-(2-methoxyethoxy)phenyl)-5-fluoropicolinate

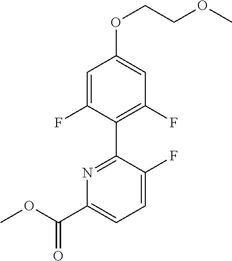

Method 1 was followed using methyl 6-bromo-5-fluoropicolinate (1.0 equiv.) and 2-(2,6-difluoro-4-(2-methoxyethoxy)phenyl)-4,4,5,5-tetramethyl-1,3,2-dioxaborolane (1.75 equiv.) at 80° C. for 1 hour to give methyl 6-(2,6-difluoro-4-(2-methoxyethoxy)phenyl)-5-fluoropicolinate in 95% yield. LC/MS=341.9 (M+H), Rₜ=0.89 min.

### Synthesis of 6-(2,6-difluoro-4-(2-methoxyethoxy)phenyl)-5-fluoropicolinic acid

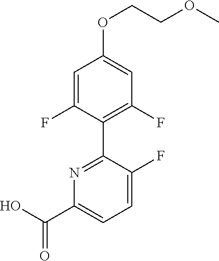

Method 2 was followed using methyl 6-(2,6-difluoro-4-(2-methoxyethoxy)phenyl)-5-fluoropicolinate to give 6-(2,6-difluoro-4-(2-methoxyethoxy)phenyl)-5-fluoropicolinic acid in 98% yield. LC/MS=327.9 (M+H), Rₜ=0.71 min.

### Method 4

### Synthesis of methyl 6-(2,6-difluoro-3-hydroxyphenyl)-5-fluoropicolinate

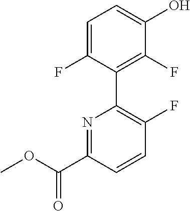

To a solution of methyl 6-(3-(benzyloxy)-2,6-difluorophenyl)-5-fluoropicolinate (1.0 equiv.) in methanol (0.1 M) was added 10% Pd/C (0.1 equiv.) in ethyl acetate. The reaction was placed under an atmosphere of hydrogen and stirred for 2 hours. Upon completion, the solution was filtered over a pad of Celite, the pad was washed with methanol, the filtrate was concentrated in vacuo to give methyl 6-(2,6-difluoro-3-hydroxyphenyl)-5-fluoropicolinate as a grey oil in 86% yield. LC/MS=284.0 (M+H), Rt=0.90 min.

### Synthesis of methyl 6-(3-(((S)-2,2-dimethyl-1,3-dioxolan-4-yl)methoxy)-2,6-difluorophenyl)-5-fluoropicolinate

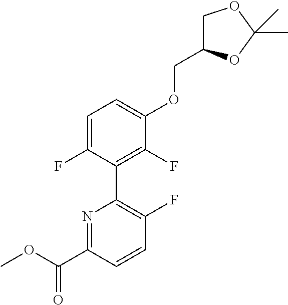

To a solution of methyl 6-(2,6-difluoro-3-hydroxyphenyl)-5-fluoropicolinate (1.0 equiv.) and (R)-(2,2-dimethyl-1,3-dioxolan-4-yl)methanol (2.0 equiv.) in THF (0.1 M) at 0° C. was added triphenylphosphine (2 equiv.) and (E)-di-tert-butyl diazene-1,2-dicarboxylate (2 equiv.). After stirring for 12 hours as the solution warmed to rt, the volatiles were removed in vacuo and upon SiO₂ purification, methyl 6-(3-((S)-2,2-dimethyl-1,3-dioxolan-4-yl)methoxy)-2,6-difluorophenyl)-5-fluoropicolinate was obtained in 96% yield. LC/MS=398.0 (M+H), Rt=0.91 min.

### Synthesis of 6-(3-((S)-2,2-dimethyl-1,3-dioxolan-4-yl)methoxy)-2,6-difluorophenyl)-5-fluoropicolinic acid

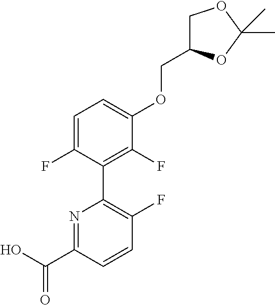

To a solution of methyl 6-(3-(((S)-2,2-dimethyl-1,3-dioxolan-4-yl)methoxy)-2,6-difluorophenyl)-5-fluoropicolinate in THF/EtOH/H₂O (2:2:1) at 0.1 M concentration was added LiOH (5 equiv.). Upon stirring for 2 hours, the pH was adjusted to pH4 by addition of 1N HCl, the solution was extracted with EtOAc, dried over MgSO₄, filtered and concentrated to yield 6-(3-(((S)-2,2-dimethyl-1,3-dioxolan-4-yl)methoxy)-2,6-difluorophenyl)-5-fluoropicolinic acid in 99% yield, LC/MS=384.0 (M+H), Rt=0.82 min.

### Synthesis of 6-(3-(((R)-2,2-dimethyl-1,3-dioxolan-4-yl)methoxy)-2,6-difluorophenyl)-5-fluoropicolinic acid

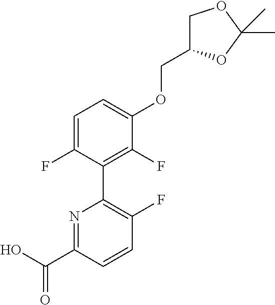

Using (S)-(2,2-dimethyl-1,3-dioxolan-4-yl)methanol and methyl 6-(2,6-difluoro-3-hydroxyphenyl)-5-fluoropicolinate and following the described procedure for preparation of 6-(3-(((S)-2,2-dimethyl-1,3-dioxolan-4-yl)methoxy)-2,6-difluorophenyl)-5-fluoropicolinic acid, 6-(3-(((R)-2,2-dimethyl-1,3-dioxolan-4-yl)methoxy)-2,6-difluorophenyl)-5-fluoropicolinic acid was prepared.

### Synthesis of methyl 6-(2,6-difluoro-3-(2-methoxyethoxy)phenyl)-5-fluoropicolinate

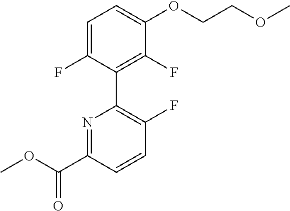

To a solution of methyl 6-(2,6-difluoro-3-hydroxyphenyl)-5-fluoropicolinate (1.0 equiv.) and cesium carbonate (2.0 equiv.) in DMF (0.4 M) was 2-methoxy-1-bromoethane (2 equiv.). After stirring for 16 hours the heterogeneous solution was diluted with water, extracted with EtOAc, dried over MgSO₄, filtered and concentrated to yield methyl 6-(2,6-difluoro-3-(2-methoxyethoxy)phenyl)-5-fluoropicolinate in 99% yield. LC/MS=342.0 (M+H), Rₜ=0.79 min.

### Synthesis of 6-(2,6-difluoro-3-(2-methoxyethoxy)phenyl)-5-fluoropicolinic acid

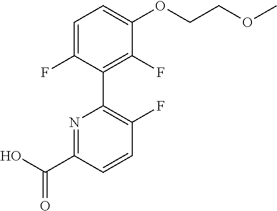

Method 2 was followed using methyl 6-(2,6-difluoro-3-(2-methoxyethoxy)phenyl)-5-fluoropicolinate to give 6-(2,6-difluoro-3-(2-methoxyethoxy)phenyl)-5-fluoropicolinic acid in 95% yield. LC/MS=328.1 (M+H), Rₜ=0.68 min.

### Synthesis of methyl 6-(3-(2-(tert-butyldimethylsilyloxy)ethoxy)-2,6-difluorophenyl)-5-fluoropicolinate

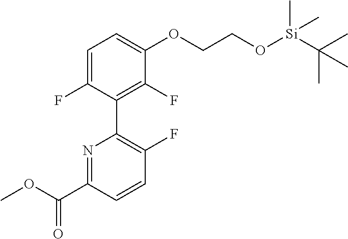

To a solution of methyl 6-(2,6-difluoro-3-hydroxyphenyl)-5-fluoropicolinate (1.0 equiv.) and cesium carbonate (4.0 equiv.) in DMF (0.4 M) was (2-bromoethoxy)(tert-butyl)dimethylsilane (2 equiv.). After stirring for 16 hours at rt and 2 hours at 60° C. the heterogeneous solution was diluted with water, extracted with EtOAc, dried over MgSO₄, filtered, concentrated and purified by ISCO SiO₂ chromatography to yield methyl 6-(3-(2-(tert-butyldimethylsilyloxy)ethoxy)-2,6-difluorophenyl)-5-fluoropicolinate in 90% yield. LC/MS=442.1 (M+H), Rₜ=1.18 min.

### Synthesis of 6-(3-(2-(tert-butyldimethylsilyloxy)ethoxy)-2,6-difluorophenyl)-5-fluoropicolinic acid

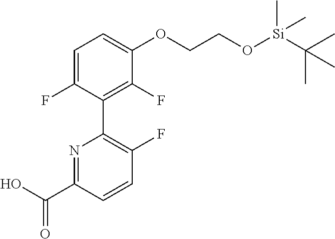

Method 2 was followed using methyl 6-(3-(2-(tert-butyldimethylsilyloxy)ethoxy)-2,6-difluorophenyl)-5-fluoropicolinate to give 6-(3-(2-(tert-butyldimethylsilyloxy)ethoxy)-2,6-difluorophenyl)-5-fluoropicolinic acid in 87% yield. LC/MS=428.1 (M+H), Rₜ=1.08 min.

### Synthesis of methyl 6-(2,6-difluoro-4-(2-hydroxypropan-2-yl)phenyl)-5-fluoropicolinate

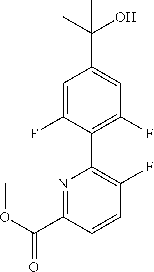

Method 1 was followed using 6-bromo-5-fluoropicolinate (1.0 equiv.) and (2-(3,5-difluoro-4-(4,4,5,5-tetramethyl-1,3,2-dioxaborolan-2-yl)phenyl)propan-2-yloxy)triisopropylsilane (1.6 equiv.) at 100° C. for 30 min in the microwave to give methyl 6-(2,6-difluoro-4-(2-hydroxypropan-2-yl)phenyl)-5-fluoropicolinate in 90% yield. LC/MS=325.9 (MH⁺), Rₜ=0.81 min. 1H NMR (400 MHz, <cdcl3>) δ ppm 1.59 (s, 6H), 4.00 (s, 3H), 7.15 (d, J=9.00 Hz, 2H), 7.62-7.68 (m, 1H), 8.23-8.29 (m, 1H).

### Synthesis of 6-(2,6-difluoro-4-(2-hydroxypropan-2-yl)phenyl)-5-fluoropicolinic acid

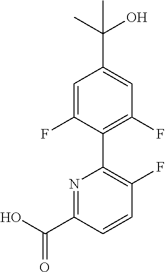

Method 2 was followed using methyl 6-(2,6-difluoro-4-(2-hydroxypropan-2-yl)phenyl)-5-fluoropicolinate to give 6-(2,6-difluoro-4-(2-hydroxypropan-2-yl)phenyl)-5-fluoropicolinic acid in 94% yield. LC/MS=312.0 (MH⁺), Rₜ=0.69 min.

### Synthesis of methyl 6-(2,6-difluoro-4-(2-methoxypropan-2-yl)phenyl)-5-fluoropicolinate

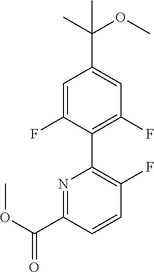

To a round-bottom flask containing methyl 6-(2,6-difluoro-4-(2-hydroxypropan-2-yl)phenyl)-5-fluoropicolinate (1.0 equiv.) in DMF (0.20 M) at 0° C. was added sodium hydride NaH (1.5 equiv.). The reaction mixture was stirred at 0° C. for 30 min and MeI (1.2 equiv.) was added into the mixture. The reaction was slowly warmed up to rt and stirred at rt for 24 h. Check LC-MS of the reaction sample and it showed only 20% conversion. Add another 1.5 equiv. NaH and 1.2 equiv. MeI to the reaction. The mixture was stirred at rt for additional 2 days. The reaction mixture was quenched with water, diluted with EtOAc and washed with sat NaHCO3, sat NaCl. The organic layer was dried over Na₂SO₄, filtered and concentrated. The crude was purified by column chromatography on silica gel (25% EtOAc/Heptane) to yield 6-(2,6-difluoro-4-(2-methoxypropan-2-yl)phenyl)-5-fluoropicolinate in 22% yield. LC/MS (m/z): 340.0 (MH⁺), Rt=0.97 min (0-95 method).

### Synthesis of 6-(2,6-difluoro-4-(2-methoxypropan-2-yl)phenyl)-5-fluoropicolinic acid

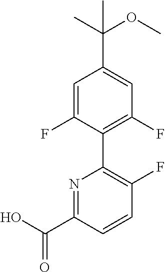

Method 2 was followed using 6-(2,6-difluoro-4-(2-methoxypropan-2-yl)phenyl)-5-fluoropicolinate to give 6-(2,6-difluoro-4-(2-methoxypropan-2-yl)phenyl)-5-fluoropicolinic acid in 93% yield. LC/MS=325.9 (MH⁺), Rₜ=0.85 min. ¹H NMR (400 MHz, <dmso>) δ ppm 1.36-1.57 (m, 6H), 2.99-3.08 (m, 3H), 3.15-3.55 (m, 2H), 7.26 (d, J=9.00 Hz, 2H), 7.98-8.11 (m, 1H), 8.16-8.28 (m, 1H).

### Synthesis of methyl 6-(4-(2-(dimethylamino)-2-oxoethoxy)-2,6-difluorophenyl)-5-fluoropicolinate

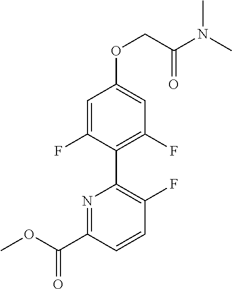

A solution of methyl 6-(2,6-difluoro-4-hydroxyphenyl)-5-fluoropicolinate (1.0 equiv.) in DMF (0.17 M) was added NaH, 60% dispersion in mineral oil (1.1 equiv.). The mixture was stirred for 30 min at ambient temperature. 2-chloro-N,N-dimethylacetamide (1.1 equiv.) was added in a dropwise fashion. The mixture was stirred overnight at ambient temperature. The reaction mixture was quenched by the addition of water. The mixture was extracted with ethyl acetate. The combined extracts were washed sequentially with water and brine, dried over sodium sulfate, filtered, and concentrated to give methyl 64442-(dimethylamino)-2-oxoethoxy)-2,6-difluorophenyl)-5-fluoropicolinate in 100% yield. LC/MS=369.2 (MH⁺), Rₜ=0.74 min.

### Synthesis of 6-(4-(2-(dimethylamino)-2-oxoethoxy)-2,6-difluorophenyl)-5-fluoropicolinic acid

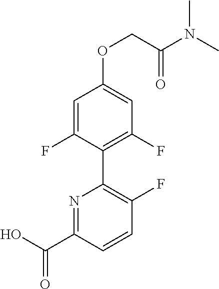

Method 2 was followed using methyl 6-(4-(2-(dimethylamino)-2-oxoethoxy)-2,6-difluorophenyl)-5-fluoropicolinate to give 6-(4-(2-(dimethylamino)-2-oxoethoxy)-2,6-difluorophenyl)-5-fluoropicolinic acid in 98% yield. LC/MS=355.2 (MH⁺), Rₜ=0.65 min.

### Synthesis of methyl 6-(2,6-difluoro-4-(pyridazin-4-yl)phenyl)-5-fluoropicolinate

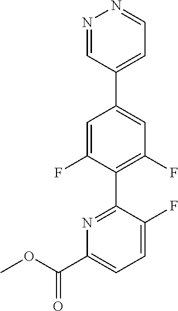

A solution of methyl 6-(2,6-difluoro-4-(4,4,5,5-tetramethyl-1,3,2-dioxaborolan-2-yl)phenyl)-5-fluoropicolinate (1.0 equiv.), 4-bromopyridazine-HBr salt (1.2 equiv.), PdCl2(dppf) (0.2 equiv.) in DME/2M Na₂CO₃ (0.05 M) was heated in the microwave at 100° C. for 20 minutes. The solution was diluted with EtOAc. The organic was washed with NaCl(sat.), separated, dried over MgSO₄, filtered, concentrated and purified by ISCO SiO₂ chromatography (0-100% EtOAc/n-heptanes, than hold at 100% EtOAc) to yield methyl 6-(2,6-difluoro-4-(pyridazin-4-yl)phenyl)-5-fluoropicolinate in 100% yield. LC/MS=346.1 (MH⁺), Rₜ=0.70 min.

### Synthesis of 6-(2,6-difluoro-4-(pyridazin-4-yl)phenyl)-5-fluoropicolinic acid

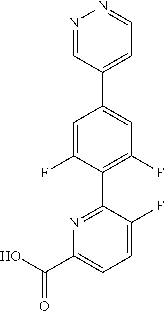

Method 2 was followed using methyl 6-(2,6-difluoro-4-(pyridazin-4-yl)phenyl)-5-fluoropicolinate to give 6-(2,6-difluoro-4-(pyridazin-4-yl)phenyl)-5-fluoropicolinic acid in 58% yield. LC/MS=332.1 (MH⁺), Rₜ=0.61 min.

### Synthesis of 4-(3,5-difluorophenyl)tetrahydro-2H-pyran-4-ol

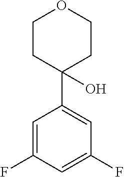

To a solution of 1-bromo-3,5-difluorobenzene in THF (0.16 M) under N₂ was added Mg turnings (1.6 equiv.). A reflux condenser was attached and the solution was submerged in a 90° C. oil bath and refluxed for 2 hours at which time the heat was removed and the solution cooled to 0° C. Dihydro-2H-pyran-4(3H)-one (1.0 equiv.) in THF was added and the solution was stirred under N₂ allowing to warm to rt for 16 hrs. The reaction was quenched by addition of sat. NH₄Cl and the solution was extracted with EtOAc, washed with brine, dried over sodium sulfate, filtered, concentrated. The crude material was purified by ISCO SiO₂ chromatography eluting with 0-100% EtOAc/n-heptanes to yield 4-(3,5-difluorophenyl)tetrahydro-2H-pyran-4-ol in 37% yield. ¹H NMR (400 MHz, <cdcl3>) δ ppm 1.63 (d, J=12.13 Hz, 2H), 2.11 (ddd, J=13.50, 11.15, 6.65 Hz, 2H), 3.84-3.90 (m, 4H), 6.72 (tt, J=8.75, 2.20 Hz, 1H), 6.97-7.05 (m, 2H).

### Synthesis of 4-(3,5-difluorophenyl)-3,6-dihydro-2H-pyran

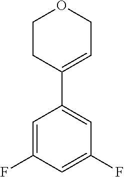

4-(3,5-difluorophenyl)tetrahydro-2H-pyran-4-ol (1.0 equiv.) was dissolved in DCM (0.2 M) and cooled to 0° C. TEA (2.8 equiv.) was added to the solution, followed by MsCl (1.3 equiv.). The reaction was stirred at rt for 2 hrs. The solution was cooled to 0° C. and DBU (3.0 equiv.) was added. The reaction was stirred at rt for 18 hrs. The solution was concentrated and the residue was purified by SiO₂ chromatography (0-100% EtOAc in Heptanes) to afford 4-(3,5-difluorophenyl)-3,6-dihydro-2H-pyran in 38% yield. ¹H NMR (400 MHz, <cdcl3>) δ ppm 2.42-2.49 (m, 2H), 3.93 (t, J=5.48 Hz, 2H), 4.32 (q, J=2.74 Hz, 2H), 6.16-6.22 (m, 1H), 6.70 (tt, J=8.80, 2.35 Hz, 1H), 6.85-6.94 (m, 2H).

### Synthesis of 4-(3,5-difluorophenyl)tetrahydro-2H-pyran

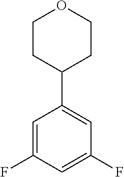

To a solution of 4-(3,5-difluorophenyl)-3,6-dihydro-2H-pyran (1.0 equiv.) in methanol (0.2 M) was added 10% Pd/C (0.05 equiv.). The reaction was placed under an atmosphere of hydrogen and stirred for 18 hours. Upon completion, the solution was filtered over a pad of Celite, the pad was washed with DCM, the filtrate was concentrated in vacuo to give 4-(3,5-difluorophenyl)tetrahydro-2H-pyran in 71% yield. ¹H NMR (400 MHz, <cdcl3>) δ ppm 1.76 (br. s., 4H), 2.75 (br. s., 1H), 3.50 (br. s., 2H), 4.08 (d, J=9.78 Hz, 2H), 6.56-6.94 (m, 3H).

### Synthesis of 2-(2,6-difluoro-4-(tetrahydro-2H-pyran-4-yl)phenyl)-4,4,5,5-tetramethyl-1,3,2-dioxaborolane

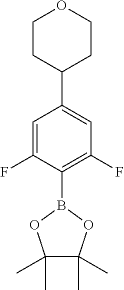

Method 3 was followed using 2-isopropoxy-4,4,5,5-tetramethyl-1,3,2-dioxaborolane (2.2 equiv.), butyllithium (1.1 equiv.) and 4-(3,5-difluorophenyl)tetrahydro-2H-pyran (1.0 equiv.) to give 2-(2,6-difluoro-4-(tetrahydro-2H-pyran-4-yl)phenyl)-4,4,5,5-tetramethyl-1,3,2-dioxaborolane in 100% yield. ¹H NMR (400 MHz, <cdcl3>) δ ppm 1.16-1.19 (m, 12H), 1.65-1.74 (m, 4H), 2.60-2.75 (m, 1H), 3.37-3.51 (m, 2H), 4.01 (dt, J=11.54, 3.42 Hz, 2H), 6.67 (d, J=8.22 Hz, 2H).

### Synthesis of methyl 6-(2,6-difluoro-4-(tetrahydro-2H-pyran-4-yl)phenyl)-5-fluoropicolinate

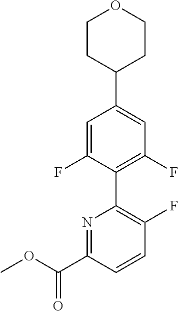

Method 1 was followed using methyl 6-bromo-5-fluoropicolinate (1.0 equiv.) and 2-(2,6-difluoro-4-(tetrahydro-2H-pyran-4-yl)phenyl)-4,4,5,5-tetramethyl-1,3,2-dioxaborolane (3.0 equiv.) at 100° C. for 20 min in microwave to give methyl 6-(2,6-difluoro-4-(tetrahydro-2H-pyran-4-yl)phenyl)-5-fluoropicolinate in 59% yield. LC/MS=352.2 (MH⁺), Rₜ=0.92 min.

### Synthesis of 6-(2,6-difluoro-4-(tetrahydro-2H-pyran-4-yl)phenyl)-5-fluoropicolinic acid

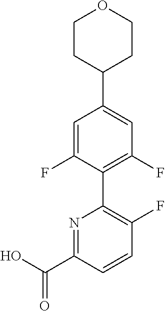

Method 2 was followed using methyl 6-(2,6-difluoro-4-(pyridazin-4-yl)phenyl)-5-fluoropicolinate to give 6-(2,6-difluoro-4-(tetrahydro-2H-pyran-4-yl)phenyl)-5-fluoropicolinic acid in 71% yield. LC/MS=338.1 (MH⁺) Rₜ=0.80 min.

### Synthesis of 3-(3,5-difluorophenyl)oxetan-3-ol

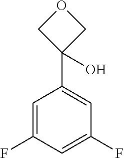

To a solution of 1-bromo-3,5-difluorobenzene in THF (0.27 M) under Ar was added Mg turnings (1.6 M). A reflux condenser was attached and the solution was submerged in a 90° C. oil bath and refluxed for two hours. The oxetan-3-one (1.0 equiv.) was added in THF via syringe. The solution was left stirring at rt under Ar overnight. The reaction solution was quenched by addition of NH₄Cl₍ₛₐₜ₎ and the solution was extracted with EtOAc, washed with NaCl_{(sat.)}, dried over MgSO₄, filtered, concentrated and purified by ISCO SiO₂ chromatography (0-100% EtOAc/n-heptanes gradient) to yield 3-(3,5-difluorophenyl)oxetan-3-ol in 56% yield. ¹H NMR (400 MHz, CHLOROFORM-d) δ ppm 4.82 (d, J=7.63 Hz, 2H), 4.91 (d, J=7.63 Hz, 2 H), 7.16-7.23 (m, 2H).

### Synthesis of 3-(3,5-difluoro-4-(4,4,5,5-tetramethyl-1,3,2-dioxaborolan-2-yl)phenyl)oxetan-3-ol

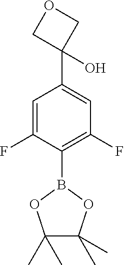

Method 3 was followed using 2-isopropoxy-4,4,5,5-tetramethyl-1,3,2-dioxaborolane (2.5 equiv.), butyllithium (2.4 equiv.) and 3-(3,5-difluorophenyl)oxetan-3-ol (1.0 equiv.) to give 3-(3,5-difluoro-4-(4,4,5,5-tetramethyl-1,3,2-dioxaborolan-2-yl)phenyl)oxetan-3-ol in 79% yield. ¹H NMR (400 MHz, <cdcl3>) δ ppm 1.34-1.42 (m, 12H), 4.79 (d, J=7.63 Hz, 2 H), 4.90 (d, J=7.34 Hz, 2H), 7.17 (d, J=8.22 Hz, 2H).

### Synthesis of methyl 6-(2,6-difluoro-4-(3-hydroxyoxetan-3-yl)phenyl)-5-fluoropicolinate

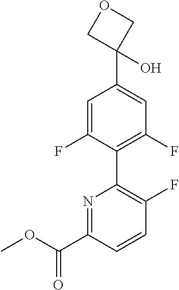

Method 1 was followed using methyl 6-bromo-5-fluoropicolinate (1.0 equiv.) and 3-(3,5-difluoro-4-(4,4,5,5-tetramethyl-1,3,2-dioxaborolan-2-yl)phenyl)oxetan-3-ol (1.4 equiv.) at 100° C. for 20 min in microwave to give methyl 6-(2,6-difluoro-4-(3-hydroxyoxetan-3-yl)phenyl)-5-fluoropicolinate in 43% yield. LC/MS=340.1 (MH⁺), Rₜ=0.69 min.

### Synthesis of 6-(2,6-difluoro-4-(3-hydroxyoxetan-3-yl)phenyl)-5-fluoropicolinic acid

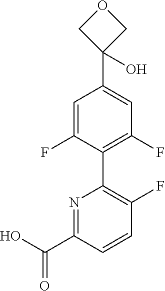

Method 2 was followed using methyl 6-(2,6-difluoro-4-(3-hydroxyoxetan-3-yl)phenyl)-5-fluoropicolinate to give 6-(2,6-difluoro-4-(3-hydroxyoxetan-3-yl)phenyl)-5-fluoropicolinic acid in 99% yield. LC/MS=325.9 (MH⁺) Rₜ=0.60 min.

### Synthesis of methyl 6-(2,6-difluoro-4-(3-methoxyoxetan-3-yl)phenyl)-5-fluoropicolinate

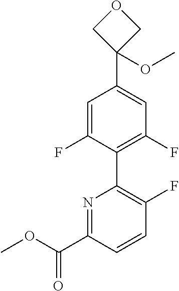

To a solution of methyl 6-(2,6-difluoro-4-(3-hydroxyoxetan-3-yl)phenyl)-5-fluoropicolinate (1.0 equiv.) in DMF (0.34 M) at 0° C. was added NaH dispersion (1.4 equiv.). The solution was stirred in the ice bath for 1 hour, at which time MeI (1.5 equiv) was added. The solution was left stirring under Ar as the bath was allowed to warm up to rt and stirred at rt overnight. The solution was diluted with H₂O, and extracted with EtOAc. The organic was washed with H₂O, NaCl_{(sat.)}, dried over MgSO₄, filtered, concentrated and purified by ISCO SiO₂ chromatography (0-100% EtOAc/n-heptanes) to yield methyl 6-(2,6-difluoro-4-(3-methoxyoxetan-3-yl)phenyl)-5-fluoropicolinate in 46% yield. LC/MS=354.0 (MH⁺) Rt=0.82 min.

### Synthesis of 6-(2,6-difluoro-4-(3-methoxyoxetan-3-yl)phenyl)-5-fluoropicolinic acid

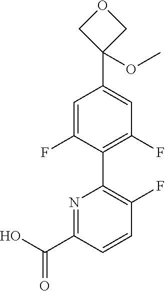

Method 2 was followed using methyl 6-(2,6-difluoro-4-(3-methoxyoxetan-3-yl)phenyl)-5-fluoropicolinate to give 6-(2,6-difluoro-4-(3-methoxyoxetan-3-yl)phenyl)-5-fluoropicolinic acid in 86% yield. LC/MS=339.9 (MH⁺), Rt=0.71 min.

### Synthesis of tert-butyl ((1S,3R,5S)-3-(3-(3-amino-6-(2,6-difluoro-4-(3-hydroxyoxetan-3-yl)phenyl)-5-fluoropicolinamido)pyridin-4-yl)-5-methylcyclohexyl) carbamate

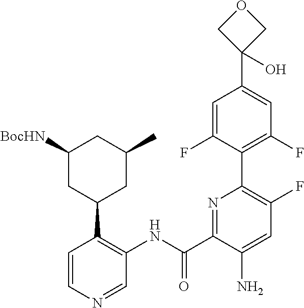

Method 1 was followed using tert-butyl ((1S,3R,5S)-3-(3-(3-amino-6-bromo-5-fluoropicolinamido)pyridin-4-yl)-5-methylcyclohexyl)carbamate (1.0 equiv.) and 3-(3,5-difluoro-4-(4,4,5,5-tetramethyl-1,3,2-dioxaborolan-2-yl)phenyl)oxetan-3-ol (2.0 equiv.) at 100° C. for 20 min in microwave to give tert-butyl ((1S,3R,5S)-3-(3-(3-amino-6-(2,6-difluoro-4-(3-hydroxyoxetan-3-yl)phenyl)-5-fluoropicolinamido)pyridin-4-yl)-5-methylcyclohexyl) carbamate. LC/MS=628.3 (MH+), Rₜ=0.84 min.

### Synthesis of methyl 6-(2,6-difluoro-4-(3-fluorooxetan-3-yl)phenyl)-5-fluoropicolinate

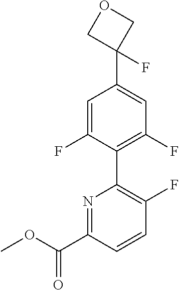

To a solution of methyl 6-(2,6-difluoro-4-(3-hydroxyoxetan-3-yl)phenyl)-5-fluoropicolinate (1.0 equiv.) in CH₂Cl₂ (0.04 M) at −78° C. under Ar was added methylDAST (1.7 equiv.). After addition, the solution was stirred under Ar at −78° C. for 10 minutes and then the bath was removed. The reaction was allowed to warm up to rt and quenched by addition of NaHCO_{3(sat.)}. The solution was diluted with EtOAc, washed with NaHCO3(sat.), NaCl(sat.), dried over MgSO4, filtered, concentrated, purified by ISCO SiO2 chromatography (24 gram column, 0-100 EtOAc/n-heptanes) to yield methyl 6-(2,6-difluoro-4-(3-fluorooxetan-3-yl)phenyl)-5-fluoropicolinate in 56% yield. LC/MS=342.0 (MH+), Rₜ=0.85 min.

### Synthesis of 6-(2,6-difluoro-4-(3-fluorooxetan-3-yl)phenyl)-5-fluoropicolinic acid

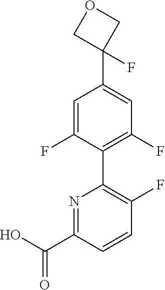

Method 2 was followed using methyl 6-(2,6-difluoro-4-(3-fluorooxetan-3-yl)phenyl)-5-fluoropicolinate to give 6-(2,6-difluoro-4-(3-fluorooxetan-3-yl)phenyl)-5-fluoropicolinic acid in 99% yield. LC/MS=327.9 (MH⁺) Rₜ=0.74 min.

### Synthesis of 4-(3,5-difluorophenyl)tetrahydro-2H-pyran-4-ol

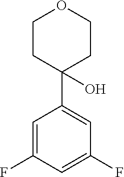

To a solution of 1-bromo-3,5-difluorobenzene (1.6 equiv.) in THF (0.26 M) under Ar was added Mg turnings (1.6 equiv.). A reflux condenser was attached and the solution was submerged in a 90° C. oil bath and refluxed for two hours. The oxetan-3-one (1.0 equiv.) was added in THF via syringe. The solution was left stirring at rt under Ar for 5 hrs. The reaction solution was quenched by addition of NH₄Cl₍ₛₐₜ₎ and the solution was extracted with EtOAc, washed with NaCl_{(sat.)}, dried over MgSO₄, filtered, concentrated and purified by ISCO SiO₂ chromatography (0-100% EtOAc/n-heptanes gradient) to yield 4-(3,5-difluorophenyl)tetrahydro-2H-pyran-4-ol in 71% yield. ¹H NMR (400 MHz, CHLOROFORM-d) δ ppm 1.59-1.68 (m, 3 H), 2.07-2.19 (m, 2H), 3.87-3.93 (m, 4H), 6.72 (tt, J=8.75, 2.20 Hz, 1H), 6.97-7.06 (m, 2 H).

### Synthesis of 4-(3,5-difluoro-4-(4,4,5,5-tetramethyl-1,3,2-dioxaborolan-2-yl)phenyl)tetrahydro-2H-pyran-4-ol

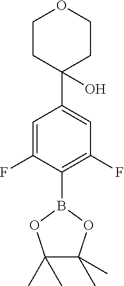

Method 3 was followed using 2-isopropoxy-4,4,5,5-tetramethyl-1,3,2-dioxaborolane (2.5 equiv.), butyllithium (2.4 equiv.) and 4-(3,5-difluorophenyl)tetrahydro-2H-pyran-4-ol (1.0 equiv.) to give 4-(3,5-difluoro-4-(4,4,5,5-tetramethyl-1,3,2-dioxaborolan-2-yl)phenyl)tetrahydro-2H-pyran-4-ol in 97% yield. ¹H NMR (400 MHz, <cdcl3>) δ ppm 1.32-1.42 (m, 12H), 1.56-1.65 (m, 2H), 2.11 (d, J=3.13 Hz, 2H), 3.86-3.92 (m, 4H), 6.99 (d, J=9.00 Hz, 2H).

### Synthesis of methyl 6-(2,6-difluoro-4-(4-hydroxytetrahydro-2H-pyran-4-yl)phenyl)-5-fluoropicolinate

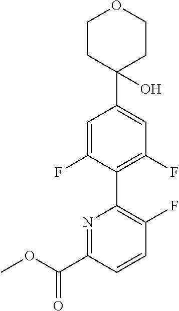

Method 1 was followed using methyl 6-bromo-5-fluoropicolinate (1.0 equiv.) and 4-(3,5-difluoro-4-(4,4,5,5-tetramethyl-1,3,2-dioxaborolan-2-yl)phenyl)tetrahydro-2H-pyran-4-ol (1.8 equiv.) at 100° C. for 20 min in microwave to give methyl 6-(2,6-difluoro-4-(4-hydroxytetrahydro-2H-pyran-4-yl)phenyl)-5-fluoropicolinate in 28% yield. LC/MS=368.0 (MH⁺), Rₜ=0.75 min.

### Synthesis of 6-(2,6-difluoro-4-(4-hydroxytetrahydro-2H-pyran-4-yl)phenyl)-5-fluoropicolinic acid

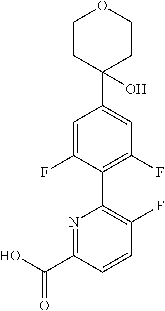

Method 2 was followed using methyl 6-(2,6-difluoro-4-(4-hydroxytetrahydro-2H-pyran-4-yl)phenyl)-5-fluoropicolinate to give 6-(2,6-difluoro-4-(4-hydroxytetrahydro-2H-pyran-4-yl)phenyl)-5-fluoropicolinic acid in 69% yield. LC/MS=354.0 (MH⁺) Rₜ=0.64 min.

### Synthesis of methyl 6-(2,6-difluoro-4-(4-fluorotetrahydro-2H-pyran-4-yl)phenyl)-5-fluoropicolinate

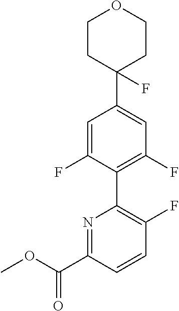

To a solution of methyl 6-(2,6-difluoro-4-(4-hydroxytetrahydro-2H-pyran-4-yl)phenyl)-5-fluoropicolinate (1.0 equiv.) in CH₂Cl₂ (0.04 M) at −78° C. under Ar was added methylDAST (2.0 equiv.). After addition, the solution was stirred under Ar at −78° C. for 10 minutes and then the bath was removed. The reaction was allowed to warm up to rt and quenched by addition of NaHCO_{3(sat.)}. The solution was diluted with EtOAc, washed with NaHCO_{3(sat.)}, NaCl_{(sat.)}, dried over MgSO4, filtered, concentrated, purified by ISCO SiO₂ chromatography (0-100 EtOAc/n-heptanes) to yield methyl 6-(2,6-difluoro-4-(4-fluorotetrahydro-2H-pyran-4-yl)phenyl)-5-fluoropicolinate in 100% yield. LC/MS=370.0 (MH⁺), Rₜ=0.94 min.

### Synthesis of 6-(2,6-difluoro-4-(4-fluorotetrahydro-2H-pyran-4-yl)phenyl)-5-fluoropicolinic acid

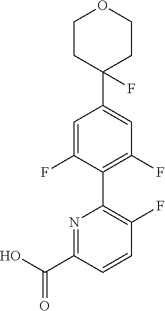

Method 2 was followed using methyl 6-(2,6-difluoro-4-(4-fluorotetrahydro-2H-pyran-4-yl)phenyl)-5-fluoropicolinate to give 6-(2,6-difluoro-4-(4-fluorotetrahydro-2H-pyran-4-yl)phenyl)-5-fluoropicolinic acid in 95% yield. LC/MS=355.9 (MH⁺), Rₜ=0.81 min.

### Synthesis of 1-(3,5-difluorophenyl)cyclobutanol

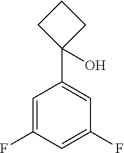

To a solution of 1-bromo-3,5-difluorobenzene (1.0 equiv.) in THF (0.26 M) under Ar was added Mg turnings (1.6 equiv.). A reflux condenser was attached and the solution was submerged in a 90° C. oil bath and refluxed for two hours. The oxetan-3-one (1.0 equiv.) was added in THF via syringe. The solution was left stirring at rt under Ar for 5 hrs. The reaction solution was quenched by addition of NH₄Cl(sat) and the solution was extracted with EtOAc, washed with NaCl(sat.), dried over MgSO4, filtered, concentrated and purified by ISCO SiO₂ chromatography (0-100% EtOAc/n-heptanes gradient) to yield 1-(3,5-difluorophenyl)cyclobutanol in 54% yield. ¹H NMR (400 MHz, CHLOROFORM-d) δ ppm 1.69-1.83 (m, 1H), 2.03-2.13 (m, 1H), 2.31-2.43 (m, 2H), 2.45-2.56 (m, 2H), 6.71 (tt, J=8.80, 2.35 Hz, 1H), 6.98-7.07 (m, 2H).

### Synthesis of 1-(3,5-difluoro-4-(4,4,5,5-tetramethyl-1,3,2-dioxaborolan-2-yl)phenyl)cyclobutanol

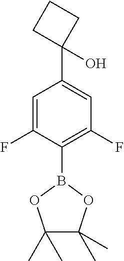

Method 3 was followed using 2-isopropoxy-4,4,5,5-tetramethyl-1,3,2-dioxaborolane (2.5 equiv.), butyllithium (2.4 equiv.) and 1-(3,5-difluorophenyl)cyclobutanol (1.0 equiv.) to give 1-(3,5-difluoro-4-(4,4,5,5-tetramethyl-1,3,2-dioxaborolan-2-yl)phenyl)cyclobutanol in 100% yield. ¹H NMR (400 MHz, <cdcl3>) δ ppm 1.23-1.25 (m, 12 H), 1.69-1.82 (m, 1H), 2.05-2.12 (m, 1H), 2.37 (br. s., 2H), 2.47 (br. s., 2H), 7.00 (d, J=8.80 Hz, 2H).

### Synthesis of methyl 6-(2,6-difluoro-4-(1-hydroxycyclobutyl)phenyl)-5-fluoropicolinate

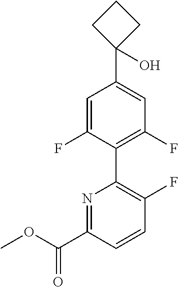

Method 1 was followed using methyl 6-bromo-5-fluoropicolinate (1.0 equiv.) and 1-(3,5-difluoro-4-(4,4,5,5-tetramethyl-1,3,2-dioxaborolan-2-yl)phenyl)cyclobutanol (1.6 equiv.) at 100° C. for 30 min in microwave to give methyl 6-(2,6-difluoro-4-(1-hydroxycyclobutyl)phenyl)-5-fluoropicolinate in 71% yield. LC/MS=338.0 (MH⁺), Rₜ=0.85 min.

### Synthesis of 6-(2,6-difluoro-4-(1-hydroxycyclobutyl)phenyl)-5-fluoropicolinic acid

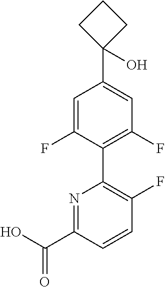

Method 2 was followed using methyl 6-(2,6-difluoro-4-(1-hydroxycyclobutyl)phenyl)-5-fluoropicolinate to give 6-(2,6-difluoro-4-(1-hydroxycyclobutyl)phenyl)-5-fluoropicolinic acid in 90% yield. LC/MS=323.9 (MH+), Rₜ=0.74 min.

### Synthesis of 3-amino-N-(4-((1R,3S,5S)-3-amino-5-methylcyclohexyl)pyridin-3-yl)-6-(2,6-difluoro-4-(1-hydroxycyclobutyl)phenyl)-5-fluoropicolinamide

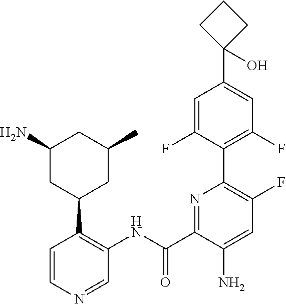

Method 1 was followed using tert-butyl ((1S,3R,5S)-3-(3-(3-amino-6-bromo-5-fluoropicolinamido)pyridin-4-yl)-5-methylcyclohexyl)carbamate (1.0 equiv.) and 1-(3,5-difluoro-4-(4,4,5,5-tetramethyl-1,3,2-dioxaborolan-2-yl)phenyl)cyclobutanol (2.0 equiv.) at 100° C. for 20 min in microwave to give tert-butyl ((1S,3R,5S)-3-(3-(3-amino-6-(2,6-difluoro-4-(1-hydroxycyclobutyl)phenyl)-5-fluoropicolinamido)pyridin-4-yl)-5-methylcyclohexyl) carbamate. LC/MS=626.3 (MH+), Rt=0.95 min. The Boc protected product was treated with 25% TFA/CH₂Cl₂ (0.04 M) for 30 mins. The volatiles were removed in vacuo, the residue was dissolved in DMSO and purified by RP-HPLC. The product fractions were lyophilized directly to yield 3-amino-N-(4-((1R,3S,5S)-3-amino-5-methylcyclohexyl)pyridin-3-yl)-6-(2,6-difluoro-4-(1-hydroxycyclobutyl)phenyl)-5-fluoropicolinamide in 28% yield. LC/MS=526.1 (MH+), Rt=0.65 min.

### Synthesis of methyl 6-(2,6-difluoro-4-((tetrahydro-2H-pyran-4-yl)oxy)phenyl)-5-fluoropicolinate

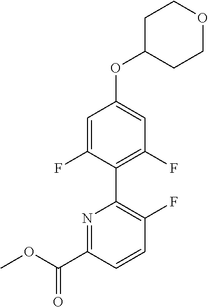

To a solution of DIAD (3.0 equiv.) and triphenylphosphine (3.0 equiv.) in THF (0.24 M) was added tetrahydro-4-pyranol (1.2 equiv.). The mixture was stirred for 10 min. methyl 6-(2,6-difluoro-4-hydroxyphenyl)-5-fluoropicolinate (1.0 equiv.) was added. The mixture was stirred at ambient temperature overnight. Additional triphenylphosphine (3.0 equiv.) and DIAD (3.0 equiv.) were added, and the mixture was stirred overnight. After overnight, the reaction was essentially complete. The mixture was concentrated and purified by flash chromatography over silica gel (heptanes:ethyl acetate gradient) to give methyl 6-(2,6-difluoro-4-((tetrahydro-2H-pyran-4-yl)oxy)phenyl)-5-fluoropicolinate in 77% yield. LC/MS=368.0 (MH+), Rt=0.95 min.

### Synthesis of 6-(2,6-difluoro-4-((tetrahydro-2H-pyran-4-yl)oxy)phenyl)-5-fluoropicolinic acid

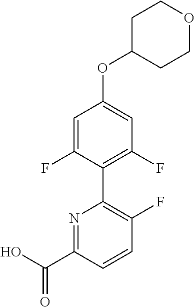

Method 2 was followed using methyl 6-(2,6-difluoro-4-((tetrahydro-2H-pyran-4-yl)oxy)phenyl)-5-fluoropicolinate to give 6-(2,6-difluoro-4-((tetrahydro-2H-pyran-4-yl)oxy)phenyl)-5-fluoropicolinic acid in 100% yield. LC/MS=353.9 (MH+), Rₜ=0.82 min.

### Synthesis of 4-(3,5-difluorophenoxy)tetrahydro-2H-pyran

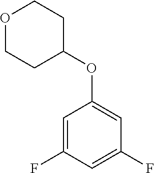

To a solution of 3,5-difluorophenol (1.0 equiv.), tetrahydro-2H-pyran-4-ol (1.2 equiv.), and triphenylphosphine (2.0 equiv.) in THF (0.33 M) at 0° C. was added DIAD (2.0 equiv.) dropwise. The reaction mixture was stirred at rt overnight. The mixture was concentrated and purified by flash chromatography over silica gel (heptanes:ethyl acetate gradient) to give 4-(3,5-difluorophenoxy)tetrahydro-2H-pyran in 90% yield. 1H NMR (400 MHz, <cdcl3>) δ ppm 1.72-1.84 (m, 2H), 1.96-2.09 (m, 2H), 3.59 (ddd, J=11.64, 8.31, 3.52 Hz, 2H), 3.90-4.04 (m, 2H), 4.44 (tt, J=7.78, 3.77 Hz, 1H), 6.32-6.53 (m, 3H).

### Synthesis of 2-(2,6-difluoro-4-((tetrahydro-2H-pyran-4-yl)oxy)phenyl)-4,4,5,5-tetramethyl-1,3,2-dioxaborolane

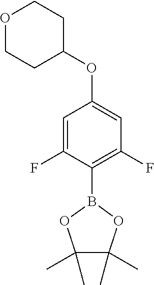

Method 3 was followed using 2-isopropoxy-4,4,5,5-tetramethyl-1,3,2-dioxaborolane (1.5 equiv.), butyllithium (1.3 equiv.) and 4-(3,5-difluorophenoxy)tetrahydro-2H-pyran (1.0 equiv.) to give 2-(2,6-difluoro-4-((tetrahydro-2H-pyran-4-yl)oxy)phenyl)-4,4,5,5-tetramethyl-1,3,2-dioxaborolane in 33% yield. 1H NMR (400 MHz, <cdcl3>) δ ppm 1.21-1.34 (m, 12H), 1.78 (dtd, J=12.72, 8.31, 8.31, 3.91 Hz, 2H), 1.93-2.09 (m, 2H), 3.59 (ddd, J=11.64, 8.31, 3.13 Hz, 2H), 3.89-4.01 (m, 2H), 4.48 (tt, J=7.78, 3.77 Hz, 1H), 6.40 (d, J=9.39 Hz, 2

H).

### Synthesis of methyl 3-amino-6-(2,6-difluoro-4-((tetrahydro-2H-pyran-4-yl)oxy)phenyl)-5-fluoropicolinate

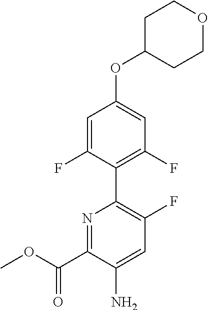

Method 1 was followed using methyl 3-amino-6-bromo-5-fluoropicolinate (1.0 equiv.) and 2-(2,6-difluoro-4-(tetrahydro-2H-pyran-4-yloxy)phenyl)-4,4,5,5-tetramethyl-1,3,2-dioxaborolane (1.5 equiv.) at 100° C. for 10 min in microwave to give methyl 3-amino-6-(2,6-difluoro-4-((tetrahydro-2H-pyran-4-yl)oxy)phenyl)-5-fluoropicolinate in 65% yield. LC/MS=383.0 (MH+), Rt=0.88 min.

### Synthesis of 3-amino-6-(2,6-difluoro-4-((tetrahydro-2H-pyran-4-yl)oxy)phenyl)-5-fluoropicolinic acid

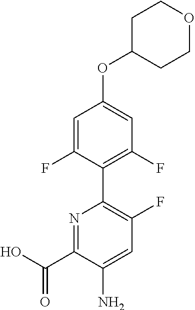

Method 2 was followed using methyl 3-amino-6-(2,6-difluoro-4-((tetrahydro-2H-pyran-4-yl)oxy)phenyl)-5-fluoropicolinate to give 3-amino-6-(2,6-difluoro-4-((tetrahydro-2H-pyran-4-yl)oxy)phenyl)-5-fluoropicolinic acid in 99% yield. LC/MS=369.0 (MH+), Rt=0.84 min.

### Synthesis of (S)-methyl 6-(2,6-difluoro-4-((tetrahydro-2H-pyran-3-yl)oxy)phenyl)-5-fluoropicolinate and (R)-methyl 6-(2,6-difluoro-4-((tetrahydro-2H-pyran-3-yl)oxy)phenyl)-5-fluoropicolinate

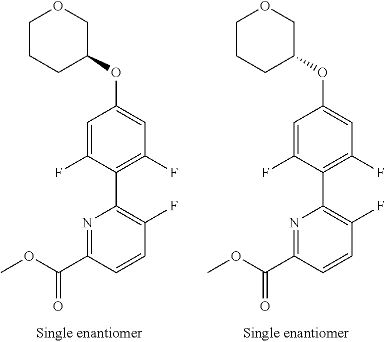

To a solution of DIAD (2.0 equiv.) and triphenylphosphine (2.0 equiv.) in THF (0.24 M) was added tetrahydro-2H-pyran-3-ol (1.2 equiv.). The mixture was stirred for 10 min. methyl 6-(2,6-difluoro-4-hydroxyphenyl)-5-fluoropicolinate (1.0 equiv.) was added. The mixture was stirred at ambient temperature overnight. Additional triphenylphosphine (2.0 equiv.) and DIAD (2.0 equiv.) were added, and the mixture was stirred overnight. The mixture was concentrated and purified by flash chromatography over silica gel (heptanes:ethyl acetate gradient) to give methyl 6-(2,6-difluoro-4-((tetrahydro-2H-pyran-3-yl)oxy)phenyl)-5-fluoropicolinate in 39% yield. Purification was completed via chiral HPLC (EtOH/heptane)=15/85, 20 mL/min, AD column) to yield (S)-methyl 6-(2,6-difluoro-4-((tetrahydro-2H-pyran-3-yl)oxy)phenyl)-5-fluoropicolinate (18% yield, 99% ee) and (R)-methyl 6-(2,6-difluoro-4-((tetrahydro-2H-pyran-3-yl)oxy)phenyl)-5-fluoropicolinate (18% yield, 99% ee). LC/MS=368.2 (MH+), Rt=0.92 min. ¹H NMR (400 MHz, CHLOROFORM-d) δ ppm 1.65 (ddd, J=12.81, 8.51, 4.11 Hz, 1H), 1.78-1.97 (m, 2H), 2.06-2.16 (m, 1H), 3.57-3.67 (m, 2H), 3.72-3.80 (m, 1H), 3.95 (dd, J=11.54, 2.15 Hz, 1H), 3.99-4.01 (m, 3H), 4.32 (dt, J=6.95, 3.37 Hz, 1H), 6.54-6.62 (m, 2H), 7.59-7.67 (m, 1H), 8.19-8.28 (m, 1H).

### Synthesis of (R)-6-(2,6-difluoro-4-((tetrahydro-2H-pyran-3-yl)oxy)phenyl)-5-fluoropicolinic acid

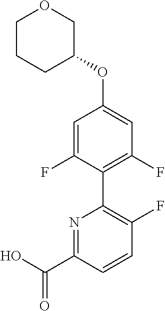

Method 2 was followed using (R)-methyl 6-(2,6-difluoro-4-((tetrahydro-2H-pyran-3-yl)oxy)phenyl)-5-fluoropicolinate to give (R)-6-(2,6-difluoro-4-((tetrahydro-2H-pyran-3-yl)oxy)phenyl)-5-fluoropicolinic acid in 93% yield. LC/MS=353.9 (MH+), Rt=0.81 min.

### Synthesis of (S)-6-(2,6-difluoro-4-((tetrahydro-2H-pyran-3-yl)oxy)phenyl)-5-fluoropicolinic acid

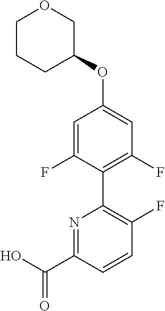

Method 2 was followed using (S)-methyl 6-(2,6-difluoro-4-((tetrahydro-2H-pyran-3-yl)oxy)phenyl)-5-fluoropicolinate to give (S)-6-(2,6-difluoro-4-((tetrahydro-2H-pyran-3-yl)oxy)phenyl)-5-fluoropicolinic acid in 94% yield. LC/MS=353.9 (MH⁺), Rₜ=0.81 min.

### Synthesis of methyl 6-(4-(ethoxymethyl)-2,6-difluorophenyl)-5-fluoropicolinate

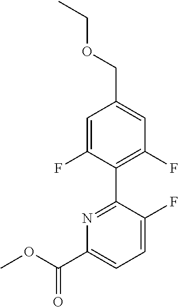

To a solution of methyl 6-(2,6-difluoro-4-(hydroxymethyl)phenyl)-5-fluoropicolinate (1.0 equiv.) in DMF (0.20 M) (colorless) at 0° C. was added sodium hydride (1.2 equiv.) and the reaction was stirred at 0° C. for 2 min. Ethyl iodide (1.2 equiv.) was added and the reaction was allowed to warm to room temperature. After 1 h, additional 1.0 equiv. of NaH was added and stirred for 15 ml. Reaction was quenched by the addition of sat. Ammonium chloride. The aqueous was acidified with conc HCl to pH3 and extracted with ethyl acetate three times. The organics were combined, dried with MgSO₄, filtered and concentrated. The crude mixture was used as is. LC/MS=326.0 (MH⁺), Rₜ=0.94 min.

### Synthesis of 6-(4-(ethoxymethyl)-2,6-difluorophenyl)-5-fluoropicolinic acid

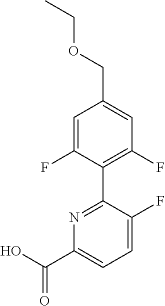

Method 2 was followed using methyl 6-(4-(ethoxymethyl)-2,6-difluorophenyl)-5-fluoropicolinate to give 6-(4-(ethoxymethyl)-2,6-difluorophenyl)-5-fluoropicolinic acid in 27% yield. LC/MS=311.9 (MH⁺), Rₜ=0.82 min.

### Synthesis of 1-(3,5-difluorophenoxy)-2-methylpropan-2-ol

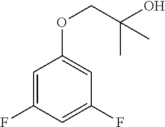

A steel bomb was charged with Phenol (1.0 equiv.), K₂CO₃ (1.0 equiv.), NaH₂PO₄ (1.0 equiv.) and 2,2-dimethyloxirane (3.0 equiv.) and then dissolved in a mixture of MeCN/Water (6/1, 0.61 M). The resulting mixture was heated at 140° C. for 6 hrs. The reaction mixture was quenched with water and diluted with EtOAc. The aqueous layer was separated then extracted with EtOAc. The combined organics were dried over MgSO₄ and concentrated in vacuo. The crude was further purified by column chromatography eluting with 100% heptanes to 10% EtOAc: heptanes to yield 1-(3,5-difluorophenoxy)-2-methylpropan-2-ol the product in 79% yield. ¹H NMR (400 MHz, <cdcl3>) δ ppm 1.34 (s, 6H), 2.07 (d, J=15.26 Hz, 1H), 3.76 (s, 2H), 6.39-6.50 (m, 3H).

### Synthesis of ((1-(3,5-difluorophenoxy)-2-methylpropan-2-yl)oxy)triethylsilane

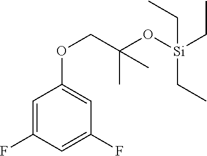

To a solution of 1-(3,5-difluorophenoxy)-2-methylpropan-2-ol (1.0 equiv.) and triethylamine (3.0 equiv.) in DCM (0.66 M) at 0° C. was added TESOTf (2.0 equiv.) dropwise. The resulting mixture was stirred at RT for 1 h. The reaction mixture was quenched with water and diluted with EtOAc. The aqeuous layer was separated then extracted with EtOAc. The combined organics were dried over MgSO₄ and concentrated in vacuo. The oil was further purified by column chromatography eluting with 100% heptanes to 10% EtOAc: heptanes to yield (1-(3,5-difluorophenoxy)-2-methylpropan-2-yloxy)triethylsilane in 100% yield. ¹H NMR (400 MHz, <cdcl3>) δ ppm 0.55-0.63 (m, 6H), 0.94-0.98 (m, 9H), 1.28-1.36 (m, 6H), 3.64-3.72 (m, 2H), 6.34-6.48 (m, 3H).

### Synthesis of ((1-(3,5-difluoro-4-(4,4,5,5-tetramethyl-1,3,2-dioxaborolan-2-yl)phenoxy)-2-methylpropan-2-yl)oxy)triethylsilane

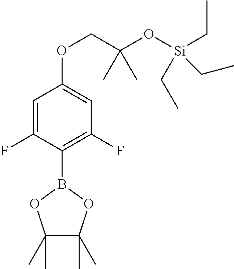

Method 3 was followed using 2-isopropoxy-4,4,5,5-tetramethyl-1,3,2-dioxaborolane (2.2 equiv.), butyllithium (1.1 equiv.) and (1-(3,5-difluorophenoxy)-2-methylpropan-2-yloxy)triethylsilane (1.0 equiv.) to give ((1-(3,5-difluoro-4-(4,4,5,5-tetramethyl-1,3,2-dioxaborolan-2-yl)phenoxy)-2-methylpropan-2-yl)oxy)triethylsilane in 100% yield. ¹H NMR (400 MHz, <cdcl3>) δ ppm 0.58 (q, J=7.83 Hz, 6H), 0.93 (t, J=7.83 Hz, 9H), 1.22-1.26 (m, 12H), 1.32 (s, 6H), 3.69 (s, 2H), 6.40 (d, J=9.39 Hz, 2H).

### Synthesis of methyl 6-(2,6-difluoro-4-(2-methyl-2-((triethylsilyl)oxy)propoxy)phenyl)-5-fluoropicolinate

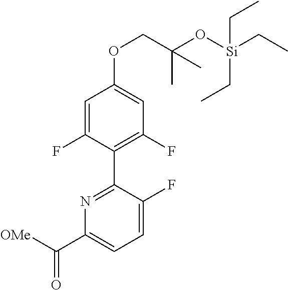

Method 1 was followed using methyl 6-bromo-5-fluoropicolinate (0.8 equiv.) and (1-(3,5-difluoro-4-(4,4,5,5-tetramethyl-1,3,2-dioxaborolan-2-yl)phenoxy)-2-methylpropan-2-yloxy)triethylsilane (1.0 equiv.) at 80° C. for 1 hr to methyl 6-(2,6-difluoro-4-(2-methyl-2-((triethylsilyl)oxy)propoxy)phenyl)-5-fluoropicolinate in 99% yield. LC/MS=470.0 (MH⁺), Rₜ=1.44 min.

### Synthesis of 6-(2,6-difluoro-4-(2-methyl-2-((triethylsilypoxy)propoxy)phenyl)-5-fluoropicolinic acid

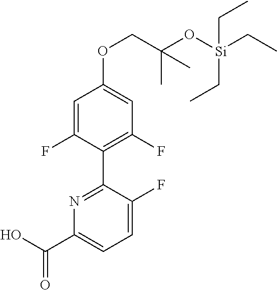

Method 2 was followed using methyl 6-(2,6-difluoro-4-(2-methyl-2-((triethylsilyl)oxy)propoxy)phenyl)-5-fluoropicolinate to give 6-(2,6-difluoro-4-(2-methyl-2-((triethylsilyl)oxy)propoxy)phenyl)-5-fluoropicolinic acid in 35% yield. LC/MS=456.0 (MH⁺), Rₜ=1.35 min.

### Synthesis of methyl 6-(2,6-difluoro-4-(2-hydroxy-2-methylpropoxy)phenyl)-5-fluoropicolinate

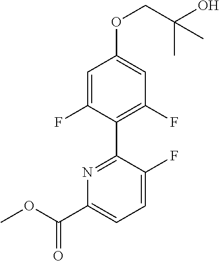

To a solution of methyl 6-(2,6-difluoro-4-(2-methyl-2-(triethylsilyloxy)propoxy)phenyl)-5-fluoropicolinate (1.0 equiv.) in THF (1.27 M) at rt was added HCl (7.5 equiv.) and MeOH (30.0 equiv.). The resulting solution was stirred at RT for 2 hrs. The reaction mixture was diluted with EtOAc and water. The aqueous layer was then extracted with EtOAc. The combined organics were dried over MgSO4 and concentrated in vacuo. The crude was further purified by flash column chromatography eluting with 100% heptanes to 30% EtOAc:heptanes to 50% EtOAc:heptanes to yield methyl 6-(2,6-difluoro-4-(2-hydroxy-2-methylpropoxy)phenyl)-5-fluoropicolinate as a yellow solid in 49% yield. LC/MS=355.9 (MH⁺), Rₜ=0.84 min.

### Synthesis of methyl 6-(2,6-difluoro-4-(2-methoxypropan-2-yl)phenyl)-5-fluoropicolinate

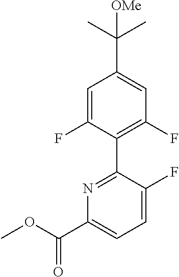

To a solution of methyl 6-(2,6-difluoro-4-(2-hydroxy-2-methylpropoxy)phenyl)-5-fluoropicolinate (1.0 equiv.) in DMF (0.2 M) at 0° C. was added NaH (7.0 equiv.) followed by MeI (12.0 equiv.). The resulting solution was allowed to warm to RT and stirred for 16 hrs. The reaction mixture was then diluted with EtOAc and water. The aqueous layer was then extracted with EtOAc, the combined organics were dried over MgSO₄ and concentrated in vacuo to yield methyl 6-(2,6-difluoro-4-(2-methoxy-2-methylpropoxy)phenyl)-5-fluoropicolinate in 87% yield. The oil was used in the subsequent hydrolysis reaction without further purification. LC/MS=369.8 (MH+), Rt=0.95 min.

### Synthesis of 6-(2,6-difluoro-4-(2-methoxypropan-2-yl)phenyl)-5-fluoropicolinic acid

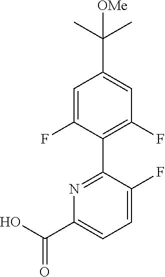

Method 2 was followed using methyl 6-(2,6-difluoro-4-(2-methoxypropan-2-yl)phenyl)-5-fluoropicolinate to give 6-(2,6-difluoro-4-(2-methoxypropan-2-yl)phenyl)-5-fluoropicolinic acid in 93% yield. LC/MS=325.9 (MH+), Rt=0.85 min. 1H NMR (400 MHz, <dmso>) δ ppm 1.36-1.57 (m, 6H), 2.99-3.08 (m, 3H), 7.26 (d, J=9.00 Hz, 2H), 7.98-8.11 (m, 1H), 8.16-8.28 (m, 1H).

### Synthesis of methyl 6-(4-(difluoromethyl)-2,6-difluorophenyl)-5-fluoropicolinate

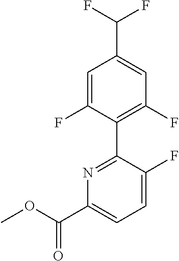

To a solution of methyl 6-(2,6-difluoro-4-formylphenyl)-5-fluoropicolinate (1.0 equiv.) in DCM (0.14 M) at 0° C. was added DAST (1.4 equiv.) dropwise. The resulting mixture was then allowed to warm to RT over 3 h. The reaction mixture was quenched with water and diluted with EtOAc. The aqueous layer was separated then extracted with EtOAc. The combined organics were dried over MgSO4 and concentrated in vacuo. The crude was further purified by column chromatography eluting with 100% heptanes to 10% EtOAc: heptanes to yield methyl 6-(4-(difluoromethyl)-2,6-difluorophenyl)-5-fluoropicolinate as a colourless solid in 88% yield. LC/MS=317.9 (MH+), Rt=0.92 min.

### Synthesis of 6-(4-(difluoromethyl)-2,6-difluorophenyl)-5-fluoropicolinic acid

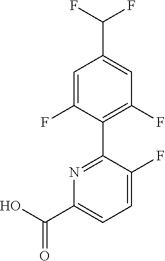

Method 2 was followed using methyl 6-(4-(difluoromethyl)-2,6-difluorophenyl)-5-fluoropicolinate to give 6-(4-(difluoromethyl)-2,6-difluorophenyl)-5-fluoropicolinic acid in 92% yield. LC/MS=303.8 (MH+), Rt=0.80 min.

### methyl 6-(2,6-difluoro-4-(oxetan-3-yloxy)phenyl)-5-fluoropicolinate

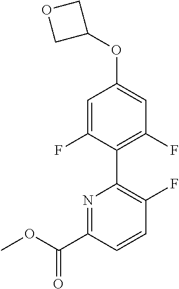

To a solution of methyl 6-(2,6-difluoro-4-hydroxyphenyl)-5-fluoropicolinate (1.0 equiv.) in DMF (0.08 M) was added potassium carbonate (10.1 equiv.) and oxetan-3-yl 4-methylbenzenesulfonate (1.3 equiv.). The mixtures were stirred at 110° C. in an oil bath for 8 hrs and cooled to room temperature. The reaction was diluted with ethyl acetate and water. The organic phase was dried with sodium sulfate, filtered and concentrated. The crude material was purified via silica gel column chromatography eluting with ethyl acetate and heptanes (0-50%). The pure fractions were concentrated to give methyl 6-(2,6-difluoro-4-(oxetan-3-yloxy)phenyl)-5-fluoropicolinate in 36% yield. LC/MS=340.0 (MH+), Rt=0.82 min. 1H NMR (400 MHz, <cdcl3>) δ ppm 4.00 (s, 3H), 4.77 (dd, J=7.63, 5.28 Hz, 2H), 5.00 (t, J=6.85 Hz, 2H), 5.22 (quin, J=5.48 Hz, 1H), 6.38 (d, J=9.00 Hz, 2H), 7.63 (t, J=8.61 Hz, 1H), 8.24 (dd, J=8.61, 3.91 Hz, 1H).

### Synthesis of 6-(2,6-difluoro-4-(oxetan-3-yloxy)phenyl)-5-fluoropicolinic acid

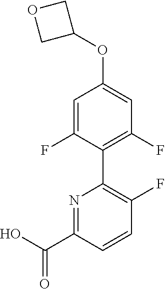

Method 2 was followed using methyl 6-(2,6-difluoro-4-(oxetan-3-yloxy)phenyl)-5-fluoropicolinate to give 6-(2,6-difluoro-4-(oxetan-3-yloxy)phenyl)-5-fluoropicolinic acid in 78% yield. LC/MS=325.9 (MH+), Rt=0.72 min.

### Synthesis of 1-(cyclopropylmethoxy)-3,5-difluorobenzene

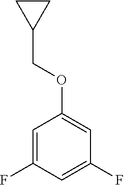

To a solution of 3,5-difluorophenol (1.0 equiv.) in DMF (0.17 M) was added potassium carbonate (2.2 equiv.) followed by (bromomethyl)cyclopropane (1.1 equiv.) and the reaction was stirred overnight at room temperature. The reaction was poured into a separatory funnel and diluted with a 3:1 (v/v) solution of EtOAc:heptanes. The organic phase was washed with water, then sat'd NaHCO3. The remaining organic phase was dried over MgSO4, filtered and concentrated in vacuo to provide 1-(cyclopropylmethoxy)-3,5-difluorobenzene in 100% yield. 1H NMR (400 MHz, <cdcl3>) δ ppm 0.35 (q, J=4.83 Hz, 2H), 0.59-0.71 (m, 2H), 1.26-1.27 (m, 1H), 3.76 (d, J=6.65 Hz, 2H), 6.32-6.48 (m, 3H).

### Synthesis of 2-(4-(cyclopropylmethoxy)-2,6-difluorophenyl)-4,4,5,5-tetramethyl-1,3,2-dioxaborolane

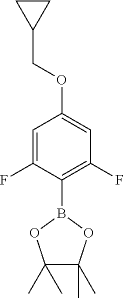

Method 3 was followed using 2-isopropoxy-4,4,5,5-tetramethyl-1,3,2-dioxaborolane (2.2 equiv.), butyllithium (1.2 equiv.) and 1-(cyclopropylmethoxy)-3,5-difluorobenzene (1.0 equiv.) to give 2-(4-(cyclopropylmethoxy)-2,6-difluorophenyl)-4,4,5,5-tetramethyl-1,3,2-dioxaborolane in 100% yield. 1H NMR (400 MHz, <cdcl3>) δ ppm 0.35 (br. s., 2H), 0.66 (d, J=6.26 Hz, 2H), 1.20-1.28 (m, 13H), 3.77 (dd, J=6.65, 2.35 Hz, 2H), 6.30-6.48 (m, 2H).

### Synthesis of methyl 6-(4-(cyclopropylmethoxy)-2,6-difluorophenyl)-5-fluoropicolinate

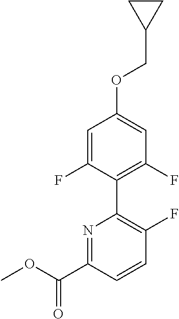

Method 1 was followed using methyl 6-bromo-5-fluoropicolinate (0.8 equiv.) and 2-(4-(cyclopropylmethoxy)-2,6-difluorophenyl)-4,4,5,5-tetramethyl-1,3,2-dioxaborolane (1.0 equiv.) at 80° C. for 2 hours to give methyl 6-(4-(cyclopropylmethoxy)-2,6-difluorophenyl)-5-fluoropicolinate in 8% yield. LC/MS=337.9 (MH+), Rt=1.04 min.

### Synthesis of 6-(4-(cyclopropylmethoxy)-2,6-difluorophenyl)-5-fluoropicolinic acid

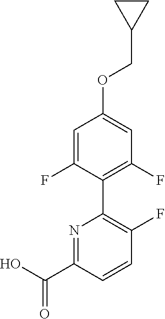

Method 2 was followed using methyl 6-(4-(ethoxymethyl)-2,6-difluorophenyl)-5-fluoropicolinate to give 6-(4-(cyclopropylmethoxy)-2,6-difluorophenyl)-5-fluoropicolinic acid in 79% yield. LC/MS=323.9 (MH+), Rt=0.93 min.

### Synthesis of 1,3-difluoro-5-isopropoxybenzene

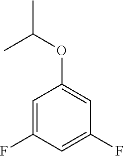

To a solution of 3,5-difluorophenol (1.0 equiv.) in DMF (0.26 M) was added potassium carbonate (2.2 equiv.) followed by 2-iodopropane (1.1 equiv.) and the reaction was stirred overnight at room temperature. The reaction was poured into a separatory funnel and diluted with a 3:1 (v/v) solution of EtOAc:heptanes. The organic phase was washed with water, then sat'd NaHCO3. The remaining organic phase was dried over MgSO4, filtered and concentrated in vacuo to provide 1,3-difluoro-5-isopropoxybenzene in 88% yield. 1H NMR (400 MHz, <cdcl3>) δ ppm 1.33 (d, J=6.26 Hz, 6H), 4.48 (dt, J=11.93, 6.16 Hz, 1H), 6.31-6.47 (m, 3H).

### Synthesis of 2-(2,6-difluoro-4-isopropoxyphenyl)-4,4,5,5-tetramethyl-1,3,2-dioxaborolane

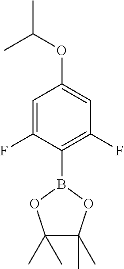

Method 3 was followed using 2-isopropoxy-4,4,5,5-tetramethyl-1,3,2-dioxaborolane (2.2 equiv.), butyllithium (1.2 equiv.) and 1,3-difluoro-5-isopropoxybenzene (1.0 equiv.) to give 2-(2,6-di fluoro-4-isopropoxyphenyl)-4,4,5,5-tetramethyl-1,3,2-dioxaborolane in 99% yield. 1H NMR (400 MHz, <cdcl3>) δ ppm 1.24 (s, 12H), 1.31-1.33 (m, 6H), 4.43-4.56 (m, 1H), 6.31-6.44 (m, 2H).

### Synthesis of methyl 6-(2,6-difluoro-4-isopropoxyphenyl)-5-fluoropicolinate

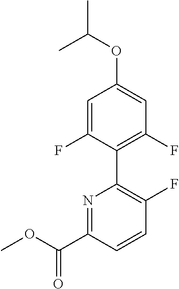

Method 1 was followed using methyl 6-bromo-5-fluoropicolinate (0.8 equiv.) and 2-(2,6-difluoro-4-isopropoxyphenyl)-4,4,5,5-tetramethyl-1,3,2-dioxaborolane (1.0 equiv.) at 70° C. for 1 hour to give methyl 6-(2,6-difluoro-4-isopropoxyphenyl)-5-fluoropicolinate in 27% yield. LC/MS=325.9 (MH+), Rt=1.04 min.

### Synthesis of 6-(2,6-difluoro-4-isopropoxyphenyl)-5-fluoropicolinic acid

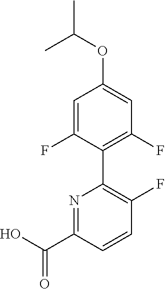

Method 2 was followed using methyl 6-(2,6-difluoro-4-isopropoxyphenyl)-5-fluoropicolinate to give 6-(2,6-difluoro-4-isopropoxyphenyl)-5-fluoropicolinic acid in 35% yield. LC/MS=311.9 (MH+), Rt=0.92 min.

### Synthesis of ((1-(3,5-difluorophenyl)vinyl)oxy)trimethylsilane

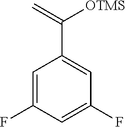

To a solution of 1-(3,5-difluorophenyl)ethanone (1.0 equiv.) in DCM (0.25 M) was added TEA (2.0 equiv.) and cooled to 0° C. TMSOTf (1.1 equiv.) was added dropwise over 5 min. The solution was stirred at 0° C. for 15 min. The solution was quenched by the addition of sat. NaHCO3 and the organics were extracted. The organic layer was dried with sodium sulfate, filtered and concentrated to give ((1-(3,5-difluorophenyl)vinyl)oxy)trimethylsilane in 99% yield. ¹H NMR (400 MHz, CHLOROFORM-d) δ ppm 0.28 (s, 9H), 4.46-4.52 (m, 1H), 4.90-4.94 (m, 1H), 7.06-7.13 (m, 2H), 7.41-7.50 (m, 1H). The product was used for the next step without further purification.

### Synthesis of (1-(3,5-difluorophenyl)cyclopropoxy)trimethylsilane

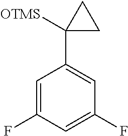

To a solution of diethylzinc (3.2 equiv.) in DCM (0.16 M) at 0° C. was slowly added diiodomethane (3.2 equiv.), followed by (10 min later) (1-(3,5-difluorophenyl)vinyloxy)trimethylsilane (1.0 equiv.). The reaction mixture was allowed to warm to rt and stirred at rt overnight. The reaction was quenched by the careful addition of sat. ammonium chloride. The layers were separated. The aqueous contained lots of salts, difficult to extracted a second time. Added DCM and filtered though a pad of Celite. The filtrate was transferred to a sep. funnel and the layers were separated. The organics were combined, dried with sodium sulfate, filtered and concentrated to give (1-(3,5-difluorophenyl)cyclopropoxy)trimethylsilane in 91% yield. 1H NMR (400 MHz, <cdcl3>) δ ppm 0.83-0.95 (m, 2H). 1.11-1.24 (m, 2H). 6.44-6.53 (m, 1H). 6.59-6.68 (m, 2H). This material was used for the next step without further purification.

### Synthesis of 1-(3,5-difluoro-4-(4,4,5,5-tetramethyl-1,3,2-dioxaborolan-2-yl)phenyl)cyclopropanol

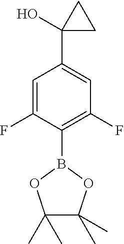

Method 3 was followed using 2-isopropoxy-4,4,5,5-tetramethyl-1,3,2-dioxaborolane (2.5 equiv.), butyllithium (2.4 equiv.) and (1-(3,5-difluorophenyl)cyclopropoxy)trimethylsilane (1.0 equiv.) to give 1-(3,5-difluoro-4-(4,4,5,5-tetramethyl-1,3,2-dioxaborolan-2-yl)phenyl)cyclopropanol in 100% yield.

### Synthesis of methyl 6-(2,6-difluoro-4-(1-hydroxycyclopropyl)phenyl)-5-fluoropicolinate

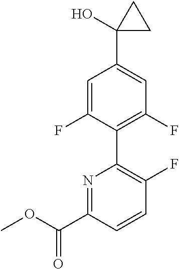

Method 1 was followed using methyl 6-bromo-5-fluoropicolinate (1.0 equiv.) and 1-(3,5-difluoro-4-(4,4,5,5-tetramethyl-1,3,2-dioxaborolan-2-yl)phenyl)cyclopropanol (1.2 equiv.) at 90° C. for 1 hour to give methyl 6-(2,6-difluoro-4-(1-hydroxycyclopropyl)phenyl)-5-fluoropicolinate in 6% yield. LC/MS=323.9 (MH+), Rt=0.79 min. 1H NMR (400 MHz, <cdcl3>) δ ppm 1.07-1.20 (m, 2H), 1.26-1.39 (m, 2H), 3.98 (s, 3H), 7.03 (d, J=9.39 Hz, 2 H), 7.91 (t, J=8.61 Hz, 1H), 8.29 (dd, J=9.00, 3.91 Hz, 1H).

### Synthesis of 6-(2,6-difluoro-4-(1-hydroxycyclopropyl)phenyl)-5-fluoropicolinic acid and 6-(2,6-difluoro-4-propionylphenyl)-5-fluoropicolinic acid

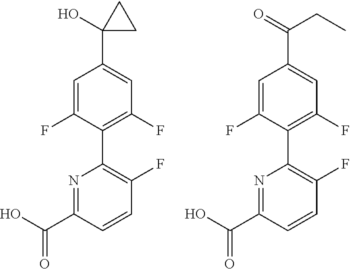

Method 2 was followed using methyl 6-(2,6-difluoro-4-(1-hydroxycyclopropyl)phenyl)-5-fluoropicolinate to give a mixture (2/3 ratio) of 6-(2,6-difluoro-4-(1-hydroxycyclopropyl)phenyl)-5-fluoropicolinic acid and 6-(2,6-difluoro-4-propionylphenyl)-5-fluoropicolinic acid in 86% yield. LC/MS=309.9 (MH+), Rt=0.66 and 0.70 min.

### Synthesis of methyl 6-(4-(difluoromethoxy)-2,6-difluorophenyl)-5-fluoropicolinate

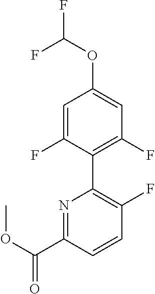

To a scintillation vial containing methyl 6-(2,6-difluoro-4-hydroxyphenyl)-5-fluoropicolinate (1.0 equiv.) and K₂CO₃ (1.2 equiv.) in DMF/water (9/1, 0.50 M) was added sodium 2-chloro-2,2-difluoroacetate (1.6 equiv.). The reaction mixture was stirred at 100° C. in an oil bath for 4 hrs. The reaction mixture was cooled down to rt, diluted with EtOAc and washed with H2O, sat NaCl. The organic layer was dried over Na₂SO₄, filtered and concentrated to give methyl 6-(4-(difluoromethoxy)-2,6-difluorophenyl)-5-fluoropicolinate. LC/MS=333.9 (MH+), Rt=0.93 min. This material was used for the next step without further purification.

### Synthesis of 6-(4-(difluoromethoxy)-2,6-difluorophenyl)-5-fluoropicolinic acid

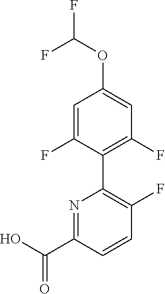

Method 2 was followed using methyl 6-(4-(difluoromethoxy)-2,6-difluorophenyl)-5-fluoropicolinate to give 6-(4-(difluoromethoxy)-2,6-difluorophenyl)-5-fluoropicolinic acid in 35% yield. LC/MS=319.9 (MH+), Rt=0.82 min.

### Synthesis of (1-(3,5-difluorophenyl)ethoxy)triisopropylsilane

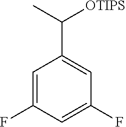

To a round-bottom flask containing 1-(3,5-difluorophenyl)ethanone (1.0 equiv.) in ethanol (0.32 M) at 0° C. was added NaBH₄ (1.15 equiv.). The homogenous reaction mixture was stirred at 0° C. for 3 hrs. The reaction mixture was quenched with water and concentrated to dryness, diluted with EtOAc and washed with sat NaCl. The organic layer was dried over Na2SO4, filtered and concentrated. The crude was used in next step without further purification. To a round-bottom flask containing the crude product and 2,6-LUTIDINE (2.0 equiv.) in DCM (0.32 M) was added TIPSOTf (1.15 equiv). The homogenous reaction mixture was stirred at 0° C. for 3 hr and then rt for 3 hrs. The reaction was quenched with sat. NH₄Cl and extracted with DCM. The crude was purified by column chromatography on silica gel (10% EtOAc/Hexane) to yield (1-(3,5-difluorophenyl)ethoxy)triisopropylsilane in 66% yield.

### Synthesis of (1-(3,5-difluoro-4-(4,4,5,5-tetramethyl-1,3,2-dioxaborolan-2-yl)phenyl)ethoxy)triisopropylsilane

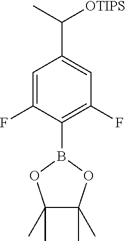

Method 3 was followed using 2-isopropoxy-4,4,5,5-tetramethyl-1,3,2-dioxaborolane (1.2 equiv.), butyllithium (1.2 equiv.) and (1-(3,5-difluorophenyl)ethoxy)triisopropylsilane (1.0 equiv.) to give (1-(3,5-difluoro-4-(4,4,5,5-tetramethyl-1,3,2-dioxaborolan-2-yl)phenyl)ethoxy)triisopropylsilane in 89% yield. 1H NMR (400 MHz, <cdcl3>) δ ppm 0.46-0.60 (m, 6H), 0.84-0.94 (m, 10H), 0.95-1.03 (m, 5H), 1.18-1.24 (m, 3H), 1.34-1.37 (m, 12H), 4.78 (d, J=6.65 Hz, 1H), 6.76-6.88 (m, 2H).

### Synthesis of tert-butyl ((1S,3R,5S)-3-(3-(6-(2,6-difluoro-4-((R)-1-hydroxyethyl)phenyl)-5-fluoropicolinamido)pyridin-4-yl)-5-methylcyclohexyl)carbamate and tert-butyl ((1S,3R,5S)-3-(3-(6-(2,6-difluoro-4-((S)-1-hydroxyethyl)phenyl)-5-fluoropicolinamido)pyridin-4-yl)-5-methylcyclohexyl)carbamate

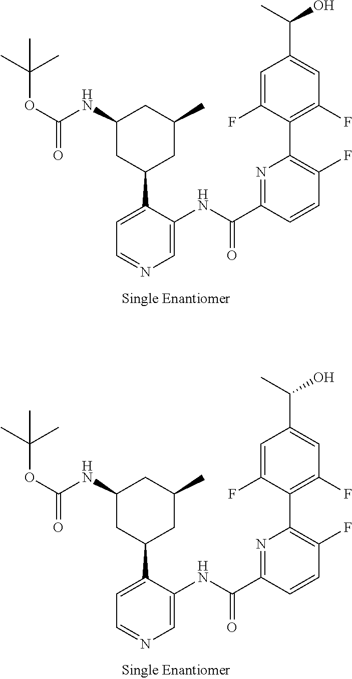

Method 1 was followed using tert-butyl (1S,3R,5S)-3-(3-(6-bromo-5-fluoropicolinamido)pyridin-4-yl)-5-methylcyclohexylcarbamate (1.0 equiv.) and (1-(3,5-difluoro-4-(4,4,5,5-tetramethyl-1,3,2-dioxaborolan-2-yl)phenyl)ethoxy)triisopropylsilane (2.5 equiv.) at 100° C. for 30 min in microwave to give tert-butyl ((1S,3R,5S)-3-(3-(6-(2,6-difluoro-4-(1-((triisopropylsilyl)oxy)ethyl)phenyl)-5-fluoropicolinamido)pyridin-4-yl)-5-methylcyclohexyl)carbamate. LC/MS=741.5 (MH+), Rt=1.37 min. The crude product was redissolved in THF (0.07 M) and TBAF (2.0 equiv.) was added. After 2 hr at rt, the mixture was concentrated and diluted in EtOAc and washed with H2O, then Brine. The organic layer was dried over Na₂SO₄ and concentrated. The residue was purified by ISCO with 0-100% EtOAc in Heptanes. Purification was completed via SFC(CO2/IPA+0.1% DEA=80/20, 15 mL/min, AD column) to yield tert-butyl ((1S,3R,5S)-3-(3-(6-(2,6-difluoro-4-((R)-1-hydroxyethyl)phenyl)-5-fluoropicolinamido)pyridin-4-yl)-5-methylcyclohexyl)carbamate (95% yield, 99% ee) and tert-butyl ((1S,3R,5S)-3-(3-(6-(2,6-difluoro-4-((S)-1-hydroxyethyl)phenyl)-5-fluoropicolinamido)pyridin-4-yl)-5-methylcyclohexyl)carbamate (95% yield, 99% ee). LC/MS=585.1 (MH+), Rt=0.87 min.

### Synthesis of (E)-methyl 6-(2,6-difluoro-4-(2-methoxyvinyl)phenyl)-5-fluoropicolinate

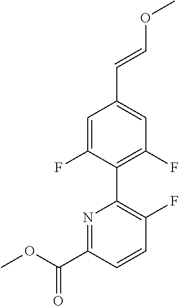

To a solution of 1.0M in THF LHMDS (1.3 equiv.) diluted in THF (0.20 M) under N2 at 0° C. was slowly added Methoxymethyltriphenylphosphonium chloride (3.1 equiv.). The solution was stirred at 0° C. for 10 min and then a solution of methyl 6-(2,6-difluoro-4-formylphenyl)-5-fluoropicolinate (1.0 equiv.) dissolved in THF (0.20 M) was added via syringe. The mixture was stirred at 0° C. allowing warming to rt for 20 h. The reaction was then heated at 65° C. for 3 h. The mixture was diluted with EtOAc and washed with brine, dried over sodium sulfate, filtered and concentrated. The crude yellow residue was purified by ISCO SiO₂ chromatography eluting with 0-100% EtOAc in Heptanes to afford (E)-methyl 6-(2,6-difluoro-4-(2-methoxyvinyl)phenyl)-5-fluoropicolinate in 55% yield.

### Synthesis of methyl 6-(2,6-difluoro-4-(2-methoxyethyl)phenyl)-5-fluoropicolinate

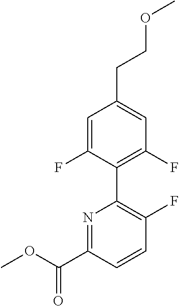

(E)-methyl 6-(2,6-difluoro-4-(2-methoxyvinyl)phenyl)-5-fluoropicolinate (1.0 equiv.) was dissolved in MeOH (0.20 M) and degassed with vacuum to Argon. Pd/C (0.05 equiv.) was added and the mixture was purged from vacuum to H2. The mixture was left under an H2 balloon for 3 hrs. The mixture was filtered through a celite plug eluting with EtOAc and concentrated. The residue was purified by ISCO using a 12 g Redisep column eluting with 0-100% EtOAC in Heptanes to give methyl 6-(2,6-difluoro-4-(2-methoxyethyl)phenyl)-5-fluoropicolinate in 36% yield. LC/MS (m/z): 326.0 (MH+), Rt=0.90 min.

### Synthesis of 6-(2,6-difluoro-4-(2-methoxyethyl)phenyl)-5-fluoropicolinic acid

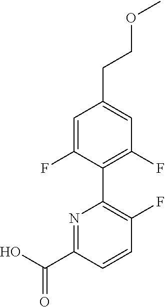

Method 2 was followed using methyl 6-(2,6-difluoro-4-(2-methoxyethyl)phenyl)-5-fluoropicolinate to give 6-(2,6-difluoro-4-(2-methoxyethyl)phenyl)-5-fluoropicolinic acid in 60% yield. LC/MS=312.0 (MH+), Rt=0.77 min.

### Synthesis of tert-butyl(3,5-difluorophenethoxy)dimethylsilane

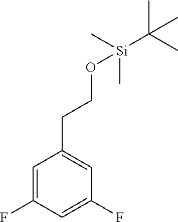

To a solution of 2-(3,5-difluorophenyl)ethanol (1.0 equiv.) in DMF (0.8 M) was added imidazole (2.2 equiv.), followed by TBDMSCl (1.1 equiv.). The reaction was stirred at rt for 3 days. The clear solution was diluted with EtOAc and washed with water, brine, dried over sodium sulfate, filtered and concentrated to tert-butyl(3,5-difluorophenethoxy)dimethylsilane in 88% yield. 1H NMR (400 MHz, <cdcl3>) δ 6.75 (dd, J=2.35, 8.61 Hz, 2H), 6.65 (tt, J=2.35, 9.00 Hz, 1H), 3.81 (t, J=6.65 Hz, 2H), 2.79 (t, J=6.65 Hz, 2H), 0.87 (s, 9H), −0.03-−0.01 (m, 6H).

### Synthesis of tert-butyl(3,5-difluoro-4-(4,4,5,5-tetramethyl-1,3,2-dioxaborolan-2-yl)phenethoxy)dimethylsilane

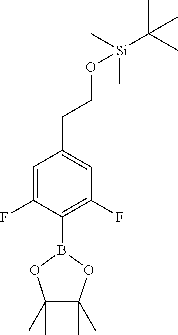

Method 3 was followed using 2-isopropoxy-4,4,5,5-tetramethyl-1,3,2-dioxaborolane (1.05 equiv.), butyllithium (1.05 equiv.) and tert-butyl(3,5-difluorophenethoxy)dimethylsilane (1.0 equiv.) to give tert-butyl(3,5-difluoro-4-(4,4,5,5-tetramethyl-1,3,2-dioxaborolan-2-yl)phenethoxy)dimethylsilane in 34% yield. 1H NMR (400 MHz, <cdcl3>) δ ppm 0.00 (s, 6H), 0.91 (s, 9H), 1.40 (s, 12H), 2.80 (td, J=6.46, 3.52 Hz, 2H), 3.82 (td, J=6.46, 3.13 Hz, 2H), 6.71-6.81 (m, 2H).

### Synthesis of methyl 6-(4-(2-((tert-butyldimethylsilyl)oxy)ethyl)-2,6-difluorophenyl)-5-fluoropicolinate

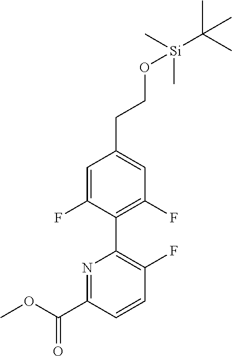

Method 1 was followed using methyl 6-bromo-5-fluoropicolinate (1.0 equiv.) and tert-butyl(3,5-difluoro-4-(4,4,5,5-tetramethyl-1,3,2-dioxaborolan-2-yl)phenethoxy)dimethylsilane (2.0 equiv.) at 100° C. for 20 min in microwave to give methyl 6-(4-(2-((tert-butyldimethylsilyl)oxy)ethyl)-2,6-difluorophenyl)-5-fluoropicolinate in 100% yield. LC/MS=426.1 (MH+), Rt=1.25 min.

### Synthesis of 6-(4-(2-((tert-butyldimethylsilyl)oxy)ethyl)-2,6-difluorophenyl)-5-fluoropicolinic acid

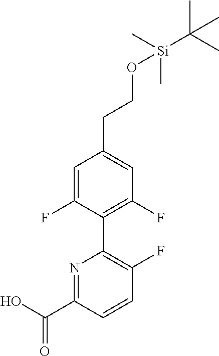

Method 2 was followed using methyl 6-(4-(2-((tert-butyldimethylsilyl)oxy)ethyl)-2,6-difluorophenyl)-5-fluoropicolinate to give 6-(4-(2-((tert-butyldimethylsilyl)oxy)ethyl)-2,6-difluorophenyl)-5-fluoropicolinic acid in 42% yield. LC/MS=412.0 (MH+), Rt=1.17 min. 1H NMR (400 MHz, <cdcl3>) δ 8.33 (dd, J=3.91, 8.61 Hz, 1H), 7.76 (t, J=8.41 Hz, 1H), 6.96 (d, J=8.61 Hz, 2H), 3.88 (t, J=6.46 Hz, 2H), 2.89 (t, J=6.26 Hz, 2H), 0.89 (s, 9H), 0.03 (s, 6H).

### Synthesis of 3-(3,5-difluorophenyl)oxetane

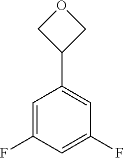

3,5-difluorophenylboronic acid (2.0 equiv.), (1R,2R)-2-aminocyclohexanol (0.06 equiv.), NaHMDS (2.0 equiv.), and nickel(II) iodide (0.06 equiv.) were dissolved in 2-propanol (0.35 M). The mixture was degassed with N2, stirred at rt for 10 min and then a solution of 3-iodooxetane (1.0 equiv.) in 2-Propanol (0.70 M) was added. The mixture was sealed and heated at 80° C. in the microwave for 20 min. The mixture was filtered through celite, eluting with EtOH and concentrated. The crude residue was purified by ISCO SiO2 chromatography eluting with 0-100% EtOAc in Heptanes to afford 3-(3,5-difluorophenyl)oxetane in 63% yield. 1H NMR (400 MHz, <cdcl3>) δ 6.88-6.96 (m, 2H), 6.72 (tt, J=2.20, 8.95 Hz, 1H), 5.08 (dd, J=6.26, 8.22 Hz, 2H), 4.71 (t, J=6.26 Hz, 2H), 4.14-4.24 (m, 1H).

### Synthesis of 2-(2,6-difluoro-4-(oxetan-3-yl)phenyl)-4,4,5,5-tetramethyl-1,3,2-dioxaborolane

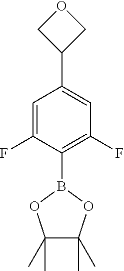

Method 3 was followed using 2-isopropoxy-4,4,5,5-tetramethyl-1,3,2-dioxaborolane (1.3 equiv.), butyllithium (1.1 equiv.) and 3-(3,5-difluorophenyl)oxetane (1.0 equiv.) to give 2-(2,6-difluoro-4-(oxetan-3-yl)phenyl)-4,4,5,5-tetramethyl-1,3,2-dioxaborolane in 8% yield. 1H NMR (400 MHz, <cdcl3>) δ ppm 6.90 (d, J=8.22 Hz, 2H), 5.07 (dd, J=6.06, 8.41 Hz, 2H), 4.70 (t, J=6.26 Hz, 2H), 4.13-4.23 (m, 1H), 1.39 (s, 12H).

### Synthesis of methyl 6-(2,6-difluoro-4-(oxetan-3-yl)phenyl)-5-fluoropicolinate

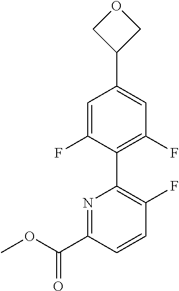

Method 1 was followed using methyl 6-bromo-5-fluoropicolinate (1.2 equiv.) and 2-(2,6-difluoro-4-(oxetan-3-yl)phenyl)-4,4,5,5-tetramethyl-1,3,2-dioxaborolane (1.0 equiv.) at 80° C. for 15 min in microwave to give methyl 6-(2,6-difluoro-4-(oxetan-3-yl)phenyl)-5-fluoropicolinate in 47% yield. LC/MS=324.0 (MH+), Rt=0.75 min.

### Synthesis of 6-(2,6-difluoro-4-(oxetan-3-yl)phenyl)-5-fluoropicolinic acid

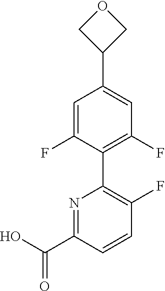

Method 2 was followed using methyl 6-(2,6-difluoro-4-(oxetan-3-yl)phenyl)-5-fluoropicolinate to give 6-(2,6-difluoro-4-(oxetan-3-yl)phenyl)-5-fluoropicolinic acid in 71% yield. LC/MS=309.9 (MH+), Rt=0.69 min.

### Synthesis of (R)-methyl 6-(2,6-difluoro-4-((tetrahydrofuran-3-yl)oxy)phenyl)-5-fluoropicolinate

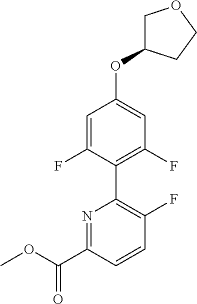

To a solution of methyl 6-(2,6-difluoro-4-hydroxyphenyl)-5-fluoropicolinate (1.0 equiv.), (S)-tetrahydrofuran-3-ol (3.0 equiv.) and triphenylphosphine (3.0 equiv.) in THF (0.20 MI at 0° C. was added DIAD (3.0 equiv.) was added. The mixture was stirred at ambient temperature overnight. The mixture was concentrated and partitioned between EtOAc and Water. The organic layer was washed with sat. NaHCO3, then brine, dried over Na2SO4 and concentrated to give (R)-methyl 6-(2,6-difluoro-4-(tetrahydrofuran-3-yloxy)phenyl)-5-fluoropicolinate in 96% yield. LC/MS=353.9 (MH+), Rt=0.88 min.

### Synthesis of (R)-6-(2,6-difluoro-4-((tetrahydrofuran-3-yl)oxy)phenyl)-5-fluoropicolinic acid

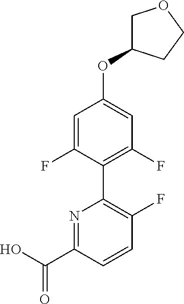

Method 2 was followed using (R)-methyl 6-(2,6-difluoro-4-((tetrahydrofuran-3-yl)oxy)phenyl)-5-fluoropicolinate to give (R)-6-(2,6-difluoro-4-((tetrahydrofuran-3-yl)oxy)phenyl)-5-fluoropicolinic acid in 52% yield. LC/MS=340.0 (MH+), Rt=0.76 min.

### Synthesis of (S)-methyl 6-(2,6-difluoro-4-((tetrahydrofuran-3-yl)oxy)phenyl)-5-fluoropicolinate

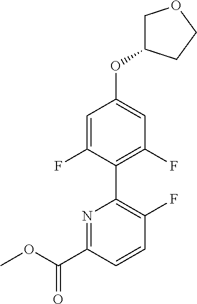

To a solution of methyl 6-(2,6-difluoro-4-hydroxyphenyl)-5-fluoropicolinate (1.0 equiv.), (R)-tetrahydrofuran-3-ol (3.0 equiv.) and triphenylphosphine (3.0 equiv.) in THF (0.20 MI at 0° C. was added DIAD (3.0 equiv.) was added. The mixture was stirred at ambient temperature overnight. The reaction mixture was used in next step without workup. LC/MS=353.9 (MH+), Rt=0.88 min.

### Synthesis of (S)-6-(2,6-difluoro-4-((tetrahydrofuran-3-yl)oxy)phenyl)-5-fluoropicolinic acid

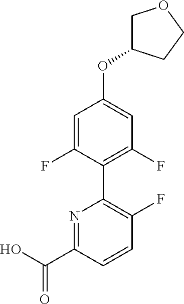

To the reaction mixture (1.0 equiv.) from previous step in THF (0.10 M) was added LiOH (4.0 equiv.). After stirred at rt for 2 hrs, the reaction mixture was concentrated and partitioned between EtOAc and Water. The aqueous solution was neutralized with 1N HCl, extracted with ethyl acetate, washed with brine, dried with sodium sulfate, filtered and concentrated to give (S)-6-(2,6-difluoro-4-((tetrahydrofuran-3-yl)oxy)phenyl)-5-fluoropicolinic acid in 52% yield. LC/MS=339.9 (MH+), Rt=0.76 min.

### Synthesis of 6,8-difluoro-4-methylchroman-4-ol

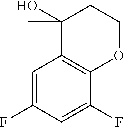

To an oven dried flask under Ar containing methylmagnesium bromide (2.0 equiv., 1.4 Min toluene/THF) at 60° C. was added a solution of 6,8-difluorochroman-4-one (1.0 equiv.) in THF (0.18 M). External heat was removed. The reaction mixture was stirred to rt for 2 hrs, poured into cold sat. NH₄Cl and extracted with EtOAc. The organic layer was washed with brine, dried over Na2SO4 and concentrated. The crude was purified by ISCO eluting from 15% to 30% EtOAc in heptanes to yield 6,8-difluoro-4-methylchroman-4-ol in 84% yield. 1H NMR (400 MHz, <cdcl3>) δ 1.62 (s, 3H), 2.11 (t, J=5.48 Hz, 2H), 4.26-4.39 (m, 2H), 6.78 (ddd, J=10.66, 8.12, 2.74 Hz, 1H), 6.99 (dt, J=9.10, 2.49 Hz, 1H).

### Synthesis of 6,8-difluoro-4-methyl-7-(4,4,5,5-tetramethyl-1,3,2-dioxaborolan-2-yl)chroman-4-ol

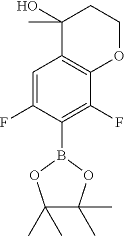

Method 3 was followed using 2-isopropoxy-4,4,5,5-tetramethyl-1,3,2-dioxaborolane (2.5 equiv.), butyllithium (2.5 equiv.) and 6,8-difluoro-4-methylchroman-4-ol (1.0 equiv.) to give 6,8-difluoro-4-methyl-7-(4,4,5,5-tetramethyl-1,3,2-dioxaborolan-2-yl)chroman-4-ol in 100% yield.

### Synthesis of methyl 6-(6,8-difluoro-4-hydroxy-4-methylchroman-7-yl)-5-fluoropicolinate

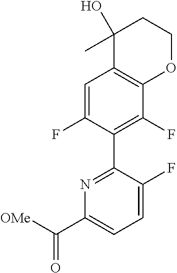

Method 1 was followed using methyl 6-bromo-5-fluoropicolinate (1.0 equiv.) and 6,8-difluoro-4-methyl-7-(4,4,5,5-tetramethyl-1,3,2-dioxaborolan-2-yl)chroman-4-ol (1.1 equiv.) at 80° C. for 2 hrs to give methyl 6-(6,8-difluoro-4-hydroxy-4-methylchroman-7-yl)-5-fluoropicolinate in 100% yield. LC/MS=353.9 (MH+), Rt=0.77 min.

### Synthesis of 6-(6,8-difluoro-4-hydroxy-4-methylchroman-7-yl)-5-fluoropicolinic acid

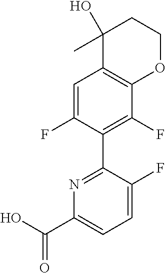

Method 2 was followed using methyl 6-(6,8-difluoro-4-hydroxy-4-methylchroman-7-yl)-5-fluoropicolinate to give 6-(6,8-difluoro-4-hydroxy-4-methylchroman-7-yl)-5-fluoropicolinic acid in 61% yield. LC/MS=339.9 (MH+), Rt=0.67 min.

### Synthesis of tert-butyl ((1S,3R,5S)-3-(3-(6-(6,8-difluoro-4-hydroxy-4-methylchroman-7-yl)-5-fluoropicolinamido)pyridin-4-yl)-5-methylcyclohexyl)carbamate

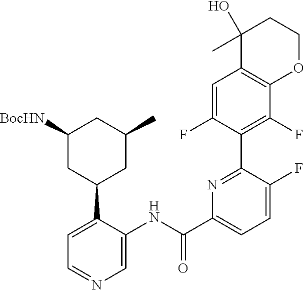

Method 5 was followed using tert-butyl ((1S,3R,5S)-3-(3-aminopyridin-4-yl)-5-methylcyclohexyl)carbamate (1.0 equiv.) and 6-(6,8-difluoro-4-hydroxy-4-methylchroman-7-yl)-5-fluoropicolinic acid (1.0 equiv.) to give tert-butyl ((1S,3R,5S)-3-(3-(6-(6,8-difluoro-4-hydroxy-4-methylchroman-7-yl)-5-fluoropicolinamido)pyridin-4-yl)-5-methylcyclohexyl)carbamate in 100% yield. LC/MS=627.1 (MH+), Rt=0.89, 0.91 min.

### Synthesis of N-(4-((1R,3S,5S)-3-amino-5-methyl cyclohexyl)pyridin-3-yl)-6-((S)-6,8-difluoro-4-hydroxy-4-methylchroman-7-yl)-5-fluoropicolinamide and N-(4-((1R,3S,5S)-3-amino-5-methylcyclohexyl)pyridin-3-yl)-6-((R)-6,8-difluoro-4-hydroxy-4-methylchroman-7-yl)-5-fluoropicolinamide

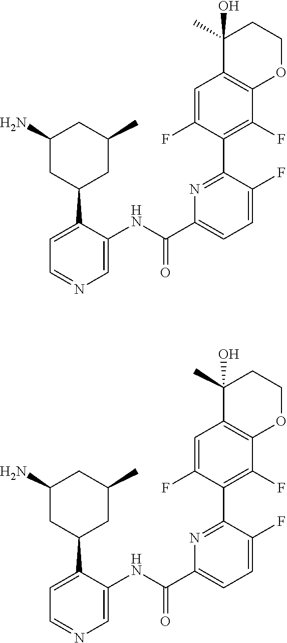

To a solution of HCl (24.0 equiv.) in dioxane (0.65 M) was added tert-butyl (1S,3R,5S)-3-(3-(6-(6,8-difluoro-4-hydroxy-4-methylchroman-7-yl)-5-fluoropicolinamido)pyridin-4-yl)-5-methylcyclohexylcarbamate (1.0 equiv.). After stirred at rt for 30 min, the reaction was concentrated and the crude was purified by reverse prep HPLC. The combined fractions was concentrated and partitioned between EtOAc and aq. NaHCO3. The organic layer was washed with brine, dried over Na2SO4 and concentrated. Purification was completed via chiral HPLC (EtOH/heptane)=20/80, 20 mL/min, AD column) to yield N-(4-((1R,3S,5S)-3-amino-5-methylcyclohexyl)pyridin-3-yl)-6-(S)-6,8-difluoro-4-hydroxy-4-methylchroman-7-yl)-5-fluoropicolinamide (10% yield) and N-(4-((1R,3S,5S)-3-amino-5-methylcyclohexyl)pyridin-3-yl)-6-(R)-6,8-difluoro-4-hydroxy-4-methylchroman-7-yl)-5-fluoropicolinamide (10% yield). LC/MS=527.1 (MH+), Rt=0.64 min.

### Synthesis of N-(4-((1R,3S,5S)-3-amino-5-methylcyclohexyl)pyridin-3-yl)-6-(6,8-difluoro-4-methyl-2H-chromen-7-yl)-5-fluoropicolinamide

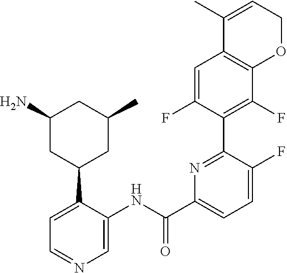

To a solution of tert-butyl (1S,3R,5S)-3-(3-(6-((R)-6,8-difluoro-4-hydroxy-4-methylchroman-7-yl)-5-fluoropicolinamido)pyridin-4-yl)-5-methylcyclohexylcarbamate (1.0 equiv.) in DCM (0.06 M) was added TFA (5 equiv.). After stirred at rt for 1 hr, the mixture was concentrated and purified by reverse HPLC to yield N-(4-((1R,3S,5S)-3-amino-5-methylcyclohexyl)pyridin-3-yl)-6-(6,8-difluoro-4-methyl-2H-chromen-7-yl)-5-fluoropicolinamide in 14% yield. LC/MS=509.1 (MH+), Rt=0.75 min.

### Synthesis of 1-(3,5-difluoro-4-(4,4,5,5-tetramethyl-1,3,2-dioxaborolan-2-yl)phenyl)-2-methylpropan-2-ol

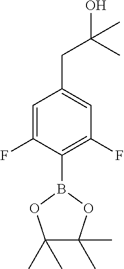

Method 3 was followed using 2-isopropoxy-4,4,5,5-tetramethyl-1,3,2-dioxaborolane (2.5 equiv.), butyllithium (2.5 equiv.) and 1-(3,5-difluorophenyl)-2-methylpropan-2-ol (1.0 equiv.) to give 1-(3,5-difluoro-4-(4,4,5,5-tetramethyl-1,3,2-dioxaborolan-2-yl)phenyl)-2-methylpropan-2-ol in 100% yield. ¹H NMR (400 MHz, CHLOROFORM-d) δ ppm 1.24-1.25 (m, 12H), 1.38 (s, 6H), 2.74 (d, J=2.74 Hz, 2H), 6.74 (d, J=8.22 Hz, 2H).

### Synthesis of methyl 6-(2,6-difluoro-4-(2-hydroxy-2-methylpropyl)phenyl)-5-fluoropicolinate

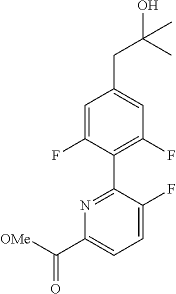

Method 1 was followed using methyl 6-bromo-5-fluoropicolinate (1.0 equiv.) and 1-(3,5-difluoro-4-(4,4,5,5-tetramethyl-1,3,2-dioxaborolan-2-yl)phenyl)-2-methylpropan-2-ol (1.0 equiv.) at 80° C. for 3 hrs to give methyl 6-(2,6-difluoro-4-(2-hydroxy-2-methylpropyl)phenyl)-5-fluoropicolinate in 100% yield. LC/MS=339.9 (MH+), Rt=0.82 min.

### Synthesis of 6-(2,6-difluoro-4-(2-hydroxy-2-methylpropyl)phenyl)-5-fluoropicolinic acid

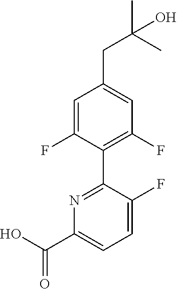

Method 2 was followed using methyl 6-(2,6-difluoro-4-(2-hydroxy-2-methylpropyl)phenyl)-5-fluoropicolinate to give 6-(2,6-difluoro-4-(2-hydroxy-2-methylpropyl)phenyl)-5-fluoropicolinic acid in 63% yield. LC/MS=325.9 (MH+), Rt=0.71 min.

### Synthesis of 1,3-difluoro-5-(2-methoxypropan-2-yl)benzene

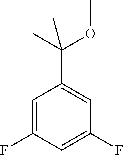

To a solution of 2-(3,5-difluorophenyl)propan-2-ol (1.0 equiv.) in DMF (0.23 M) at 0° C. was added NaH (1.1 equiv.). After 1 hr, MeI (1.1 equiv.) was added. The ice bath was removed and the reaction mixture was stirred at rt for 2 hrs. The reaction was quenched with water, and partitioned between EtOAc and water. The organic layer was washed with brine, dried over Na2SO4 and concentrated. The crude was purified by ISCO chromatography (eluting with 6% ether in hepatanes) to give 1,3-difluoro-5-(2-methoxypropan-2-yl)benzene in 82% yield. 1H NMR (400 MHz, CHLOROFORM-d) δ ppm 1.47-1.52 (m, 6H), 3.10 (s, 3H), 6.65-6.73 (m, 1H), 6.92 (dd, J=9.00, 2.35 Hz, 2H).

### Synthesis of 2-(2,6-difluoro-4-(2-methoxypropan-2-yl)phenyl)-4,4,5,5-tetramethyl-1,3,2-dioxaborolane

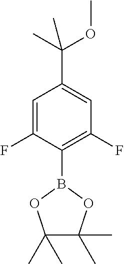

Method 3 was followed using 2-isopropoxy-4,4,5,5-tetramethyl-1,3,2-dioxaborolane (2.5 equiv.), butyllithium (2.5 equiv.) and 1,3-difluoro-5-(2-methoxypropan-2-yl)benzene (1.0 equiv.) to give 2-(2,6-difluoro-4-(2-methoxypropan-2-yl)phenyl)-4,4,5,5-tetramethyl-1,3,2-dioxaborolane in 100% yield. 1H NMR (400 MHz, CHLOROFORM-d) δ ppm 1.36-1.40 (m, 12H), 1.48 (s, 6H), 3.07 (s, 3H), 6.89 (d, J=9.00 Hz, 2H).

### Synthesis of methyl 3-amino-6-(2,6-difluoro-4-(2-methoxypropan-2-yl)phenyl)-5-fluoropicolinate

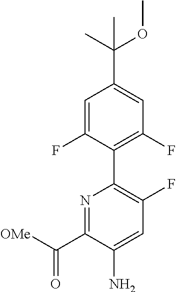

Method 1 was followed using methyl 3-amino-6-bromo-5-fluoropicolinate (1.0 equiv.) and 2-(2,6-difluoro-4-(2-methoxypropan-2-yl)phenyl)-4,4,5,5-tetramethyl-1,3,2-dioxaborolane (2.0 equiv.) at 100° C. for 20 min in microwave to give methyl 3-amino-6-(2,6-difluoro-4-(2-methoxypropan-2-yl)phenyl)-5-fluoropicolinatein 100% yield. LC/MS=355.1 (MH+), Rt=0.92 min.

### Synthesis of 3-amino-6-(2,6-difluoro-4-(2-methoxypropan-2-yl)phenyl)-5-fluoropicolinic acid

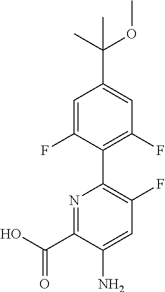

Method 2 was followed using methyl 3-amino-6-(2,6-difluoro-4-(2-methoxypropan-2-yl)phenyl)-5-fluoropicolinate to give 3-amino-6-(2,6-difluoro-4-(2-methoxypropan-2-yl)phenyl)-5-fluoropicolinic acid in 45% yield. LC/MS=341.0 (MH+), Rt=0.87 min.

### Synthesis of methyl 3-amino-6-(2,6-difluoro-4-(2-methoxyethoxy)phenyl)-5-fluoropicolinate

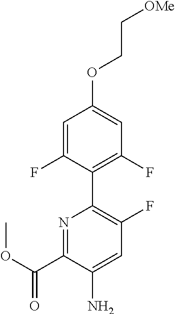

Method 1 was followed using methyl 3-amino-6-bromo-5-fluoropicolinate (1.0 equiv.) and 2-(2,6-difluoro-4-(2-methoxyethoxy)phenyl)-4,4,5,5-tetramethyl-1,3,2-dioxaborolane (1.5 equiv.) at 100° C. for 20 min in microwave to give methyl 3-amino-6-(2,6-difluoro-4-(2-methoxyethoxy)phenyl)-5-fluoropicolinate in 36% yield. LC/MS=357.2 (MH+), Rt=0.82 min. 1H NMR (400 MHz, <cdcl3>) δ ppm 3.46 (s, 3H), 3.76 (dd, J=5.28, 3.72 Hz, 2 H), 3.95 (s, 3H), 4.12 (dd, J=5.48, 3.91 Hz, 2H), 6.01 (br. s., 2H), 6.49-6.63 (m, 2H), 6.82 (d, J=9.78 Hz, 1H).

### Synthesis of 3-amino-6-(2,6-difluoro-4-(2-methoxyethoxy)phenyl)-5-fluoropicolinic acid

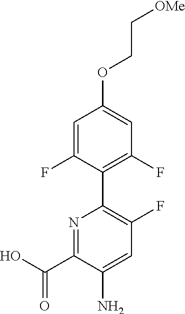

Method 2 was followed using methyl 3-amino-6-(2,6-difluoro-4-(2-methoxyethoxy)phenyl)-5-fluoropicolinate to give 3-amino-6-(2,6-difluoro-4-(2-methoxyethoxy)phenyl)-5-fluoropicolinic acid in 98% yield. LC/MS=343.0 (MH+), Rt=0.82 min.

### Synthesis of methyl 3-amino-6-(2,6-difluoro-4-(2-hydroxypropan-2-yl)phenyl)-5-fluoropicolinate

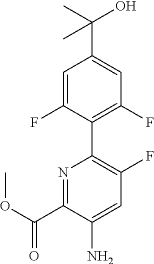

Method 1 was followed using methyl 3-amino-6-bromo-5-fluoropicolinate (1.0 equiv.) and 2-(3,5-difluoro-4-(4,4,5,5-tetramethyl-1,3,2-dioxaborolan-2-yl)phenyl)propan-2-ol (2.0 equiv.) at 100° C. for 20 min in microwave to give methyl 3-amino-6-(2,6-difluoro-4-(2-hydroxypropan-2-yl)phenyl)-5-fluoropicolinate in 87% yield. LC/MS=340.9 (MH+), Rt=0.77 min.

### Synthesis of 3-amino-6-(2,6-difluoro-4-(2-hydroxypropan-2-yl)phenyl)-5-fluoropicolinic acid

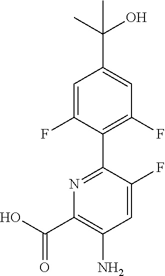

Method 2 was followed using methyl 3-amino-6-(2,6-difluoro-4-(2-hydroxypropan-2-yl)phenyl)-5-fluoropicolinate to give 3-amino-6-(2,6-difluoro-4-(2-hydroxypropan-2-yl)phenyl)-5-fluoropicolinic acid in 98% yield. LC/MS=326.8 (MH+), Rt=0.68 min. 1H NMR (400 MHz, <cdcl3>) δ ppm 2.10 (s, 6H), 6.92 (d, J=9.78 Hz, 1H), 7.09-7.19 (m, 2H).

### Synthesis of 3-(3,5-difluorophenyl)-3-methoxyoxetane

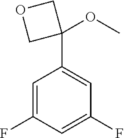

A solution of 3-(3,5-difluorophenyl)oxetan-3-ol (1.0 equiv.) in DMF (0.23 M) was cooled in an ice water bath. NaH, 60% dispersion in mineral oil (1.1 equiv.) was added. The mixture was stirred for 1 hr. iodomethane (1.1 equiv.) was added in a dropwise fashion. The ice bath was removed, and the mixture was stirred for 2 hr at ambient temperature. The reaction mixture was quenched by the addition of water. The mixture was extracted with ether. The combined extracts were washed sequentially with water and brine, dried over sodium sulfate, filtered, and concentrated. The crude material was purified by flash chromatography over silica gel (2:1 pentane:ether) to give 3-(3,5-difluorophenyl)-3-methoxyoxetane in 83% yield. ¹H NMR (400 MHz, CHLOROFORM-d) δ ppm 3.18 (s, 3H), 4.70 (d, J=7.04 Hz, 2H), 4.92 (d, J=7.43 Hz, 2H), 6.80 (tt, J=8.66, 2.30 Hz, 1H), 6.99-7.08 (m, 2H).

### Synthesis of 2-(2,6-difluoro-4-(3-methoxyoxetan-3-yl)phenyl)-4,4,5,5-tetramethyl-1,3,2-dioxaborolane

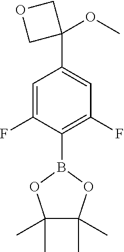

Method 3 was followed using 2-isopropoxy-4,4,5,5-tetramethyl-1,3,2-dioxaborolane (1.3 equiv.), butyllithium (1.3 equiv.) and 3-(3,5-difluorophenyl)-3-methoxyoxetane (1.0 equiv.) to give 2-(2,6-difluoro-4-(3-methoxyoxetan-3-yl)phenyl)-4,4,5,5-tetramethyl-1,3,2-dioxaborolane in 100% yield. 1H NMR (400 MHz, CHLOROFORM-d) δ ppm 1.22-1.26 (m, 12H), 3.16 (s, 3H), 4.67-4.73 (m, 2H), 4.89-4.94 (m, 2H), 7.00 (d, J=8.22 Hz, 2H).

### Synthesis of methyl 3-amino-6-(2,6-difluoro-4-(3-methoxyoxetan-3-yl)phenyl)-5-fluoropicolinate

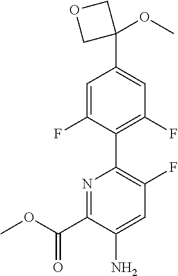

Method 1 was followed using methyl 3-amino-6-bromo-5-fluoropicolinate (1.0 equiv.) and 2-(2,6-difluoro-4-(3-methoxyoxetan-3-yl)phenyl)-4,4,5,5-tetramethyl-1,3,2-dioxaborolane (2.5 equiv.) at 90° C. for 1 hr to give methyl 3-amino-6-(2,6-difluoro-4-(3-methoxyoxetan-3-yl)phenyl)-5-fluoropicolinate in 100% yield. LC/MS=368.9 (MH+), Rt=0.79 min.

### Synthesis of 3-amino-6-(2,6-difluoro-4-(3-methoxyoxetan-3-yl)phenyl)-5-fluoropicolinic acid

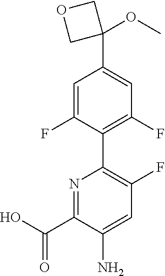

Method 2 was followed using methyl 3-amino-6-(2,6-difluoro-4-(3-methoxyoxetan-3-yl)phenyl)-5-fluoropicolinate to give 3-amino-6-(2,6-difluoro-4-(3-methoxyoxetan-3-yl)phenyl)-5-fluoropicolinic acid in 97% yield. LC/MS=354.9 (MH+), Rt=0.74 min.

### Synthesis of methyl 3-amino-6-(2,6-difluoro-4-isopropoxyphenyl)-5-fluoropicolinate

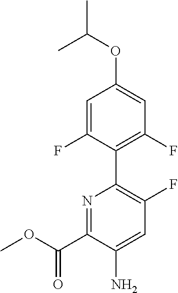

Method 1 was followed using methyl 3-amino-6-bromo-5-fluoropicolinate (1.0 equiv.) and 2-(2,6-difluoro-4-isopropoxyphenyl)-4,4,5,5-tetramethyl-1,3,2-dioxaborolane (1.6 equiv.) at 70° C. for 1 hr to give methyl 3-amino-6-(2,6-difluoro-4-isopropoxyphenyl)-5-fluoropicolinate in 44% yield. LC/MS=340.9 (MH+), Rt=0.98 min.

### Synthesis of 3-amino-6-(2,6-difluoro-4-isopropoxyphenyl)-5-fluoropicolinic acid

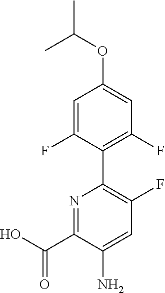

Method 2 was followed using methyl 3-amino-6-(2,6-difluoro-4-isopropoxyphenyl)-5-fluoropicolinate to give 3-amino-6-(2,6-difluoro-4-isopropoxyphenyl)-5-fluoropicolinic acid in 84% yield. LC/MS=327.0 (MH+), Rt=0.94 min.

### Synthesis of methyl 6-(2,6-difluoro-4-(2-(2-oxopyrrolidin-1-yl)ethoxy)phenyl)-5-fluoropicolinate

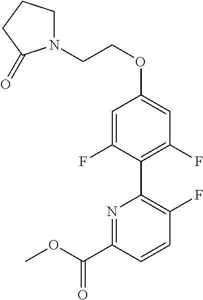

To a solution of triphenylphosphine (1.5 equiv.), methyl 6-(2,6-difluoro-4-hydroxyphenyl)-5-fluoropicolinate (1.0 equiv.) and 1-(2-hydroxyethyl)pyrrolidin-2-one (1.2 equiv.) in THF (0.14 M) at 0° C. was added DIAD (1.5 equiv.) dropwise. The reaction was allowed to warm to rt and stirred for 6 hrs. The reaction mixture was concentrated under vacuo and purified via ISCO (ethyl acetate and heptanes 0-100%) to give methyl 6-(2,6-difluoro-4-(2-(2-oxopyrrolidin-1-yl)ethoxy)phenyl)-5-fluoropicolinate in 96% yield. LC/MS=395.0 (MH+), Rt=0.80 min. ¹H NMR (400 MHz, <cdcl3>) δ ppm 1.97-2.14 (m, 2H), 2.31-2.50 (m, 2H), 3.57 (t, J=7.04 Hz, 2H), 3.71 (t, J=5.09 Hz, 2H), 4.00 (s, 3H), 4.08-4.20 (m, 3H), 6.56 (d, J=9.00 Hz, 2H), 7.63 (t, J=8.41 Hz, 1H), 8.24 (dd, J=8.61, 3.91 Hz, 1H).

### Synthesis of 6-(2,6-difluoro-4-(2-(2-oxopyrrolidin-1-yl)ethoxy)phenyl)-5-fluoropicolinic acid

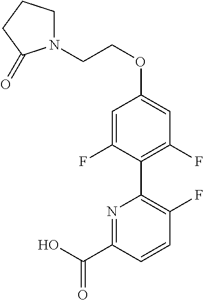

Method 2 was followed using methyl 6-(2,6-difluoro-4-(2-(2-oxopyrrolidin-1-yl)ethoxy)phenyl)-5-fluoropicolinate to give 6-(2,6-difluoro-4-(2-(2-oxopyrrolidin-1-yl)ethoxy)phenyl)-5-fluoropicolinic acid in 70% yield. LC/MS=381.0 (MH+), Rt=0.70 min.

### Synthesis of tert-butyl ((1S,3R,5S)-3-(3-(6-(2,6-difluoro-3-formylphenyl)-5-fluoropicolinamido)pyridin-4-yl)-5-methylcyclohexyl)carbamate

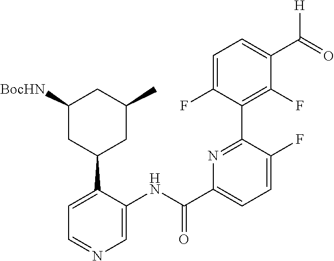

Method 1 was followed using tert-butyl ((1S,3R,5S)-3-(3-(6-bromo-5-fluoropicolinamido)pyridin-4-yl)-5-methylcyclohexyl)carbamate (1.0 equiv.) and (2,6-difluoro-3-formylphenyl)boronic acid (5.0 equiv.) at 100° C. for 30 min in microwave to give tert-butyl ((1S,3R,5S)-3-(3-(6-(2,6-difluoro-3-formylphenyl)-5-fluoropicolinamido)pyridin-4-yl)-5-methylcyclohexyl)carbamate in 100% yield. LC/MS=569.2 (MH+), Rt=0.89 min.

### Synthesis of tert-butyl ((1S,3R,5S)-3-(3-(6-(2,6-difluoro-3-(hydroxymethyl)phenyl)-5-fluoropicolinamido)pyridin-4-yl)-5-methylcyclohexyl)carbamate

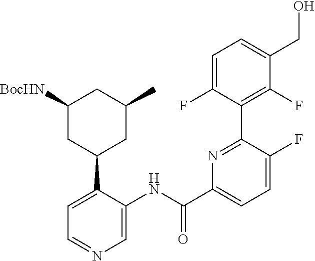

To a solution of tert-butyl ((1S,3R,5S)-3-(3-(6-(2,6-difluoro-3-formylphenyl)-5-fluoropicolinamido)pyridin-4-yl)-5-methylcyclohexyl)carbamate (1.0 equiv.) in MeOH (0.04 M) at 0° C. was added NaBH4 (2.0 equiv.). After 5 min at 0° C., the reaction was quenched by addition of H2O and the volatiles were removed in vacuo. The reaction was diluted with EtOAc and was washed with NaCl(sat.), was dried over MgSO4, filtered and concentrated. The residue was purified by ISCO SiO2 chromatography (0-100% EtOAc/n-heptanes) to yield tert-butyl ((1S,3R,5S)-3-(3-(6-(2,6-difluoro-3-(hydroxymethyl)phenyl)-5-fluoropicolinamido)pyridin-4-yl)-5-methylcyclohexyl)carbamate in 47% yield. LC/MS=571.1 (MH+), Rt=0.82 min.

### Synthesis of methyl 6-(4-(bromomethyl)-2,6-difluorophenyl)-5-fluoropicolinate

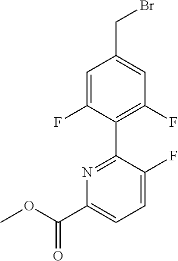

A solution of bromine (1.0 equiv.) in DCM (0.20 M) was added to triphenylphosphine (1.0 equiv.). The mixture became homogeneous and colorless and was stirred for an additional 30 min. This heterogeneous mixture was added to methyl 6-(2,6-difluoro-4-(hydroxymethyl)phenyl)-5-fluoropicolinate (1.0 equiv.). The light yellow solution was stirred at 50° C. for 3 hrs. The reaction mixture was concentrated and purified by flash chromatography over silica gel to give methyl 6-(4-(bromomethyl)-2,6-difluorophenyl)-5-fluoropicolinate in 71% yield. LC/MS=362.1 (MH+), Rt=0.92 min.

### Synthesis of methyl 6-(4-(cyanomethyl)-2,6-difluorophenyl)-5-fluoropicolinate

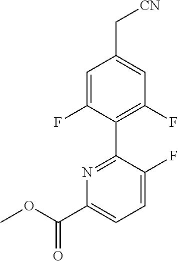

A solution of sodium cyanide (1.4 equiv.) in water (0.65 M) was stirred at 50° C.

A solution of methyl 6-(4-(bromomethyl)-2,6-difluorophenyl)-5-fluoropicolinate (1.0 equiv.) in ACN (0.07 M) was added in a dropwise fashion over 15 min. The colorless solution was stirred at 50° C. for 2 hrs. The cooled reaction mixture was concentrated. Water was added, and the product was extracted with ethyl acetate. The combined extracts were dried over sodium sulfate, filtered, and concentrated to give methyl 6-(4-(cyanomethyl)-2,6-difluorophenyl)-5-fluoropicolinate in 89% yield. LC/MS=307.1 (MH+), Rt=0.77 min.

### Synthesis of methyl 6-(4-(2-cyanopropan-2-yl)-2,6-difluorophenyl)-5-fluoropicolinate

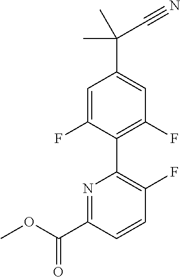

Sodium hydride (2.2 equiv.) was added to a solution of methyl 6-(4-(cyanomethyl)-2,6-difluorophenyl)-5-fluoropicolinate (1.0 equiv.) in DMSO (0.26 M). The red mixture was stirred for 15 min at ambient temperature. iodomethane (2.1 equiv.) was added in a dropwise fashion. The reaction mixture was stirred for 20 min at ambient temperature. The reaction mixture was diluted with water and extracted with ethyl acetate. The combined organics were washed sequentially with water and brine, dried over sodium sulfate, filtered, concentrated, and purified by flash chromatography (heptanes:ethyl acetate gradient) over silica gel to give methyl 6-(4-(2-cyanopropan-2-yl)-2,6-difluorophenyl)-5-fluoropicolinate in 35% yield. LC/MS=335.1 (MH+), Rt=0.90 min.

### Synthesis of 6-(4-(2-cyanopropan-2-yl)-2,6-difluorophenyl)-5-fluoropicolinic acid

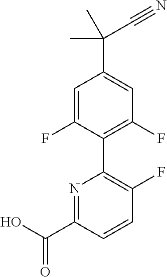

Method 2 was followed using methyl 6-(4-(2-cyanopropan-2-yl)-2,6-difluorophenyl)-5-fluoropicolinate to give 6-(4-(2-cyanopropan-2-yl)-2,6-difluorophenyl)-5-fluoropicolinic acid in 99% yield. LC/MS=321.2 (MH+), Rt=0.79 min.

### Synthesis of methyl 6-(4-(4-cyanotetrahydro-2H-pyran-4-yl)-2,6-difluorophenyl)-5-fluoropicolinate

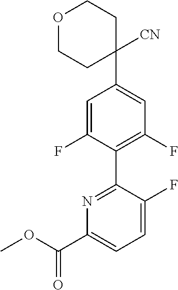

Sodium hydride (2.2 equiv.) was added to a solution of methyl 6-(4-(cyanomethyl)-2,6-difluorophenyl)-5-fluoropicolinate (1.0 equiv.) in DMSO (0.51 M). The red mixture was stirred for 15 min at ambient temperature. bis(2-bromoethyl)ether (1.1 equiv.) was added in a dropwise fashion. After stirred at rt for 30 min, the mixture was diluted with water and extracted with ethyl acetate. The combined extracts were dried over sodium sulfate, filtered, concentrated and purified by flash chromatography (heptanes:ethyl acetate gradient) over silica gel to give methyl 6-(4-(4-cyanotetrahydro-2H-pyran-4-yl)-2,6-difluorophenyl)-5-fluoropicolinate in 15% yield. LC/MS=377.2 (MH+), Rt=0.85 min.

### Synthesis of 6-(4-(4-cyanotetrahydro-2H-pyran-4-yl)-2,6-difluorophenyl)-5-fluoropicolinic acid

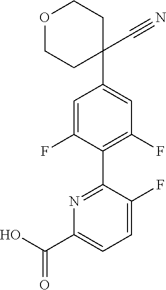

Method 2 was followed using methyl 6-(4-(4-cyanotetrahydro-2H-pyran-4-yl)-2,6-difluorophenyl)-5-fluoropicolinate to give 6-(4-(4-cyanotetrahydro-2H-pyran-4-yl)-2,6-difluorophenyl)-5-fluoropicolinic acid in 96% yield. LC/MS=363.2 (MH+), Rt=0.74 min.

### Synthesis of 4-(3,5-difluorophenyl)morpholine

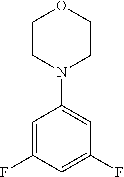

Tert-amyl alcohol was degassed by bubbling N2 through it for 15 min. 1-bromo-3,5-difluorobenzene (1.0 equiv.), Pd₂(dba)₃(0.03 equiv.), X-Phos (0.14 equiv.), potassium carbonate (1.0 equiv.) and morpholine (0.92 equiv.) were added and the mixture heated to 100° C. for 18 hrs under N2. The solution was diluted with water and ether. The aqueous was extracted with ether. The combined organics were dried over sodium sulfate, filtered and concentrated to afford a red heterogeneous mixture. The crude oil was purified by ISCO SiO2 chromatography, eluting with 0-30% Ether in Pentanes, then eluting with 0-100% DCM in Pentanes to afford 4-(3,5-difluorophenyl)morpholine in 30% yield. ¹H NMR (400 MHz, CHLOROFORM-d) δ ppm 3.14 (d, J=9.78 Hz, 3H), 3.83 (d, J=5.09 Hz, 4H), 6.28 (tt, J=8.90, 2.05 Hz, 1H), 6.32-6.40 (m, 2H).

### Synthesis of 4-(3,5-difluoro-4-(4,4,5,5-tetramethyl-1,3,2-dioxaborolan-2-yl)phenyl)morpholine

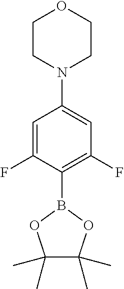

Method 3 was followed using 2-isopropoxy-4,4,5,5-tetramethyl-1,3,2-dioxaborolane (2.1 equiv.), butyllithium (1.0 equiv.) and 4-(3,5-difluorophenyl)morpholine (1.0 equiv.) to give 4-(3,5-difluoro-4-(4,4,5,5-tetramethyl-1,3,2-dioxaborolan-2-yl)phenyl)morpholine in 100% yield. 1H NMR (400 MHz, CHLOROFORM-d) δ ppm 6.26-6.34 (m, 2H), 3.80-3.84 (m, 4H), 3.18-3.23 (m, 4H), 1.36 (s, 12H).

### Synthesis of methyl 6-(2,6-difluoro-4-morpholinophenyl)-5-fluoropicolinate

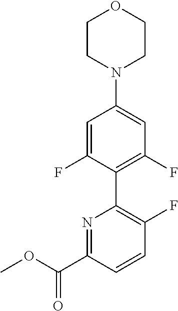

Method 1 was followed using methyl 6-bromo-5-fluoropicolinate (1.0 equiv.) and 4-(3,5-difluoro-4-(4,4,5,5-tetramethyl-1,3,2-dioxaborolan-2-yl)phenyl)morpholine (1.5 equiv.) at 100° C. for 30 min in microwave to give methyl 6-(2,6-difluoro-4-morpholinophenyl)-5-fluoropicolinate in 75% yield. LC/MS=353.3 (MH+), Rt=0.86 min. 1H NMR (400 MHz, <cdcl3>) δ 8.21 (dd, J=3.91, 8.61 Hz, 1H), 7.61 (t, J=8.41 Hz, 1H), 6.43-6.52 (m, 2H), 4.00 (s, 3H), 3.83-3.89 (m, 4H), 3.19-3.25 (m, 4H).

### Synthesis of 6-(2,6-difluoro-4-morpholinophenyl)-5-fluoropicolinic acid

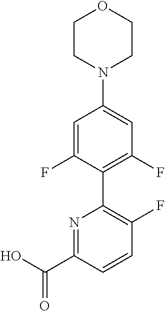

Method 2 was followed using methyl 6-(2,6-difluoro-4-morpholinophenyl)-5-fluoropicolinate to give 6-(2,6-difluoro-4-morpholinophenyl)-5-fluoropicolinic acid in 68% yield. LC/MS=339.1 (MH+), Rt=0.75 min. 1H NMR (400 MHz, <dmso>) δ 13.40 (br. s., 1H), 8.17 (dd, J=3.91, 8.61 Hz, 1H), 8.00 (t, J=8.80 Hz, 1H), 6.78-6.87 (m, 2H), 3.70-3.76 (m, 4H), 3.26-3.30 (m, 4H).

### Synthesis of 1,3-difluoro-5-(isopropoxymethyl)benzene

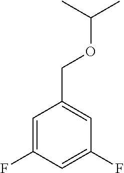

2-propanol (1.0 equiv.) was dissolved in DMF (0.20 M). Sodium hydride, 60% in mineral oil (1.1 equiv.) was added. The reaction mixture was stirred at ambient temperature for 1 hr. 3,5-difluorobenzyl bromide (1.1 equiv.) was added in a dropwise fashion. The mixture was stirred overnight at ambient temperature. The reaction mixture was quenched by the addition of water. The mixture was extracted with ether. The combined extracts were washed sequentially with water and brine, dried over sodium sulfate, filtered, and concentrated. The crude material was purified by flash chromatography over silica gel (4:1 pentane:ether) to give 1,3-difluoro-5-(isopropoxymethyl)benzene in 54% yield. 1H NMR (400 MHz, CHLOROFORM-d) δ ppm 1.22 (d, J=5.87 Hz, 6H), 3.68 (spt, J=6.13 Hz, 1H), 4.48 (s, 2H), 6.69 (tt, J=9.00, 2.35 Hz, 1H), 6.83-6.92 (m, 2H).

### Synthesis of 2-(2,6-difluoro-4-(isopropoxymethyl)phenyl)-4,4,5,5-tetramethyl-1,3,2-dioxaborolane

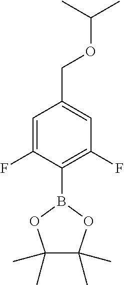

Method 3 was followed using 2-isopropoxy-4,4,5,5-tetramethyl-1,3,2-dioxaborolane (1.5 equiv.), butyllithium (1.5 equiv.) and 1,3-difluoro-5-(isopropoxymethyl)benzene (1.0 equiv.) to give 2-(2,6-difluoro-4-(isopropoxymethyl)phenyl)-4,4,5,5-tetramethyl-1,3,2-dioxaborolane in 95% yield.

### Synthesis of methyl 6-(2,6-difluoro-4-(isopropoxymethyl)phenyl)-5-fluoropicolinate

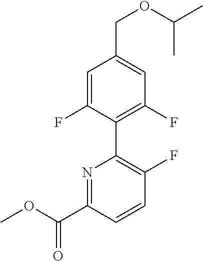

Method 1 was followed using methyl 6-bromo-5-fluoropicolinate (1.0 equiv.) and 2-(2,6-difluoro-4-(isopropoxymethyl)phenyl)-4,4,5,5-tetramethyl-1,3,2-dioxaborolane (2.5 equiv.) at 90° C. for 1 hr to give methyl 6-(2,6-difluoro-4-(isopropoxymethyl)phenyl)-5-fluoropicolinate in 61% yield. LC/MS=340.2 (MH+), Rt=0.99 min.

### Synthesis of 6-(2,6-difluoro-4-(isopropoxymethyl)phenyl)-5-fluoropicolinic acid

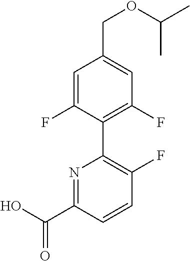

Method 2 was followed using methyl 6-(2,6-difluoro-4-(isopropoxymethyl)phenyl)-5-fluoropicolinate to give 6-(2,6-difluoro-4-(isopropoxymethyl)phenyl)-5-fluoropicolinic acid in 96% yield. LC/MS=326.2 (MH+), Rt=0.87 min.

### Synthesis of 4-((3,5-difluorobenzyl)oxy)tetrahydro-2H-pyran

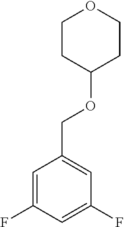

Tetrahydro-2H-pyran-4-ol (1.0 equiv.) was dissolved in DMF (0.20 M). Sodium hydride, 60% in mineral oil (1.1 equiv.) was added. The reaction mixture was stirred at ambient temperature for 1 hr. 3,5-difluorobenzyl bromide (1.1 equiv.) was added in a dropwise fashion. The mixture was stirred overnight at ambient temperature. The reaction mixture was quenched by the addition of water. The mixture was extracted with ether. The combined extracts were washed sequentially with water and brine, dried over sodium sulfate, filtered, and concentrated. The crude material was purified by flash chromatography over silica gel (5:2 pentane:ether) to give 4-((3,5-difluorobenzyl)oxy)tetrahydro-2H-pyran in 49% yield. ¹H NMR (400 MHz, CHLOROFORM-d) δ ppm 1.61-1.72 (m, 2H), 1.89-1.98 (m, 2H), 3.46 (ddd, J=11.64, 9.49, 2.74 Hz, 2H), 3.59 (tt, J=8.66, 4.26 Hz, 1H), 3.97 (dt, J=11.74, 4.50 Hz, 2H), 4.54 (s, 2H), 6.71 (tt, J=8.95, 2.20 Hz, 1H), 6.83-6.92 (m, 2H).

### Synthesis of 2-(2,6-difluoro-4-(((tetrahydro-2H-pyran-4-yl)oxy)methyl)phenyl)-4,4,5,5-tetramethyl-1,3,2-dioxaborolane

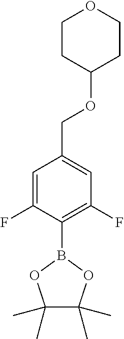

Method 3 was followed using 2-isopropoxy-4,4,5,5-tetramethyl-1,3,2-dioxaborolane (1.6 equiv.), butyllithium (1.6 equiv.) and 4-((3,5-difluorobenzyl)oxy)tetrahydro-2H-pyran (1.0 equiv.) to give 2-(2,6-difluoro-4-(((tetrahydro-2H-pyran-4-yl)oxy)methyl)phenyl)-4,4,5,5-tetramethyl-1,3,2-dioxaborolane in 97% yield.

### Synthesis of methyl 6-(2,6-difluoro-4-(((tetrahydro-2H-pyran-4-yl)oxy)methyl)phenyl)-5-fluoropicolinate

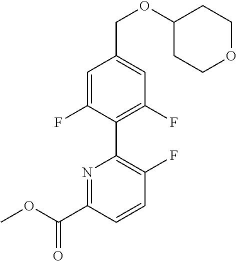

Method 1 was followed using methyl 6-bromo-5-fluoropicolinate (1.0 equiv.) and 2-(2,6-difluoro-4-((tetrahydro-2H-pyran-4-yloxy)methyl)phenyl)-4,4,5,5-tetramethyl-1,3,2-dioxaborolane (2.5 equiv.) at 90° C. for 1 hr to give methyl 6-(2,6-difluoro-4-(((tetrahydro-2H-pyran-4-yl)oxy)methyl)phenyl)-5-fluoropicolinate in 98% yield. LC/MS=382.2 (MH+), Rt=0.88 min.

### Synthesis of 6-(2,6-difluoro-4-(((tetrahydro-2H-pyran-4-yl)oxy)methyl)phenyl)-5-fluoropicolinic acid

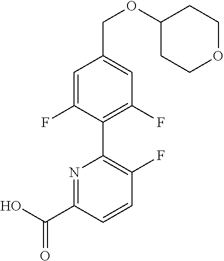

Method 2 was followed using methyl 6-(2,6-difluoro-4-(((tetrahydro-2H-pyran-4-yl)oxy)methyl)phenyl)-5-fluoropicolinate to give 6-(2,6-difluoro-4-(((tetrahydro-2H-pyran-4-yl)oxy)methyl)phenyl)-5-fluoropicolinic acid in 97% yield. LC/MS=368.1 (MH+), Rt=0.77 min.

### Synthesis of methyl 6-(4-(2-(dimethylamino)-2-oxoethoxy)-2,6-difluorophenyl)-5-fluoropicolinate

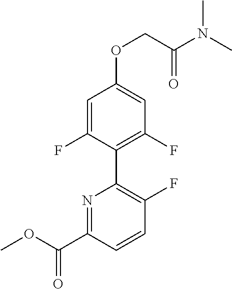

A solution of methyl 6-(2,6-difluoro-4-hydroxyphenyl)-5-fluoropicolinate (1.0 equiv.) in DMF (0.17 M) was added NaH, 60% dispersion in mineral oil (1.1 equiv.). The mixture was stirred for 30 min at ambient temperature. 2-chloro-N,N-dimethylacetamide (1.1 equiv.) was added in a dropwise fashion. The mixture was stirred overnight at ambient temperature. The reaction mixture was quenched by the addition of water. The mixture was extracted with ethyl acetate. The combined extracts were washed sequentially with water and brine, dried over sodium sulfate, filtered, and concentrated to give methyl 64442-(dimethylamino)-2-oxoethoxy)-2,6-difluorophenyl)-5-fluoropicolinate in 100% yield. LC/MS=369.2 (MH+), Rt=0.74 min.

### Synthesis of 6-(4-(2-(dimethylamino)-2-oxoethoxy)-2,6-difluorophenyl)-5-fluoropicolinic acid

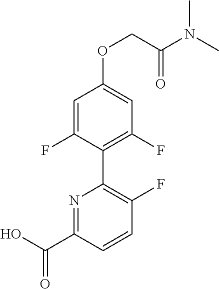

Method 2 was followed using methyl 6-(4-(2-(dimethylamino)-2-oxoethoxy)-2,6-difluorophenyl)-5-fluoropicolinate to give 6-(4-(2-(dimethylamino)-2-oxoethoxy)-2,6-difluorophenyl)-5-fluoropicolinic acid in 98% yield. LC/MS=355.2 (MH+), Rt=0.65 min.

### Synthesis of methyl 6-(2,6-difluoro-4-((2-oxopyrrolidin-1-yl)methyl)phenyl)-5-fluoropicolinate

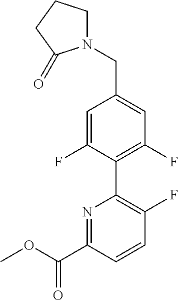

To a solution of methyl 6-(2,6-difluoro-4-formylphenyl)-5-fluoropicolinate (1.0 equiv.) in MeOH (0.10 M) was added methyl 4-aminobutanoate (1.2 equiv.), followed by TEA (1.4 equiv.). The homogeneous solution was stirred at rt for 30 min, then sodium borohydride (1.0 equiv.) was added. The reaction was heated to 45° C. for 2 days. Upon cooling to rt, the mixture was diluted with water, concentrated the volatiles in vacuo and partitioned between ethyl acetate and water. The organics were dried with sodium sulfate, filtered and concentrated to yield methyl 6-(2,6-difluoro-4-((2-oxopyrrolidin-1-yl)methyl)phenyl)-5-fluoropicolinate in 100% yield. The crude material was used for the next step without further purification. LC/MS=365.2 (MH+), Rt=0.75 min.

### Synthesis of 6-(2,6-difluoro-4-((2-oxopyrrolidin-1-yl)methyl)phenyl)-5-fluoropicolinic acid

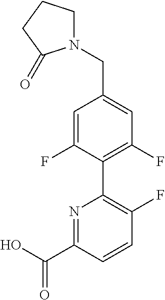

Method 2 was followed using methyl 6-(2,6-difluoro-4-((2-oxopyrrolidin-1-yl)methyl)phenyl)-5-fluoropicolinate to give 6-(2,6-difluoro-4-((2-oxopyrrolidin-1-yl)methyl)phenyl)-5-fluoropicolinic acid in 75% yield. LC/MS=351.1 (MH+), Rt=0.65 min.

### Synthesis of 1-(3,5-difluorophenyl)cyclopentanol

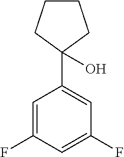

To a solution of Mg (6.7 equiv.) in THF (0.14 M) under nitrogen at 0° C. was added 1,4-dibromo butane (3.5 equiv.) dropwise. The reaction was allowed to warm to rt. After stirring for 1 hr at rt, the reaction was cooled to 0° C. and methyl 3,5-difluorobenzoate (1.0 equiv.) in THF (0.14 M) was added dropwise. The cloudy solution became clear and allowed to warm to rt. After 1 hr, the reaction was quenched by the addition of NH4Cl (sat.) and extracted with ethyl acetate. The organic phase was dried with sodium sulfate, filtered and concentrated. The crude material was purified via ISCO SiO2 chromatography (ethyl acetate and heptanes 0-20% ethyl acetate). The pure fractions were concentrated to give 1-(3,5-difluorophenyl)cyclopentanolin 100% yield. 1H NMR (400 MHz, <cdcl3>) δ ppm 1.77-2.11 (m, 8H), 6.67 (tt, J=8.80, 2.35 Hz, 1H), 6.92-7.08 (m, 2H).

### Synthesis of 1-(3,5-difluoro-4-(4,4,5,5-tetramethyl-1,3,2-dioxaborolan-2-yl)phenyl)cyclopentanol

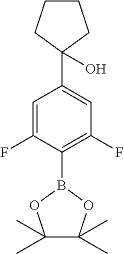

Method 3 was followed using 2-isopropoxy-4,4,5,5-tetramethyl-1,3,2-dioxaborolane (2.5 equiv.), butyllithium (2.4 equiv.) and 1-(3,5-difluorophenyl)cyclopentanol (1.0 equiv.) to give 1-(3,5-difluoro-4-(4,4,5,5-tetramethyl-1,3,2-dioxaborolan-2-yl)phenyl)cyclopentanol in 100% yield. 1H NMR (400 MHz, <cdcl3>) δ ppm 1.24 (s, 12H), 1.80-2.04 (m, 8H), 6.97 (d, J=9.00 Hz, 2H).

### Synthesis of methyl 6-(2,6-difluoro-4-(1-hydroxycyclopentyl)phenyl)-5-fluoropicolinate

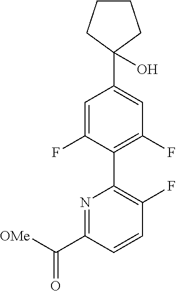

Method 1 was followed using methyl 6-bromo-5-fluoropicolinate (1.0 equiv.) and 1-(3,5-difluoro-4-(4,4,5,5-tetramethyl-1,3,2-dioxaborolan-2-yl)phenyl)cyclopentanol (1.3 equiv.) at 100° C. for 20 min in microwave to give methyl 6-(2,6-difluoro-4-(1-hydroxycyclopentyl)phenyl)-5-fluoropicolinate in 97% yield. LC/MS=352.2 (MH+), Rt=0.88 min. 1H NMR (400 MHz, <cdcl3>) δ ppm 1.80-2.12 (m, 8H), 4.00 (s, 3H), 7.16 (d, J=9.39 Hz, 2H), 7.65 (t, J=8.41 Hz, 1H), 8.26 (dd, J=8.61, 3.91 Hz, 1H).

### Synthesis of 6-(2,6-difluoro-4-(1-hydroxycyclopentyl)phenyl)-5-fluoropicolinic acid

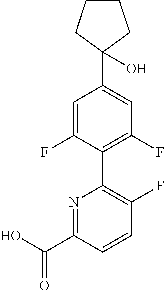

Method 2 was followed using methyl 6-(2,6-difluoro-4-(1-hydroxycyclopentyl)phenyl)-5-fluoropicolinate to give 6-(2,6-difluoro-4-(1-hydroxycyclopentyl)phenyl)-5-fluoropicolinic acid in 83% yield. LC/MS=338.2 (MH+), Rt=0.78 min.

### Synthesis of 1-(2-ethoxypropan-2-yl)-3,5-difluorobenzene

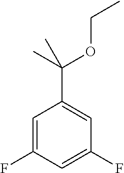

To a solution of 2-(3,5-difluorophenyl)propan-2-ol (1.0 equiv.) in DMF (0.23 M) at 0° C. was added NaH (1.1 equiv.). After stirred at 0° C. for 1 hr, ethyl iodide (1.1 equiv.) was added to the reaction mixture. The ice bath was removed and the reaction was stirred at rt for 3 hrs, quenched with water, partitioned between EtOAc and H₂O. The organic layer was washed with brine and dried with Na2SO4 and concentrated. The crude was purified by ISCO SiO2 chromatography (eluting with 6% ether in hepatanes) to give 1-(2-ethoxypropan-2-yl)-3,5-difluorobenzene in 47% yield. 1H NMR (400 MHz, <cdcl3>) δ ppm 1.18 (t, J=7.04 Hz, 3H), 1.50 (s, 6H), 3.23 (q, J=7.04 Hz, 2H), 6.68 (t, J=2.35 Hz, 1H), 6.93 (dd, J=9.00, 2.35 Hz, 2H).

### Synthesis of 2-(4-(2-ethoxypropan-2-yl)-2,6-difluorophenyl)-4,4,5,5-tetramethyl-1,3,2-dioxaborolane

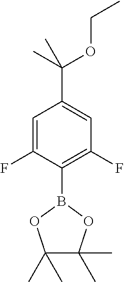

Method 3 was followed using 2-isopropoxy-4,4,5,5-tetramethyl-1,3,2-dioxaborolane (2.5 equiv.), butyllithium (2.5 equiv.) and 1-(2-ethoxypropan-2-yl)-3,5-difluorobenzene (1.0 equiv.) to give 2-(4-(2-ethoxypropan-2-yl)-2,6-difluorophenyl)-4,4,5,5-tetramethyl-1,3,2-dioxaborolane in 100% yield. 1H NMR (400 MHz, CHLOROFORM-d) δ ppm 1.15 (t, J=6.85 Hz, 3H), 1.38 (s, 12H), 1.48 (s, 6H), 3.20 (d, J=7.04 Hz, 2H), 6.90 (d, J=9.00 Hz, 2H).

### Synthesis of methyl 6-(4-(2-ethoxypropan-2-yl)-2,6-difluorophenyl)-5-fluoropicolinate

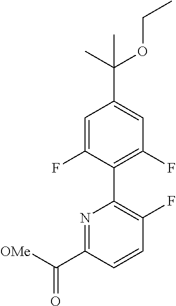

Method 1 was followed using methyl 6-bromo-5-fluoropicolinate (1.1 equiv.) and 2-(4-(2-ethoxypropan-2-yl)-2,6-difluorophenyl)-4,4,5,5-tetramethyl-1,3,2-dioxaborolane (1.0 equiv.) at 100° C. for 20 min in microwave to give methyl 6-(4-(2-ethoxypropan-2-yl)-2,6-difluorophenyl)-5-fluoropicolinate in 100% yield. LC/MS=354.1 (MH+), Rt=1.02 min.

### Synthesis of 6-(4-(2-ethoxypropan-2-yl)-2,6-difluorophenyl)-5-fluoropicolinic acid

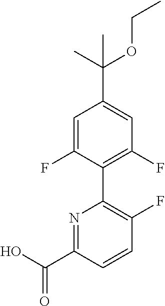

Method 2 was followed using methyl 6-(4-(2-ethoxypropan-2-yl)-2,6-difluorophenyl)-5-fluoropicolinate to give 6-(4-(2-ethoxypropan-2-yl)-2,6-difluorophenyl)-5-fluoropicolinic acid in 75% yield. LC/MS=340.1 (MH+), Rt=0.89 min.

### Synthesis of 4-(3,5-difluorophenyl)-3,5-dimethylisoxazole

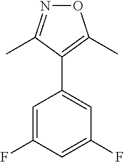

4-bromo-3,5-dimethylisoxazole (1.0 equiv.), 3,5-difluorophenylboronic acid (1.3 equiv.), and PdCl2(dppf).CH2Cl2 adduct (0.1 equiv.) were combined in a microwave vial and 1,4-Dioxane (0.3 M) was added followed by 2M sodium carbonate (2.0 equiv.). The mixture was purged with N2, sealed and heated at 120° C. for 40 min in the microwave. The mixture was partitioned between EtOAc and brine. The organic layer was dried over sodium sulfate, filtered and concentrated to afford a black solid. The crude black material was purified by ISCO SiO₂ chromatography eluting with 0-100% DCM in Heptanes to afford 4-(3,5-difluorophenyl)-3,5-dimethylisoxazole in 60% yield. LC/MS (m/z): 210.1 (MH+), Rt=0.88 min. 1H NMR (400 MHz, <cdcl3>) δ 6.73-6.87 (m, 3H), 2.43 (s, 3H), 2.29 (s, 3H).

### Synthesis of 4-(3,5-difluoro-4-(4,4,5,5-tetramethyl-1,3,2-dioxaborolan-2-yl)phenyl)-3,5-dimethylisoxazole

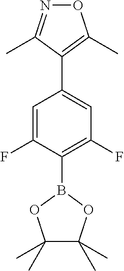

Method 3 was followed using 2-isopropoxy-4,4,5,5-tetramethyl-1,3,2-dioxaborolane (2.0 equiv.), butyllithium (1.05 equiv.) and 4-(3,5-difluorophenyl)-3,5-dimethylisoxazole (1.0 equiv.) to give 4-(3,5-difluoro-4-(4,4,5,5-tetramethyl-1,3,2-dioxaborolan-2-yl)phenyl)-3,5-dimethylisoxazole in 97% yield. 1H NMR (400 MHz, CHLOROFORM-d) δ ppm 1.38-1.42 (s, 12H), 2.28 (s, 3H), 2.43 (s, 3H), 6.76 (d, J=8.22 Hz, 2H).

### Synthesis of methyl 6-(4-(3,5-dimethylisoxazol-4-yl)-2,6-difluorophenyl)-5-fluoropicolinate

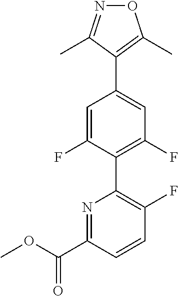

Method 1 was followed using methyl 6-bromo-5-fluoropicolinate (1.0 equiv.) and 4-(3,5-difluoro-4-(4,4,5,5-tetramethyl-1,3,2-dioxaborolan-2-yl)phenyl)-3,5-dimethylisoxazole (2.5 equiv.) at 80° C. for 15 min in microwave to give methyl 6-(4-(3,5-dimethylisoxazol-4-yl)-2,6-difluorophenyl)-5-fluoropicolinate in 89% yield. LC/MS=363.1 (MH+), Rt=0.90 min.

### Synthesis of 6-(4-(3,5-dimethylisoxazol-4-yl)-2,6-difluorophenyl)-5-fluoropicolinic acid

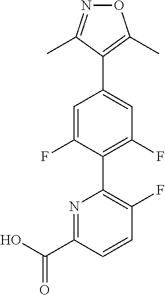

Method 2 was followed using methyl 6-(4-(3,5-dimethylisoxazol-4-yl)-2,6-difluorophenyl)-5-fluoropicolinate to give 6-(4-(3,5-dimethylisoxazol-4-yl)-2,6-difluorophenyl)-5-fluoropicolinic acid in 63% yield. LC/MS=349.2 (MH+), Rt=0.80 min.

### Synthesis of tert-butyl 2-(3,5-difluorophenyl)-2-methylpropanoate

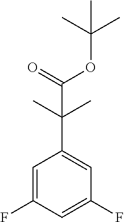

To a solution of 2-(3,5-difluorophenyl)-2-methylpropanoic acid (1.0 equiv.) dissolved in DCM (0.20 M) was added oxalyl chloride (1.8 equiv.) followed by 5 drops of DMF. The mixture was stirred at rt for 30 min and then the solvents were removed in vacuo. The residue was taken up in THF (0.20 M) and cooled to 0° C. on an ice bath. Potassium tert-butoxide (1.2 equiv., 1M solution in THF) was added drop wise over 10 min. The reaction was stirred for 18 hrs. The reaction was diluted with ether and washed with water, brine, dried over sodium sulfate, filtered and concentrated to yield tert-butyl 2-(3,5-difluorophenyl)-2-methylpropanoate in 97% yield. 1H NMR (400 MHz, CHLOROFORM-d) δ ppm 1.39 (s, 9H), 1.50 (s, 6H), 6.67 (s, 1H), 6.86 (dd, J=9.00, 1.96 Hz, 2H).

### Synthesis of tert-butyl 2-(3,5-difluoro-4-(4,4,5,5-tetramethyl-1,3,2-dioxaborolan-2-yl)phenyl)-2-methylpropanoate

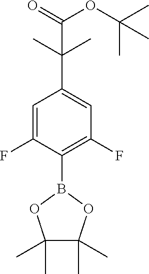

Method 3 was followed using 2-isopropoxy-4,4,5,5-tetramethyl-1,3,2-dioxaborolane (2.2 equiv.), butyllithium (1.1 equiv.) and tert-butyl 2-(3,5-difluorophenyl)-2-methylpropanoate (1.0 equiv.) to give tert-butyl 2-(3,5-difluoro-4-(4,4,5,5-tetramethyl-1,3,2-dioxaborolan-2-yl)phenyl)-2-methylpropanoate in 100% yield. 1H NMR (400 MHz, CHLOROFORM-d) δ ppm 1.27 (s, 9H), 1.36 (s, 12H), 1.48 (s, 6H), 6.83 (d, J=9.39 Hz, 2H).

### Synthesis of methyl 6-(4-(1-(tert-butoxy)-2-methyl-1-oxopropan-2-yl)-2,6-difluorophenyl)-5-fluoropicolinate

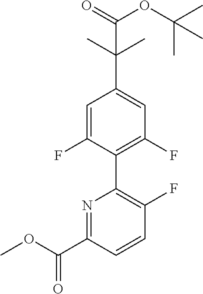

Method 1 was followed using methyl 6-bromo-5-fluoropicolinate (1.0 equiv.) and tert-butyl 2-(3,5-difluoro-4-(4,4,5,5-tetramethyl-1,3,2-dioxaborolan-2-yl)phenyl)-2-methylpropanoate (2.0 equiv.) at 80° C. for 15 min in microwave to give methyl 6-(4-(1-(tert-butoxy)-2-methyl-1-oxopropan-2-yl)-2,6-difluorophenyl)-5-fluoropicolinate in 73% yield. LC/MS=410.1 (MH+), Rt=1.11 min.

### Synthesis of 6-(4-(1-(tert-butoxy)-2-methyl-1-oxopropan-2-yl)-2,6-difluorophenyl)-5-fluoropicolinic acid

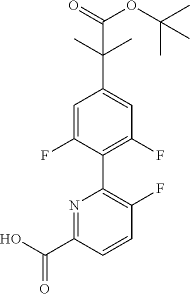

Method 2 was followed using methyl 6-(4-(1-(tert-butoxy)-2-methyl-1-oxopropan-2-yl)-2,6-difluorophenyl)-5-fluoropicolinate to give 6-(4-(1-(tert-butoxy)-2-methyl-1-oxopropan-2-yl)-2,6-difluorophenyl)-5-fluoropicolinic acid in 82% yield. LC/MS=396.1 (MH+), Rt=1.00 min.

### Synthesis of methyl 6-(2,6-difluoro-4-(3-methoxypropoxy)phenyl)-5-fluoropicolinate

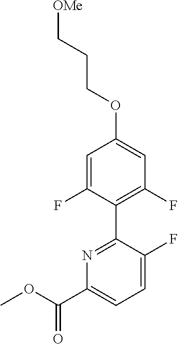

To a solution of triphenylphosphine (2.0 equiv.), methyl 6-(2,6-difluoro-4-hydroxyphenyl)-5-fluoropicolinate (1.0 equiv.) and 3-methoxypropan-1-ol (1.2 equiv.) in THF (0.14 M) was added DIAD (2.0 equiv.) dropwise. The mixture was allowed to stir overnight at rt. The reaction was concentrated to dryness and purified via silica gel column chromatography (ISCO, ethyl acetate and heptanes 0-50% ethyl acetate). The pure fractions were concentrated to yield methyl 6-(2,6-difluoro-4-(3-methoxypropoxy)phenyl)-5-fluoropicolinate in 100% yield. LC/MS=356.1 (MH+), Rt=0.93 min.

### Synthesis of 6-(2,6-difluoro-4-(3-methoxypropoxy)phenyl)-5-fluoropicolinic acid

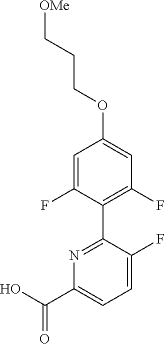

Method 2 was followed using methyl 6-(2,6-difluoro-4-(3-methoxypropoxy)phenyl)-5-fluoropicolinate to give 6-(2,6-difluoro-4-(3-methoxypropoxy)phenyl)-5-fluoropicolinic acid 64% yield. LC/MS=342.1 (MH+), Rt=0.83 min.

### Synthesis of 2-(5,7-difluoro-2,3-dihydrobenzofuran-6-yl)-4,4,5,5-tetramethyl-1,3,2-dioxaborolane

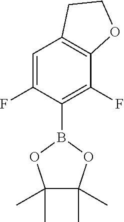

Method 3 was followed using 2-isopropoxy-4,4,5,5-tetramethyl-1,3,2-dioxaborolane (1.3 equiv.), butyllithium (1.3 equiv.) and 5,7-difluoro-2,3-dihydrobenzofuran (1.0 equiv.) to give 2-(5,7-difluoro-2,3-dihydrobenzofuran-6-yl)-4,4,5,5-tetramethyl-1,3,2-dioxaborolane in 30% yield. 1H NMR (400 MHz, CHLOROFORM-d) δ ppm 1.37 (s, 12H), 3.24 (td, J=8.71, 4.11 Hz, 2H), 4.51-4.78 (m, 2H) 6.70 (d, J=7.43 Hz, 1H).

### Synthesis of methyl 6-(5,7-difluoro-2,3-dihydrobenzofuran-6-yl)-5-fluoropicolinate

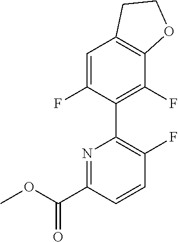

Method 1 was followed using methyl 6-bromo-5-fluoropicolinate (1.0 equiv.) and 2-(5,7-difluoro-2,3-dihydrobenzofuran-6-yl)-4,4,5,5-tetramethyl-1,3,2-dioxaborolane (1.5 equiv.) at 90° C. for 90 min in oil bath to give methyl 6-(5,7-difluoro-2,3-dihydrobenzofuran-6-yl)-5-fluoropicolinate in 90% yield. LC/MS=310.1 (MH+), Rt=0.86 min.

### Synthesis of 6-(5,7-difluoro-2,3-dihydrobenzofuran-6-yl)-5-fluoropicolinic acid

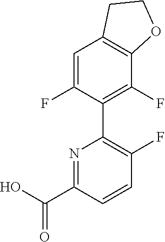

Method 2 was followed using methyl 6-(5,7-difluoro-2,3-dihydrobenzofuran-6-yl)-5-fluoropicolinate to give 6-(5,7-difluoro-2,3-dihydrobenzofuran-6-yl)-5-fluoropicolinic acid 90% yield. LC/MS=296.1 (MH+), Rt=0.73 min.

### Synthesis of methyl 6-(2,6-difluoro-4-((tetrahydro-2H-pyran-4-yl)methoxy)phenyl)-5-fluoropicolinate

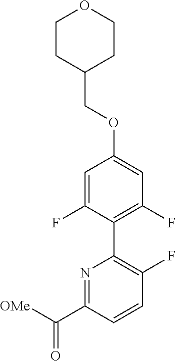

A mixture of methyl 6-(2,6-difluoro-4-hydroxyphenyl)-5-fluoropicolinate (1.0 equiv.), 4-(bromomethyl)tetrahydro-2H-pyran (2.0 equiv.) and K₂CO₃ (4.0 equiv.) in DMF (0.20 M) was heated at 100° C. for 20 min in microwave. The reaction mixture was cooled off to rt and partitioned between EtOAc and H₂O. The organic layer was washed with brine, dried over Na2SO4 and concentrated to give methyl 6-(2,6-difluoro-4-((tetrahydro-2H-pyran-4-yl)methoxy)phenyl)-5-fluoropicolinate in 100% yield. LC/MS=382.0 (MH+), Rt=0.97 min.

### Synthesis of 6-(2,6-difluoro-4-((tetrahydro-2H-pyran-4-yl)methoxy)phenyl)-5-fluoropicolinic acid

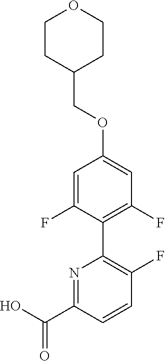

Method 2 was followed using methyl 6-(2,6-difluoro-4-((tetrahydro-2H-pyran-4-yl)methoxy)phenyl)-5-fluoropicolinate to give 6-(2,6-difluoro-4-((tetrahydro-2H-pyran-4-yl)methoxy)phenyl)-5-fluoropicolinic acid in 81% yield. LC/MS=368.0 (MH+), Rt=0.85 min.

### Synthesis of 4-(bromomethyl)tetrahydro-2H-pyran-4-carbonitrile

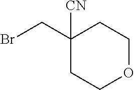

To a solution of triphenylphosphine (1.0 equiv.) in DCM (0.20 M) was added bromine (1.0 equiv.). The mixture became nearly colorless and was stirred for an additional 40 min. To the resulting heterogeneous mixture was added 4-(hydroxymethyl)tetrahydro-2H-pyran-4-carbonitrile (1.0 equiv.). The light yellow solution was stirred at ambient temperature for 2 days and heated at 50° C. for 3 days. The reaction mixture was diluted with DCM and washed with water. The aqueous phase was extracted with additional DCM. The combined organics were dried over sodium sulfate, filtered, and concentrated to give 4-(bromomethyl)tetrahydro-2H-pyran-4-carbonitrile in 35% yield.

### Synthesis of methyl 6-(4-((4-cyanotetrahydro-2H-pyran-4-yl)methoxy)-2,6-difluorophenyl)-5-fluoropicolinate

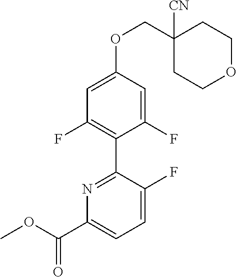

To a solution of methyl 6-(2,6-difluoro-4-hydroxyphenyl)-5-fluoropicolinate (1.0 equiv.) in DMF (0.50 M) was added potassium carbonate (5.0 equiv.) and 4-(bromomethyl)tetrahydro-2H-pyran-4-carbonitrile (1.5 equiv.). The mixture was stirred at 70° C. for 7 days. The cooled reaction mixture was diluted with ethyl acetate and filtered. The filtrate was washed with water. The aqueous phase was extracted with additional ethyl acetate. The combined organic phases were dried over sodium sulfate, filtered, and concentrated. The residue was purified by flash chromatography to give methyl 6-(4-((4-cyanotetrahydro-2H-pyran-4-yl)methoxy)-2,6-difluorophenyl)-5-fluoropicolinate in 9% yield. LC/MS=406.9 (MH+), Rt=0.89 min.

### Synthesis of 6-(4-((4-cyanotetrahydro-2H-pyran-4-yl)methoxy)-2,6-difluorophenyl)-5-fluoropicolinic acid

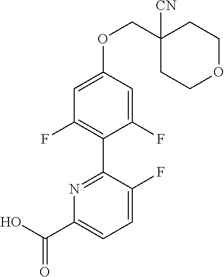

Method 2 was followed using methyl 6-(4-((4-cyanotetrahydro-2H-pyran-4-yl)methoxy)-2,6-difluorophenyl)-5-fluoropicolinate to give 6-(4-((4-cyanotetrahydro-2H-pyran-4-yl)methoxy)-2,6-difluorophenyl)-5-fluoropicolinic acid in 75% yield. LC/MS=393.0 (MH+), Rt=0.80 min.

### Synthesis of methyl 6-(2,6-difluoro-4-hydroxyphenyl)picolinate

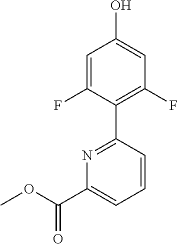

Method 1 was followed using methyl 6-bromopicolinate (1.0 equiv.) and tert-butyl(3,5-difluoro-4-(4,4,5,5-tetramethyl-1,3,2-dioxaborolan-2-yl)phenoxy)dimethylsilane (1.5 equiv.) at 75° C. for 1 hr in an oil bath to give methyl 6-(2,6-difluoro-4-hydroxyphenyl)picolinate in 23% yield. LC/MS=266.1 (MH+), Rt=0.66 min.

### Synthesis of Methyl 6-(2,6-difluoro-4-(tetrahydro-2H-pyran-4-yloxy)phenyl)picolinate

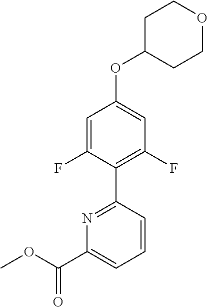

A solution of tetrahydro-2H-pyran-4-ol (1.9 equiv.), methyl 6-(2,6-difluoro-4-hydroxyphenyl)picolinate (1.0 equiv.) and Ph₃P (3.0 equiv.) in THF (0.15 M) was cooled to 0° C. at which time the DIAD (3.0 equiv.) was added via pipette. The solution was capped and after stirring for 10 minutes the ice bath was removed and the solution was left stirring overnight. The volatiles were removed in vacuo, and the residue was purified by ISCO SiO₂ chromatography (0-100% EtOAc/n-heptanes) to yield methyl 6-(2,6-difluoro-4-(tetrahydro-2H-pyran-4-yloxy)phenyl)picolinate in 74% yield. LC/MS=350.1 (MH+), Rt=0.85 min.

### Synthesis of 6-(2,6-difluoro-4-((tetrahydro-2H-pyran-4-yl)oxy)phenyl)picolinic acid

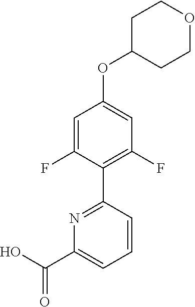

Method 2 was followed using methyl 6-(2,6-difluoro-4-(tetrahydro-2H-pyran-4-yloxy)phenyl)picolinate to give 6-(2,6-difluoro-4-((tetrahydro-2H-pyran-4-yl)oxy)phenyl)picolinic acid in 83% yield. LC/MS=336.1 (MH+), Rt=0.72 min.

### Synthesis of methyl 6-(2,6-difluoro-4-(2-methoxyethoxy)phenyl)picolinate

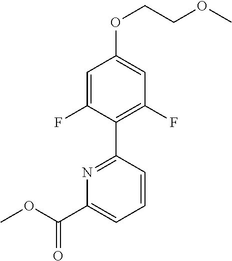

To a heterogeneous solution of methyl 6-(2,6-difluoro-4-hydroxyphenyl)picolinate (1.0 equiv.) and K₂CO₃ (5.0 equiv.) in DMF (0.75 M) was added 1-bromo-2-methoxyethane (3.0 equiv.). The solution was capped and left stirring at rt overnight. The solution was partitioned between EtOAc and water. The organic layer was washed with NaCl(sat.), dried over MgSO4, filtered, concentrated, purified by ISCO SiO₂ chromatography (0-100% EtOAc/n-heptanes) to yield methyl 6-(2,6-difluoro-4-(2-methoxyethoxy)phenyl)picolinate in 94% yield. LC/MS=324.1 (MH+), Rt=0.79 min.

### Synthesis of 6-(2,6-difluoro-4-(2-methoxyethoxy)phenyl)picolinic acid

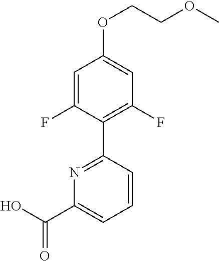

Method 2 was followed using methyl 6-(2,6-difluoro-4-(2-methoxyethoxy)phenyl)picolinate to give 6-(2,6-difluoro-4-(2-methoxyethoxy)phenyl)picolinic acid in 75% yield. LC/MS=310.1 (MH+), Rt=0.65 min.

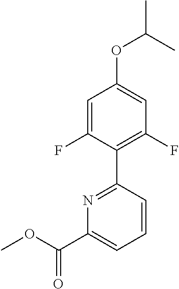

To a heterogeneous solution of methyl 6-(2,6-difluoro-4-hydroxyphenyl)picolinate (1.0 equiv.) and K₂CO₃ (5.0 equiv.) in DMF (0.75 M) was added 2-iodopropane (3.0 equiv.). The solution was capped and left stirring at rt overnight. The solution was partitioned between EtOAc and water. The organic layer was washed with NaCl(sat.), dried over MgSO4, filtered, concentrated, purified by ISCO SiO2 chromatography (0-100% EtOAc/n-heptanes) to yield methyl 6-(2,6-difluoro-4-isopropoxyphenyl)picolinate in 86% yield. LC/MS=308.1 (MH+), Rt=0.93 min.

### Synthesis of 6-(2,6-difluoro-4-isopropoxyphenyl)picolinic acid

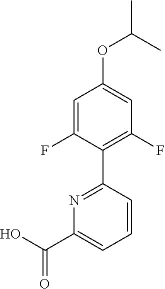

Method 2 was followed using methyl 6-(2,6-difluoro-4-isopropoxyphenyl)picolinate to give 6-(2,6-difluoro-4-isopropoxyphenyl)picolinic acid in 96% yield. LC/MS=294.1 (MH+), Rt=0.82 min.

### Synthesis of methyl 6-(2,6-difluoro-4-morpholinophenyl)picolinate

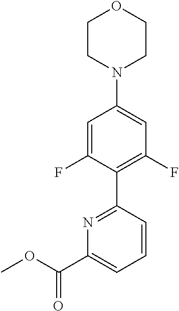

A solution of methyl 6-bromopicolinate (1.0 equiv.), 4-(3,5-difluoro-4-(4,4,5,5-tetramethyl-1,3,2-dioxaborolan-2-yl)phenyl)morpholine (1.3 equiv.) and PdCl2(ddpf) (0.15 equiv.) in 3:1 DME/2M Na₂CO₃ (0.17 M) were heated in the microwave for 20 minutes at 120° C. The solution was diluted with EtOAc, washed with NaCl(sat.). The aqueous was reextracted with EtOAc and the combined organics were dried over MgSO4, filtered, concentrated, purified by ISCO SiO₂ chromatography (0-100% EtOAc/n-heptanes) to yield methyl 6-(2,6-difluoro-4-morpholinophenyl)picolinate in 40% yield. LC/MS=335.3 (MH+), Rt=0.77 min.

### Synthesis of 6-(2,6-difluoro-4-morpholinophenyl)picolinic acid

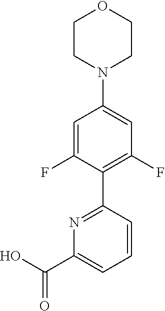

Method 2 was followed using methyl 6-(2,6-difluoro-4-morpholinophenyl)picolinate to give 6-(2,6-difluoro-4-morpholinophenyl)picolinic acid in 49% yield. LC/MS=321.1 (MH+), Rt=0.62 min.

### Synthesis of methyl 6-(2,6-difluoro-4-(4-hydroxytetrahydro-2H-pyran-4-yl)phenyl)picolinate

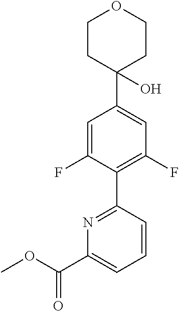

Method 1 was followed using methyl 6-bromopicolinate (1.0 equiv.) and 4-(3,5-difluoro-4-(4,4,5,5-tetramethyl-1,3,2-dioxaborolan-2-yl)phenyl)tetrahydro-2H-pyran-4-ol (1.0 equiv.) at 80° C. for 20 min in microwave to give methyl 6-(2,6-difluoro-4-(4-hydroxytetrahydro-2H-pyran-4-yl)phenyl)picolinate in 44% yield. LC/MS=350.3 (MH+), Rt=0.69 min.

### Synthesis of methyl 6-(4-(3,6-dihydro-2H-pyran-4-yl)-2,6-difluorophenyl)picolinate

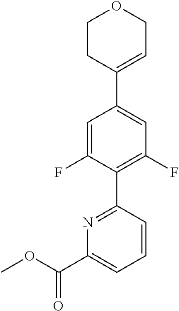

To a solution of methyl 6-(2,6-difluoro-4-(4-hydroxytetrahydro-2H-pyran-4-yl)phenyl)picolinate (1.0 equiv.) in DCM (0.57 M) was added TFA (35.0 equiv.). The reaction was heated in the microwave at 110° C. for 90 min. The reaction mixture was concentrated to dryness and purified via ISCO SiO₂ chromatography (0-100% ethyl acetate) to yield methyl 6-(4-(3,6-dihydro-2H-pyran-4-yl)-2,6-difluorophenyl)picolinate in 74% yield. LC/MS=332.1 (MH+), Rt=0.87 min.

### Synthesis of methyl 6-(2,6-difluoro-4-(tetrahydro-2H-pyran-4-yl)phenyl)picolinate

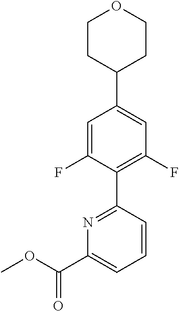

To a degassed solution of methyl 6-(4-(3,6-dihydro-2H-pyran-4-yl)-2,6-difluorophenyl)picolinate (1.0 equiv.) in 3/1 Methanol/EtOAc (0.10 M) was added Pd/C (0.2 equiv.) and the reaction was stirred under a hydrogen balloon for 8 hrs. The reaction mixture was filtered through a pad of Celite and washed with ethyl acetate. The filtrate was concentrated to yield methyl 6-(2,6-difluoro-4-(tetrahydro-2H-pyran-4-yl)phenyl)picolinate. LC/MS=334.0 (MH+), Rt=0.85 min.

### Synthesis of 6-(2,6-difluoro-4-(tetrahydro-2H-pyran-4-yl)phenyl)picolinic acid

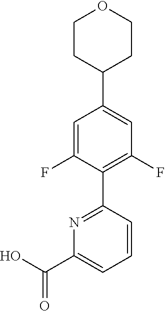

Method 2 was followed using methyl 6-(2,6-difluoro-4-(tetrahydro-2H-pyran-4-yl)phenyl)picolinate to give 6-(2,6-difluoro-4-(tetrahydro-2H-pyran-4-yl)phenyl)picolinic acid in 26% yield. LC/MS=320.0 (MH+), Rt=0.74 min.

### Synthesis of methyl 6-(4-(2-ethoxyethoxy)-2,6-difluorophenyl)-5-fluoropicolinate

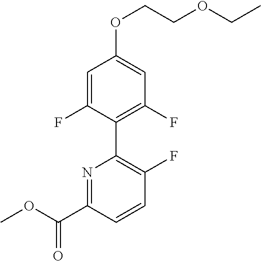

A solution of 2-ethoxyethanol (1.2 equiv.), DIAD (3.0 equiv.) and Ph3P (3.0 equiv.) in THF (0.20 M) was stirred at rt for 10 min and then methyl 6-(2,6-difluoro-4-hydroxyphenyl)-5-fluoropicolinate (1.0 equiv.) was added. The solution was left stirring overnight. The volatiles were removed in vacuo, and the residue was purified by ISCO SiO2 chromatography (120 gram column, 0-100% EtOAc/n-heptanes) to yield methyl 6-(4-(2-ethoxyethoxy)-2,6-difluorophenyl)-5-fluoropicolinate in 100% yield. LC/MS=356.2 (M+H), Rₜ=0.92 min.

### Synthesis of 6-(4-(2-ethoxyethoxy)-2,6-difluorophenyl)-5-fluoropicolinic acid

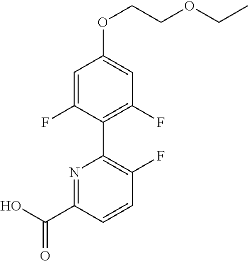

Method 2 was followed using methyl 6-(4-(2-ethoxyethoxy)-2,6-difluorophenyl)-5-fluoropicolinate to give 6-(4-(2-ethoxyethoxy)-2,6-difluorophenyl)-5-fluoropicolinic acid in 90% yield. LC/MS=342.1 (M+H), Rt=0.82 min.

### Synthesis of methyl 2′,6,6′-trifluoro-4′-(trifluoromethylsulfonyloxy)biphenyl-3-carboxylate

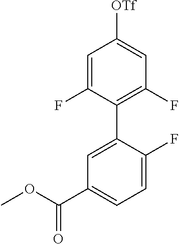

To a solution of methyl 2′,6,6′-trifluoro-4′-hydroxybiphenyl-3-carboxylate (1.0 equiv.) in DCM (0.35 M) at 0° C. was added pyridine (1.5 equiv.) and allowed to stir for 5 mins, followed by the addition of TriflicAnhydride (1.1 equiv.). The reaction was allowed to stir warming to RT. The reaction was quenched with NaHCO3(sat), extracted in DCM and the organics were washed with water and brine. The organics were dried over Na2SO4, filtered, and concentrated to yield methyl 2′,6,6′-trifluoro-4′-(trifluoromethylsulfonyloxy)biphenyl-3-carboxylate in 81% yield.

### Synthesis of methyl 6-(4-(3,6-dihydro-2H-thiopyran-4-yl)-2,6-difluorophenyl)-5-fluoropicolinate

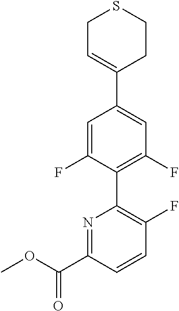

To a degassed solution of methyl 6-(2,6-difluoro-4-(trifluoromethylsulfonyloxy)phenyl)-5-fluoropicolinate (1.0 equiv.) and 3,6-dihydro-2H-thiopyran-4-ylboronic acid (1.5 equiv.) in DME/2M Na2CO3 (3/1, 0.10 M) was added PdCl2(dppf).CH2Cl2 adduct (0.10 equiv.). The reaction was heated to 90° C. in an oil bath for 15 min. The reaction mixture was partitioned with water and EtOAc; the organics were dried over MgSO4, filtered, and concentrated. The crude was purified via ISCO. Pure fractions were combined and concentrated to yield methyl 6-(4-(3,6-dihydro-2H-thiopyran-4-yl)-2,6-difluorophenyl)-5-fluoropicolinate in 60% yield. LC/MS=366.1 (M+H), Rt=1.00 min.

### Synthesis of methyl 6-(4-(1,1-dioxido-3,6-dihydro-2H-thiopyran-4-yl)-2,6-difluorophenyl)-5-fluoropicolinate

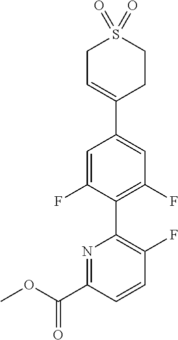

To a solution of methyl 6-(4-(3,6-dihydro-2H-thiopyran-4-yl)-2,6-difluorophenyl)-5-fluoropicolinate (1.0 equiv.) in DCM (0.10 M) at rt was added oxone (6.0 equiv.) in one portion. The resulting mixture was stirred at RT overnight, and then refluxed at 40° C. for 4 hrs. 10.0 equiv. of oxone were added and the reaction was allowed to stir at 40° C. over the weekend. The reaction mixture was then diluted with DCM and washed with water the aqueous layer was then separated and extracted with DCM. The combined organic were then dried over MgSO4 and concentrated in vacuo to yield methyl 6-(4-(1,1-dioxido-3,6-dihydro-2H-thiopyran-4-yl)-2,6-difluorophenyl)-5-fluoropicolinate in 100% yield. LC/MS=398.0 (M+H), Rt=0.76 min.

### Synthesis of 6-(4-(1,1-dioxido-3,6-dihydro-2H-thiopyran-4-yl)-2,6-difluorophenyl)-5-fluoropicolinic acid

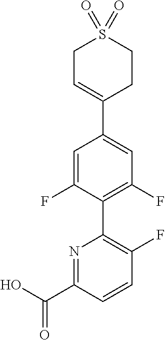

Method 2 was followed using methyl 6-(4-(1,1-dioxido-3,6-dihydro-2H-thiopyran-4-yl)-2,6-difluorophenyl)-5-fluoropicolinate to give 6-(4-(1,1-dioxidotetrahydro-2H-thiopyran-4-yl)-2,6-difluorophenyl)-5-fluoropicolinic acid in 74% yield. LC/MS=384.0 (M+H), Rt=0.64 min.

### Synthesis of 6-(4-(1,1-dioxidotetrahydro-2H-thiopyran-4-yl)-2,6-difluorophenyl)-5-fluoropicolinic acid

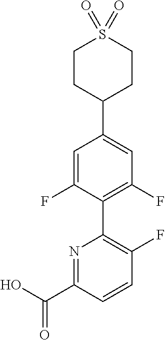

To a degassed solution of 6-(4-(1,1-dioxido-3,6-dihydro-2H-thiopyran-4-yl)-2,6-difluorophenyl)-5-fluoropicolinic acid (1.0 equiv.) in EtOH (0.10 M) was added Pd/C (0.1 equiv.). The mixture was stirred at rt under H2 for 16 hrs. Add Pd/C (0.1 equiv.) and the reaction was stirred for additional 16 hrs. The reaction was taken up and filtered through a syringe filter. The combined organics were concentrated to yield 6-(4-(1,1-dioxidotetrahydro-2H-thiopyran-4-yl)-2,6-difluorophenyl)-5-fluoropicolinic acid in 100% yield. LC/MS=386.0 (M+H), Rt=0.64 min.

### Synthesis of methyl 6-(2,6-difluoro-3-formylphenyl)-5-fluoropicolinate

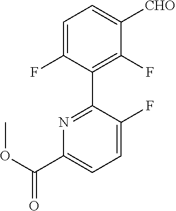

Method 1 was followed using methyl 6-bromo-5-fluoropicolinate (1.0 equiv.) and 2,6-difluoro-3-formylphenylboronic acid (1.5 equiv.) at 80° C. in an oil bath for 1 hr to give methyl 6-(2,6-difluoro-3-formylphenyl)-5-fluoropicolinate in 35% yield. LC/MS=295.9 (M+H), Rt=0.75 min.

### Synthesis of methyl 6-(2,6-difluoro-3-vinylphenyl)-5-fluoropicolinate

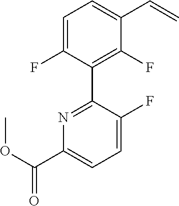

To a solution of methyl 6-(2,6-difluoro-3-formylphenyl)-5-fluoropicolinate (1.0 equiv.) and METHYLTRIPHENYLPHOSPHONIUM BROMIDE (1.5 equiv.) in THF (0.11 M) at 0° C. under an atmosphere of nitrogen was added sodium hydride (3.0 equiv). The reaction was allowed to stir at rt overnight. The reaction was worked up by partitioning between water and ethyl acetate, the organic phase was dried with sodium sulfate, filtered and concentrated. The crude material was purified via silica gel (ISCO, heptanes and ethyl acetate 0-100%). The pure fractions were concentrated to give methyl 6-(2,6-difluoro-3-vinylphenyl)-5-fluoropicolinate in 59% yield. LC/MS=294.2 (M+H), Rt=0.92 min. 1H NMR (400 MHz, <cdcl3>) δ ppm 4.01 (s, 3H), 5.40 (d, J=11.35 Hz, 1H), 5.80 (d, J=18.00 Hz, 1H), 6.84 (dd, J=17.61, 11.35 Hz, 1H), 6.94-7.08 (m, 1H), 7.48-7.74 (m, 2H), 8.28 (dd, J=8.61, 3.91 Hz, 1 H).

### Synthesis of 6-(2,6-difluoro-3-vinylphenyl)-5-fluoropicolinic acid

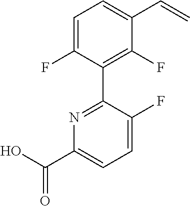

Method 2 was followed using methyl 6-(2,6-difluoro-3-vinylphenyl)-5-fluoropicolinate to give 6-(2,6-difluoro-3-vinylphenyl)-5-fluoropicolinic acid in 99% yield. LC/MS=280.0 (M+H), Rt=0.80 min. 1H NMR (300 MHz, <cd3od>) δ ppm 5.43 (d, J=11.14 Hz, 1H), 5.90 (d, J=17.58 Hz, 1H), 6.87 (dd, J=17.73, 11.28 Hz, 1H), 7.13 (td, J=8.79, 1.47 Hz, 1H), 7.78 (td, J=8.64, 6.45 Hz, 1H), 7.92 (t, J=8.64 Hz, 1H), 8.32 (dd, J=8.50, 4.10 Hz, 1H).

### Synthesis of methyl 6-(3-(allyloxy)-2,6-difluorophenyl)-5-fluoropicolinate

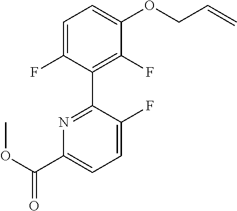

To a solution of methyl 6-(2,6-difluoro-3-hydroxyphenyl)-5-fluoropicolinate (1.0 equiv.) in DMF (0.14 M) was added potassium carbonate (3.0 equiv.) and allyl bromide (1.1 equiv.). The mixture was stirred at 70° C. for 2 hrs. The cooled reaction mixture was diluted with ethyl acetate, and filtered. The filtrate was concentrated to give methyl 6-(3-(allyloxy)-2,6-difluorophenyl)-5-fluoropicolinate in 100% yield. LC/MS=324.2 (M+H), Rt=0.91 min. 1H NMR (300 MHz, <cdcl3>) δ ppm 4.01 (s, 3H), 4.62 (dt, J=5.27, 1.47 Hz, 2H), 5.24-5.51 (m, 2 H), 6.05 (ddt, J=17.25, 10.59, 5.27, 5.27 Hz, 1H), 6.83-6.96 (m, 1H), 7.05 (td, J=9.01, 5.13 Hz, 1H), 7.66 (t, J=8.50 Hz, 1H), 8.27 (dd, J=8.64, 3.96 Hz, 1H).

### Synthesis of 6-(3-(allyloxy)-2,6-difluorophenyl)-5-fluoropicolinic acid

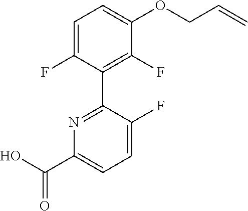

Method 2 was followed using methyl 6-(3-(allyloxy)-2,6-difluorophenyl)-5-fluoropicolinate to give 6-(3-(allyloxy)-2,6-difluorophenyl)-5-fluoropicolinic acid in 95% yield. LC/MS=310.0 (M+H), Rt=0.80 min. 1H NMR (300 MHz, <cd3od>) δ ppm 4.65 (d, J=5.27 Hz, 2H), 5.24-5.55 (m, 2H), 5.93-6.21 (m, 1H), 6.87-7.03 (m, 1H), 7.12 (td, J=9.08, 5.27 Hz, 1H), 7.78 (t, J=8.35 Hz, 1H), 8.35 (dd, J=8.50, 4.10 Hz, 1H).

### Synthesis of methyl 6-(2,6-difluoro-4-(2,2,2-trifluoroethoxy)phenyl)-5-fluoropicolinate

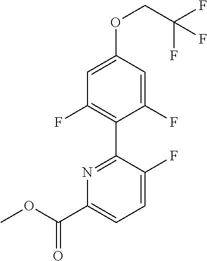

To a solution of methyl 6-(2,6-difluoro-4-hydroxyphenyl)-5-fluoropicolinate (1.0 equiv.) in DMF (0.35 M) was added potassium carbonate (3.0 equiv.) and 2,2,2-trifluoroethyl trifluoromethanesulfonate (1.2 equiv.). The mixture was stirred at ambient temperature for 3 hrs. The reaction mixture was diluted with ethyl acetate, and filtered. The filtrate was washed with water and brine, concentrated, and purified by flash chromatography to give methyl 6-(2,6-difluoro-4-(2,2,2-trifluoroethoxy)phenyl)-5-fluoropicolinate in 100% yield. LC/MS=366.0 (M+H), Rt=0.95 min.

### Synthesis of 6-(2,6-difluoro-4-(2,2,2-trifluoroethoxy)phenyl)-5-fluoropicolinic acid

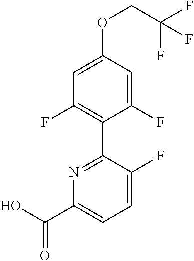

Method 2 was followed using methyl 6-(2,6-difluoro-4-(2,2,2-trifluoroethoxy)phenyl)-5-fluoropicolinate to give 6-(2,6-difluoro-4-(2,2,2-trifluoroethoxy)phenyl)-5-fluoropicolinic acid in 100% yield. LC/MS=352.1 (M+H), Rt=0.85 min.

### Synthesis of methyl 6-(2,6-difluoro-4-(prop-1-en-2-yl)phenyl)-5-fluoropicolinate

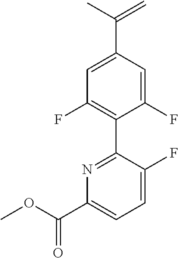

To a degassed solution of methyl 6-(2,6-difluoro-4-(trifluoromethylsulfonyloxy)phenyl)-5-fluoropicolinate (1.0 equiv.) in DME/2M Na2CO3 (3/1, 0.09 M) was added 4,4,5,5-tetramethyl-2-(prop-1-en-2-yl)-1,3,2-dioxaborolane (1.5 equiv.) and PdCl2(dppf)-CH2Cl2Adduct (0.1 equiv.), followed by. The reaction was heated to 90° C. in an oil bath for 15 min. The mixture was cooled to rt and partitioned between water and ethyl acetate. The organic phase was dried with sodium sulfate, filtered and concentrated. The crude material was purified via silica gel column chromatography (Analogix, eluting with 0-100% ethyl acetate). The pure fractions were concentrated to yield methyl 6-(2,6-difluoro-4-(prop-1-en-2-yl)phenyl)-5-fluoropicolinate. LC/MS=308.2 (M+H), Rt=0.99 min. 1H NMR (400 MHz, <cdcl3>) δ ppm 2.15 (s, 3H), 4.01 (s, 3H), 5.23 (s, 1H), 5.47 (s, 1H), 7.11 (d, J=9.39 Hz, 2 H), 7.65 (t, J=8.41 Hz, 1H), 8.26 (dd, J=8.61, 3.91 Hz, 1H).

### Synthesis of methyl 6-(2,6-difluoro-4-isopropylphenyl)-5-fluoropicolinate

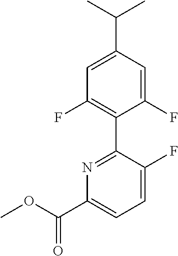

To a degassed solution of methyl 6-(2,6-difluoro-4-(prop-1-en-2-yl)phenyl)-5-fluoropicolinate (1.0 equiv.) in MeOH (0.09 M) was added Pd/C (0.1 equiv.) and the reaction was stirred at rt under an atmosphere of hydrogen. After overnight stirring, filtered through a pad of Celite and washed with Methanol. The filtrate was concentrated and dried under vacuo to give methyl 6-(2,6-difluoro-4-isopropylphenyl)-5-fluoropicolinate. LC/MS=310.0 (M+H), Rt=1.00 min.

### Synthesis of 6-(2,6-difluoro-4-isopropylphenyl)-5-fluoropicolinic acid

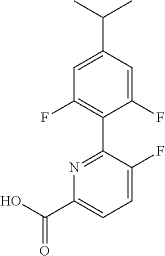

Method 2 was followed using methyl 6-(2,6-difluoro-4-isopropylphenyl)-5-fluoropicolinate to give 6-(2,6-difluoro-4-isopropylphenyl)-5-fluoropicolinic acid in 100% yield. LC/MS=296.2 (M+H), Rt=0.89 min.

### Synthesis of 3-(3,5-difluorophenyl)tetrahydrofuran-3-ol

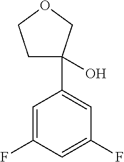

To a solution of 1-bromo-3,5-difluorobenzene (1.6 equiv.) in THF (0.10 M) was added Mg (1.6 equiv.). The mixture was placed in a 90° C. oil bath and refluxed for 3 hrs. The mixture was then cooled to rt and dihydrofuran-3(2H)-one (1.0 equiv.) in THF (0.10 M) was added dropwise via syringe. After stirred at rt for 3 days, the mixture was quenched with sat. NaHCO3 and extracted with EtOAc. The organic layer was washed with brine, dried over Na2SO4 and concentrated. The crude was purified by ISCO separation (0 to 50% EtOAc in hepatane) to give 3-(3,5-difluorophenyl)tetrahydrofuran-3-ol in 50% yield.

### Synthesis of 3-(3,5-difluoro-4-(4,4,5,5-tetramethyl-1,3,2-dioxaborolan-2-yl)phenyl)tetrahydrofuran-3-ol

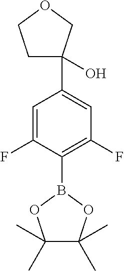

Method 3 was followed using 2-isopropoxy-4,4,5,5-tetramethyl-1,3,2-dioxaborolane (2.2 equiv.), butyllithium (2.2 equiv.) and 3-(3,5-difluorophenyl)tetrahydrofuran-3-ol (1.0 equiv.) to give 3-(3,5-difluoro-4-(4,4,5,5-tetramethyl-1,3,2-dioxaborolan-2-yl)phenyl)tetrahydrofuran-3-ol in 100% yield.

### Synthesis of methyl 6-(2,6-difluoro-4-(3-hydroxytetrahydrofuran-3-yl)phenyl)-5-fluoropicolinate

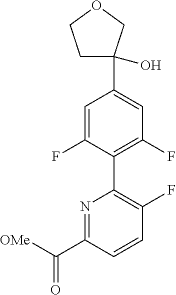

Method 1 was followed using methyl 6-bromo-5-fluoropicolinate (1.2 equiv.) and 3-(3,5-difluoro-4-(4,4,5,5-tetramethyl-1,3,2-dioxaborolan-2-yl)phenyl)tetrahydro furan-3-ol (1.0 equiv.) at 100° C. in microwave for 20 min to give methyl 6-(2,6-difluoro-4-(3-hydroxytetrahydrofuran-3-yl)phenyl)-5-fluoropicolinate in 100% yield. LC/MS=354.1 (M+H), Rt=0.68 min.

### Synthesis of methyl 6-(4-(4,5-dihydrofuran-3-yl)-2,6-difluorophenyl)-5-fluoropicolinate and methyl 6-(4-(2,5-dihydrofuran-3-yl)-2,6-difluorophenyl)-5-fluoropicolinate

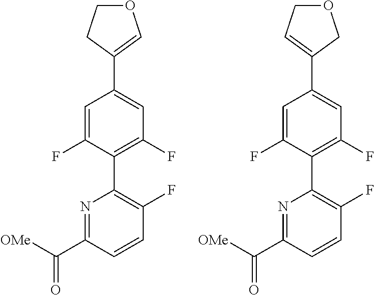

To a solution of methyl 6-(2,6-difluoro-4-(3-hydroxytetrahydrofuran-3-yl)phenyl)-5-fluoropicolinate (1.0 equiv.) in DCM (2.4 M) was added TFA (17.0 equiv.). The mixture was heated at 120° C. in microwave for 2 hrs and concentrated. The crude was purified by ISCO separation (0 to 60% EtOAc in heptanes) to give a mixture of methyl 6-(4-(4,5-dihydro furan-3-yl)-2,6-difluorophenyl)-5-fluoropicolinate and methyl 6-(4-(2,5-dihydro furan-3-yl)-2,6-difluorophenyl)-5-fluoropicolinate in 45% yield. LC/MS=336.2 (M+H), Rt=0.89, 0.97 min.

### Synthesis of methyl 6-(2,6-difluoro-4-(tetrahydrofuran-3-yl)phenyl)-5-fluoropicolinate

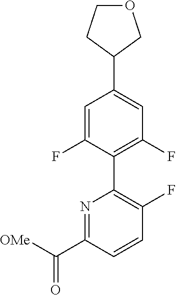

To a degassed solution of methyl 6-(4-(4,5-dihydrofuran-3-yl)-2,6-difluorophenyl)-5-fluoropicolinate and methyl 6-(4-(2,5-dihydrofuran-3-yl)-2,6-difluorophenyl)-5-fluoropicolinate in MeOH (0.10 M) was added Pd/C (0.15 equiv.). The mixture was purged with H2 and then stirred under H2 at rt overnight. The mixture was diluted with DCM and filtered through syringe filter. The filtrated was concentrated to give methyl 6-(2,6-difluoro-4-(tetrahydrofuran-3-yl)phenyl)-5-fluoropicolinate in 84% yield. LC/MS=338.0 (M+H), Rt=0.88 min.

### Synthesis of 6-(2,6-difluoro-4-(tetrahydrofuran-3-yl)phenyl)-5-fluoropicolinic acid

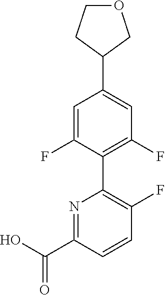

Method 2 was followed using methyl 6-(2,6-difluoro-4-(tetrahydrofuran-3-yl)phenyl)-5-fluoropicolinate to give 6-(2,6-difluoro-4-(tetrahydrofuran-3-yl)phenyl)-5-fluoropicolinic acid in 74% yield. LC/MS=323.9 (M+H), Rt=0.75 min.

### Method 5

A homogeneous solution of 1 eq each of amine, carboxylic acid, HOAT and EDC in DMF, at a concentration of 0.5 M, was left standing for 24 hours at which time water and ethyl acetate were added. The organic phase was dried with sodium sulfate and purified via silica gel column chromatography eluting with ethyl acetate and hexanes to give the desired protected amide product. Alternatively the crude reaction mixture was directly purified by HPLC. Upon lyophilization, the TFA salt of the protected amide product was obtained. Alternatively, the HPLC fractions could be added to EtOAc and solid Na₂CO₃, separated and washed with NaCl_{(sat.)}. Upon drying over MgSO₄, filtering and removing the volatiles in vacuo, the protected amide product was obtained as a free base. Alternatively, the crude reaction mixture was used for the deprotection step without further purification.

If an N-Boc protected amine was present, it was removed by treating with excess 4M HCl/dioxane for 14 hours or by treating with 25% TFA/CH₂Cl₂ for 2 hours. Upon removal of the volatiles in vacuo, the material was purified by RP HPLC yielding after lyophilization the amide product as the TFA salt. Alternatively, the HPLC fractions could be added to EtOAc and solid Na₂CO₃, separated and washed with NaCl_{(sat.)}. Upon drying over MgSO₄, filtering and removing the volatiles in vacuo the free base was obtained. Upon dissolving in MeCN/H₂O, adding 1 eq. of 1 N HCl and lyophilizing, the HCl salt of the amide product was obtained.

If an N-Boc1,2 amino alcohol cyclic carbamate was present, prior to Boc deprotection the cyclic carbamate could be cleaved by treating with Cs₂CO₃ (0.5 eq) in ethanol at a concentration of 0.1 M for three hours. After removal of volatiles in vacuo, the Boc amino group was deprotected as described above. Alternatively, the carbamate could be cleaved by treating with LiOH (3 eq) in THF at a concentration of 0.1 M for four hours prior to Boc deprotection.

If an N-Boc, OAc group were present, prior to Boc deprotection, the acetate group could be cleaved by treating with K₂CO₃ (2.0 equiv.) in ethanol at a concentration of 0.1 M for 24 hours.

If an N-phthalimide group was present, the amine was deprotected by treating with hydrazine in MeOH at 65° C. for three hours. Upon cooling and filtering off the white precipitate, the filtrate was concentrated and purified by RP HPLC to yield the amino amide product.

If a TBDMS ether was present, it was deprotected prior to Boc removal by treating with 6N HCl, THF, methanol (1:2:1) at room temperature for 12 h. After removal of volatiles in vacuo, the Boc amino group was deprotected as described above. Alternatively, the TBDMS ether and Boc group could be both deprotected with 6N HCl, THF, methanol (1:2:1) if left at rt for 24 hours, or heated at 60° C. for 3 hours.

If a OMe group was present, it was deprotected by treating with 1 M BBr₃ in DCM (2.0 equiv.) for 24 hours. Water was added dropwise and the volatiles were removed in vacuo. The material was purified via reverse phase HPLC as described above. If a OBn group was present, it was deprotected by treatment with 10% Pd/C (0.2 equiv.) under an atmosphere of hydrogen in ethyl acetate and methanol (1:2). Upon completion, the reaction was filtered through Celite, washed with methanol, and the filtrate was concentrated in vacuo. If a CO₂Me group was present, it could be converted to the corresponding CO₂H following Method 2.

Following the procedures of Method 5, the following compounds were prepared:

TABLE 1

| | | LC/MS | | |
|---|---|---|---|---|
| | | (M + H | LC/MS | |
| Ex | | on | (Rf on | |
| # | Structure | UPLC) | UPLC) | Chemical Name |
| 1 | 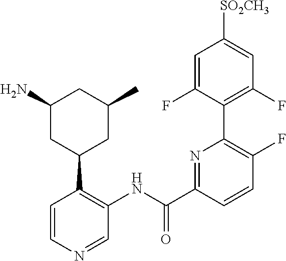 | 519.0 | 0.57 | N-(4-((1R,3S,5S)-3- amino-5- methylcyclohexyl)pyridin- 3-yl)-6-(2,6-difluoro-4- (methylsulfonyl)phenyl)- 5-fluoropicolinamide |
| 2 | 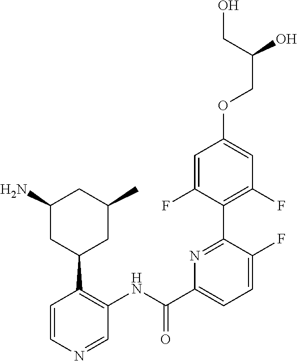 | 531.1 | 0.55 | N-(4-((1R,3S,5S)-3- amino-5- methylcyclohexyl)pyridin- 3-yl)-6-(4-((R)-2,3- dihydroxypropoxy)-2,6- difluorophenyl)-5- fluoropicolinamide |
| 3 | 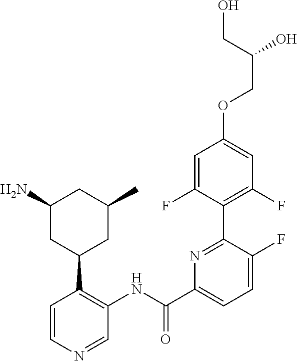 | 531.1 | 0.55 | N-(4-((1R,3S,5S)-3- amino-5- methylcyclohexyl)pyridin- 3-yl)-6-(4-((S)-2,3- dihydroxypropoxy)-2,6- difluorophenyl)-5- fluoropicolinamide |
| 4 | 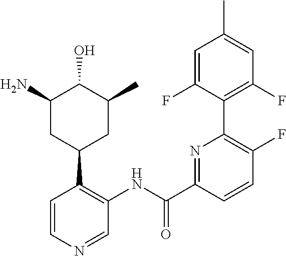 | 471.1 | 0.63 | N-(4-((1R,3R,4R,5S)-3- amino-4-hydroxy-5- methylcyclohexyl)pyridin- 3-yl)-6-(2,6-difluoro-4- methylphenyl)-5- fluoropicolinamide |
| 5 | 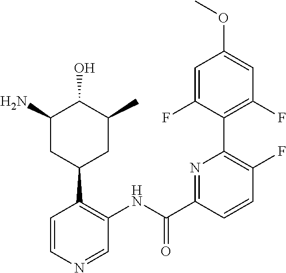 | 487.1 | 0.61 | N-(4-((1R,3R,4R,5S)-3- amino-4-hydroxy-5- methylcyclohexyl)pyridin- 3-yl)-6-(2,6-difluoro-4- methoxyphenyl)-5- fluoropicolinamide |
| 6 | 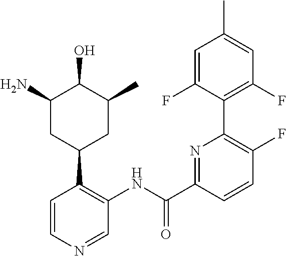 | 471.1 | 0.62 | N-(4-((1R,3R,4S,5S)-3- amino-4-hydroxy-5- methylcyclohexyl)pyridin- 3-yl)-6-(2,6-difluoro-4- methylphenyl)-5- fluoropicolinamide |
| 7 | 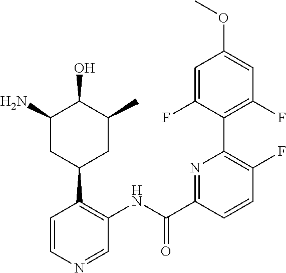 | 487.1 | 0.61 | N-(4-((1R,3R,4S,5S)-3- amino-4-hydroxy-5- methylcyclohexyl)pyridin- 3-yl)-6-(2,6-difluoro-4- methoxyphenyl)-5- fluoropicolinamide |
| 8 | 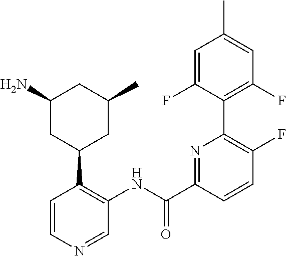 | 455.1 | 0.66 | N-(4-((1R,3S,5S)-3- amino-5- methylcyclohexyl)pyridin- 3-yl)-6-(2,6-difluoro-4- methylphenyl)-5- fluoropicolinamide |
| 9 | 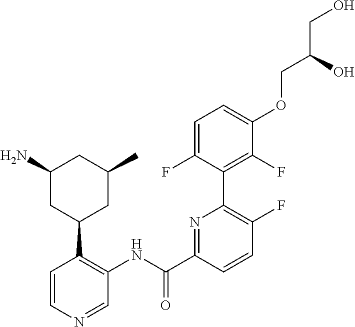 | 531.2 | 0.52 | N-(4-((1R,3S,5S)-3- amino-5- methylcyclohexyl)pyridin- 3-yl)-6-(3-((R)-2,3- dihydroxypropoxy)-2,6- difluorophenyl)-5- fluoropicolinamide |
| 10 | 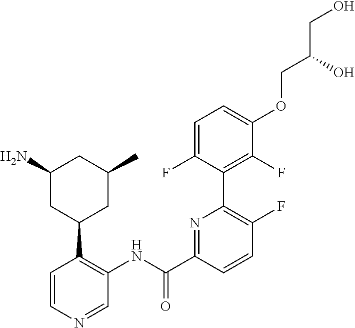 | 531.2 | 0.50 | N-(4-((1R,3S,5S)-3- amino-5- methylcyclohexyl)pyridin- 3-yl)-6-(3-((S)-2,3- dihydroxypropoxy)-2,6- difluorophenyl)-5- fluoropicolinamide |
| 11 | 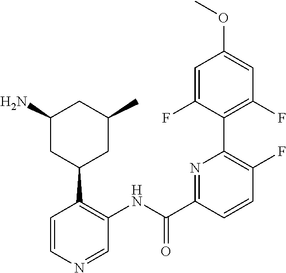 | 471.1 | 0.64 | N-(4-((1R,3S,5S)-3- amino-5- methylcyclohexyl)pyridin- 3-yl)-6-(2,6-difluoro-4- methoxyphenyl)-5- fluoropicolinamide |
| 12 | 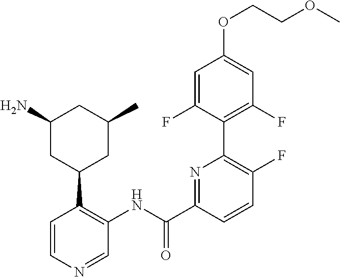 | 515.5 | 0.65 | N-(4-((1R,3S,5S)-3- amino-5- methylcyclohexyl)pyridin- 3-yl)-6-(2,6-difluoro-4-(2- methoxyethoxy)phenyl)- 5-fluoropicolinamide |
| 13 | 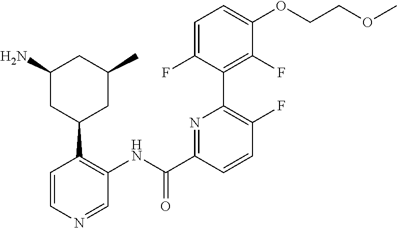 | 515.1 | 0.61 | N-(4-((1R,3S,5S)-3- amino-5- methylcyclohexyl)pyridin- 3-yl)-6-(2,6-difluoro-3-(2- methoxyethoxy)phenyl)- 5-fluoropicolinamide |
| 14 | 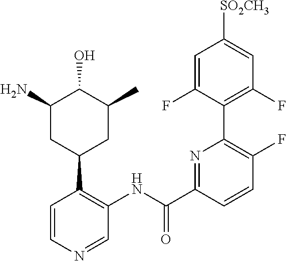 | 535.0 | 0.56 | N-(4-((1R,3R,4R,5S)-3- amino-4-hydroxy-5- methylcyclohexyl)pyridin- 3-yl)-6-(2,6-difluoro-4- (methylsulfonyl)phenyl)- 5-fluoropicolinamide |
| 15 | 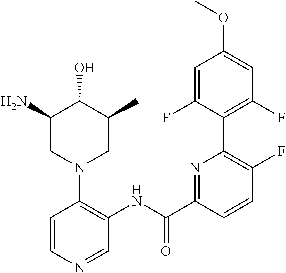 | 488.0 | 0.59 | N-(4-((3R,4R,5S)-3- amino-4-hydroxy-5- methylpiperidin-1- yl)pyridin-3-yl)-6-(2,6- difluoro-4- methoxyphenyl)-5- fluoropicolinamide |
| 16 | 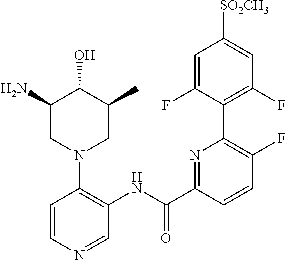 | 535.9 | 0.54 | N-(4-((3R,4R,5S)-3- amino-4-hydroxy-5- methylpiperidin-1- yl)pyridin-3-yl)-6-(2,6- difluoro-4- (methylsulfonyl)phenyl)- 5-fluoropicolinamide |
| 17 | 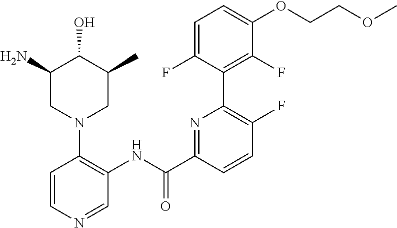 | 532.1 | 0.55 | N-(4-((3R,4R,5S)-3- amino-4-hydroxy-5- methylpiperidin-1- yl)pyridin-3-yl)-6-(2,6- difluoro-3-(2- methoxyethoxy)phenyl)- 5-fluoropicolinamide |
| 18 | 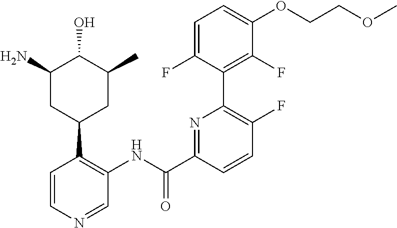 | 531.1 | 0.57 | N-(4-((1R,3R,4R,5S)-3- amino-4-hydroxy-5- methylcyclohexyl)pyridin- 3-yl)-6-(2,6-difluoro-3-(2- methoxyethoxy)phenyl)- 5-fluoropicolinamide |
| 19 | 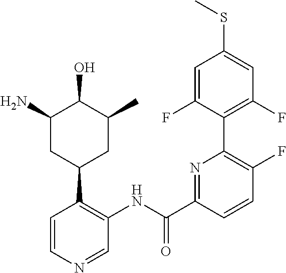 | 503.0 | 0.67 | N-(4-((1R,3R,4S,5S)-3- amino-4-hydroxy-5- methylcyclohexyl)pyridin- 3-yl)-6-(2,6-difluoro-4- (methylthio)phenyl)-5- fluoropicolinamide |
| 20 | 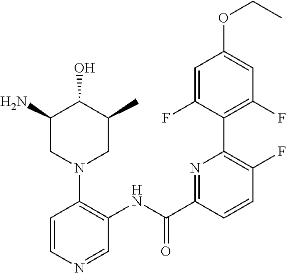 | 502.2 | 0.64 | N-(4-((3R,4R,5S)-3- amino-4-hydroxy-5- methylpiperidin-1- yl)pyridin-3-yl)-6-(4- ethoxy-2,6- difluorophenyl)-5- fluoropicolinamide |
| 21 | 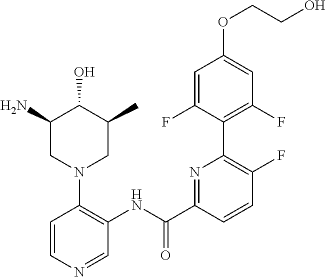 | 518.0 | 0.51 | N-(4-((3R,4R,5S)-3- amino-4-hydroxy-5- methylpiperidin-1- yl)pyridin-3-yl)-6-(2,6- difluoro-4-(2- hydroxyethoxy)phenyl)-5- fluoropicolinamide |
| 22 | 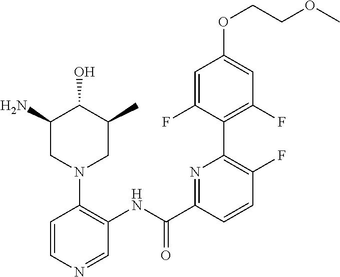 | 532.0 | 0.60 | N-(4-((3R,4R,5S)-3- amino-4-hydroxy-5- methylpiperidin-1- yl)pyridin-3-yl)-6-(2,6- difluoro-4-(2- methoxyethoxy)phenyl)- 5-fluoropicolinamide |
| 23 | 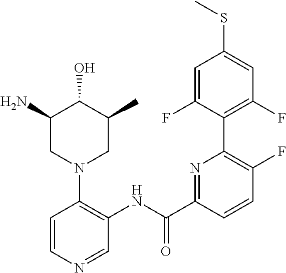 | 503.9 | 0.63 | N-(4-((3R,4R,5S)-3- amino-4-hydroxy-5- methylpiperidin-1- yl)pyridin-3-yl)-6-(2,6- difluoro-4- (methylthio)phenyl)-5- fluoropicolinamide |
| 24 | 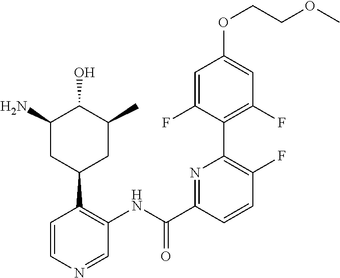 | 531.1 | 0.60 | N-(4-((1R,3R,4R,5S)-3- amino-4-hydroxy-5- methylcyclohexyl)pyridin- 3-yl)-6-(2,6-difluoro-4-(2- methoxyethoxy)phenyl)- 5-fluoropicolinamide |
| 25 | 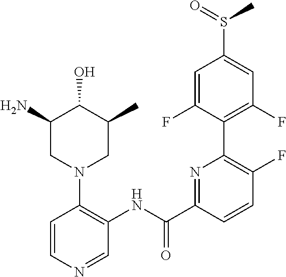 | 520.1 | 0.52 | N-(4-((3R,4R,5S)-3- amino-4-hydroxy-5- methylpiperidin-1- yl)pyridin-3-yl)-6-(2,6- difluoro-4-((S)- methylsulfinyl)phenyl)-5- fluoropicolinamide |
| 26 | 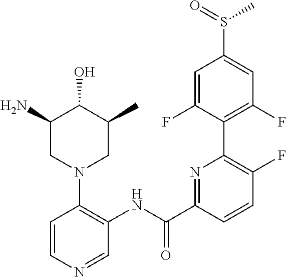 | 520.1 | 0.53 | N-(4-((3R,4R,5S)-3- amino-4-hydroxy-5- methylpiperidin-1- yl)pyridin-3-yl)-6-(2,6- difluoro-4-((R)- methylsulfinyl)phenyl)-5- fluoropicolinamide |
| 27 | 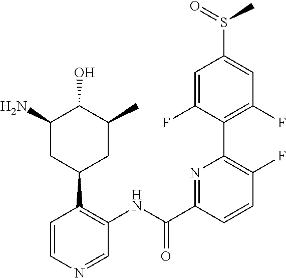 | 519.0 | 0.58 | N-(4-((1R,3R,4R,5S)-3- amino-4-hydroxy-5- methylcyclohexyl)pyridin- 3-yl)-6-(2,6-difluoro-4- ((S)- methylsulfinyl)phenyl)-5- fluoropicolinamide |
| 28 | 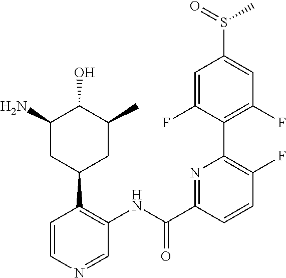 | 519.0 | 0.59 | N-(4-((1R,3R,4R,5S)-3- amino-4-hydroxy-5- methylcyclohexyl)pyridin- 3-yl)-6-(2,6-difluoro-4- methylsulfinyl)phenyl)-5- fluoropicolinamide |
| 29 | 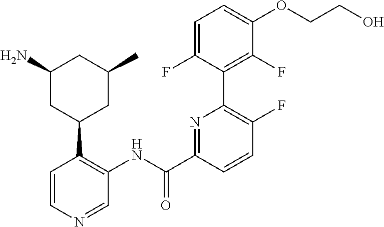 | 501.1 | 0.56 | N-(4-((1R,3S,5S)-3- amino-5- methylcyclohexyl)pyridin- 3-yl)-6-(2,6-difluoro-3-(2- hydroxyethoxy)phenyl)-5- fluoropicolinamide |
| 30 | 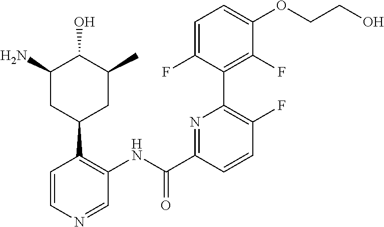 | 517.0 | 0.52 | N-(4-((1R,3R,4R,5S)-3- amino-4-hydroxy-5- methylcyclohexyl)pyridin- 3-yl)-6-(2,6-difluoro-3-(2- hydroxyethoxy)phenyl)-5- fluoropicolinamide |
| 31 | 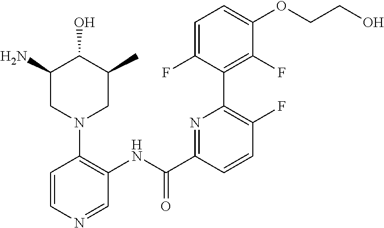 | 518.0 | 0.50 | N-(4-((3R,4R,5S)-3- amino-4-hydroxy-5- methylpiperidin-1- yl)pyridin-3-yl)-6-(2,6- difluoro-3-(2- hydroxyethoxy)phenyl)-5- fluoropicolinamide |
| 32 | 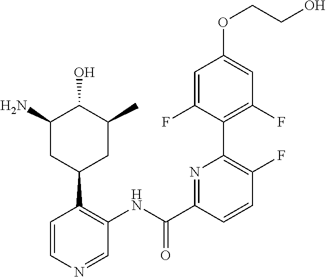 | 517.0 | 0.56 | N-(4-((1R,3R,4R,5S)-3- amino-4-hydroxy-5- methylcyclohexyl)pyridin- 3-yl)-6-(2,6-difluoro-4-(2- hydroxyethoxy)phenyl)-5- fluoropicolinamide |
| 33 | 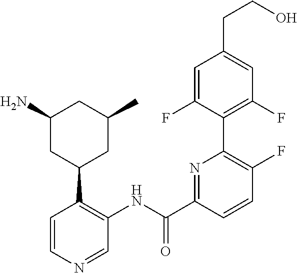 | 485.1 | 0.57 | N-(4-((1R,3S,5S)-3- amino-5- methylcyclohexyl)pyridin- 3-yl)-6-(2,6-difluoro-4-(2- hydroxyethyl)phenyl)-5- fluoropicolinamide |
| 34 | 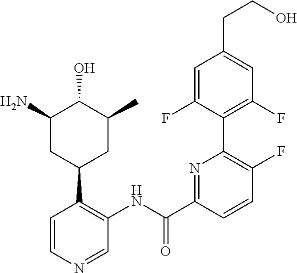 | 501.0 | 0.56 | N-(4-((1R,3R,4R,5S)-3- amino-4-hydroxy-5- methylcyclohexyl)pyridin- 3-yl)-6-(2,6-difluoro-4-(2- hydroxyethyl)phenyl)-5- fluoropicolinamide |
| 35 | 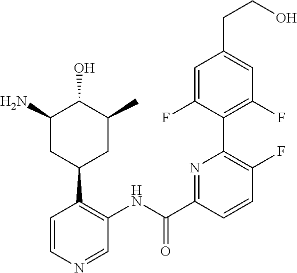 | 502.1 | 0.51 | N-(4-((3R,4R,5S)-3- amino-4-hydroxy-5- methylpiperidin-1- yl)pyridin-3-yl)-6-(2,6- difluoro-4-(2- hydroxyethyl)phenyl)-5- fluoropicolinamide |
| 36 | 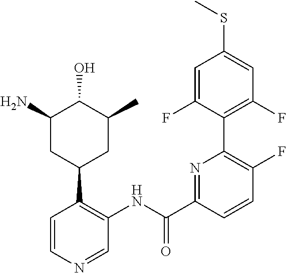 | 503.0 | 0.68 | N-(4-((1R,3R,4R,5S)-3- amino-4-hydroxy-5- methylcyclohexyl)pyridin- 3-yl)-6-(2,6-difluoro-4- (methylthio)phenyl)-5- fluoropicolinamide |
| 37 | 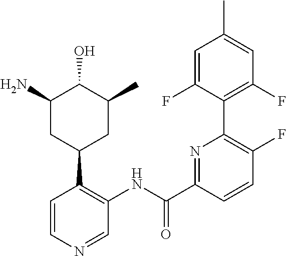 | 472.1 | 0.59 | N-(4-((3R,4R,5S)-3- amino-4-hydroxy-5- methylpiperidin-1- yl)pyridin-3-yl)-6-(2,6- difluoro-4-methylphenyl)- 5-fluoropicolinamide |
| 38 | 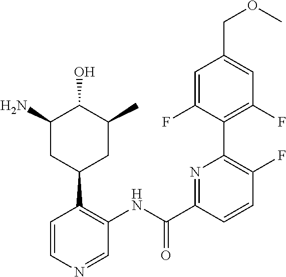 | 502.0 | 0.57 | N-(4-((3R,4R,5S)-3- amino-4-hydroxy-5- methylpiperidin-1- yl)pyridin-3-yl)-6-(2,6- difluoro-4- (methoxymethyl)phenyl)- 5-fluoropicolinamide |
| 39 | 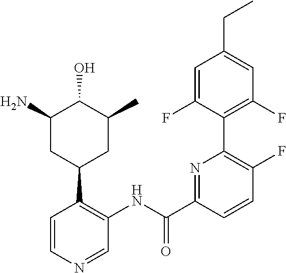 | 485.0 | 0.66 | N-(4-((1R,3R,4R,5S)-3- amino-4-hydroxy-5- methylcyclohexyl)pyridin- 3-yl)-6-(4-ethyl-2,6- difluorophenyl)-5- fluoropicolinamide |
| 40 | 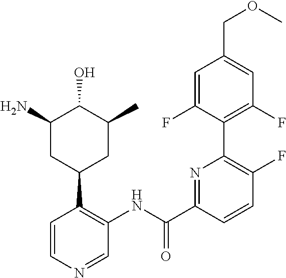 | 501.1 | 0.61 | N-(4-((1R,3R,4R,5S)-3- amino-4-hydroxy-5- methylcyclohexyl)pyridin- 3-yl)-6-(2,6-difluoro-4- (methoxymethyl)phenyl)- 5-fluoropicolinamide |
| 41 | 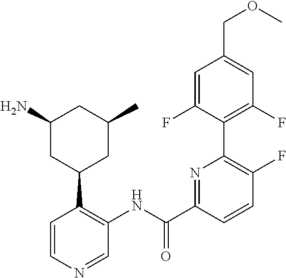 | 485.1 | 0.63 | N-(4-((1R,3S,5S)-3- amino-5- methylcyclohexyl)pyridin- 3-yl)-6-(2,6-difluoro-4- (methoxymethyl)phenyl)- 5-fluoropicolinamide |
| 42 | 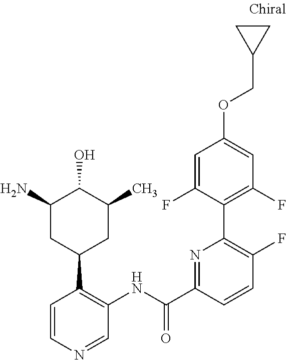 | 527.1 | 0.70 | N-(4-((1R,3R,4R,5S)-3- amino-4-hydroxy-5- methylcyclohexyl)pyridin- 3-yl)-6-(4- (cyclopropylmethoxy)- 2,6-difluorophenyl)-5- fluoropicolinamide |
| 43 | 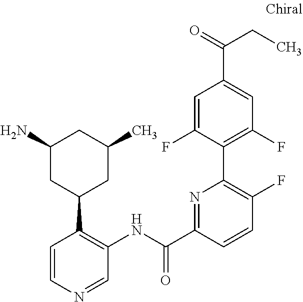 | 497.0 | 0.70 | N-(4-((1R,3S,5S)-3- amino-5- methylcyclohexyl)pyridin- 3-yl)-6-(2,6-difluoro-4- propionylphenyl)-5- fluoropicolinamide |
| 44 | 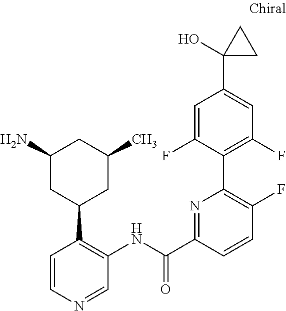 | 497.1 | 0.64 | N-(4-((1R,3S,5S)-3- amino-5- methylcyclohexyl)pyridin- 3-yl)-6-(2,6-difluoro-4-(1- hydroxycyclopropyl) phenyl)-5- fluoropicolinamide |
| 45 | 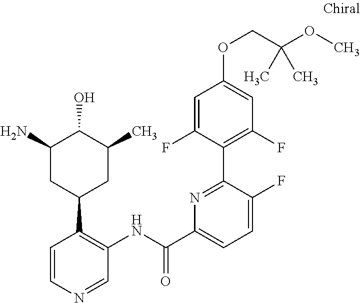 | 559.0 | 0.72 | N-(4-((1R,3R,4R,5S)-3- amino-4-hydroxy-5- methylcyclohexyl)pyridin- 3-yl)-6-(2,6-difluoro-4-(2- methoxy-2- methylpropoxy)phenyl)-5- fluoropicolinamide |
| 46 | 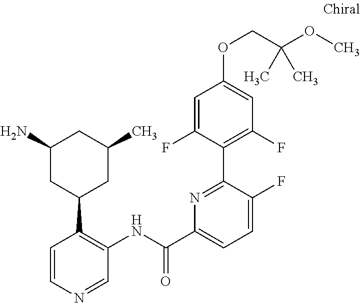 | 543.1 | 0.76 | N-(4-((1R,3S,5S)-3- amino-5- methylcyclohexyl)pyridin- 3-yl)-6-(2,6-difluoro-4-(2- methoxy-2- methylpropoxy)phenyl)-5- fluoropicolinamide |
| 47 | 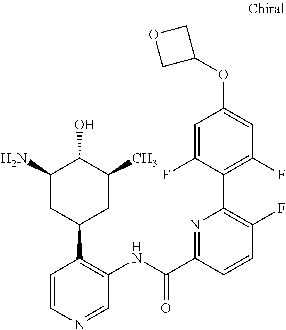 | 529.1 | 0.60 | N-(4-((1R,3R,4R,5S)-3- amino-4-hydroxy-5- methylcyclohexyl)pyridin- 3-yl)-6-(2,6-difluoro-4- (oxetan-3-yloxy)phenyl)- 5-fluoropicolinamide |
| 48 | 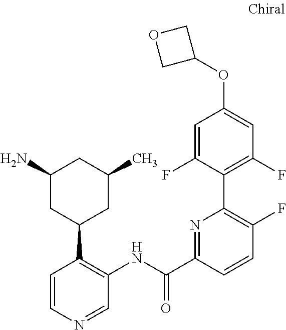 | 513.2 | 0.59 | N-(4-((1R,3S,5S)-3- amino-5- methylcyclohexyl)pyridin- 3-yl)-6-(2,6-difluoro-4- (oxetan-3-yloxy)phenyl)- 5-fluoropicolinamide |
| 49 | 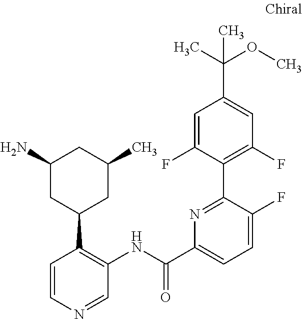 | 513.1 | 0.71 | N-(4-((1R,3S,5S)-3- amino-5- methylcyclohexyl)pyridin- 3-yl)-6-(2,6-difluoro-4-(2- methoxypropan-2- yl)phenyl)-5- fluoropicolinamide |
| 50 | 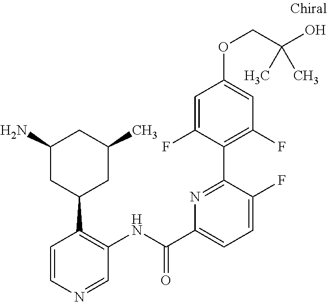 | 529.0 | 0.67 | N-(4-((1R,3S,5S)-3- amino-5- methylcyclohexyl)pyridin- 3-yl)-6-(2,6-difluoro-4-(2- hydroxy-2- methylpropoxy)phenyl)-5- fluoropicolinamide |
| 51 | 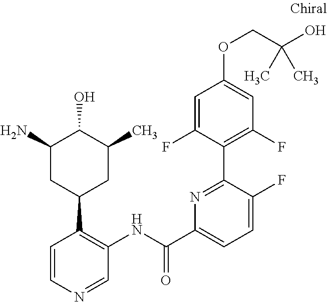 | 545.0 | 0.62 | N-(4-((1R,3R,4R,5S)-3- amino-4-hydroxy-5- methylcyclohexyl)pyridin- 3-yl)-6-(2,6-difluoro-4-(2- hydroxy-2- methylpropoxy)phenyl)-5- fluoropicolinamide |
| 52 | 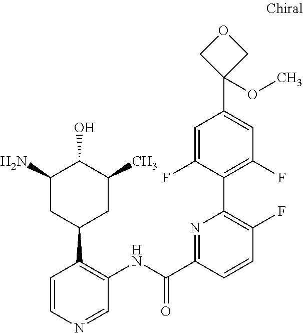 | 543.2 | 0.59 | N-(4-((1R,3R,4R,5S)-3- amino-4-hydroxy-5- methylcyclohexyl)pyridin- 3-yl)-6-(2,6-difluoro-4-(3- methoxyoxetan-3- yl)phenyl)-5- fluoropicolinamide |
| 53 | 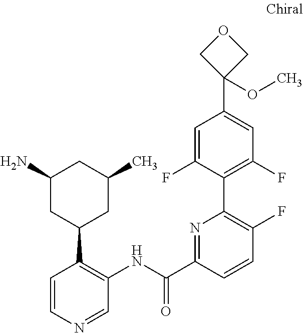 | 527.2 | 0.62 | N-(4-((1R,3S,5S)-3- amino-5- methylcyclohexyl)pyridin- 3-yl)-6-(2,6-difluoro-4-(3- methoxyoxetan-3- yl)phenyl)-5- fluoropicolinamide |
| 54 | 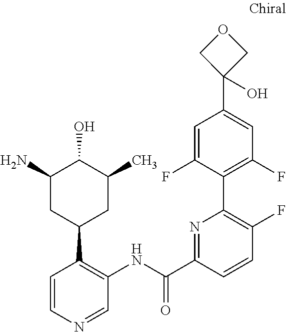 | 529.1 | 0.54 | N-(4-((1R,3R,4R,5S)-3- amino-4-hydroxy-5- methylcyclohexyl)pyridin- 3-yl)-6-(2,6-difluoro-4-(3- hydroxyoxetan-3- yl)phenyl)-5- fluoropicolinamide |
| 55 | 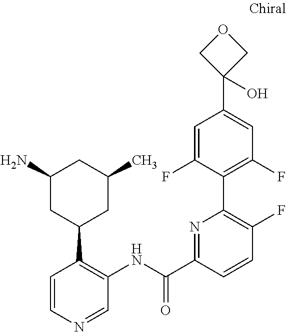 | 513.1 | 0.57 | N-(4-((1R,3S,5S)-3- amino-5- methylcyclohexyl)pyridin- 3-yl)-6-(2,6-difluoro-4-(3- hydroxyoxetan-3- yl)phenyl)-5- fluoropicolinamide |
| 56 | 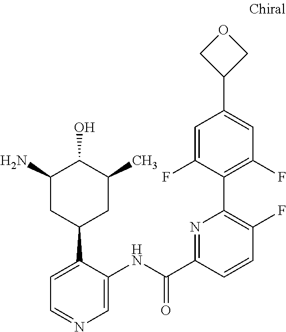 | 513.1 | 0.57 | N-(4-((1R,3R,4R,5S)-3- amino-4-hydroxy-5- methylcyclohexyl)pyridin- 3-yl)-6-(2,6-difluoro-4- (oxetan-3-yl)phenyl)-5- fluoropicolinamide |
| 57 | 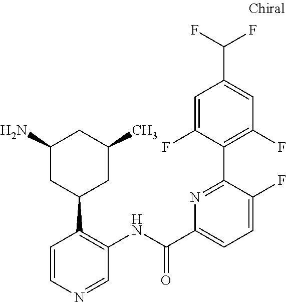 | 491.0 | 0.68 | N-(4-((1R,3S,5S)-3- amino-5- methylcyclohexyl)pyridin- 3-yl)-6-(4- (difluoromethyl)-2,6- difluorophenyl)-5- fluoropicolinamide |
| 58 | 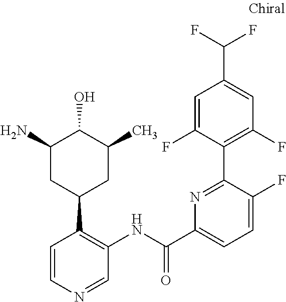 | 507.1 | 0.64 | N-(4-((1R,3R,4R,5S)-3- amino-4-hydroxy-5- methylcyclohexyl)pyridin- 3-yl)-6-(4- (difluoromethyl)-2,6- difluorophenyl)-5- fluoropicolinamide |
| 59 | 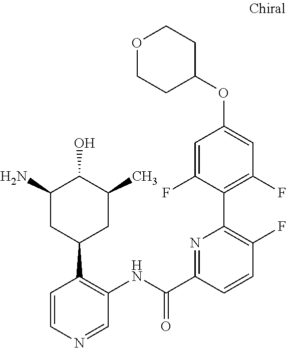 | 557.0 | 0.68 | N-(4-((1R(3R,4R,5S)-3- amino-4-hydroxy-5- methylcyclohexyl)pyridin- 3-yl)-6-(2,6-difluoro-4- (tetrahydro-2H-pyran-4- yloxy)phenyl)-5- fluoropicolinamide |
| 60 | 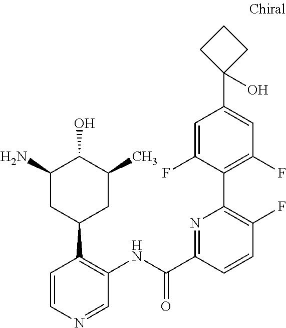 | 527.1 | 0.61 | N-(4-((1R,3R,4R,5S)-3- amino-4-hydroxy-5- methylcyclohexyl)pyridin- 3-yl)-6-(2,6-difluoro-4-(1- hydroxycyclobutyl)phenyl)- 5-fluoropicolinamide |
| 61 | 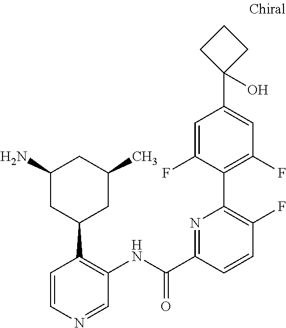 | 511.1 | 0.64 | N-(4-((1R,3S,5S)-3- amino-5- methylcyclohexyl)pyridin- 3-yl)-6-(2,6-difluoro-4-(1- hydroxycyclobutyl)phenyl)- 5-fluoropicolinamide |
| 62 | 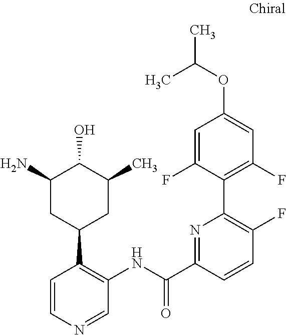 | 515.1 | 0.69 | N-(4-((1R,3R,4R,5S)-3- amino-4-hydroxy-5- methylcyclohexyl)pyridin- 3-yl)-6-(2,6-difluoro-4- isopropoxyphenyl)-5- fluoropicolinamide |
| 63 | 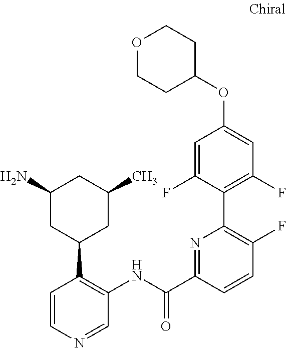 | 541.1 | 0.67 | N-(4-((1R,3S,5S)-3- amino-5- methylcyclohexyl)pyridin- 3-yl)-6-(2,6-difluoro-4- ((tetrahydro-2H-pyran-4- yl)oxy)phenyl)-5- fluoropicolinamide |
| 364 | 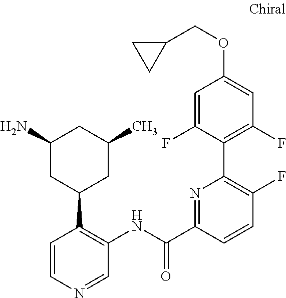 | 511.1 | 0.74 | N-(4-((1R,3S,5S)-3- amino-5- methylcyclohexyl)pyridin- 3-yl)-6-(4- (cyclopropylmethoxy)- 2,6-difluorophenyl)-5- fluoropicolinamide |
| 65 | 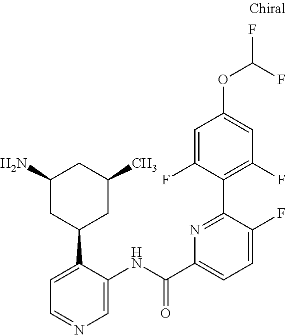 | 507.0 | 0.67 | N-(4-((1R,3S,5S)-3- amino-5- methylcyclohexyl)pyridin- 3-yl)-6-(4- (difluoromethoxy)-2,6- difluorophenyl)-5- fluoropicolinamide |
| 66 | 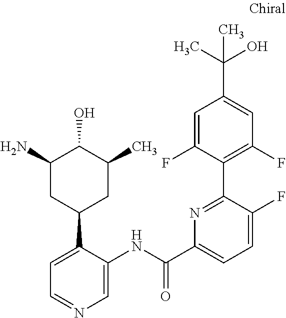 | 515.1 | 0.59 | N-(4-((1R,3R,4R,5S)-3- amino-4-hydroxy-5- methylcyclohexyl)pyridin- 3-yl)-6-(2,6-difluoro-4-(2- hydroxypropan-2- yl)phenyl)-5- fluoropicolinamide |
| 67 | 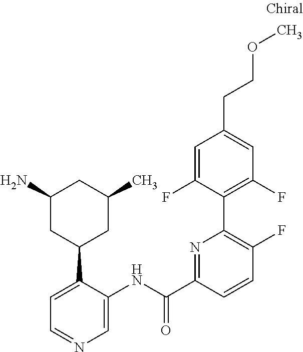 | 499.1 | 0.65 | N-(4-((1R,3S,5S)-3- amino-5- methylcyclohexyl)pyridin- 3-yl)-6-(2,6-difluoro-4-(2- methoxyethyl)phenyl)-5- fiuoropicolinamide |
| 68 | 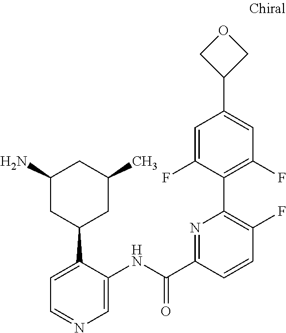 | 497.1 | 0.61 | N-(4-((1R,3S,5S)-3- amino-5- methylcyclohexyl)pyridin- 3-yl)-6-(2,6-difluoro-4- (oxetan-3-yl)phenyl)-5- fluoropicolinamide |
| 69 | 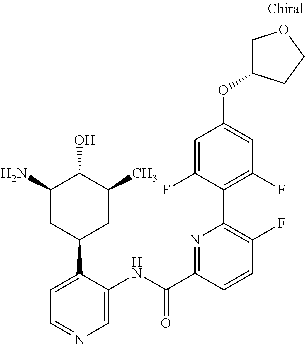 | 543.1 | 0.61 | N-(4-((1R,3R,4R,5S)-3- amino-4-hydroxy-5- methylcyclohexyl)pyridin- 3-yl)-6-(2,6-difluoro-4- ((R)-tetrahydrofuran-3- yloxy)phenyl)-5- fluoropicolinamide |
| 70 | 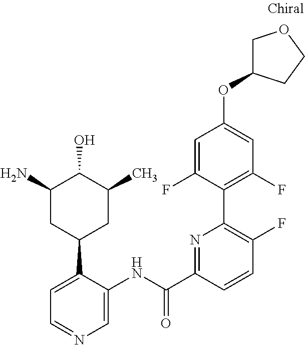 | 543.1 | 0.61 | N-(4-((1R,3R,4R,5S)-3- amino-4-hydroxy-5- methylcyclohexyl)pyridin- 3-yl)-6-(2,6-difluoro-4- ((S)-tetrahydrofuran-3- yloxy)phenyl)-5- fluoropicolinamide |
| 71 | 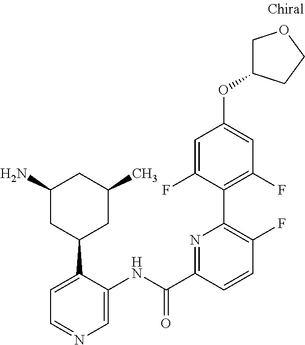 | 527.1 | 0.65 | N-(4-((1R,3S,5S)-3- amino-5- methylcyclohexyl)pyridin- 3-yl)-6-(2,6-difluoro-4- ((R)-tetrahydrofuran-3- yloxy)phenyl)-5- fluoropicolinamide |
| 72 | 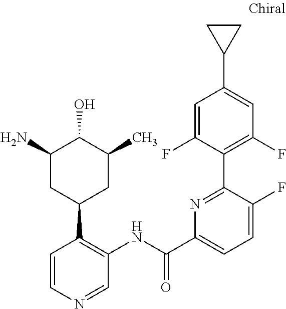 | 597.0 | 0.69 | N-(4-((1R,3R,4R,5S)-3- amino-4-hydroxy-5- methylcyclohexyl)pyridin- 3-yl)-6-(4-cyclopropyl- 2,6-difluorophenyl)-5- fluoropicolinamide |
| 73 | 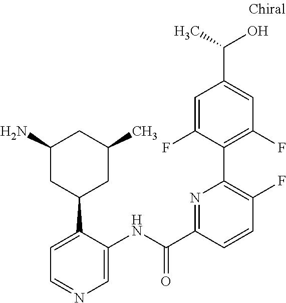 | 485.0 | 0.60 | N-(4-((1R,3S,5S)-3- amino-5- methylcyclohexyl)pyridin- 3-yl)-6-(2,6-difluoro-4- ((S)-1- hydroxyethyl)phenyl)-5- fluoropicolinamide |
| 74 | 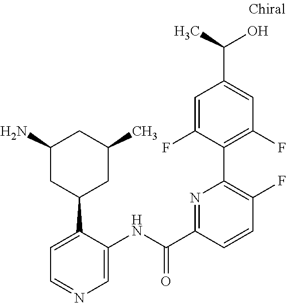 | 485.0 | 0.60 | N-(4-((1R,3S,5S)-3- amino-5- methylcyclohexyl)pyridin- 3-yl)-6-(2,6-difluoro-4- ((R)-1- hydroxyethyl)phenyl)-5- fluoropicolinamide |
| 75 | 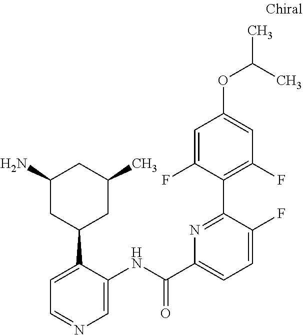 | 499.1 | 0.76 | N-(4-((1R,3S,5S)-3- amino-5- methylcyclohexyl)pyridin- 3-yl)-6-(2,6-difluoro-4- isopropoxyphenyl)-5- fluoropicolinamide |
| 76 | 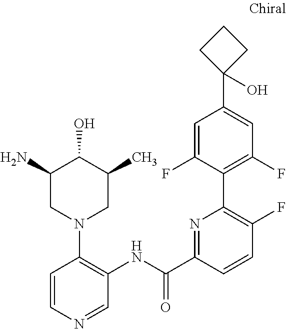 | 528.1 | 0.59 | N-(4-((3R,4R,5S)-3- amino-4-hydroxy-5- methylpiperidin-1- yl)pyridin-3-yl)-6-(2,6- difluoro-4-(1- hydroxycyclobutyl)phenyl)- 5-fluoropicolinamide |
| 77 | 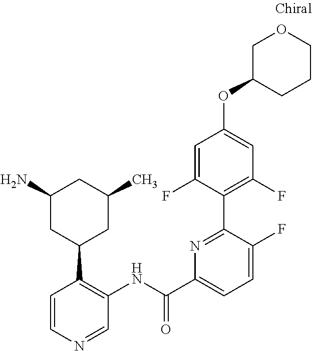 | 541.1 | 0.70 | N-(4-((1R,3S,5S)-3- amino-5- methylcyclohexyl)pyridin- 3-yl)-6-(2,6-difluoro-4- ((R)-tetrahydro-2H-pyran- 3-yloxy)phenyl)-5- fluoropicolinamide |
| 78 | 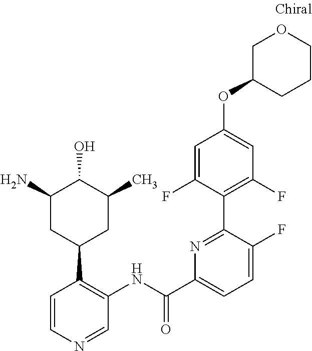 | 557.1 | 0.67 | N-(4-((1R,3R,4R,5S)-3- amino-4-hydroxy-5- methylcyclohexyl)pyridin- 3-yl)-6-(2,6-difluoro-4- ((R)-tetrahydro-2H-pyran- 3-yloxy)phenyl)-5- fluoropicolinamide |
| 79 | 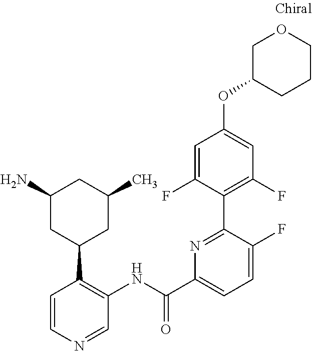 | 541.1 | 0.70 | N-(4-((1R,3S,5S)-3- amino-5- methylcyclohexyl)pyridin- 3-yl)-6-(2,6-difluoro-4- ((S)-tetrahydro-2H-pyran- 3-yloxy)phenyl)-5- fluoropicolinamide |
| 80 | 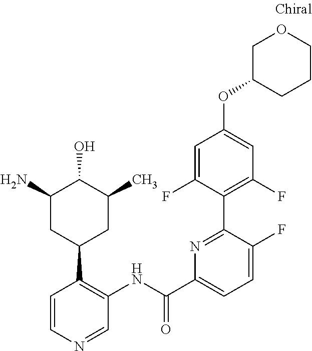 | 557.1 | 0.68 | N-(4-((1R,3R,4R,5S)-3- amino-4-hydroxy-5- methylcyclohexyl)pyridin- 3-yl)-6-(2,6-difluoro-4- ((S)-tetrahydro-2H-pyran- 3-yloxy)phenyl)-5- fluoropicolinamide |
| 81 | 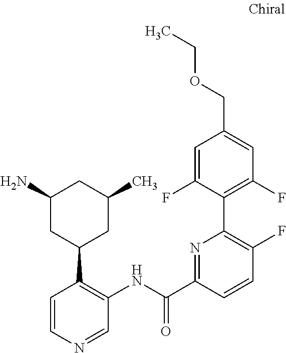 | 499.1 | 0.71 | N-(4-((1R,3S,5S)-3- amino-5- methylcyclohexyl)pyridin- 3-yl)-6-(4-(ethoxymethyl)- 2,6-difluorophenyl)-5- fluoropicolinamide |
| 82 | 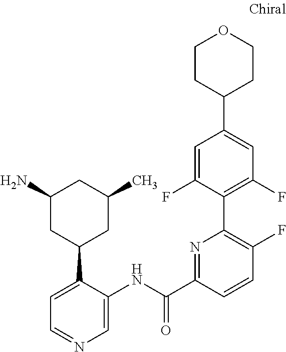 | 525.3 | 0.72 | N-(4-((1R,3S,5S)-3- amino-5- methylcyclohexyl)pyridin- 3-yl)-6-(2,6-difluoro-4- (tetrahydro-2H-pyran-4- yl)phenyl)-5- fluoropicolinamide |
| 83 | 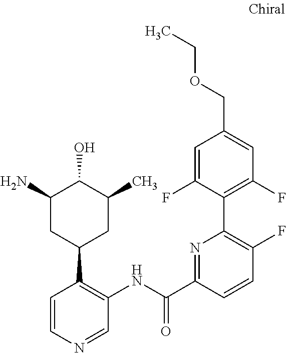 | 515.2 | 0.65 | N-(4-((1R,3R,4R,5S)-3- amino-4-hydroxy-5- methylcyclohexyl)pyridin- 3-yl)-6-(4-(ethoxymethyl)- 2,6-difluorophenyl)-5- fluoropicolinamide |
| 84 | 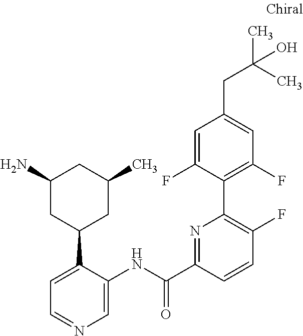 | 513.1 | 0.65 | N-(4-((1R,3S,5S)-3- amino-5- methylcyclohexyl)pyridin- 3-yl)-6-(2,6-difluoro-4-(2- hydroxy-2- methylpropyl)phenyl)-5- fluoropicolinamide |
| 85 | 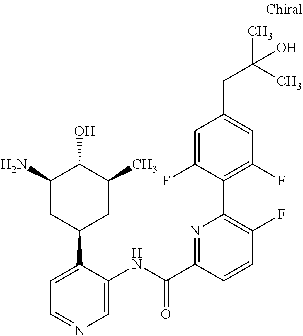 | 529.1 | 0.61 | N-(4-((1R,3R,4R,5S)-3- amino-4-hydroxy-5- methylcyclohexyl)pyridin- 3-yl)-6-(2,6-difluoro-4-(2- hydroxy-2- methylpropyl)phenyl)-5- fluoropicolinamide |
| 86 | 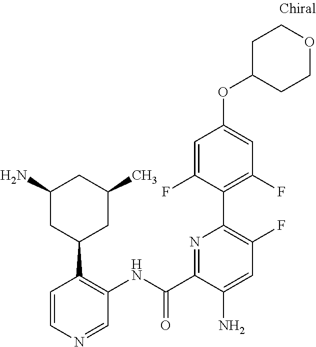 | 556.1 | 0.71 | 3-amino-N-(4- ((1R,3S,5S)-3-amino-5- methylcyclohexyl)pyridin- 3-yl)-6-(2,6-difluoro-4- (tetrahydro-2H-pyran-4- yloxy)phenyl)-5- fluoropicolinamide |
| 87 | 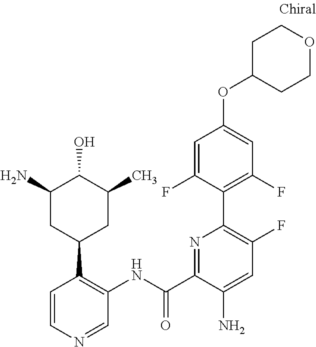 | 572.2 | 0.80 | 3-amino-N-(4- ((1R,3R,4R,5S)-3-amino- 4-hydroxy-5- methylcyclohexyl)pyridin- 3-yl)-6-(2,6-difluoro-4- (tetrahydro-2H-pyran-4- yloxy)phenyl)-5- fluoropicolinamide |
| 88 | 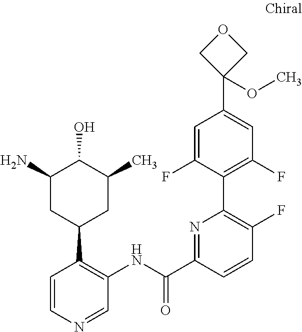 | 544.2 | 0.63 | N-(4-((3R,4R,5S)-3- amino-4-hydroxy-5- methylpiperidin-1- yl)pyridin-3-yl)-6-(2,6- difluoro-4-(3- methoxyoxetan-3- yl)phenyl)-5- fluoropicolinamide |
| 89 | 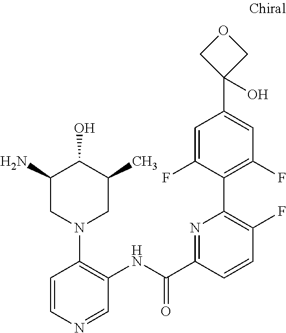 | 530.1 | 0.57 | N-(4-((3R,4R,5S)-3- amino-4-hydroxy-5- methylpiperidin-1- yl)pyridin-3-yl)-6-(2,6- difluoro-4-(3- hydroxyoxetan-3- yl)phenyl)-5- fluoropicolinamide |
| 90 | 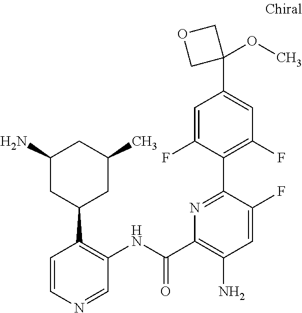 | 542.2 | 0.69 | 3-amino-N-(4- ((1R,3S,5S)-3-amino-5- methylcyclohexyl)pyridin- 3-yl)-6-(2,6-difluoro-4-(3- methoxyoxetan-3- yl)phenyl)-5- fluoropicolinamide |
| 91 | 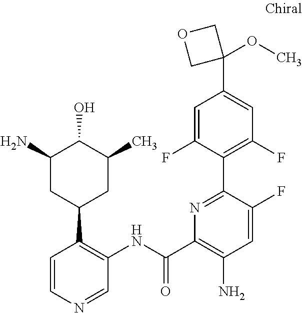 | 558.1 | 0.65 | 3-amino-N-(4- ((1R,3R,4R,5S)-3-amino- 4-hydroxy-5- methylcyclohexyl)pyridin- 3-yl)-6-(2,6-difluoro-4-(3- methoxyoxetan-3- yl)phenyl)-5- fluoropicolinamide |
| 92 | 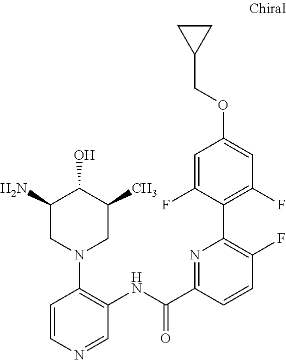 | 528.1 | 0.73 | N-(4-((3R,4R,5S)-3- amino-4-hydroxy-5- methylpiperidin-1- yl)pyridin-3-yl)-6-(4- (cyclopropylmethoxy)- 2,6-difluorophenyl)-5- fluoropicolinamide |
| 93 | 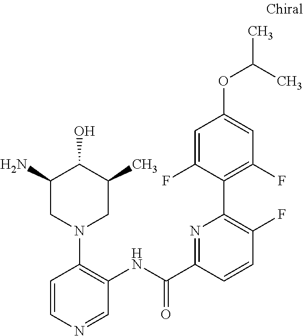 | 516.2 | 0.68 | N-(4-((3R,4R,5S)-3- amino-4-hydroxy-5- methylpiperidin-1- yl)pyridin-3-yl)-6-(2,6- difluoro-4- isopropoxyphenyl)-5- fluoropicolinamide |
| 94 | 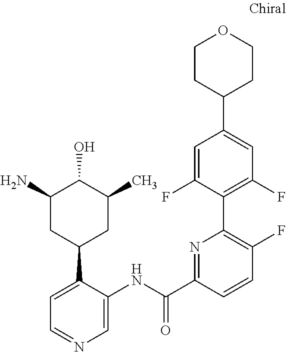 | 541.3 | 0.66 | N-(4-((1R,3R,4R,5S)-3- amino-4-hydroxy-5- methylcyclohexyl)pyridin- 3-yl)-6-(2,6-difluoro-4- (tetrahydro-2H-pyran-4- yl)phenyl)-5- fluoropicolinamide |
| 96 | 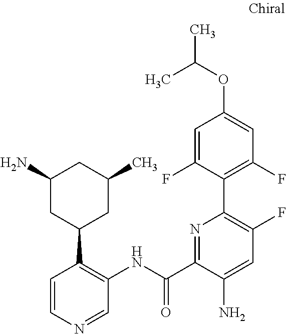 | 514.2 | 0.82 | 3-amino-N-(4- ((1R,3S,5S)-3-amino-5- methylcyclohexyl)pyridin- 3-yl)-6-(2,6-difluoro-4- isopropoxyphenyl)-5- fluoropicolinamide |
| 97 | 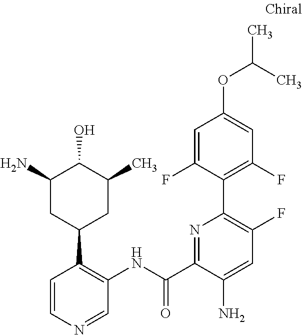 | 530.2 | 0.78 | N-(4-((1R,3R,4R,5S)-3- amino-4-hydroxy-5- methylcyclohexyl)pyridin- 3-yl)-6-(2,6-difluoro-4- isopropoxyphenyl)-5- fluoropicolinamide |
| 98 | 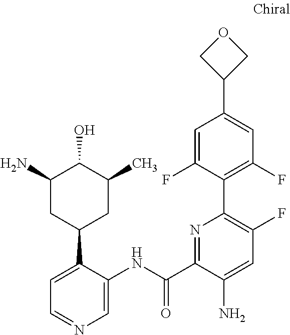 | 528.1 | 0.57 | 3-amino-N-(4- ((1R,3R,4R,5S)-3-amino- 4-hydroxy-5- methylcyclohexyl)pyridin- 3-yl)-6-(2,6-difluoro-4- (oxetan-3-yl)phenyl)-5- fluoropicolinamide |
| 99 | 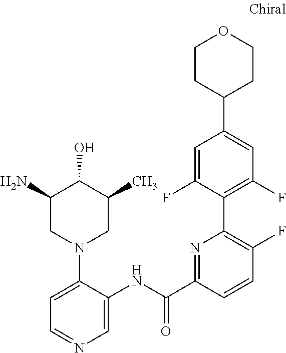 | 542.2 | 0.63 | N-(4-((3R,4R,5S)-3- amino-4-hydroxy-5- methylpiperidin-1- yl)pyridin-3-yl)-6-(2,6- difluoro-4-(tetrahydro-2H- pyran-4-yl)phenyl)-5- fluoropicolinamide |
| 100 | 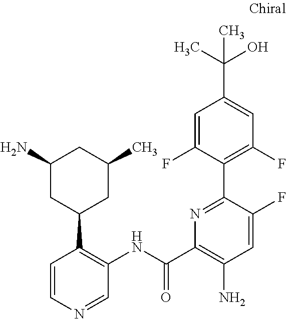 | 514.0 | 0.62 | 3-amino-N-(4- ((1R,3S,5S)-3-amino-5- methylcyclohexyl)pyridin- 3-yl)-6-(2,6-difluoro-4-(2- hydroxypropan-2- yl)phenyl)-5- fluoropicolinamide |
| 101 | 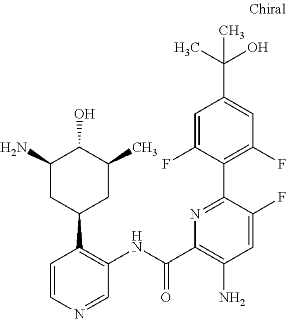 | 530.2 | 0.62 | 3-amino-N-(4- ((1R,3R,4R,5S)-3-amino- 4-hydroxy-5- methylcyclohexyl)pyridin- 3-yl)-6-(2,6-difluoro-4-(2- hydroxypropan-2- yl)phenyl)-5- fluoropicolinamide |
| 102 | 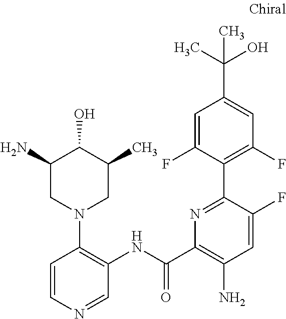 | 531.1 | 0.64 | 3-amino-N-(4- ((3R,4R,5S)-3-amino-4- hydroxy-5- methylpiperidin-1- yl)pyridin-3-yl)-6-(2,6- difluoro-4-(2- hydroxypropan-2- yl)phenyl)-5- fluoropicolinamide |
| 103 | 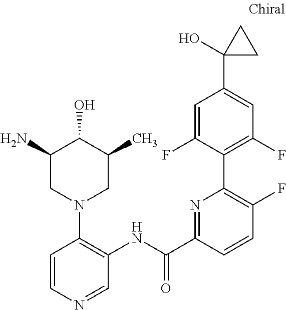 | 514.1 | 0.58 | N-(4-((3R,4R,5S)-3- amino-4-hydroxy-5- methylpiperidin-1- yl)pyridin-3-yl)-6-(2,6- difluoro-4-(1- hydroxycyclopropyl) phenyl)-5- fluoropicolinamide |
| 104 | 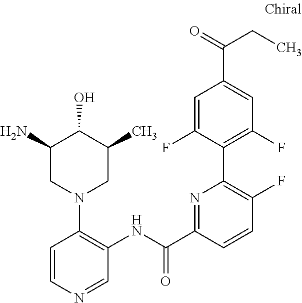 | 514.1 | 0.62 | N-(4-((3R,4R,5S)-3- amino-4-hydroxy-5- methylpiperidin-1- yl)pyridin-3-yl)-6-(2,6- difluoro-4- propionylphenyl)-5- fluoropicolinamide |
| 105 | 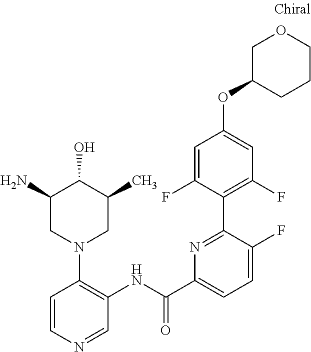 | 558.1 | 0.63 | N-(4-((3R,4R,5S)-3- amino-4-hydroxy-5- methylpiperidin-1- yl)pyridin-3-yl)-6-(2,6- difluoro-4-((R)- tetrahydro-2H-pyran-3- yloxy)phenyl)-5- fluoropicolinamide |
| 106 | 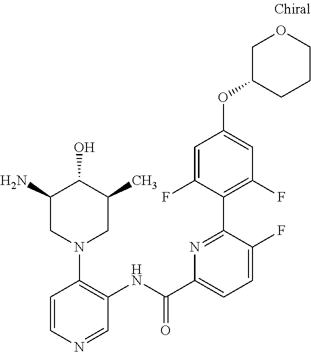 | 558.0 | 0.62 | N-(4-((3R,4R,5S)-3- amino-4-hydroxy-5- methylpiperidin-1- yl)pyridin-3-yl)-6-(2,6- difluoro-4-((S)-tetrahydro- 2H-pyran-3- yloxy)phenyl)-5- fluoropicolinamide |
| 107 | 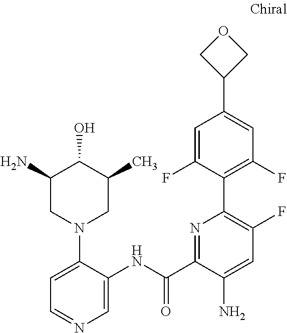 | 529.1 | 0.57 | 3-amino-N-(4- ((3R,4R,5S)-3-amino-4- hydroxy-5- methylpiperidin-1- yl)pyridin-3-yl)-6-(2,6- difluoro-4-(oxetan-3- yl)phenyl)-5- fluoropicolinamide |
| 108 | 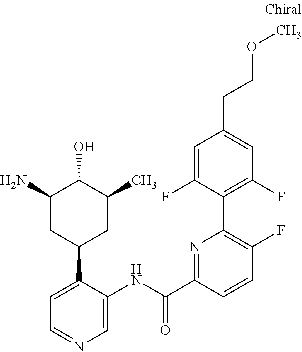 | 515.2 | 0.63 | N-(4-((1R,3R,4R,5S)-3- amino-4-hydroxy-5- methylcyclohexyl)pyridin- 3-yl)-6-(2,6-difluoro-4-(2- methoxyethyl)phenyl)-5- fluoropicolinamide |
| 109 | 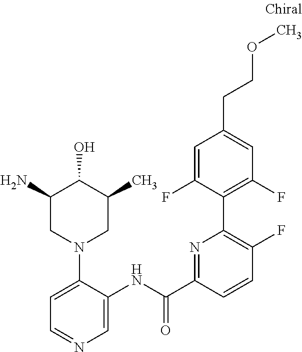 | 516.4 | 0.58 | N-(4-((3R,4R,5S)-3- amino-4-hydroxy-5- methylpiperidin-1- yl)pyridin-3-yl)-6-(2,6- difluoro-4-(2- methoxyethyl)phenyl)-5- fluoropicolinamide |
| 110 | 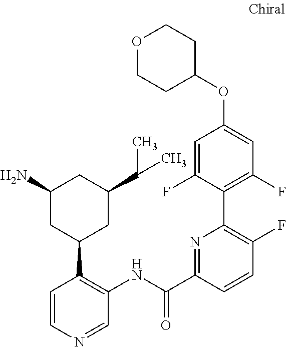 | 569.1 | 0.73 | N-(4-((1R,3S,5S)-3- amino-5- isopropylcyclohexyl) pyridin-3-yl)-6-(2,6- difluoro-4-(tetrahydro- 2H-pyran-4-yloxy) phenyl)-5- fluoropicolinamide |
| 111 | 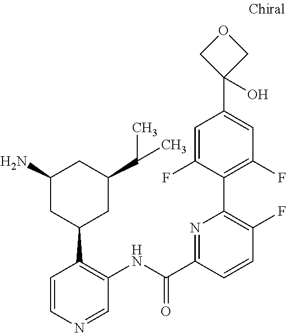 | 541.1 | 0.62 | N-(4-((1R,3S,5S)-3- amino-5- isopropylcyclohexyl) pyridin-3-yl)-6-(2,6- difluoro-4-(3- hydroxyoxetan-3- yl)phenyl)-5- fluoropicolinamide |
| 112 | 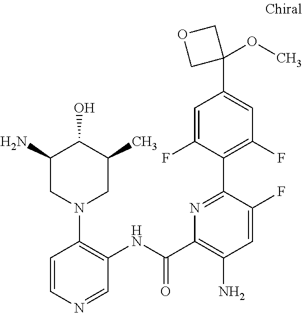 | 559.1 | 0.59 | 3-amino-N-(4- ((3R,4R,5S)-3-amino-4- hydroxy-5- methylpiperidin-1- yl)pyridin-3-yl)-6-(2,6- difluoro-4-(3- methoxyoxetan-3- yl)phenyl)-5- fluoropicolinamide |
| 113 | 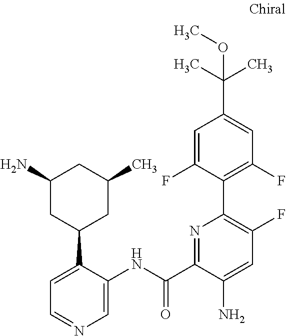 | 528.1 | 0.70 | 3-amino-N-(4- ((1R,3S,5S)-3-amino-5- methylcyclohexyl)pyridin- 3-yl)-6-(2,6-difluoro-4-(2- methoxypropan-2- yl)phenyl)-5- fluoropicolinamide |
| 114 | 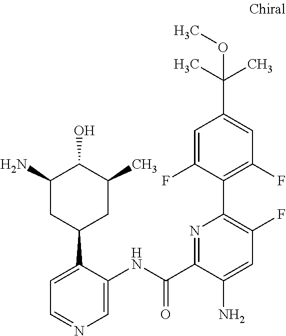 | 544.1 | 0.66 | 3-amino-N-(4- ((1R,3R,4R,5S)-3-amino- 4-hydroxy-5- methylcyclohexyl)pyridin- 3-yl)-6-(2,6-difluoro-4-(2- methoxypropan-2- yl)phenyl)-5- fluoropicolinamide |
| 115 | 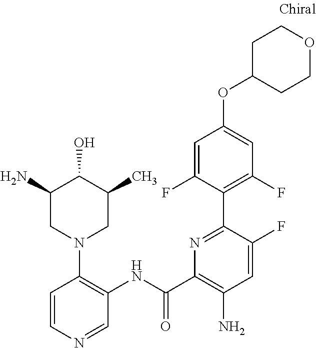 | 573.1 | 0.62 | 3-amino-N-(4- ((3R,4R,5S)-3-amino-4- hydroxy-5- methylpiperidin-1- yl)pyridin-3-yl)-6-(2,6- difluoro-4-(tetrahydro-2H- pyran-4-yloxy)phenyl)-5- fluoropicolinamide |
| 116 | 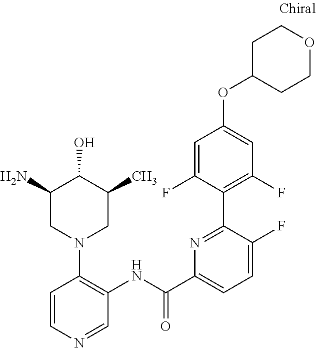 | 558.0 | 0.63 | N-(4-((3R,4R,5S)-3- amino-4-hydroxy-5- methylpiperidin-1- yl)pyridin-3-yl)-6-(2,6- difluoro-4-(tetrahydro-2H- pyran-4-yloxy)phenyl)-5- fluoropicolinamide |
| 117 | 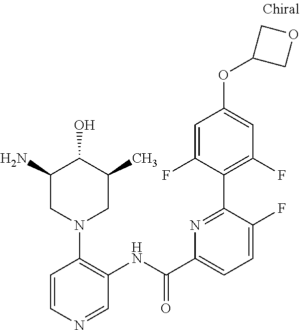 | 530.1 | 0.59 | N-(4-((3R,4R,5S)-3- amino-4-hydroxy-5- methylpiperidin-1- yl)pyridin-3-yl)-6-(2,6- difluoro-4-(oxetan-3- yloxy)phenyl)-5- fluoropicolinamide |
| 118 | 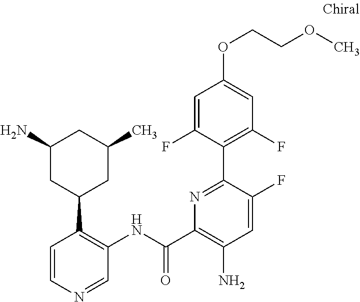 | 547.1 | 0.60 | 3-amino-N-(4- ((1R,3S,5S)-3-amino-5- methylcyclohexyl)pyridin- 3-yl)-6-(2,6-difluoro-4-(2- methoxyethoxy)phenyl)- 5-fluoropicolinamide |
| 119 | 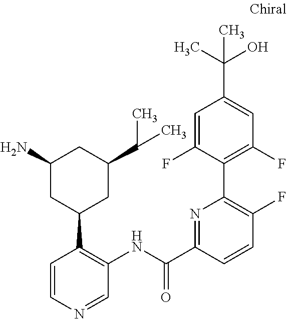 | 527.2 | 0.67 | N-(4-((1R,3S,5S)-3- amino-5- isopropylcyclohexyl) pyridin-3-yl)-6-(2,6- difluoro-4- (2-hydroxypropan-2- yl)phenyl)-5- fluoropicolinamide |
| 120 | 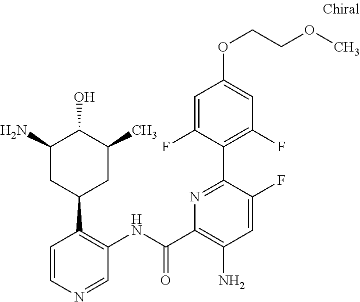 | 546.0 | 0.61 | 3-amino-N-(4- ((1R,3R,4R,5S)-3-amino- 4-hydroxy-5- methylcyclohexyl)pyridin- 3-yl)-6-(2,6-difluoro-4-(2- methoxyethoxy)phenyl)- 5-fluoropicolinamide |
| 121 | 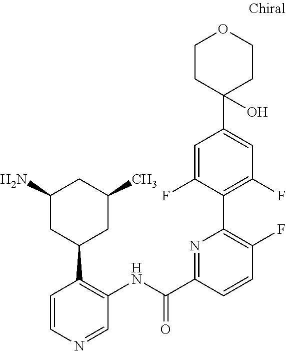 | 541.1 | 0.59 | N-(4-((1R,3S,5S)-3- amino-5- methylcyclohexyl)pyridin- 3-yl)-6-(2,6-difluoro-4-(4- hydroxytetrahydro-2H- pyran-4-yl)phenyl)-5- fluoropicolinamide |
| 122 | 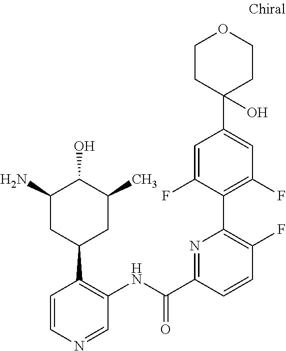 | 557.1 | 0.56 | N-(4-((1R,3R,4R,5S)-3- amino-4-hydroxy-5- methylcyclohexyl)pyridin- 3-yl)-6-(2,6-difluoro-4-(4- hydroxytetrahydro-2H- pyran-4-yl)phenyl)-5- fluoropicolinamide |
| 123 | 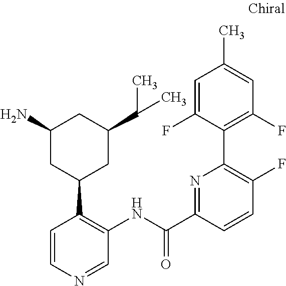 | 483.1 | 0.73 | N-(4-((1R,3S,5S)-3- amino-5- isopropylcyclohexyl) pyridin-3-yl)-6-(2,6- difluoro-4- methylphenyl)-5- fluoropicolinamide |
| 124 | 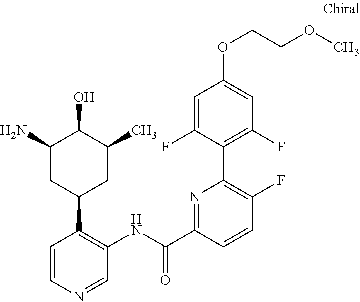 | 531.1 | 0.61 | N-(4-((1R,3R,4S,5S)-3- amino-4-hydroxy-5- methylcyclohexyl)pyridin- 3-yl)-6-(2,6-difluoro-4-(2- methoxyethoxy)phenyl)- 5-fluoropicolinamide |
| 125 | 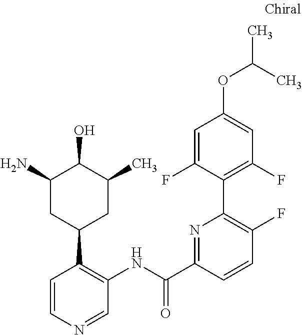 | 515.1 | 0.71 | N-(4-((1R,3R,4S,5S)-3- amino-4-hydroxy-5- methylcyclohexyl)pyridin- 3-yl)-6-(2,6-difluoro-4- isopropoxyphenyl)-5- fluoropicolinamide |
| 126 | 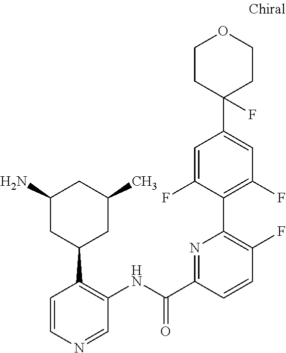 | 543.4 | 0.66 | N-(4-((1R,3S,5S)-3- amino-5- methylcyclohexyl)pyridin- 3-yl)-6-(216-difluoro-4-(4- fluorotetrahydro-2H- pyran-4-yl)phenyl)-5- fluoropicolinamide |
| 127 | 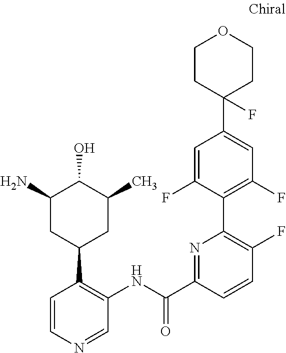 | 559.1 | 0.65 | N-(4-((1R,3R,4R,5S)-3- amino-4-hydroxy-5- methylcyclohexyl)pyridin- 3-yl)-6-(2,6-difluoro-4-(4- fluorotetrahydro-2H- pyran-4-yl)phenyl)-5- fluoropicolinamide |
| 129 | 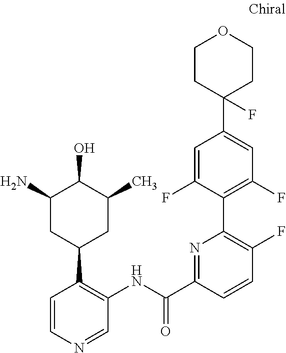 | 559.1 | 0.65 | N-(4-((1R,3R,4S,5S)-3- amino-4-hydroxy-5- methylcyclohexyl)pyridin- 3-yl)-6-(2,6-difluoro-4-(4- fluorotetrahydro-2H- pyran-4-yl)phenyl)-5- fluoropicolinamide |
| 130 | 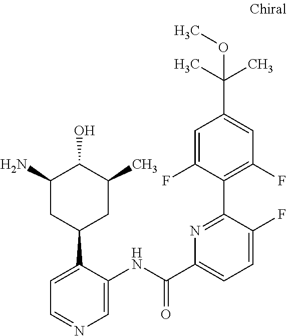 | 530.1 | 0.64 | N-(4-((3R,4R,5S)-3- amino-4-hydroxy-5- methylpiperidin-1- yl)pyridin-3-yl)-6-(2,6- difluoro-4-(2- methoxypropan-2- yl)phenyl)-5- fluoropicolinamide |
| 131 | 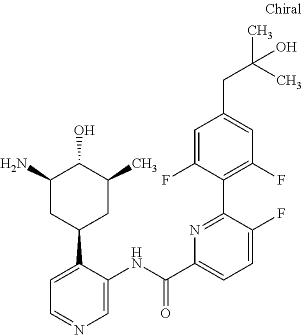 | 530.1 | 0.59 | N-(4-((3R,4R,5S)-3- amino-4-hydroxy-5- methylpiperidin-1- yl)pyridin-3-yl)-6-(2,6- difluoro-4-(2-hydroxy-2- methylpropyl)phenyl)-5- fluoropicolinamide |
| 132 | 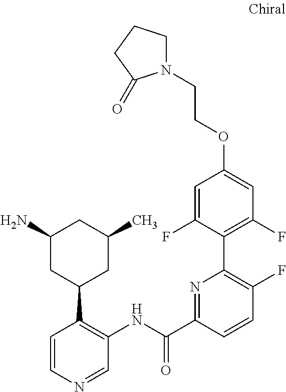 | 568.4 | 0.63 | N-(4-((1R,3S,5S)-3- amino-5- methylcyclohexyl)pyridin- 3-yl)-6-(2,6-difluoro-4-(2- (2-oxopyrrolidin-1- yl)ethoxy)phenyl)-5- fluoropicolinamide |
| 133 | 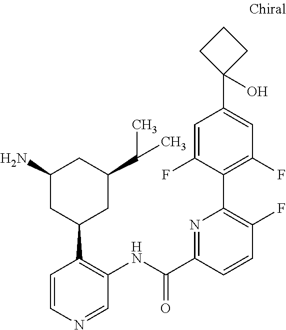 | 539.3 | 0.70 | N-(4-((1R,3S,5S)-3- amino-5- isopropylcyclohexyl) pyridin-3-yl)-6-(2,6- difluoro-4-(1- hydroxycyclobulyl)phenyl)- 5-fluoropicolinamide |
| 134 | 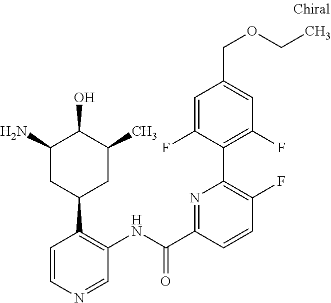 | 515.1 | 0.64 | N-(4-((1R,3R,4S,5S)-3- amino-4-hydroxy-5- methylcyclohexyl)pyridin- 3-yl)-6-(4-(ethoxymethyl)- 2,6-difluorophenyl)-5- fluoropicolinamide |
| 135 | 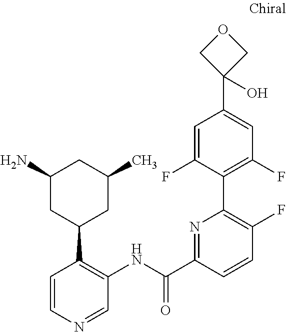 | 515.3 | 0.64 | N-(4-((1R,3S,5S)-3- amino-5- methylcyclohexyl)pyridin- 3-yl)-6-(2,6-difluoro-4-(3- fluorooxetan-3- yl)phenyl)-5- fluoropicolinamide |
| 136 | 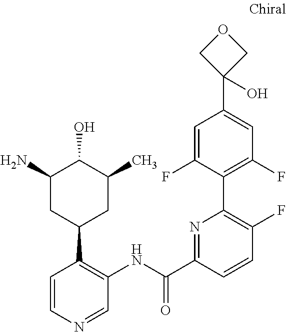 | 531.3 | 0.61 | N-(4-((1R,3R,4R,5S)-3- amino-4-hydroxy-5- methylcyclohexyl)pyridin- 3-yl)-6-(2,6-difluoro-4-(3- fluorooxetan-3- yl)phenyl)-5- fluoropicolinamide |
| 137 | 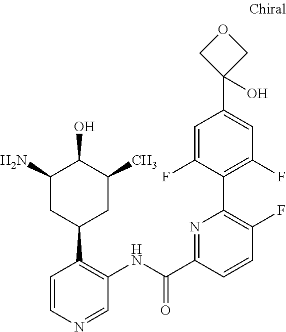 | 531.3 | 0.61 | N-(4-((1R,3R,4S,5S)-3- amino-4-hydroxy-5- methylcyclohexyl)pyridin- 3-yl)-6-(2,6-difluoro-4-(3- fluorooxetan-3- yl)phenyl)-5- fluoropicolinamide |
| 138 | 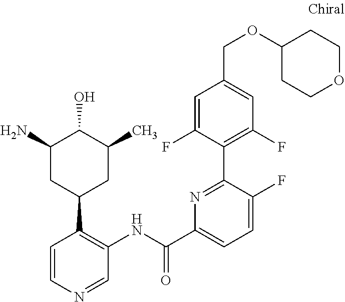 | 571.3 | 0.66 | N-(4-((1R,3R,4R,5S)-3- amino-4-hydroxy-5- methylcyclohexyl)pyridin- 3-yl)-6-(2,6-difluoro-4- ((tetrahydro-2H-pyran-4- yloxy)methyl)phenyl)-5- fluoropicolinamide |
| 139 | 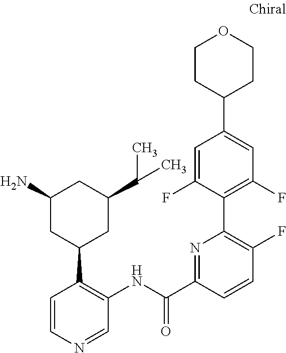 | 553.3 | 0.74 | N-(4-((1R,3S,5S)-3- amino-5- isopropylcyclohexyl) pyridin-3-yl)-6-(2,6- difluoro-4- (tetrahydro-2H-pyran-4- yl)phenyl)-5- fluoropicolinamide |
| 140 | 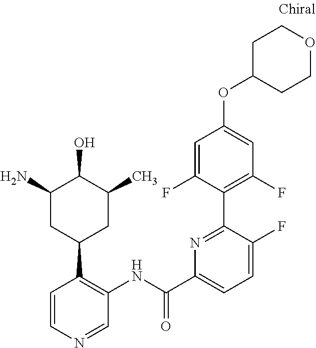 | 557.3 | 0.68 | N-(4-((1R,3R,4S,5S)-3- amino-4-hydroxy-5- methylcyclohexyl)pyridin- 3-yl)-6-(2,6-difluoro-4- (tetrahydro-2H-pyran-4- yloxy)phenyl)-5- fluoropicolinamide |
| 141 | 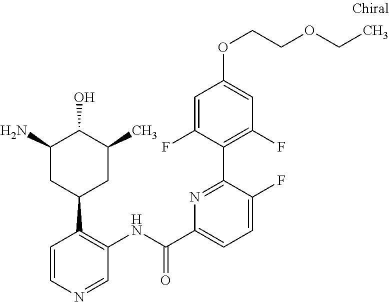 | 545.3 | 0.68 | N-(4-((1R,3R,4R,5S)-3- amino-4-hydroxy-5- methylcyclohexyl)pyridin- 3-yl)-6-(4-(2- ethoxyethoxy)-2,6- difluorophenyl)-5- fluoropicolinamide |
| 142 | 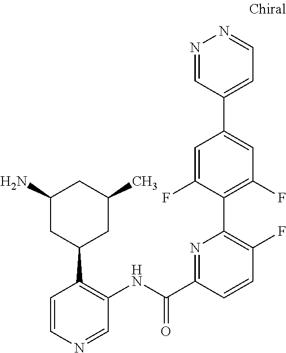 | 519.20 | 0.60 | N-(4-((1R,3S,5S)-3- amino-5- methylcyclohexyl)pyridin- 3-yl)-6-(2,6-difluoro-4- (pyridazin-4-yl)phenyl)-5- fluoropicolinamide |
| 143 | 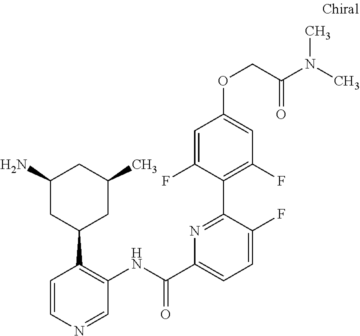 | 542.4 | 0.63 | N-(4-((1R,3S,5S)-3- amino-5- methylcyclohexyl)pyridin- 3-yl)-6-(4-(2- (dimethylamino)-2- oxoethoxy)-2,6- difluorophenyl)-5- fluoropicolinamide |
| 144 | 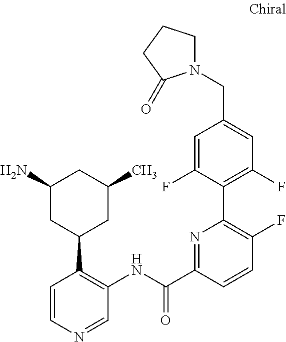 | 538.2 | 0.62 | N-(4-((1R,3S,5S)-3- amino-5- methylcyclohexyl)pyridin- 3-yl)-6-(2,6-difluoro-4-((2- oxopyrrolidin-1- yl)methyl)phenyl)-5- fluoropicolinamide |
| 145 | 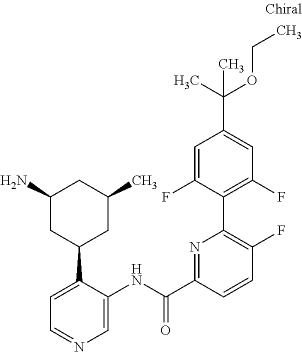 | 527.3 | 0.75 | N-(4-((1R,3S,5S)-3- amino-5- methylcyclohexyl)pyridin- 3-yl)-6-(4-(2- ethoxypropan-2-yl)-2,6- difluorophenyl)-5- fluoropicolinamide |
| 146 | 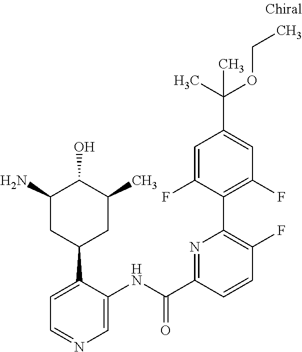 | 543.2 | 0.72 | N-(4-((1R,3R,4R,5S)-3- amino-4-hydroxy-5- methylcyclohexyl)pyridin- 3-yl)-6-(4-(2- ethoxypropan-2-yl)-2,6- difluorophenyl)-5- fluoropicolinamide |
| 147 | 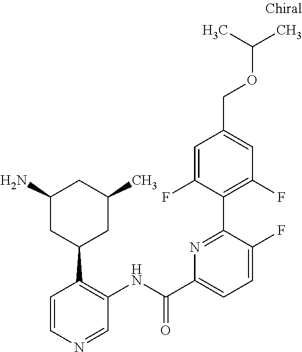 | 513.4 | 0.73 | N-(4-((1R,3S,5S)-3- amino-5- methylcyclohexyl)pyridin- 3-yl)-6-(2,6-difluoro-4- (isopropoxymethyl)phenyl)- 5-fluoropicolinamide |
| 148 | 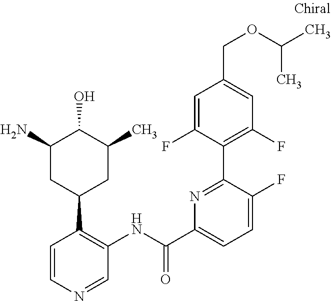 | 529.4 | 0.69 | N-(4-((1R,3R,4R,5S)-3- amino-4-hydroxy-5- methylcyclohexyl)pyridin- 3-yl)-6-(2,6-difluoro-4- (isopropoxymethyl)phenyl)- 5-fluoropicolinamide |
| 149 | 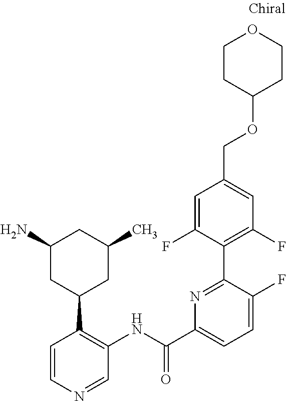 | 555.2 | 0.69 | N-(4-((1R,3S,5S)-3- amino-5- methylcyclohexyl)pyridin- 3-yl)-6-(2,6-difluoro-4- ((tetrahydro-2H-pyran-4- yloxy)methyl)phenyl)-5- fluoropicolinamide |
| 150 | 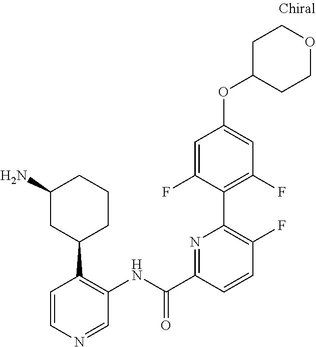 | 527.2 | 0.69 | N-(4-((1R,3S)-3- aminocyclohexyl)pyridin- 3-yl)-6-(2,6-difluoro-4- (tetrahydro-2H-pyran-4- yloxy)phenyl)-5- fluoropicolinamide |
| 151 | 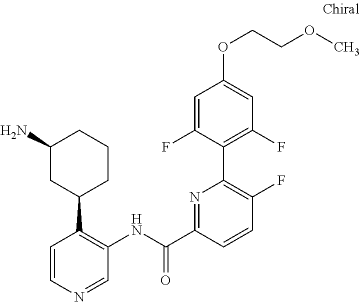 | 501.2 | 0.65 | N-(4-((1R,3S)-3- aminocyclohexyl)pyridin- 3-yl)-6-(2,6-difluoro-4-(2- methoxyethoxy)phenyl)- 5-fluoropicolinamide |
| 152 | 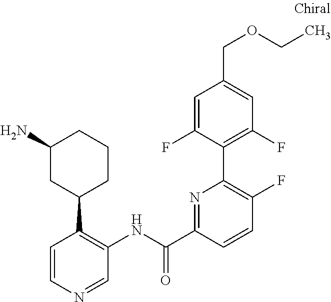 | 485.2 | 0.65 | N-(4-((1R,3S)-3- aminocyclohexyl)pyridin- 3-yl)-6-(4-(ethoxymethyl)- 2,6-difluorophenyl)-5- fluoropicolinamide |
| 153 | 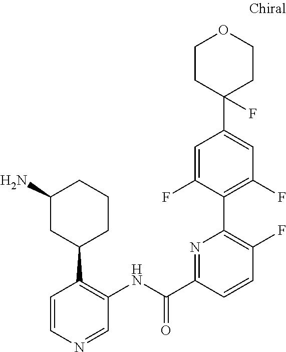 | 529.2 | 0.68 | N-(4-((1R,3S)-3- aminocyclohexyl)pyridin- 3-yl)-6-(2,6-difluoro-4-(4- fluorotetrahydro-2H- pyran-4-yl)phenyl)-5- fluoropicolinamide |
| 154 | 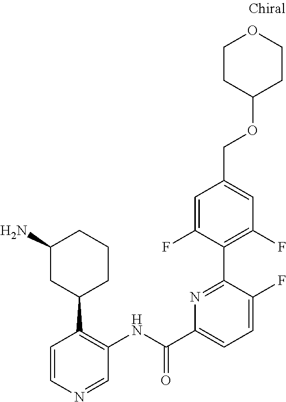 | 541.3 | 0.68 | N-(4-((1R,3S)-3- aminocyclohexyl)pyridin- 3-yl)-6-(2,6-difluoro-4- ((tetrahydro-2H-pyran-4- yloxy)methyl)phenyl)-5- fluoropicolinamide |
| 155 | 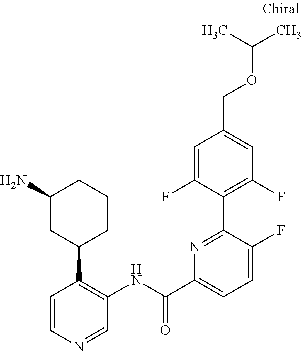 | 499.2 | 0.75 | N-(4-((1R,3S)-3- aminocyclohexyl)pyridin- 3-yl)-6-(2,6-difluoro-4- (isopropoxymethyl)phenyl)- 5-fluoropicolinamide |
| 156 | 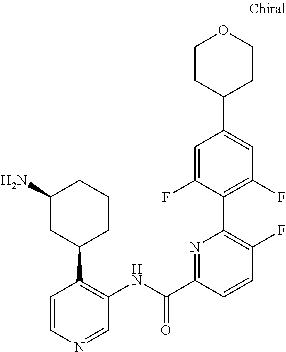 | 511.2 | 0.70 | N-(4-((1R,3S)-3- aminocyclohexyl)pyridin- 3-yl)-6-(2,6-difluoro-4- (tetrahydro-2H-pyran-4- yl)phenyl)-5- fluoropicolinamide |
| 157 | 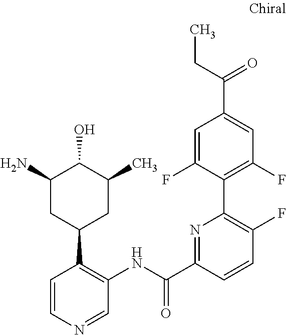 | 513.2 | 0.62 | N-(4-((1R,3R,4R,5S)-3- amino-4-hydroxy-5- methylcyclohexyl)pyridin- 3-yl)-6-(2,6-difluoro-4- propionylphenyl)-5- fluoropicolinamide |
| 158 | 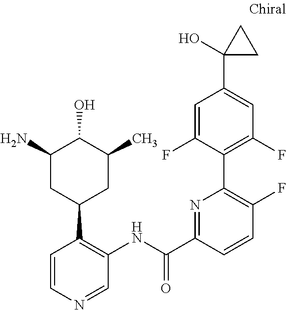 | 513.2 | 0.56 | N-(4-((1R,3R,4R,5S)-3- amino-4-hydroxy-5- methylcyclohexyl)pyridin- 3-yl)-6-(2,6-difluoro-4-(1- hydroxycyclopropyl)phenyl)- 5-fluoropicolinamide |
| 159 | 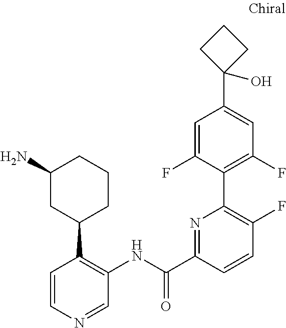 | 497.2 | 0.73 | N-(4-((1R,3S)-3- aminocyclohexyl)pyridin- 3-yl)-6-(2,6-difluoro-4-(1- hydroxycyclobutyl)phenyl)- 5-fluoropicolinamide |
| 160 | 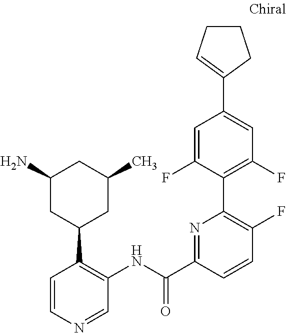 | 507.3 | 0.77 | N-(4-((1R,3R,4R,5S)-3- amino-4-hydroxy-5- methylcyclohexyl)pyridin- 3-yl)-6-(2,6-difluoro-4-(1- hydroxycyclopentyl) phenyl)-5- fluoropicolinamide |
| 161 | 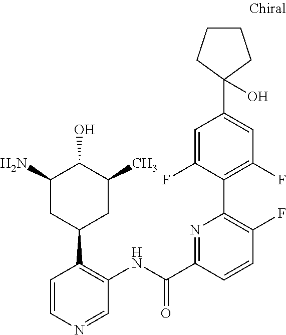 | 541.3 | 0.63 | N-(4-((1R,3R,4R,5S)-3- amino-4-hydroxy-5- methylcyclohexyl)pyridin- 3-yl)-6-(2,6-difluoro-4-(1- hydroxycyclopentyl) phenyl)-5- fluoropicolinamide |
| 162 | 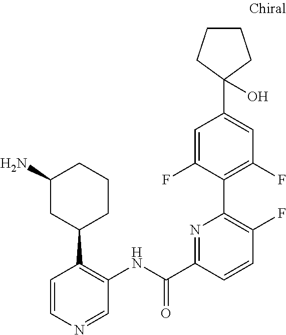 | 511.1 | 0.68 | N-(4-((1R,3S)-3- aminocyclohexyl)pyridin- 3-yl)-6-(2,6-difluoro-4-(1- hydroxycyclopentyl)phenyl)- 5-fluoropicolinamide |
| 163 | 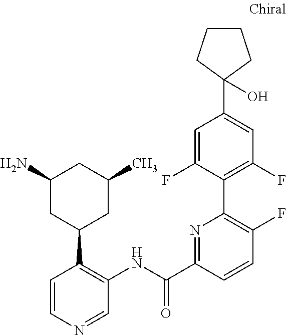 | 525.4 | 0.69 | N-(4-((1R,3S,5S)-3- amino-5- methylcyclohexyl)pyridin- 3-yl)-6-(2,6-difluoro-4-(1- hydroxycyclopentyl) phenyl)-5- fluoropicolinamide |
| 164 | 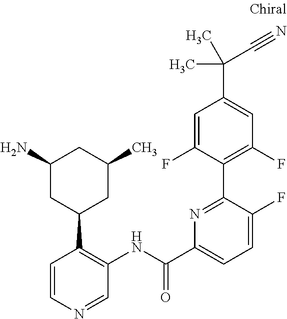 | 508.2 | 0.70 | N-(4-((1R,3S,5S)-3- amino-5- methylcyclohexyl)pyridin- 3-yl)-6-(4-(2- cyanopropan-2-yl)-2,6- difluorophenyl)-5- fluoropicolinamide |
| 165 | 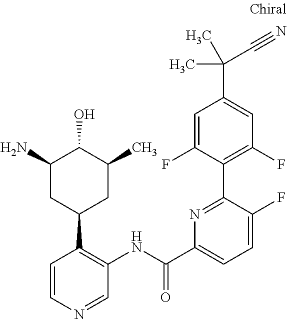 | 524.2 | 0.68 | N-(4-((1R,3R,4R,5S)-3- amino-4-hydroxy-5- methylcyclohexyl)pyridin- 3-yl)-6-(4-(2- cyanopropan-2-yl)-2,6- difluorophenyl)-5- fluoropicolinamide |
| 166 | 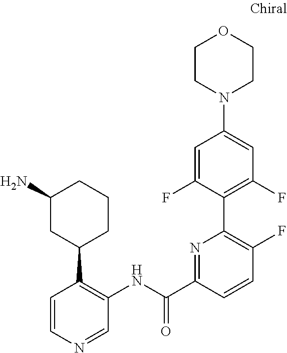 | 512.3 | 0.66 | N-(4-((1R,3S)-3- aminocyclohexyl)pyridin- 3-yl)-6-(2,6-difluoro-4- morpholinophenyl)-5- fluoropicolinamide |
| 167 | 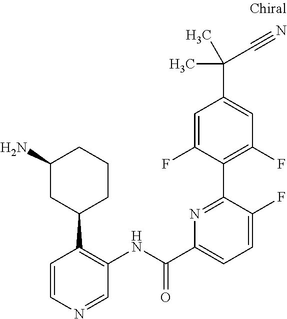 | 494.2 | 0.69 | N-(4-((1R,3S)-3- aminocyclohexyl)pyridin- 3-yl)-6-(4-(2- cyanopropan-2-yl)-2,6- difluorophenyl)-5- fluoropicolinamide |
| 168 | 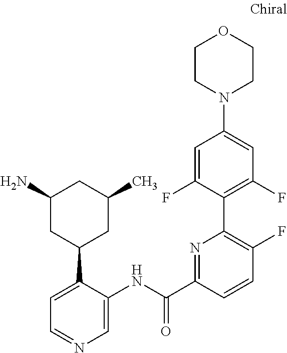 | 526.4 | 0.70 | N-(4-((1R,3S,5S)-3- amino-5- methylcyclohexyl)pyridin- 3-yl)-6-(2,6-difluoro-4- morpholinophenyl)-5- fluoropicolinamide |
| 169 | 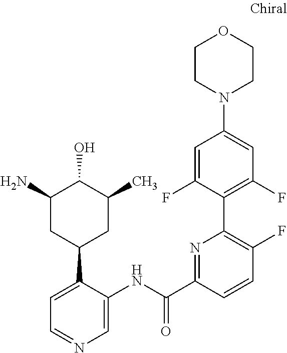 | 542.3 | 0.66 | N-(4-((1R,3R,4R,5S)-3- amino-4-hydroxy-5- methylcyclohexyl)pyridin- 3-yl)-6-(2,6-difluoro-4- morpholinophenyl)-5- fluoropicolinamide |
| 170 | 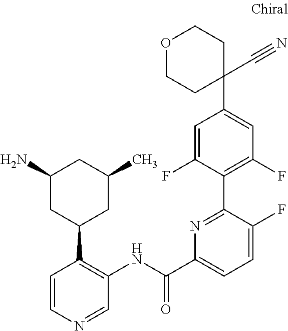 | 550.3 | 0.67 | N-(4-((1R,3S,5S)-3- amino-5- methylcyclohexyl)pyridin- 3-yl)-6-(4-(4- cyanotetrahydro-2H- pyran-4-yl)-2,6- difluorophenyl)-5- fluoropicolinamide |
| 171 | 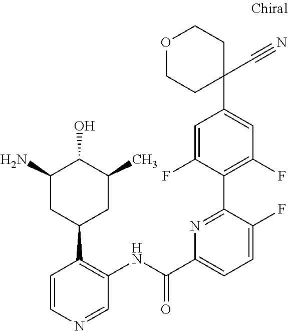 | 566.3 | 0.64 | N-(4-((1R,3R,4R,5S)-3- amino-4-hydroxy-5- methylcyclohexyl)pyridin- 3-yl)-6-(4-(4- cyanotetrahydro-2H- pyran-4-yl)-2,6- difluorophenyl)-5- fluoropicolinamide |
| 172 | 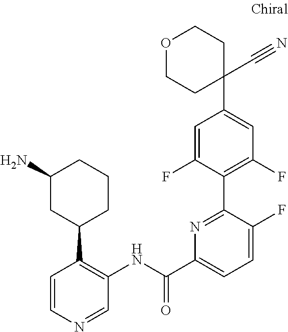 | 536.2 | 0.64 | N-(4-((1R,3S)-3- aminocyclohexyl)pyridin- 3-yl)-6-(4-(4- cyanotetrahydro-2H- pyran-4-yl)-2,6- difluorophenyl)-5- fluoropicolinamide |
| 173 | 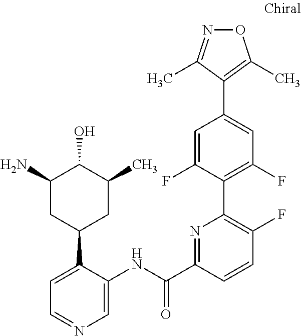 | 552.3 | 0.68 | N-(4-((1R,3R,4R,5S)-3- amino-4-hydroxy-5- methylcyclohexyl)pyridin- 3-yl)-6-(4-(3,5- dimethylisoxazol-4-yl)- 2,6-difluorophenyl)-5- fluoropicolinamide |
| 174 | 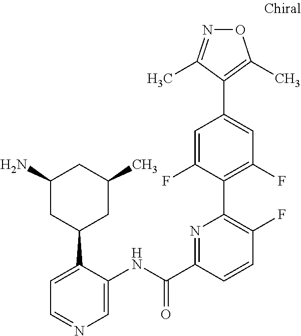 | 536.2 | 0.72 | N-(4-((1R,3S,5S)-3- amino-5- methylcyclohexyl)pyridin- 3-yl)-6-(4-(3,5- dimethylisoxazol-4-yl)- 2,6-difluorophenyl)-5- fluoropicolinamide |
| 175 | 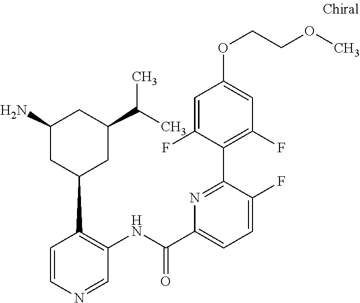 | 543.2 | 0.69 | N-(4-((1R,3S,5S)-3- amino-5- isopropylcyclohexyl)pyridin- 3-yl)-6-(2,6-difluoro-4- (2-methoxyethoxy)phenyl)- 5-fluoropicolinamide |
| 176 | 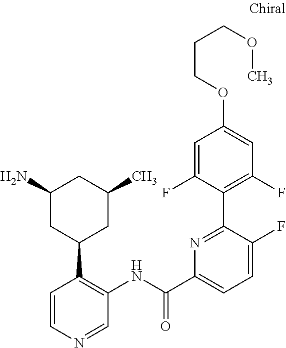 | 529.3 | 0.70 | N-(4-((1R,3S,5S)-3- amino-5- methylcyclohexyl)pyridin- 3-yl)-6-(2,6-difluoro-4-(3- methoxypropoxy)phenyl)- 5-fluoropicolinamide |
| 177 | 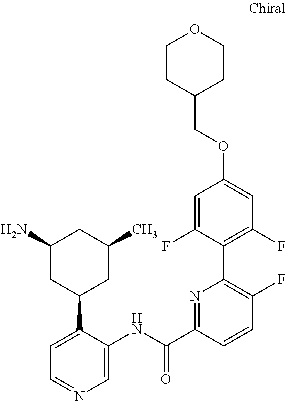 | 555.2 | 0.72 | N-(4-((1R,3S,5S)-3- amino-5- methylcyclohexyl)pyridin- 3-yl)-6-(2,6-difluoro-4- ((tetrahydro-2H-pyran-4- yl)methoxy)phenyl)-5- fluoropicolinamide |
| 178 | 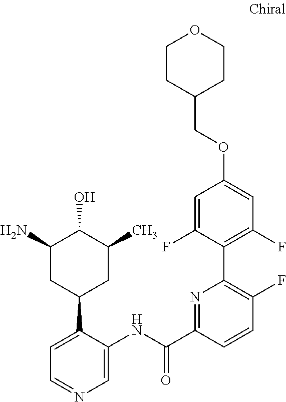 | 571.3 | 0.71 | N-(4-((1R,3R,4R,5S)-3- amino-4-hydroxy-5- methylcyclohexyl)pyridin- 3-yl)-6-(2,6-difluoro-4- ((tetrahydro-2H-pyran-4- yl)methoxy)phenyl)-5- fluoropicolinamide |
| 179 | 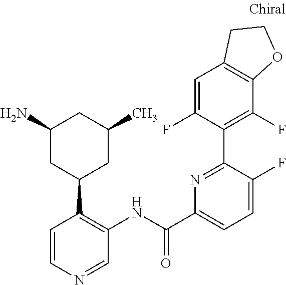 | 483.2 | 0.65 | N-(4-((1R,3S,5S)-3- amino-5- methylcyclohexyl)pyridin- 3-yl)-6-(5,7-difluoro-2,3- dihydrobenzofuran-6-yl)- 5-fluoropicolinamide |
| 180 | 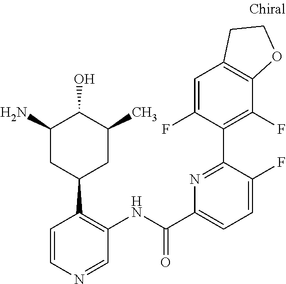 | 499.2 | 0.61 | N-(4-((1R,3R,4R,5S)-3- amino-4-hydroxy-5- methylcyclohexyl)pyridin- 3-yl)-6-(5,7-difluoro-2,3- dihydrobenzofuran-6-yl)- 5-fluoropicolinamide |
| 181 | 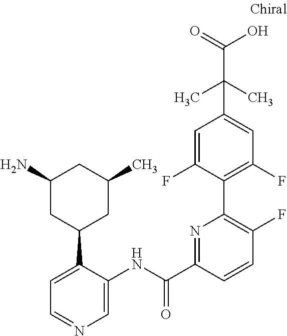 | 527.3 | 0.66 | 2-(4-(6-(4-((1R,3S,5S)-3- amino-5- methylcyclohexyl)pyridin- 3-ylcarbamoyl)-3- fluoropyridin-2-yl)-3,5- difluorophenyl)-2- methylpropanoic acid |
| 182 | 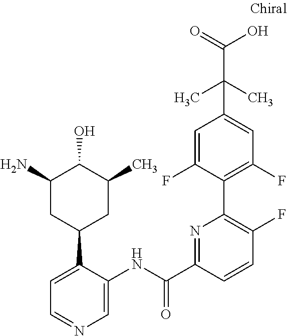 | 543.4 | 0.62 | 2-(4-(6-(4- ((1R,3R,4R,5S)-3-amino- 4-hydroxy-5- methylcyclohexyl)pyridin- 3-ylcarbamoyl)-3- fluoropyridin-2-yl)-3,5- difluorophenyl)-2- methylpropanoic acid |
| 183 | 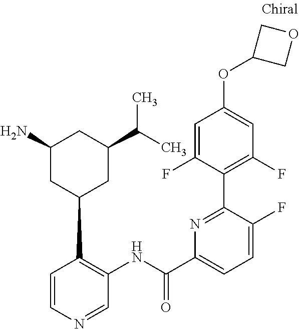 | 541.2 | 0.67 | N-(4-((1R,3S,5S)-3- amino-5- isopropylcyclohexyl) pyridin-3-yl)-6-(2,6- difluoro-4- (oxetan-3-yloxy)phenyl)- 5-fluoropicolinamide |
| 184 | 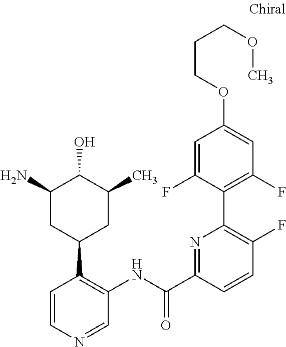 | 545.2 | 0.70 | N-(4-((1R,3R,4R,5S)-3- amino-4-hydroxy-5- methylcyclohexyl)pyridin- 3-yl)-6-(2,6-difluoro-4-(3- methoxypropoxy)phenyl)- 5-fluoropicolinamide |
| 185 | 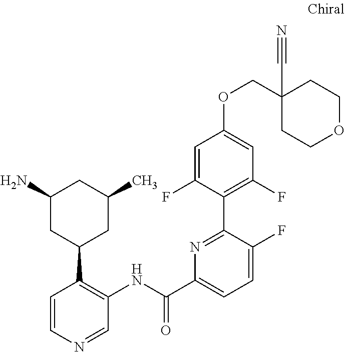 | 580.3 | 0.71 | N-(4-((1R,3S,5S)-3- amino-5- methylcyclohexyl)pyridin- 3-yl)-6-(4-((4- cyanotetrahydro-2H- pyran-4-yl)methoxy)-2,6- difluorophenyl)-5- fluoropicolinamide |
| 186 | 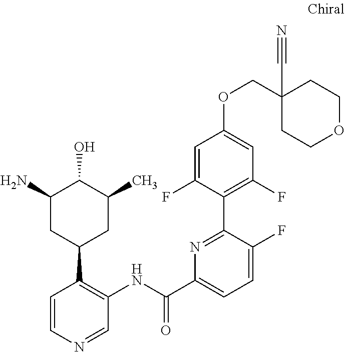 | 596.2 | 0.68 | N-(4-((1R,3R,4R,5S)-3- amino-4-hydroxy-5- methylcyclohexyl)pyridin- 3-yl)-6-(4-((4- cyanotetrahydro-2H- pyran-4-yl)methoxy)-2,6- difluorophenyl)-5- fluoropicolinamide |
| 187 | 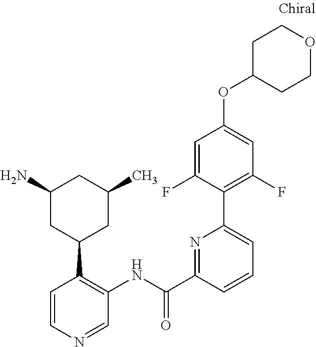 | 523.4 | 0.66 | N-(4-((1R,3S,5S)-3- amino-5- methylcyclohexyl)pyridin- 3-yl)-6-(2,6-difluoro-4- (tetrahydro-2H-pyran-4- yloxy)phenyl)picolinamide |
| 188 | 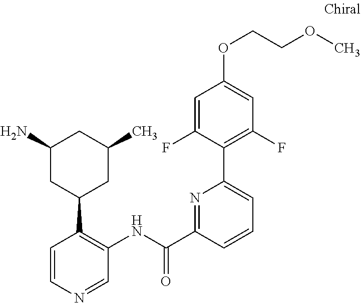 | 497.4 | 0.63 | N-(4-((1R,3S,5S)-3- amino-5- methylcyclohexyl)pyridin- 3-yl)-6-(2,6-difluoro-4-(2- methoxyethoxy)phenyl) picolinamide |
| 189 | 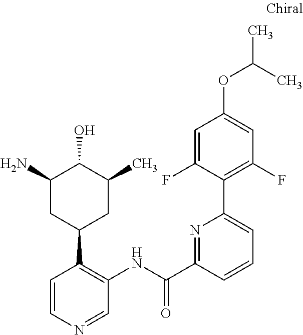 | 497.4 | 0.65 | N-(4-((1R,3R,4R,5S)-3- amino-4-hydroxy-5- methylcyclohexyl)pyridin- 3-yl)-6-(2,6-difluoro-4- isopropoxyphenyl) picolinamide |
| 190 | 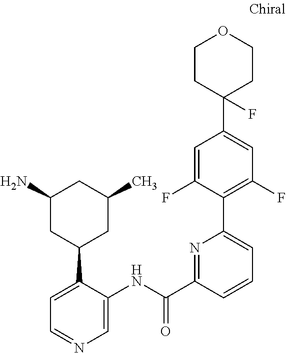 | 525.3 | 0.70 | N-(4-((1R,3S,5S)-3- amino-5- methylcyclohexyl)pyridin- 3-yl)-6-(2,6-difluoro-4-(4- fluorotetrahydro-2H- pyran-4- yl)phenyl)picolinamide |
| 191 | 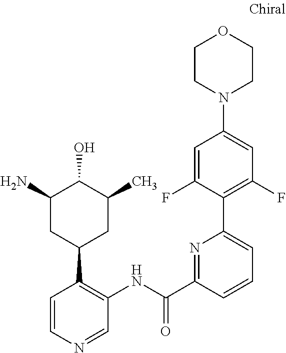 | 524.4 | 0.61 | N-(4-((1R,3R,4R,5S)-3- amino-4-hydroxy-5- methylcyclohexyl)pyridin- 3-yl)-6-(2,6-difluoro-4- morpholinophenyl) picolinamide |
| 192 | 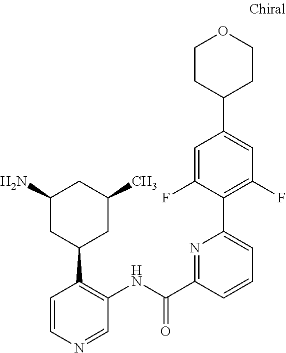 | 507.2 | 0.77 | N-(4-((1R,3S,5S)-3- amino-5- methylcyclohexyl)pyridin- 3-yl)-6-(2,6-difluoro-4- (tetrahydro-2H-pyran-4- yl)phenyl)picolinamide |
| 193 | 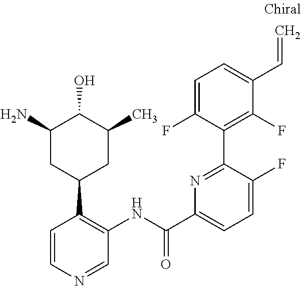 | 483.2 | 0.70 | N-(4-((1R,3R,4R,5S)-3- amino-4-hydroxy-5- methylcyclohexyl)pyridin- 3-yl)-6-(2,6-difluoro-3- vinylphenyl)-5- fluoropicolinamide |
| 194 | 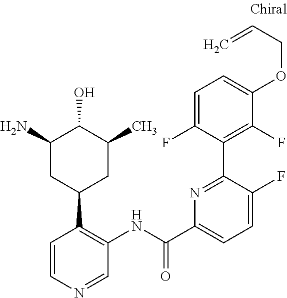 | 513.2 | 0.71 | 6-(3-(allyloxy)-2.6- difluorophenyl)-N-(4- ((1R,3R,4R,5S)-3-amino- 4-hydroxy-5- methylcyclohexyl)pyridin- 3-yl)-5-fluoropicolinamide |
| 195 | 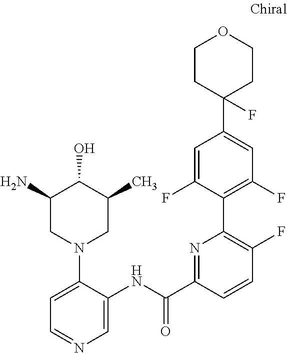 | 560.4 | 0.62 | N-(4-((3R,4R,5S)-3- amino-4-hydroxy-5- methylpiperidin-1- yl)pyridin-3-yl)-6-(2,6- difluoro-4-(4- fluorotetrahydro-2H- pyran-4-yl)phenyl)-5- fluoropicolinamide |
| 196 | 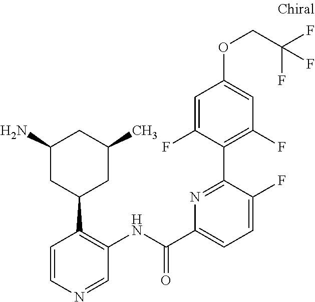 | 539.2 | 0.77 | N-(4-((1R,3S,5S)-3- amino-5- methylcyclohexyl)pyridin- 3-yl)-6-(2,6-difluoro-4- (2,2,2- trifluoroethoxy)phenyl)-5- fluoropicolinamide |
| 197 | 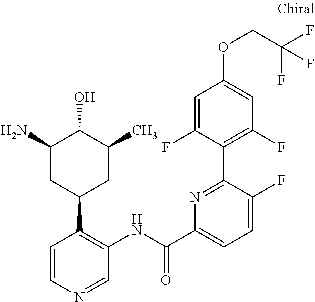 | 555.2 | 0.73 | N-(4-((1R,3R,4R,5S)-3- amino-4-hydroxy-5- methylcyclohexyl)pyridin- 3-yl)-6-(2,6-difluoro-4- (2,2,2- trifluoroethoxy)phenyl)-5- fluoropicolinamide |
| 198 | 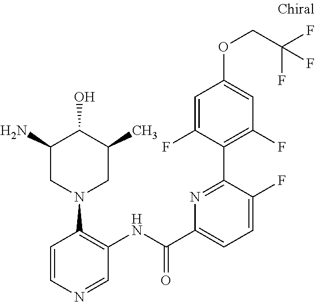 | 556.2 | 0.72 | N-(4-((3R,4R,5S)-3- amino-4-hydroxy-5- methylpiperidin-1- yl)pyridin-3-yl)-6-(2,6- difluoro-4-(2,2,2- trifluoroethoxy)phenyl)-5- fluoropicolinamide |
| 199 | 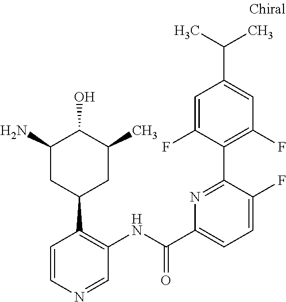 | 499.4 | 0.70 | N-(4-((1R,3R,4R,5S)-3- amino-4-hydroxy-5- methylcyclohexyl)pyridin- 3-yl)-6-(2,6-difluoro-4- isopropylphenyl)-5- fluoropicolinamide |
| 200 | 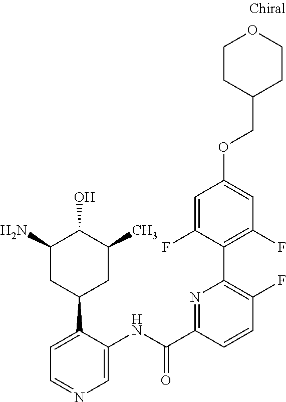 | 572.2 | 0.67 | N-(4-((3R,4R,5S)-3- amino-4-hydroxy-5- methylpiperidin-1- yl)pyridin-3-yl)-6-(2,6- difluoro-4-((tetrahydro- 2H-pyran-4- yl)methoxy)phenyl)-5- fluoropicolinamide |
| 201 | 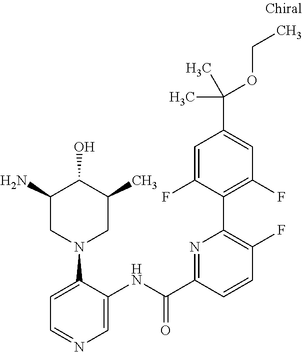 | 544.3 | 0.69 | N-(4-((3R,4R,5S)-3- amino-4-hydroxy-5- methylpiperidin-1- yl)pyridin-3-yl)-6-(4-(2- ethoxypropan-2-yl)-2,6- difluorophenyl)-5- fluoropicolinamide |
| 202 | 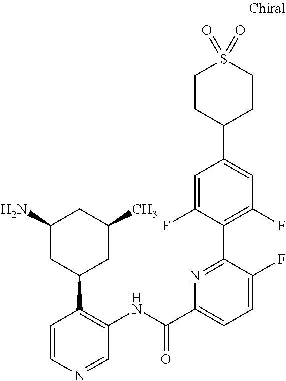 | 573.2 | 0.63 | N-(4-((1R,3S,5S)-3- amino-5- methylcyclohexyl)pyridin- 3-yl)-6-(4-(1,1- dioxidotetrahydro-2H- thiopyran-4-yl)-2,6- difluorophenyl)-5- fluoropicolinamide |
| 203 | 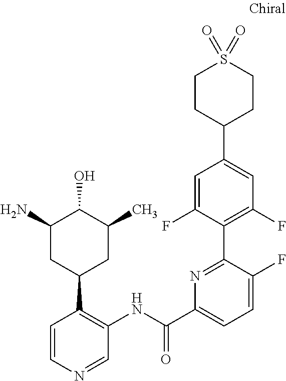 | 589.2 | 0.59 | N-(4-((1R,3R,4R,5S)-3- amino-4-hydroxy-5- methylcyclohexyl)pyridin- 3-yl)-6-(4-(1,1- dioxidotetrahydro-2H- thiopyran-4-yl)-2,6- difluorophenyl)-5- fluoropicolinamide |
| 204 | 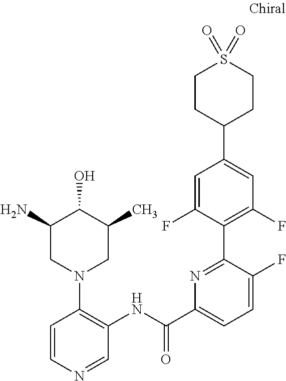 | 590.2 | 0.59 | N-(4-((3R,4R,5S)-3- amino-4-hydroxy-5- methylpiperidin-1- yl)pyridin-3-yl)-6-(4-(1,1- dioxidotetrahydro-2H- thiopyran-4-yl)-2,6- difluorophenyl)-5- fluoropicolinamide |
| 205 | 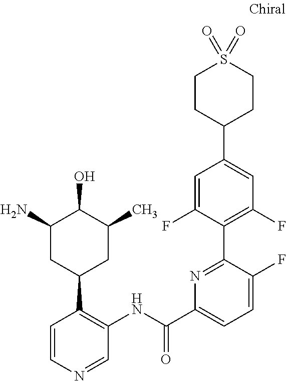 | 589.2 | 0.60 | N-(4-((1R,3R,4S,5S)-3- amino-4-hydroxy-5- methylcyclohexyl)pyridin- 3-yl)-6-(4-(1,1- dioxidotetrahydro-2H- thiopyran-4-yl)-2,6- difluorophenyl)-5- fluoropicolinamide |
| 206 | 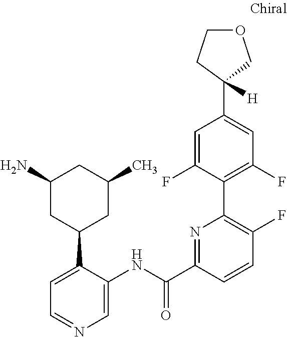 | 511.1 | 0.69 | N-(4-((1R,3S,5S)-3- amino-5- methylcyclohexyl)pyridin- 3-yl)-6-(2,6-difluoro-4- ((S)-tetrahydrofuran-3- yl)phenyl)-5- fluoropicolinamide |
| 207 | 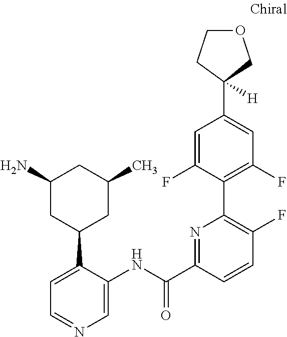 | 511.1 | 0.71 | N-(4-((1R,3S,5S)-3- amino-5- methylcyclohexyl)pyridin- 3-yl)-6-(2,6-difluoro-4- ((R)-tetrahydrofuran-3- yl)phenyl)-5- fluoropicolinamide |

### Synthesis of tert-butyl ((1S,3R,5S)-3-(3-(6-(2,6-difluoro-4-(tetrahydrofuran-3-yl)phenyl)-5-fluoropicolinamido)pyridin-4-yl)-5-methylcyclohexyl)carbamate

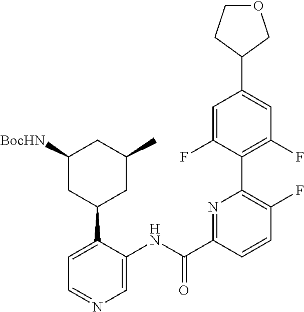

Method 5 was followed using tert-butyl ((1S,3R,5S)-3-(3-aminopyridin-4-yl)-5-methylcyclohexyl)carbamate (1.0 equiv.) and 6-(2,6-difluoro-4-(tetrahydrofuran-3-yl)phenyl)-5-fluoropicolinic acid to give tert-butyl ((1S,3R,5S)-3-(3-(6-(2,6-difluoro-4-(tetrahydrofuran-3-yl)phenyl)-5-fluoropicolinamido)pyridin-4-yl)-5-methylcyclohexyl)carbamate in 100% yield. LC/MS=611.2 (M+H), Rt=0.94 min.

### Synthesis of N-(4-((1R,3S,5S)-3-amino-5-methylcyclohexyl)pyridin-3-yl)-6-(2,6-difluoro-4-((S)-tetrahydrofuran-3-yl)phenyl)-5-fluoropicolinamide and N-(4-((1R,3S,5S)-3-amino-5-methylcyclohexyl)pyridin-3-yl)-6-(2,6-difluoro-4-((R)-tetrahydrofuran-3-yl)phenyl)-5-fluoropicolinamide

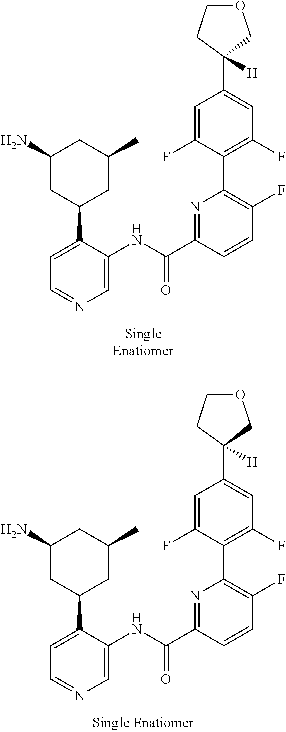

To a solution of tert-butyl ((1S,3R,5S)-3-(3-(6-(2,6-difluoro-4-(tetrahydrofuran-3-yl)phenyl)-5-fluoropicolinamido)pyridin-4-yl)-5-methylcyclohexyl)carbamate (1.0 equiv.) in DCM (0.05 M) at rt was added TFA (30 equiv.). After 1 hr, the reaction was concentrated and partitioned between EtOAc and NaHCO3. The organic layer was washed with brine, dried over Na2SO4 and concentrated. Purification was completed via chiral HPLC (EtOH/heptane)=20/80, mL/min, AD column) to yield N-(4-((1R,3S,5S)-3-amino-5-methylcyclohexyl)pyridin-3-yl)-6-(2,6-difluoro-4-((S)-tetrahydrofuran-3-yl)phenyl)-5-fluoropicolinamide (17% yield, 99% ee) and N-(4-((1R,3S,5S)-3-amino-5-methylcyclohexyl)pyridin-3-yl)-6-(2,6-difluoro-4-((R)-tetrahydrofuran-3-yl)phenyl)-5-fluoropicolinamide (17% yield, 99% ee). LC/MS=511.1 (MH+), Rt=0.70 min.

### Synthesis of (1R,2R,4R,6S)-4-(3-(6-bromo-5-fluoropicolinamido)pyridin-4-yl)-2-(tert-butoxycarbonylamino)-6-methylcyclohexyl acetate

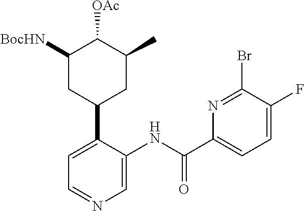

Following Method 5, (1R,2R,4R,6S)-4-(3-aminopyridin-4-yl)-2-(tert-butoxycarbonylamino)-6-methylcyclohexyl acetate and 6-bromo-5-fluoropicolinic acid were coupled and following addition of EtOAc and washing with H₂O, NaCl_{(sat.)} and drying over MgSO₄, (1R,2R,4R,6S)-4-(3-(6-bromo-5-fluoropicolinamido)pyridin-4-yl)-2-(tert-butoxycarbonylamino)-6-methylcyclohexyl acetate was obtained. LCMS (m/z): 567.2 (MH⁺), Rₜ=0.82 min.

### Synthesis of (+/−)-tert-butyl 5-(3-(6-bromo-5-fluoropicolinamido)pyridin-4-yl)-7-methyl-2-oxohexahydrobenzo[d]oxazole-3(2H)-carboxylate

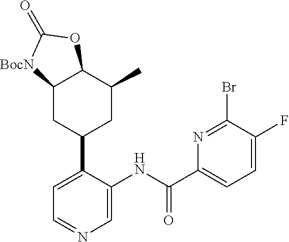

Following Method 5, (+/−)-tert-butyl 5-(3-aminopyridin-4-yl)-7-methyl-2-oxohexahydrobenzo[d]oxazole-3(2H)-carboxylate and 6-bromo-5-fluoropicolinic acid were coupled and following addition of EtOAc and washing with H₂O, NaCl_{(sat.)} and drying over MgSO₄, (+/−)-tert-butyl 5-(3-(6-bromo-5-fluoropicolinamido)pyridin-4-yl)-7-methyl-2-oxohexahydrobenzo[d]oxazole-3(2H)-carboxylate was obtained. LCMS (m/z): 549.2/551.2 (MH⁺), Rₜ=0.78 min.

### Synthesis of 6-bromo-N-(4-((1R,3S)-3-(1,3-dioxoisoindolin-2-yl)-cyclohexyl)pyridin-3-yl)-5-fluoropicolinamide

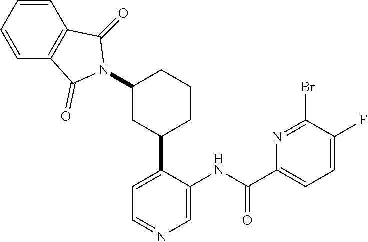

Following Method 5,2-(3-(3-aminopyridin-4-yl)cyclohexyl)isoindoline-1,3-dione and 6-bromo-5-fluoropicolinic acid were coupled and following addition of EtOAc and washing with H₂O, NaCl_{(sat.)} and drying over MgSO₄, 6-bromo-N-(4-((1R,3S)-3-(1,3-dioxoisoindolin-2-yl)cyclohexyl)pyridin-3-yl)-5-fluoropicolinamide was obtained. LCMS (m/z): 523.2/525.2 (MH⁺); LC Rₜ=3.31 min.

### Synthesis of tert-butyl (1S,3R,5S)-3-(3-(6-bromo-5-fluoropicolinamido)-pyridin-4-yl)-5-methylcyclohexylcarbamate

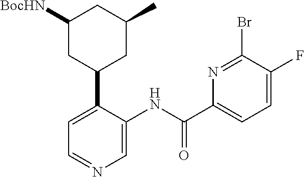

Following Method 5, tert-butyl (1S,3R,5S)-3-(3-aminopyridin-4-yl)-5-methylcyclohexylcarbamate and 6-bromo-5-fluoropicolinic acid were coupled and following addition of EtOAc and washing with H₂O, NaCl_{(sat.)} and drying over MgSO₄, tert-butyl (1S,3R,5S)-3-(3-(6-bromo-5-fluoropicolinamido)pyridin-4-yl)-5-methylcyclohexylcarbamate was obtained. LCMS (m/z): 507.1/509.1 (MH⁺) Rₜ=0.90 min.

### Synthesis of tert-butyl (3R,4R,5S)-1-(3-(6-bromo-5-fluoropicolinamido)pyridin-4-yl)-4-(tert-butyldimethylsilyloxy)-5-methylpiperidin-3-ylcarbamate

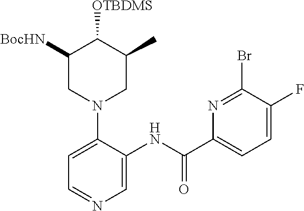

Following Method 5, tert-butyl (3R,4R,5S)-1-(3-aminopyridin-4-yl)-4-(tert-butyldimethylsilyloxy)-5-methylpiperidin-3-ylcarbamate and 6-bromo-5-fluoropicolinic acid were coupled and following addition of EtOAc and washing with H₂O, NaCl_{(sat.)} and drying over MgSO₄, tert-butyl (3R,4R,5S)-1-(3-(6-bromo-5-fluoropicolinamido)pyridin-4-yl)-4-(tert-butyldimethylsilyloxy)-5-methylpiperidin-3-ylcarbamate was obtained. LCMS (m/z): 638.2/640.2 (MH⁺), Rₜ=1.09 min.

### Method 6

### Synthesis of N-(4-((1R,3S,5S)-3-amino-5-methylcyclohexyl)pyridin-3-yl)-6-(3-ethoxy-2,6-difluorophenyl)-5-fluoropicolinamide

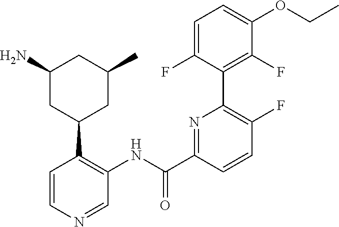

To a solution of tert-butyl (1S,3R,5S)-3-(3-(6-bromo-5-fluoropicolinamido)pyridin-4-yl)-5-methylcyclohexylcarbamate (1.0 equiv.) in a microwave vial was added 3-ethoxy-2,6-difluorophenylboronic acid (5.0 equiv.), KF (5.5 equiv.) and Pd₂(dba)₃ (0.2 equiv.) followed by THF and water (10:1, 0.03 M). To this mixture was added P(t-Bu)₃ (0.4 equiv.) and the reaction was heated in the microwave at 100° C. for 30 min. The organic phase was then separated, the aqueous layer was washed with ethyl acetate, and the organics were combined and concentrated in vacuo. The crude mixture was purified via prep-HPLC, the product fractions were lyophilized and the resulting BOC group was deprotected as described in Method 4 yielding, after RP HPLC purification and lyophilization, N-(4-((1R,3S,5S)-3-amino-5-methylcyclohexyl)pyridin-3-yl)-6-(3-ethoxy-2,6-difluorophenyl)-5-fluoropicolinamide as the TFA salt. LCMS (m/z): 475.0 (MH+); Rₜ=0.68 min.

The following compounds were prepared using Method 6 for Suzuki reaction and Method 5 for deprotection:

TABLE 2

| | | LC/MS | | |
|---|---|---|---|---|
| | | (M + H | LC/MS | |
| Ex | | on | (Rf on | |
| # | Structure | UPLC) | UPLC) | Chemical Name |
| 208 | 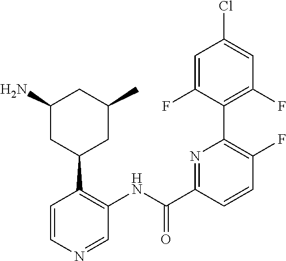 | 475.0 | 0.68 | N-(4-((1R,3S,5S)-3-amino-5- methylcyclohexyl)pyridin-3- yl)-6-(4-chloro-2,6- difluorophenyl)-5- fluoropicolinamide |
| 209 | 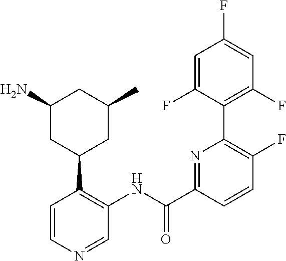 | 459.1 | 0.62 | N-(4-((1R,3S,5S)-3-amino-5- methylcyclohexyl)pyridin-3- yl)-5-fluoro-6-(2,4,6- trifluorophenyl)picolinamide |
| 210 | 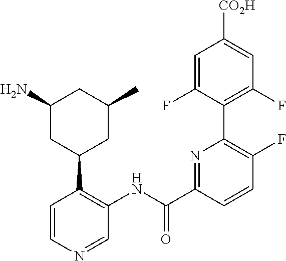 | 485.2 | 0.57 | 4-(6-(4-((1R,3S,5S)-3-amino- 5-methylcyclohexyl)pyridin-3- ylcarbamoyl)-3-fluoropyridin- 2-yl)-3,5-difluorobenzoic acid |
| 211 | 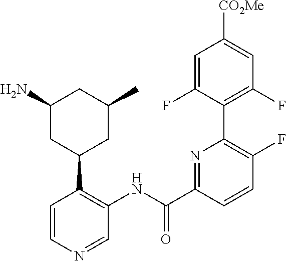 | 499.1 | 0.64 | methyl 4-(6-(4-((1R,3S,5S)-3- amino-5- methylcyclohexyl)pyridin-3- ylcarbamoyl)-3-fluoropyridin- 2-yl)-3,5-difluorobenzoate |
| 212 | 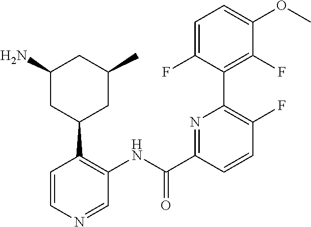 | 471.2 | 0.61 | N-(4-((1R,3S,5S)-3-amino-5- methylcyclohexyl)pyridin-3- yl)-6-(2,6-difluoro-3- methoxyphenyl)-5- fluoropicolinamide |
| 213 | 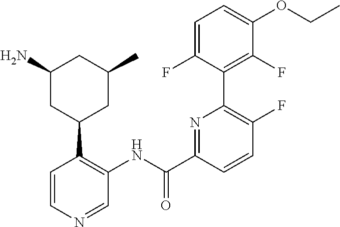 | 485.1 | 0.69 | N-(4-((1R,3S,5S)-3-amino-5- methylcyclohexyl)pyridin-3- yl)-6-(3-ethoxy-2,6- difluorophenyl)-5- fluoropicolinamide |
| 214 | 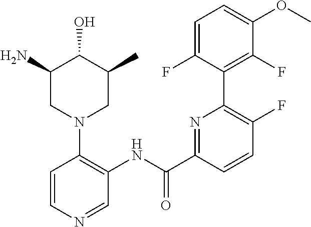 | 488.0 | 0.58 | N-(4-((3R,4R,5S)-3-amino-4- hydroxy-5-methylpiperidin-1- yl)pyridin-3-yl)-6-(2,6- difluoro-3-methoxyphenyl)-5- fluoropicolinamide |
| 215 | 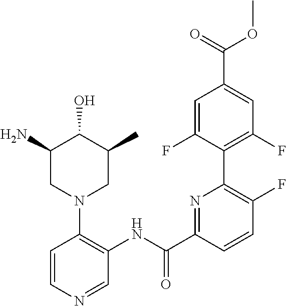 | 516.2 | 0.60 | methyl 4-(6-((4-((3R,4R,5S)- 3-amino-4-hydroxy-5- methylpiperidin-1-yl)pyridin- 3-yl)carbamoyl)-3- fluoropyridin-2-yl)-3,5- difluorobenzoate |
| 216 | 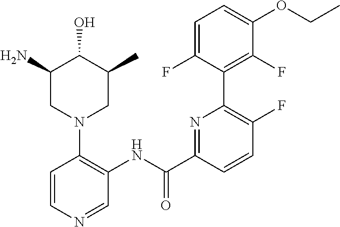 | 502.0 | 0.60 | N-(4-((3R,4R,5S)-3-amino-4- hydroxy-5-methylpiperidin-1- yl)pyridin-3-yl)-6-(3-ethoxy- 2,6-difluorophenyl)-5- fluoropicolinamide |
| 217 | 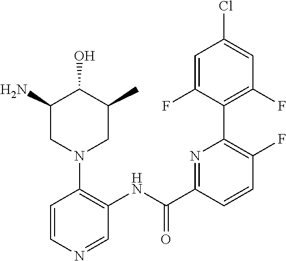 | 492.0 | 0.60 | N-(4-((3R,4R,5S)-3-amino-4- hydroxy-5-methylpiperidin-1- yl)pyridin-3-yl)-6-(4-chloro- 2,6-difluorophenyl)-5- fluoropicolinamide |
| 218 | 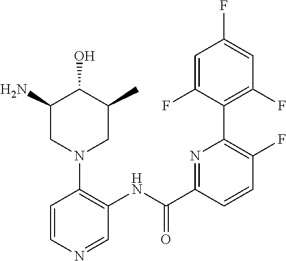 | 476.0 | 0.56 | N-(4-((3R,4R,5S)-3-amino-4- hydroxy-5-methylpiperidin-1- yl)pyridin-3-yl)-5-fluoro-6- (2,4,6- trifluorophenyl)picolinamide |
| 219 | 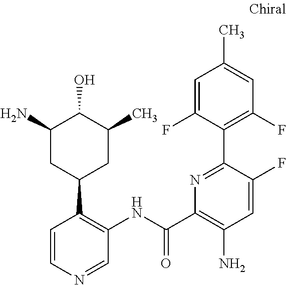 | 486.1 | 0.66 | 3-amino-N-(4- ((1R,3R,4R,5S)-3-amino-4- hydroxy-5- methylcyclohexyl)pyridin-3- yl)-6-(2,6-difluoro-4- methylphenyl)-5- fluoropicolinamide |
| 220 | 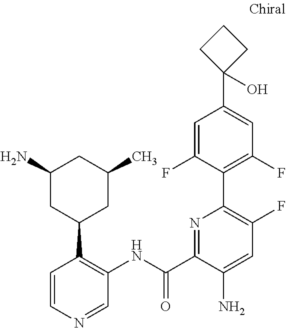 | 526.1 | 0.65 | 3-amino-N-(4-((1R,3S,5S)-3- amino-5- methylcyclohexyl)pyridin-3- yl)-6-(2,6-difluoro-4-(1- hydroxycyclobutyl)phenyl)-5- fluoropicolinamide |
| 221 | 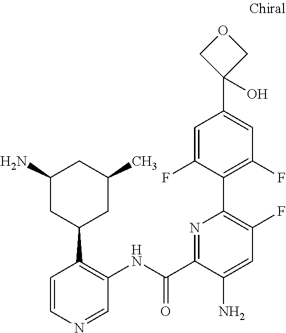 | 528.1 | 0.57 | 3-amino-N-(4-((1R,3S,5S)-3- amino-5- methylcyclohexyl)pyridin-3- yl)-6-(2,6-difluoro-4-(3- hydroxyoxetan-3-yl)phenyl)- 5-fluoropicolinamide |
| 222 | 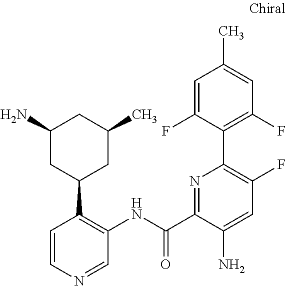 | 470.1 | 0.66 | 3-amino-N-(4-((1R,3S,5S)-3- amino-5- methylcyclohexyl)pyridin-3- yl)-6-(2,6-difluoro-4- methylphenyl)-5- fluoropicolinamide |

### Synthesis of tert-butyl tert-butyl (1S,3R,5S)-3-(3-(6-(2,6-difluoro-4-(methylthio)phenyl)-5-fluoropicolinamido)pyridin-4-yl)-5-methylcyclohexylcarbamate

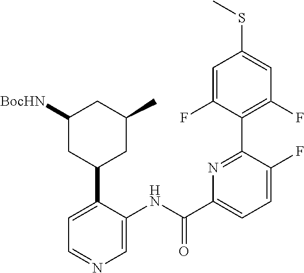

Method 6 was followed using tert-butyl (1S,3R,5S)-3-(3-(6-bromo-5-fluoropicolinamido)pyridin-4-yl)-5-methylcyclohexylcarbamate (1.0 equiv.) and 2-(2,6-difluoro-4-(methylthio)phenyl)-4,4,5,5-tetramethyl-1,3,2-dioxaborolane (2.5 equiv.) with microwave heating at 100° C. for 30 minutes to give tert-butyl (1S,3R,5S)-3-(3-(6-(2,6-difluoro-4-(methylthio)phenyl)-5-fluoropicolinamido)pyridin-4-yl)-5-methylcyclohexylcarbamatein 87% yield. LC/MS=587.1 (M+H), Rt=1.01 min.

### Synthesis of tert-butyl (1S,3R,5S)-3-(3-(6-(2,6-difluoro-4-((R)-methylsulfinyl)phenyl)-5-fluoropicolinamido)pyridin-4-yl)-5-methylcyclohexylcarbamate and tert-butyl (1S,3R,5S)-3-(3-(6-(2,6-difluoro-4-((S)-methylsulfinyl)phenyl)-5-fluoropicolinamido)pyridin-4-yl)-5-methylcyclohexylcarbamate

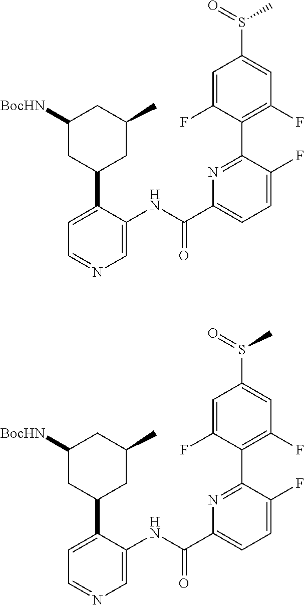

To a solution of tert-butyl (1S,3R,5S)-3-(3-(5-fluoro-6-(4-(methylthio)phenyl)picolinamido)pyridin-4-yl)-5-methylcyclohexylcarbamate in CH₂Cl₂ (0.1 M) at rt was added oxone (1.0 equiv). After stirring for 24 hours, an additional equivalent of oxone was added. After stirring for 16 more hours, 1 more equivalent of oxone was added. After stirring for 12 hours, the solution was diluted with EtOAc, washed with NaHCO_{3(sat.)}, brine, dried over MgSO₄, filtered, concentrated and purified by SiO2 chromatography to yield the diasteromeric sulfoxides in 75%. The diastereomers were separated using a chiral AD column (Heptane:EtOH 80/20) to yield tert-butyl (1S,3R,5S)-3-(3-(6-(2,6-difluoro-4-((R)-methylsulfinyl)phenyl)-5-fluoropicolinamido)pyridin-4-yl)-5-methylcyclohexylcarbamate and tert-butyl (1S,3R,5S)-3-(3-(6-(2,6-difluoro-4-((S)-methylsulfinyl)phenyl)-5-fluoropicolinamido)pyridin-4-yl)-5-methylcyclohexylcarbamate. LC/MS=603.2 (M+H), Rt=0.78 min for both diastereomers.

### Synthesis of tert-butyl (1S,3R,5S)-3-(3-(6-(2,6-difluoro-4-formylphenyl)-5-fluoropicolinamido)pyridin-4-yl)-5-methylcyclohexylcarbamate

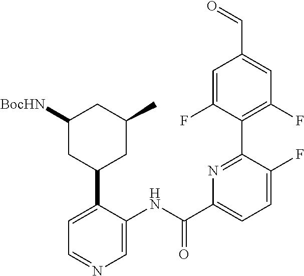

Method 6 was followed using tert-butyl (1S,3R,5S)-3-(3-(6-bromo-5-fluoropicolinamido)pyridin-4-yl)-5-methylcyclohexylcarbamate (1.0 equiv.) and 3,5-difluoro-4-(4,4,5,5-tetramethyl-1,3,2-dioxaborolan-2-yl)benzaldehyde (2.5 equiv.) with microwave heating at 100° C. for 30 minutes to give tert-butyl (1S,3R,5S)-3-(3-(6-(2,6-difluoro-4-formylphenyl)-5-fluoropicolinamido)pyridin-4-yl)-5-methylcyclohexylcarbamate in 67% yield. LC/MS=569.1 (M+H), Rt=0.88 min.

### Synthesis of tert-butyl (1S,3R,5S)-3-(3-(6-(2,6-difluoro-4-(hydroxymethyl)phenyl)-5-fluoropicolinamido)pyridin-4-yl)-5-methylcyclohexylcarbamate

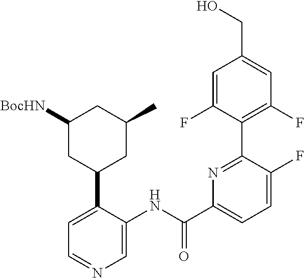

To a solution of tert-butyl (1S,3R,5S)-3-(3-(6-(2,6-difluoro-4-formylphenyl)-5-fluoropicolinamido)pyridin-4-yl)-5-methylcyclohexylcarbamate in MeOH (0.1 M) at 0° C. was added sodium borohydride (2.0 equiv). After stirring for 10 minutes, water was added to quench and the volatiles were removed in vacuo. The residue was dissolved in EtOAc, was washed with NaCl_{(sat.)}, dried over MgSO₄, filtered, concentrated and purified by SiO₂ chromatography to yield tert-butyl (1S,3R,5S)-3-(3-(6-(2,6-difluoro-4-(hydroxymethyl)phenyl)-5-fluoropicolinamido)pyridin-4-yl)-5-methylcyclohexylcarbamate in 60% yield. LC/MS=571.2 (M+H), Rₜ=0.69 min.

### Synthesis of tert-butyl (1S,3R,5S)-3-(3-(6-(2,6-difluoro-4-vinylphenyl)-5-fluoropicolinamido)pyridin-4-yl)-5-methylcyclohexylcarbamate

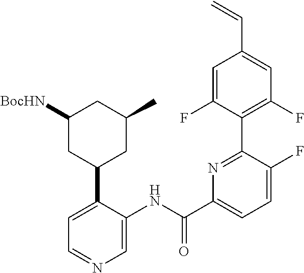

A solution of methyltriphenylphosphonium bromide (3.5 equiv.) in THF (0.1 M) was treated with potassium t-butoxide (3.5 equiv.) After stirring at rt for 2 hours, the solution was cooled in a −78° C. bath, and a solution of tert-butyl (1S,3R,5S)-3-(3-(6-(2,6-difluoro-4-formylphenyl)-5-fluoropicolinamido)pyridin-4-yl)-5-methylcyclohexylcarbamate in THF was added dropwise. The solution stirred for 3 hours as it warmed to rt. The reaction was diluted with EtOAc, washed with water, brine, dried over Na₂SO₄, filtered, concentrated, purified by SiO₂ chromatography to yield tert-butyl (1S,3R,5S)-3-(3-(6-(2,6-difluoro-4-vinylphenyl)-5-fluoropicolinamido)pyridin-4-yl)-5-methylcyclohexylcarbamate in 46% yield as a white solid. LC/MS=567.2 (M+H), Rₜ=0.99 min. ¹H NMR (400 MHz, <cdcl₃>) 9.89 (s, 1H), 9.32 (s, 1H), 8.38-8.44 (m, 2H), 7.76 (t, 1H), 7.13-7.19 (m, 3H), 6.72 (dd, 1H), 5.92 (d, 1H), 5.48 (d, 1H), 4.44 (br. s., 1H), 3.60 (br. s., 1H), 2.90 (t, 1H), 2.13 (d, 1H), 2.00 (d, 1H), 1.82 (d, 1H), 1.50-1.60 (m, 1H), 1.40-1.45 (m, 9H), 1.30-1.38 (m, 1H), 0.95 (q, 1H), 0.85 (d, 3H), 0.74-0.82 (m, 1H).

### Synthesis of tert-butyl (1S,3R,5S)-3-(3-(6-(4-((S)-1,2-dihydroxyethyl)-2,6-difluorophenyl)-5-fluoropicolinamido)pyridin-4-yl)-5-methylcyclohexylcarbamate and tert-butyl (1S,3R,5S)-3-(3-(6-(4-((R)-1,2-dihydroxyethyl)-2,6-difluorophenyl)-5-fluoropicolinamido)pyridin-4-yl)-5-methylcyclohexylcarbamate

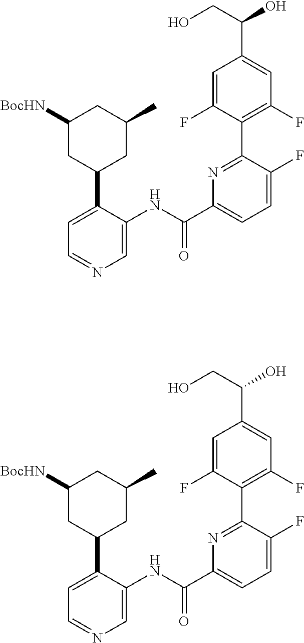

To a solution of tert-butyl (1S,3R,5S)-3-(3-(6-(2,6-difluoro-4-vinylphenyl)-5-fluoropicolinamido)pyridin-4-yl)-5-methylcyclohexylcarbamate (1.0 equiv) in 4:1 acetone/H₂O (0.05 M) was added NMO (2.0 equiv) and 2.5% OsO₄ (0.04 equiv.). After stirring for 24 hours the solution was concentrated and purified directly by SiO₂ chromatography to yield product as a diastereomeric mixture. The diastereomers were separated using a chiral AD-H column (Heptane:EtOH 90/10) to yield tert-butyl (1S,3R,5S)-3-(3-(6-(4-((S)-1,2-dihydroxyethyl)-2,6-difluorophenyl)-5-fluoropicolinamido)pyridin-4-yl)-5-methylcyclohexylcarbamate and tert-butyl (1S,3R,5S)-3-(3-(6-(4-((R)-1,2-dihydroxyethyl)-2,6-difluorophenyl)-5-fluoropicolinamido)pyridin-4-yl)-5-methylcyclohexylcarbamate in 32% and 25% yield. LC/MS=601.3 (M+H), Rₜ=0.74 min for both diastereomers.

### Synthesis of tert-butyl (1S,3R,5S)-3-(3-(6-(4-ethyl-2,6-difluorophenyl)-5-fluoropicolinamido)pyridin-4-yl)-5-methylcyclohexylcarbamate

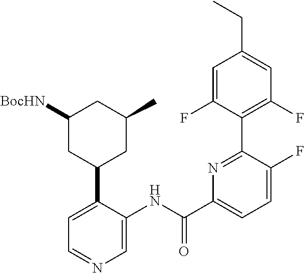

To a solution of tert-butyl (1S,3R,5S)-3-(3-(6-(2,6-difluoro-4-vinylphenyl)-5-fluoropicolinamido)pyridin-4-yl)-5-methylcyclohexylcarbamate (1.0 equiv.) in methanol (0.1 M) was added 10% Pd/C (0.1 equiv.). The reaction was placed under an atmosphere of hydrogen and stirred for 18 hours. Upon completion, the solution was filtered over a pad of Celite, the pad was washed with methanol, the filtrate was concentrated in vacuo to give tert-butyl (1S,3R,5S)-3-(3-(6-(4-ethyl-2,6-difluorophenyl)-5-fluoropicolinamido)pyridin-4-yl)-5-methylcyclohexylcarbamate as an oil in 93% yield. LC/MS=569.2 (M+H), Rₜ=1.01 min.

### Synthesis of tert-butyl (3R,4R,5S)-4-(tert-butyldimethylsilyloxy)-1-(3-(6-(2,6-difluoro-4-vinylphenyl)-5-fluoropicolinamido)pyridin-4-yl)-5-methylpiperidin-3-ylcarbamate

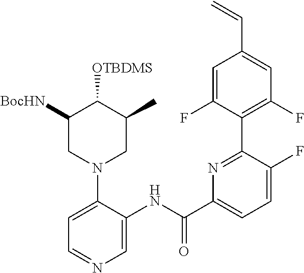

Following Method 5, tert-butyl (3R,4R,5S)-1-(3-aminopyridin-4-yl)-4-(tert-butyldimethylsilyloxy)-5-methylpiperidin-3-ylcarbamate and 6-(2,6-difluoro-4-vinylphenyl)-5-fluoropicolinic acid were coupled and following addition of EtOAc and washing with H₂O, NaCl_{(sat.)}, drying over MgSO₄ and purification by ISCO SiO₂ chromatography, tert-butyl (3R,4R,5S)-4-(tert-butyldimethylsilyloxy)-1-(3-(6-(2,6-difluoro-4-vinylphenyl)-5-fluoropicolinamido)pyridin-4-yl)-5-methylpiperidin-3-ylcarbamate was obtained in 67% yield. LCMS (m/z): 698.3 (MH⁺) Rₜ=1.18 min.

### Synthesis of tert-butyl (3R,4R,5S)-4-(tert-butyldimethylsilyloxy)-1-(3-(6-(4-ethyl-2,6-difluorophenyl)-5-fluoropicolinamido)pyridin-4-yl)-5-methylpiperidin-3-ylcarbamate

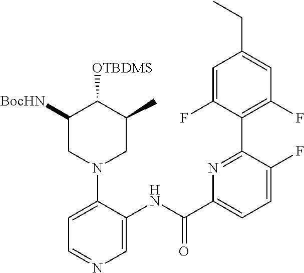

To a solution of tert-butyl (3R,4R,5S)-4-(tert-butyldimethylsilyloxy)-1-(3-(6-(2,6-difluoro-4-vinylphenyl)-5-fluoropicolinamido)pyridin-4-yl)-5-methylpiperidin-3-ylcarbamate (1.0 equiv.) in methanol (0.1 M) was added 10% Pd/C (0.1 equiv.). The reaction was placed under an atmosphere of hydrogen and stirred for 3 hours. Upon completion, the solution was filtered over a pad of Celite, the pad was washed with methanol, the filtrate was concentrated in vacuo to give tert-butyl (3R,4R,5S)-4-(tert-butyldimethylsilyloxy)-1-(3-(6-(4-ethyl-2,6-difluorophenyl)-5-fluoropicolinamido)pyridin-4-yl)-5-methylpiperidin-3-ylcarbamate as a solid in 99% yield. LC/MS=700.4 (M+H), Rₜ=1.20 min.

### Synthesis of tert-butyl (3R,4R,5S)-4-(tert-butyldimethylsilyloxy)-1-(3-(6-(4-((S)-1,2-dihydroxyethyl)-2,6-difluorophenyl)-5-fluoropicolinamido)pyridin-4-yl)-5-methylpiperidin-3-ylcarbamate and tert-butyl (3R,4R,5S)-4-(tert-butyldimethylsilyloxy)-1-(3-(6-(4-((R)-1,2-dihydroxyethyl)-2,6-difluorophenyl)-5-fluoropicolinamido)pyridin-4-yl)-5-methylpiperidin-3-ylcarbamate

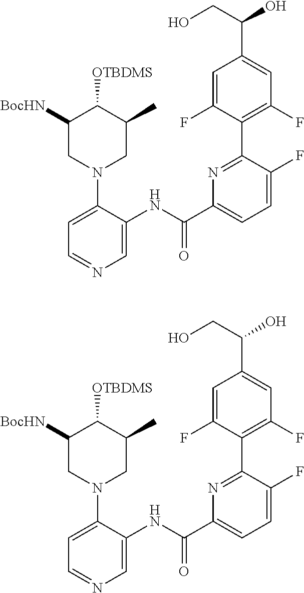

To a solution of tert-butyl (3R,4R,5S)-4-(tert-butyldimethylsilyloxy)-1-(3-(6-(4-ethyl-2,6-difluorophenyl)-5-fluoropicolinamido)pyridin-4-yl)-5-methylpiperidin-3-ylcarbamate (1.0 equiv) in 4:1 acetone/H₂O (0.05 M) was added NMO (4.0 equiv) and 2.5% OsO₄ (0.08 equiv.). After stirring for 37 hours the solution was concentrated and purified directly by SiO₂ chromatography to yield tert-butyl (3R,4R,5S)-4-(tert-butyldimethylsilyloxy)-1-(3-(6-(4-((S)-1,2-dihydroxyethyl)-2,6-difluorophenyl)-5-fluoropicolinamido)pyridin-4-yl)-5-methylpiperidin-3-ylcarbamate and tert-butyl (3R,4R,5S)-4-(tert-butyldimethylsilyloxy)-1-(3-(6-(4-((R)-1,2-dihydroxyethyl)-2,6-difluorophenyl)-5-fluoropicolinamido)pyridin-4-yl)-5-methylpiperidin-3-ylcarbamate as a mixture. LC/MS=732.4 (M+H), Rₜ=0.96 min for both diastereomers.

### Synthesis of tert-butyl (1S,3R,5S)-3-(3-(6-(2,6-difluoro-4-hydroxyphenyl)-5-fluoropicolinamido)pyridin-4-yl)-5-methylcyclohexylcarbamate

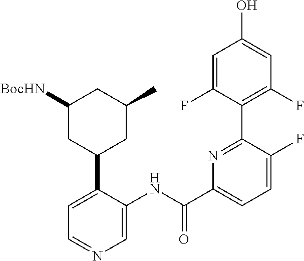

Method 6 was followed using tert-butyl (1S,3R,5S)-3-(3-(6-bromo-5-fluoropicolinamido)pyridin-4-yl)-5-methylcyclohexylcarbamate (1.0 equiv.) and tert-butyl(3,5-difluoro-4-(4,4,5,5-tetramethyl-1,3,2-dioxaborolan-2-yl)phenoxy)dimethylsilane (2.5 equiv.) with microwave heating at 100° C. for 30 minutes to give tert-butyl (1S,3R,5S)-3-(3-(6-(2,6-difluoro-4-formylphenyl)-5-fluoropicolinamido)pyridin-4-yl)-5-methylcyclohexylcarbamate in 54% yield. The TBDMS group falls off during the Suzuki reaction. If the deprotection is not complete, adding KF and H₂O and additional microwave heating at 100° C. can drive the silyl deprotection to completion. LC/MS=557.2 (M+H), Rₜ=0.84 min.

### Synthesis of tert-butyl (1S,3R,5S)-3-(3-(6-(4-ethoxy-2,6-difluorophenyl)-5-fluoropicolinamido)pyridin-4-yl)-5-methylcyclohexylcarbamate

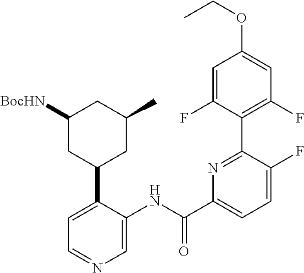

To a solution of tert-butyl (1S,3R,5S)-3-(3-(6-(2,6-difluoro-4-formylphenyl)-5-fluoropicolinamido)pyridin-4-yl)-5-methylcyclohexylcarbamate (1.0 equiv.) and K₂CO₃ (3.0 equiv.) in DMF (0.1 M) was added diethylsulfate (1.0 equiv.). The heterogeneous solution was heated at 80° C. for 1 hour. Upon cooling, the reaction was diluted with EtOAc, washed with water, brine, dried over MgSO₄, filtered and concentrated to yield tert-butyl (1S,3R,5S)-3-(3-(6-(4-ethoxy-2,6-difluorophenyl)-5-fluoropicolinamido)pyridin-4-yl)-5-methylcyclohexylcarbamate. LC/MS=585.2 (M+H), Rₜ=1.06 min.

### Synthesis of tert-butyl (1S,3R,5S)-3-(3-(6-(2,6-difluoro-4-(2-hydroxyethoxy)phenyl)-5-fluoropicolinamido)pyridin-4-yl)-5-methylcyclohexylcarbamate

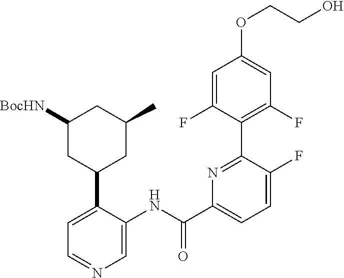

To a solution of tert-butyl (1S,3R,5S)-3-(3-(6-(2,6-difluoro-4-formylphenyl)-5-fluoropicolinamido)pyridin-4-yl)-5-methylcyclohexylcarbamate (1.0 equiv.), triphenylphosphine (3.0 equiv.), 2-(benzyloxy)ethanol (3.0 equiv.) in THF (0.1 M) at 0° C. was added DIAD (3.0 equiv.). After warming to rt and stirring for 48 hours the volatiles were removed in vacuo and the residue was purified by SiO₂ chromatography to yield the benzyl ether product [LC/MS=691.3 9 (M+H), Rₜ=1.07 min]. To a solution of the benzyl ether (1.0 equiv.) in methanol (0.1 M) was added 10% Pd/C (0.4 equiv.). The reaction was placed under an atmosphere of hydrogen and stirred for 18 hours at which time additional 10% Pd/C was added and the reaction was recharged with an atmosphere of hydrogen. Upon completion, the solution was filtered over a pad of Celite, the pad was washed with methanol, the filtrate was concentrated in vacuo to give tert-butyl (1S,3R,5S)-3-(3-(6-(2,6-difluoro-4-(2-hydroxyethoxy)phenyl)-5-fluoropicolinamido)pyridin-4-yl)-5-methylcyclohexylcarbamate. LC/MS=601.2 (M+H), Rₜ=0.83 min.

### Synthesis of tert-butyl (1S,3R,5S)-3-(3-(6-(2,6-difluoro-4-(2-(triisopropylsilyloxy)propan-2-yl)phenyl)-5-fluoropicolinamido)pyridin-4-yl)-5-methylcyclohexylcarbamate

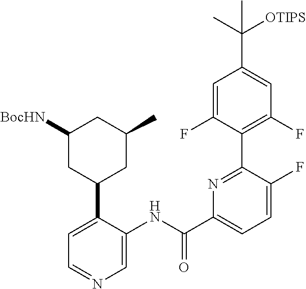

Method 6 was followed using tert-butyl (1S,3R,5S)-3-(3-(6-bromo-5-fluoropicolinamido)pyridin-4-yl)-5-methylcyclohexylcarbamate (1.0 equiv.) and (2-(3,5-difluoro-4-(4,4,5,5-tetramethyl-1,3,2-dioxaborolan-2-yl)phenyl)propan-2-yloxy)triisopropylsilane (2.5 equiv.) with microwave heating at 100° C. for 30 minutes to give tert-butyl (1S,3R,5S)-3-(3-(6-(2,6-difluoro-4-(2-(triisopropylsilyloxy)propan-2-yl)phenyl)-5-fluoropicolinamido)pyridin-4-yl)-5-methylcyclohexylcarbamate. LC/MS=755.4 (M+H), Rt=0.98 min.

### Synthesis of N-(4-((1R,3S,5S)-3-amino-5-methylcyclohexyl)pyridin-3-yl)-6-(4-((S)-2,3-dihydroxypropoxy)-2,6-difluorophenyl)-5-fluoropicolinamide

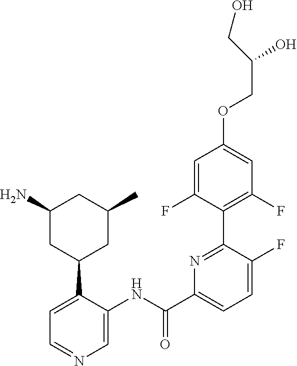

To a solution of triphenylphospine (3.0 equiv.), (R)-(2,2-dimethyl-1,3-dioxolan-4-yl)methanol (3.0 equiv.) and tert-butyl (1S,3R,5S)-3-(3-(6-(2,6-difluoro-4-hydroxyphenyl)-5-fluoropicolinamido)pyridin-4-yl)-5-methylcyclohexylcarbamate (1.0 equiv.) in THF (0.11 M) at 0° C. was added DIAD (3.0 equiv.) dropwise. The reaction was allowed to warm to rt and stirred for 16 hrs. The reaction mixture was concentrated under vacuo and purified via ISCO SiO2 chromatography (ethyl acetate and heptanes 0-100%) to give tert-butyl ((1S,3R,5S)-3-(3-(6-(4-(((R)-2,2-dimethyl-1,3-dioxolan-4-yl)methoxy)-2,6-difluorophenyl)-5-fluoropicolinamido)pyridin-4-yl)-5-methylcyclohexyl)carbamate. LC/MS=671.4 (MH+), Rt=0.97 min. The product was treated with 25% TFA/CH2Cl2 (0.05 M) for 2 hours at which time the volatiles were removed in vacuo. The residue was dissolved in 2:1 TFA/H2O (0.05 M) and left standing at rt at overnight. Toluene was added and the volatiles were removed in vacuo and the residue was purified by Reverse phase HPLC to yield N-(4-((1R,3S,5S)-3-amino-5-methylcyclohexyl)pyridin-3-yl)-6-(4-((S)-2,3-dihydroxypropoxy)-2,6-difluorophenyl)-5-fluoropicolinamide in 49% yield. LC/MS=531.1 (MH+), Rt=0.55 min.

### Synthesis of N-(4-((1R,3S,5S)-3-amino-5-methylcyclohexyl)pyridin-3-yl)-6-(4-((R)-2,3-dihydroxypropoxy)-2,6-difluorophenyl)-5-fluoropicolinamide

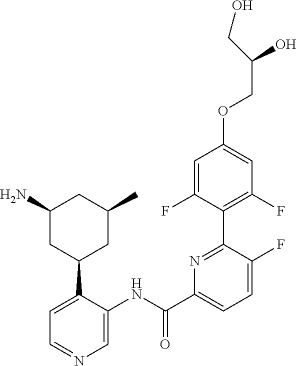

To a solution of triphenylphospine (2.8 equiv.), (S)-(2,2-dimethyl-1,3-dioxolan-4-yl)methanol (2.8 equiv.) and tert-butyl (1S,3R,5S)-3-(3-(6-(2,6-difluoro-4-hydroxyphenyl)-5-fluoropicolinamido)pyridin-4-yl)-5-methylcyclohexylcarbamate (1.0 equiv.) in THF (0.11 M) at 0° C. was added DIAD (2.8 equiv.) dropwise. The reaction was allowed to warm to rt and stirred for 16 hrs. The reaction mixture was concentrated under vacuo and purified via ISCO SiO₂ chromatography (ethyl acetate and heptanes 0-100%) to give tert-butyl ((1S,3R,5S)-3-(3-(6-(4-(((S)-2,2-dimethyl-1,3-dioxolan-4-yl)methoxy)-2,6-difluorophenyl)-5-fluoropicolinamido)pyridin-4-yl)-5-methylcyclohexyl)carbamate LC/MS=671.4 (MH+), Rt=0.99 min. The product was treated with 25% TFA/CH2Cl2 (0.05 M) for 2 hours at which time the volatiles were removed in vacuo. The residue was dissolved in 2:1 TFA/H₂O (0.05 M) and left standing at rt at overnight. Toluene was added and the volatiles were removed in vacuo and the residue was purified by Reverse phase HPLC to yield N-(4-((1R,3S,5S)-3-amino-5-methylcyclohexyl)pyridin-3-yl)-6-(4-((R)-2,3-dihydroxypropoxy)-2,6-difluorophenyl)-5-fluoropicolinamide in 21% yield. LC/MS=531.1 (MH+), Rt=0.55 min.

### Method 7

### Synthesis of N-(4-((1R,3S,5S)-3-amino-5-methylcyclohexyl)pyridin-3-yl)-5-fluoro-6-(4-(methylthio)phenyl)picolinamide

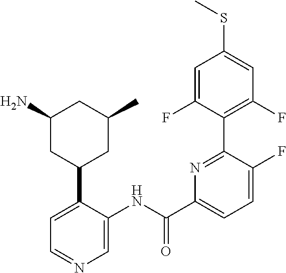

tert-butyl (1S,3R,5S)-3-(3-(5-fluoro-6-(4-(methylthio)phenyl)picolinamido)pyridin-4-yl)-5-methylcyclohexylcarbamate was deprotected by treating with 25% TFA/CH₂Cl₂ for 1 hour. Upon removal of volatiles in vacuo and purification by RP-HPLC, N-(4-((1R,3S,5S)-3-amino-5-methylcyclohexyl)pyridin-3-yl)-5-fluoro-6-(4-(methylthio)phenyl)picolinamide was obtained. LC/MS=487.1 (M+H), Rₜ=0.68 min.

The following compounds were prepared using Method 7 or the additional deprotection conditions described in Method 5:

TABLE 3

| | | LC/MS | | |
|---|---|---|---|---|
| | | (M + H | LC/MS | |
| Ex | | on | (Rf on | |
| # | Structure | UPLC) | UPLC) | Chemical Name |
| 223 | 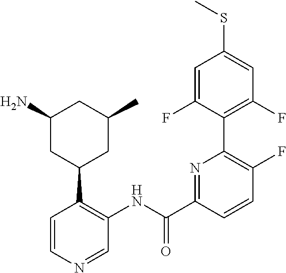 | 487.1 | 0.68 | N-(4-((1R,3S,5S)-3-amino-5- methylcyclohexyl)pyridin-3- yl)-6-(2,6-difluoro-4- (methylthio)phenyl)-5- fluoropicolinamide |
| 224 | 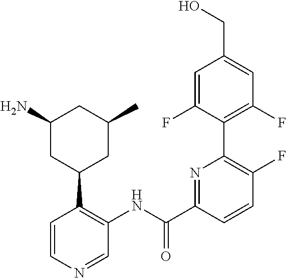 | 471.1 | 0.55 | N-(4-((1R,3S,5S)-3-amino-5- methylcyclohexyl)pyridin-3- yl)-6-(2,6-difluoro-4- (hydroxymethyl)phenyl)-5- fluoropicolinamide |
| 225 | 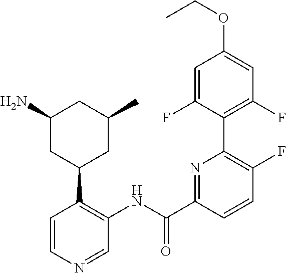 | 485.1 | 0.71 | N-(4-((1R,3S,5S)-3-amino-5- methylcyclohexyl)pyridin-3- yl)-6-(4-ethoxy-2,6- difluorophenyl)-5- fluoropicolinamide |
| 226 | 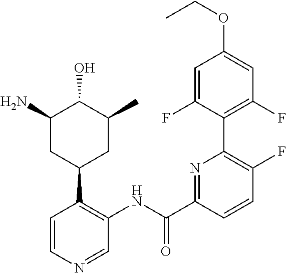 | 501.1 | 0.66 | N-(4-((1R,3R,4R,5S)-3- amino-4-hydroxy-5- methylcyclohexyl)pyridin-3- yl)-6-(4-ethoxy-2,6- difluorophenyl)-5- fluoropicolinamide |
| 227 | 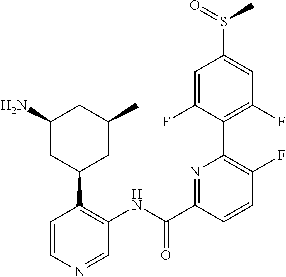 | 503.0 | 0.54 | N-(4-((1R,3S,5S)-3-amino-5- methylcyclohexyl)pyridin-3- yl)-6-(2,6-difluoro-4-((S)- methylsulfinyl)phenyl)-5- fluoropicolinamide |
| 228 | 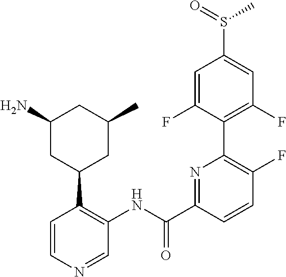 | 503.0 | 0.54 | N-(4-((1R,3S,5S)-3-amino-5- methylcyclohexyl)pyridin-3- yl)-6-(2,6-difluoro-4-((R)- methylsulfinyl)phenyl)-5- fluoropicolinamide |
| 229 | 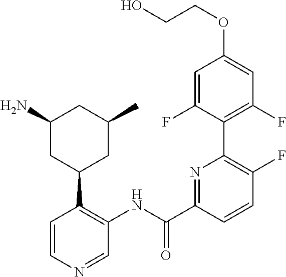 | 501.1 | 0.59 | N-(4-((1R,3S,5S)-3-amino-5- methylcyclohexyl)pyridin-3- yl)-6-(2,6-difluoro-4-(2- hydroxyethoxy)phenyl)-5- fluoropicolinamide |
| 230 | 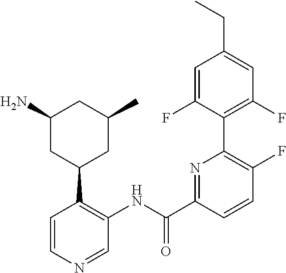 | 469.1 | 0.70 | N-(4-((1R,3S,5S)-3-amino-5- methylcyclohexyl)pyridin-3- yl)-6-(4-ethyl-2,6- difluorophenyl)-5- fluoropicolinamide |
| 231 | 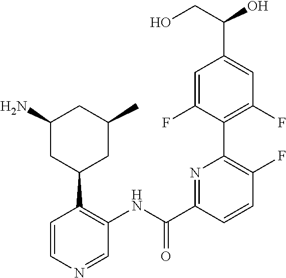 | 501.1 | 0.53 | N-(4-((1R,3S,5S)-3-amino-5- methylcyclohexyl)pyridin-3- yl)-6-(4-((S)-1,2- dihydroxyethyl)-2,6- difluorophenyl)-5- fluoropicolinamide |
| 232 | 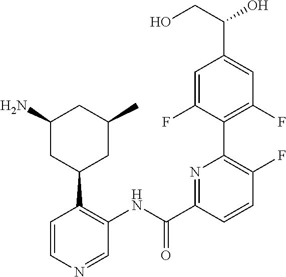 | 501.1 | 0.53 | N-(4-((1R,3S,5S)-3-amino-5- methylcyclohexyl)pyridin-3- yl)-6-(4-((R)-1,2- dihydroxyethyl)-2,6- difluorophenyl)-5- fluoropicolinamide |
| 233 | 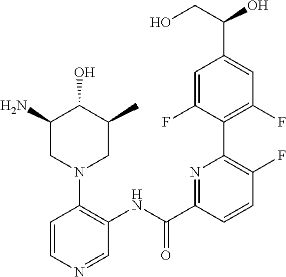 | 518.0 | 0.46 | N-(4-((3R,4R,5S)-3-amino-4- hydroxy-5-methylpiperidin-1- yl)pyridin-3-yl)-6-(4-((S)-1,2- dihydroxyethyl)-2,6- difluorophenyl)-5- fluoropicolinamide |
| 234 | 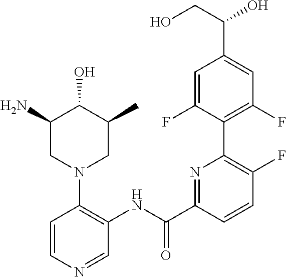 | 518.0 | 0.46 | N-(4-((3R,4R,5S)-3-amino-4- hydroxy-5-methylpiperidin-1- yl)pyridin-3-yl)-6-(4-((R)-1,2- dihydroxyethyl)-2,6- difluorophenyl)-5- fluoropicolinamide |
| 235 | 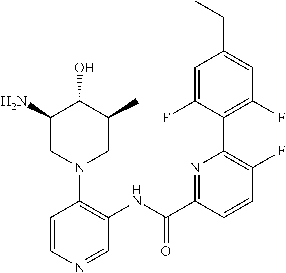 | 486.1 | 0.67 | N-(4-((3R,4R,5S)-3-amino-4- hydroxy-5-methylpiperidin-1- yl)pyridin-3-yl)-6-(4-ethyl-2,6- difluorophenyl)-5- fluoropicolinamide |
| 236 | 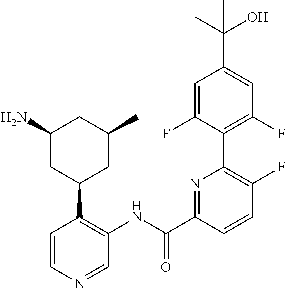 | 499.0 | 0.61 | N-(4-((1R,3S,5S)-3-amino-5- methylcyclohexyl)pyridin-3- yl)-6-(2,6-difluoro-4-(2- hydroxypropan-2-yl)phenyl)- 5-fluoropicolinamide |
| 237 | 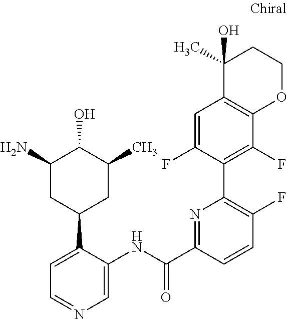 | 543.1 | 0.60 | N-(4-((1R,3R,4R,5S)-3- amino-4-hydroxy-5- methylcyclohexyl)pyridin-3- yl)-6-((S)-6,8-difluoro-4- hydroxy-4-methylchroman-7- yl)-5-fluoropicolinamide |
| 238 | 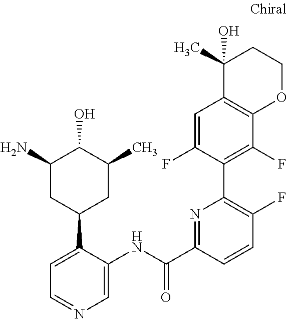 | 543.1 | 0.59 | N-(4-((1R,3R,4R,5S)-3- amino-4-hydroxy-5- methylcyclohexyl)pyridin-3- yl)-6-((R)-6,8-difluoro-4- hydroxy-4-methylchroman-7- yl)-5-fluoropicolinamide |
| 239 | 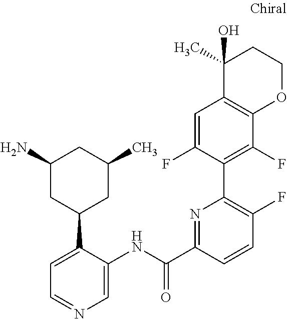 | 527.1 | 0.62 | N-(4-((1R,3S,5S)-3-amino-5- methylcyclohexyl)pyridin-3- yl)-6-((S)-6,8-difluoro-4- hydroxy-4-methylchroman-7- yl)-5-fluoropicolinamide |
| 240 | 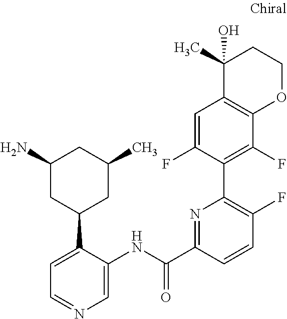 | 527.1 | 0.62 | N-(4-((1R,3S,5S)-3-amino-5- methylcyclohexyl)pyridin-3- yl)-6-((R)-6,8-difluoro-4- hydroxy-4-methylchroman-7- yl)-5-fluoropicolinamide |
| 241 | 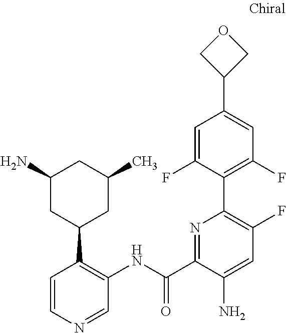 | 512.1 | 0.64 | 3-amino-N-(4-((1R,3S,5S)-3- amino-5- methylcyclohexyl)pyridin-3- yl)-6-(2,6-difluoro-4-(oxetan- 3-yl)phenyl)-5- fluoropicolinamide |

In addition to LC/MS and LC characterization, representative compounds were analyzed by ¹H-NMR. The following are typical spectra of the compounds of the invention.

| Example # | ¹H-NMR data |
|---|---|
| 82 | (400 MHz, <cd3od>) δ 9.10 (s, 1H), 8.39 (dd, J = 3.91 Hz, 1H), 8.34 (d, J = 5.09 Hz, |
| | 1H), 8.00 (t, J = 8.80 Hz, 1H), 7.42 (d, J = 5.09 Hz, 1H), 7.12-7.18 (m, |
| | 2H), 4.05-4.11 (m, 2H), 3.56-3.64 (m, 2H), 2.88-3.03 (m, 2H), 2.73 (tt, J = 3.91, |
| | 11.15 Hz, 1H), 1.98 (d, J = 12.13 Hz, 1H), 1.77-1.90 (m, 5H), 1.44-1.56 |
| | (m, 1H), 1.33 (q, J = 12.13 Hz, 1H), 1.04 (q, J = 12.13 Hz, 1H) 0.89 (d, J = 6.26 Hz, |
| | 3H), 0.79-0.87 (m, 1H) |
| 126 | 400 MHz, <dmso>) δ ppm 0.67 (q, J = 11.74 Hz, 1 H) 0.78 (d, J = 6.65 Hz, 3 H) |
| | 0.90 (q, J = 11.87 Hz, 1 H) 0.98-1.07 (m, 1 H) 1.28-1.41 (m, 1 H) |
| | 1.56-1.80 (m, 4 H) 1.81-1.94 (m, 2 H) 2.10-2.35 (m, 2 H) 2.48-2.59 (m, 1 H) 2.76 (t, |
| | J = 11.93 Hz, 1 H) 3.67 (t, J = 11.15 Hz, 2 H) 3.87 (dd, J = 11.35, 5.09 Hz, 2 H) |
| | 7.28 (d, J = 5.09 Hz, 1 H) 7.36-7.48 (m, 2 H) 8.18 (t, J = 8.80 Hz, 1 H) |
| | 8.27-8.38 (m, 2 H) 8.74 (s, 1 H) 10.27 (br. s., 1 H) |
| 100 | 400 MHz, <dmso>) δ ppm 0.66-0.79 (m, 3 H) 1.47 (s, 7 H) 2.56-2.72 (m, |
| | 1H) 2.81-3.17 (m, 3 H) 3.63-3.92 (m, 1 H) 5.69 (br. s., 1H) 7.14-7.33 (m, 4 |
| | H) 7.34-7.51 (m, 1 H) 7.92-8.14 (m, 2 H) 8.37 (d, J = 6.65 Hz, 1 H) 8.90 (br. |
| | s., 1 H) 9.97-10.17 (m, 1 H) |
| 188 | (400 MHz, <CDCl3>) δ ppm 10.16 (s, 1H), 9.41 (s, 1H), 8.38 (d, J = 5.2, 1H), |
| | 8.28 (d, J = 8.0, 0.8, 1H), 8.01 (dd, J = 8.0, 8.0, 1H), 7.70 (dd, J = 8.0, 0.8, 1H), |
| | 7.19 (d, J = 5.2, 1H), 6.64 (d, J = 10.0, 2H), 4.15-4.18 (m, 2H), 3.78-3.80 (m, 2H), |
| | 3.47 (s, 3H), 2.83-2.92 (m, 1H), 2.79-2.83 (m, 1H), 1.98-2.02 (m, 1H), |
| | 1.88-1.91 (m, 1H), 1.80-1.84 (m, 1H), 1.59-1.64 (m, 1H), 1.25 (q, J = 12.4, 1H), |
| | 1.04 (q, J = 12.0, 1H), 0.93 (d, J = 6.8, 3H), 0.82 (q, J = 12.0, 1H). |
| 187 | (400 MHz, <CDCl3>) δ ppm 10.15 (s, 1H), 9.40 (s, 1H), 8.38 (d, J = 5.2, 1H), |
| | 8.29 (d, J = 8.0, 1.2, 1H), 8.01 (dd, J = 8.0, 8.0, 1H), 7.70 (dd, J = 8.0, 0.8, 1H), |
| | 7.19 (d, J = 5.2, 1H), 6.61 (d, J = 10.0, 2H), 4.52-4.55 (m, 1H), 3.97-4.02 (m, 2H), |
| | 3.59-3.65 (m, 2H), 2.83-2.92 (m, 1H), 2.79-2.83 (m, 1H), 2.04-2.11 (m, 2H), |
| | 1.98-2.01 (m, 1H), 1.80-1.89 (m, 4H), 1.59-1.64 (m, 1H), 1.24 (q, J = 12.4, 1H), |
| | 1.04 (q, J = 12.0, 1H), 0.92 (d, J = 6.8, 3H), 0.82 (q, J = 12.0, 1H). |
| 83 | (400 MHz, <cd3od>) δ ppm 8.95 (s, 1 H), 8.33-8.42 (m, 2 H), 8.00 (t, J = 8.61 Hz, |
| | 1 H), 7.43 (d, J = 5.09 Hz, 1 H), 7.20 (d, J = 8.61 Hz, 2H), 4.62 (s, 2 H), |
| | 3.63 (q, J = 7.04 Hz, 2 H), 2.98-3.08 (m, 1 H), 2.81-2.89 (m, 1 H), 2.61-2.70 (m, 1 |
| | H), 1.96-2.05 (m, 1 H), 1.85 (dd, J = 12.91, 2.74 Hz, 1 H), 1.42-1.59 (m, 2 H), |
| | 1.28 (t, J = 7.04 Hz, 3 H), 1.00 (d, J = 6.65 Hz, 3 H) |
| 87 | (400 MHz, <cd3od>) δ ppm 1.01 (d, J = 6.65 Hz, 3 H) 1.22-1.42 (m, 1 H) |
| | 1.51 (dd, J = 9.39, 3.13 Hz, 1 H) 1.66-1.83 (m, 3 H) 1.87-2.25 (m, 4 H) |
| | 2.95-3.19 (m, 4 H) 3.54-3.67 (m, 2 H) 3.89-3.99 (m, 2 H) 4.57-4.71 (m, 1 H) 6.77 (d, |
| | J = 9.78 Hz, 2 H) 7.09 (d, J = 11.35 Hz, 1 H) 7.61 (d, J = 5.48 Hz, 1 H) 8.42 (d, |
| | J = 5.48 Hz, 1 H) 9.30 (s, 1 H) |
| 157 | (400 MHz, <cd3od>) δ ppm 1.01 (d, J = 6.26 Hz, 3 H) 1.20 (t, J = 7.24 Hz, 3 H) |
| | 1.29-1.42 (m, 1 H) 1.50 (ddd, J = 9.39, 6.46, 2.54 Hz, 1 H) 1.77 (q, J = 12.39 Hz, |
| | 1 H) 1.92 (dd, J = 13.11, 2.93 Hz, 1 H) 2.15-2.23 (m, 1 H) 2.95-3.05 (m, 1 |
| | H) 3.07-3.23 (m, 4 H) 7.65 (d, J = 5.48 Hz, 1 H) 7.81 (d, J = 8.61 Hz, 2 H) |
| | 8.06 (t, J = 8.80 Hz, 1 H) 8.44 (dd, J = 8.61, 3.91 Hz, 1 H) 8.49 (d, J = 5.87 Hz, 1 H) |
| | 9.07 (s, 1 H) |
| 132 | (400 MHz, <cd3od>) δ ppm 0.98 (d, J = 6.65 Hz, 3 H) 1.17 (qd, J = 12.19, 8.41 Hz, |
| | 2 H) 1.58-1.72 (m, 2 H) 1.87-1.97 (m, 1 H) 1.99-2.12 (m, 3 H) 2.20 (d, |
| | J = 11.74 Hz, 1 H) 2.39 (t, J = 8.02 Hz, 2 H) 3.10-3.26 (m, 2 H) 3.60 (t, J = 7.04 Hz, |
| | 2 H) 3.70 (t, J = 5.48 Hz, 2 H) 4.24 (t, J = 5.48 Hz, 2 H) 6.84 (d, J = 9.78 Hz, 2 |
| | H) 7.92 (d, J = 6.26 Hz, 1 H) 8.00 (t, J = 8.80 Hz, 1 H) 8.39 (dd, J = 8.61, 3.91 Hz, |
| | 1 H) 8.60 (d, J = 5.87 Hz, 1 H) 9.38 (s, 1 H) |
| 160 | (400 MHz, <cd3od>) δ ppm 0.97 (d, J = 6.26 Hz, 3 H) 1.05-1.21 (m, 3 H) |
| | 1.48-1.68 (m, 1 H) 1.81-2.23 (m, 4 H) 2.59 (td, J = 7.43, 2.35 Hz, 2 H) |
| | 2.67-2.81 (m, 2 H) 2.97-3.25 (m, 2 H) 6.48 (t, J = 1.76 Hz, 1 H) 7.25 (d, J = 9.78 Hz, 2 H) |
| | 7.61 (d, J = 5.48 Hz, 1 H) 8.00 (t, J = 8.80 Hz, 1 H) 8.38 (dd, J = 8.61, 3.91 Hz, 1 |
| | H) 8.48 (d, J = 5.48 Hz, 1 H) 9.03 (s, 1 H) |
| 86 | (400 MHz, <cd3od>) δ ppm 0.95 (d, J = 6.65 Hz, 3 H) 1.12 (q, J = 11.87 Hz, 2 H) |
| | 1.59 (q, J = 12.00 Hz, 2 H) 1.68-1.81 (m, 2 H) 1.90 (d, J = 12.91 Hz, 1 H) |
| | 1.97-2.10 (m, 3 H) 2.15 (d, J = 11.35 Hz, 1 H) 2.93-3.25 (m, 2 H) 3.60 (ddd, |
| | J = 11.64, 8.71, 2.74 Hz, 2 H) 3.86-4.04 (m, 2 H) 4.66 (dt, J = 8.02, 4.21 Hz, 1 |
| | H) 6.77 (d, J = 10.17 Hz, 2 H) 7.09 (d, J = 11.35 Hz, 1 H) 7.62 (d, J = 5.87 Hz, 1 |
| | H) 8.44 (d, J = 5.87 Hz, 1 H) 9.24 (s, 1 H) |
| 93 | (400 MHz, <cd3od>) δ ppm 0.84 (d, J = 6.65 Hz, 3 H) 1.36 (d, J = 5.87 Hz, 6 H) |
| | 1.75 (br. s., 1 H) 2.70 (t, J = 12.72 Hz, 1 H) 3.05-3.23 (m, 3 H) 3.76 (d, |
| | J = 12.91 Hz, 1 H) 3.95 (d, J = 10.56 Hz, 1 H) 4.69 (dt, J = 12.13, 6.06 Hz, 1 H) |
| | 6.74 (d, J = 10.17 Hz, 2 H) 7.45 (d, J = 6.26 Hz, 1 H) 7.98 (t, J = 8.61 Hz, 1 H) |
| | 8.36 (d, J = 6.65 Hz, 1 H) 8.39 (dd, J = 8.61, 3.91 Hz, 1 H) 9.20 (s, 1 H) |
| 103 | (400 MHz, <cd3od>) δ ppm 0.83 (d, J = 6.65 Hz, 2 H) 1.12-1.21 (m, 1 H) |
| | 1.26-1.37 (m, 1 H) 1.72 (br. s., 1 H) 2.58-2.71 (m, 1 H) 3.03-3.20 (m, 2 H) |
| | 3.69 (d, J = 12.91 Hz, 1 H) 3.87 (d, J = 10.96 Hz, 1 H) 7.07 (d, J = 10.17 Hz, 1 H) |
| | 7.42 (d, J = 6.65 Hz, 1 H) 8.01 (t, J = 8.80 Hz, 1 H) 8.35 (d, J = 6.65 Hz, 1 H) 8.42 (dd, |
| | J = 8.61, 3.91 Hz, 1 H) 9.23 (s, 1 H) |
| 133 | (400 MHz, <cd3od>) δ ppm 0.82 (dd, J = 6.65, 1.17 Hz, 6 H) 1.09-1.25 (m, 2 |
| | H) 1.28-1.40 (m, 1 H) 1.49 (dq, J = 12.72, 6.59 Hz, 1 H) 1.61 (q, J = 12.00 Hz, 1 |
| | H) 1.75-1.89 (m, 1 H) 1.95 (d, J = 12.91 Hz, 1 H) 2.02-2.23 (m, 3 H) |
| | 2.36-2.58 (m, 4 H) 3.01-3.13 (m, 1 H) 3.21 (ddt, J = 11.93, 7.92, 3.77, 3.77 Hz, 1 H) |
| | 7.34 (d, J = 9.78 Hz, 2 H) 7.65 (d, J = 5.48 Hz, 1 H) 8.01 (t, J = 8.61 Hz, 1 H) |
| | 8.39 (dd, J = 8.61, 3.91 Hz, 1 H) 8.50 (d, J = 5.48 Hz, 1 H) 9.05 (s, 1 H) |
| 110 | (400 MHz, <cd3od>) δ ppm 0.81 (dd, J = 7.83, 7.04 Hz, 6 H) 1.08-1.26 (m, 2 |
| | H) 1.34 (ddd, J = 11.54, 5.28, 2.35 Hz, 1 H) 1.43-1.53 (m, 1 H) 1.59-1.81 (m, |
| | 3 H) 1.97 (d, J = 12.52 Hz, 1 H) 2.02-2.13 (m, 3 H) 2.19 (d, J = 12.13 Hz, 1 H) |
| | 3.04-3.16 (m, 1 H) 3.19-3.26 (m, 1 H) 3.61 (ddd, J = 11.74, 8.80, 2.54 Hz, 2 |
| | H) 3.88-4.01 (m, 2 H) 4.62-4.75 (m, 1 H) 6.84 (d, J = 10.17 Hz, 2 H) 7.76 (d, |
| | J = 5.48 Hz, 1 H) 7.98 (t, J = 8.61 Hz, 1 H) 8.37 (dd, J = 8.61, 3.91 Hz, 1 H) |
| | 8.54 (d, J = 5.48 Hz, 1 H) 9.19 (s, 1 H) |
| 111 | (400 MHz, <cd3od>) δ ppm 0.81 (dd, J = 6.65, 2.74 Hz, 6 H) 1.17 (quin, |
| | J = 12.03 Hz, 2 H) 1.29-1.39 (m, 1 H) 1.47 (dt, J = 13.01, 6.60 Hz, 1 H) 1.63 (q, |
| | J = 12.13 Hz, 1 H) 1.96 (d, J = 13.30 Hz, 1 H) 2.08 (d, J = 12.13 Hz, 1 H) 2.18 (d, |
| | J = 11.74 Hz, 1 H) 3.01-3.14 (m, 1 H) 3.18-3.26 (m, 1 H) 4.76 (d, J = 6.65 Hz, |
| | 2 H) 4.94 (d, J = 7.04 Hz, 2 H) 7.52 (d, J = 9.39 Hz, 2 H) 7.69 (d, J = 5.48 Hz, 1 H) |
| | 8.02 (t, J = 8.61 Hz, 1 H) 8.41 (dd, J = 8.80, 4.11 Hz, 1 H) 8.52 (d, J = 5.87 Hz, 1 |
| | H) 9.08 (s, 1 H) |
| 104 | (400 MHz, <cd3od>) δ ppm 0.77 (d, J = 6.65 Hz, 3 H) 1.21 (t, J = 7.04 Hz, 3 H) |
| | 1.58-1.77 (m, 1 H) 2.56-2.73 (m, 1 H) 3.00-3.20 (m, 5 H) 3.71 (dt, J = 12.91, |
| | 3.33 Hz, 1 H) 3.83-3.96 (m, 1 H) 7.46 (d, J = 6.65 Hz, 1 H) 7.80 (d, J = 8.61 Hz, |
| | 2 H) 8.08 (t, J = 8.61 Hz, 1 H) 8.37 (d, J = 6.26 Hz, 1 H) 8.48 (dd, J = 8.61, 3.91 Hz, |
| | 1 H) 9.28 (s, 1 H) |
| 92 | (400 MHz, <cd3od>) δ ppm 0.30 (q, J = 4.96 Hz, 2 H) 0.52-0.62 (m, 2 H) |
| | 0.76 (d, J = 6.65 Hz, 3 H) 1.13-1.27 (m, 1 H) 1.67 (br. s., 1 H) 2.52-2.64 (m, 1 H) |
| | 2.96-3.14 (m, 3 H) 3.62 (d, J = 12.91 Hz, 1 H) 3.75-3.88 (m, 3 H) 6.68 (d, |
| | J = 10.17 Hz, 2 H) 7.35 (d, J = 6.65 Hz, 1 H) 7.91 (t, J = 8.80 Hz, 1 H) 8.28 (d, |
| | J = 6.26 Hz, 1 H) 8.32 (dd, J = 8.61, 3.91 Hz, 1 H) 9.18 (s, 1 H) |
| 169 | (400 MHz, <cd3od>) δ 9.62 (s, 1H), 8.66 (d, J = 5.87 Hz, 1H), 8.39 (dd, J = 3.52, |
| | 8.61 Hz, 1H), 8.08 (d, J = 6.26 Hz, 1H), 8.01 (t, J = 8.80 Hz, 1H), 6.77 (d, |
| | J = 11.74 Hz, 2H), 3.83-3.89 (m, 4H), 3.32-3.37 (m, 5H), 3.26 (t, J = 9.78 Hz, |
| | 1H), 3.08-3.18 (m, 1H), 2.24-2.34 (m, 1H), 1.91-2.07 (m, 2H), |
| | 1.63 (dtd, J = 3.33, 6.02, 9.29 Hz, 1H), 1.48 (q, J = 12.39 Hz, 1H), 1.06 (d, J = 6.65 Hz, |
| | 3H) |
| 94 | (400 MHz, <cd3od>) δ 9.04 (s, 1H), 8.34-8.43 (m, 2H), 8.01 (t, J = 8.80 Hz, |
| | 1H), 7.45 (d, J = 5.09 Hz, 1H), 7.17 (d, J = 9.00 Hz, 2H), 4.05-4.12 (m, 2H), |
| | 3.55-3.65 (m, 2H), 2.92-3.10 (m, 2H), 2.87 (t, J = 9.39 Hz, 1H), 2.69 (ddd, J = 4.11, |
| | 9.29, 11.64 Hz, 1H), 1.98-2.07 (m, 1H), 1.81-1.92 (m, 4H), |
| | 1.44-1.62 (m, 2H), 1.25-1.39 (m, 2H), 1.00 (d, J = 6.26 Hz, 3H) |
| 88 | (400 mHz, DMSO-_{d6}) δ 10.35 (s, 1H), 8.93 (m, 1H), 8.36-8.40 (m, 2H), |
| | 8.24 (dd, J = 8.8, 8.8, 1H), 7.98-8.08 (m, 2H), 7.45 (d, J = 9.2, 1H), 7.31 (d, J = 6.8, |
| | 1H), 5.68 (bs, 1H), 4.75-4.82 (m, 4H), 3.84 (d, J = 10.8, 1H), 3.73 (d, J = 12.4, |
| | 1H), 3.14 (s, 3H), 2.98-3.09 (m, 2H), 2.86-2.94 (bs, 1H), 2.61 (t, J = 12.8, |
| | 1H), 1.44-1.56 (m, 1H), 0.70 (d, J = 6.8, 3H). |
| 76 | (400 mHz, DMSO-_{d6}) δ 10.35 (s, 1H), 8.83 (m, 1H), 8.32-8.36 (m, 2H), |
| | 8.22 (dd, J = 8.8, 8.8, 1H), 7.98-8.08 (m, 3H), 7.36 (d, J = 9.6, 1H), 7.29 (d, J = 6.8, |
| | 1H), 5.83 (bs, 1H), 5.67 (bs, 1H), 3.88 (d, J = 10.8, 1H), 3.76 (d, J = 12.4, 1H), |
| | 3.07 (t, J = 9.6, 1H), 2.99 (d, J = 11.6, 1H), 2.88-2.92 (m, 1H), 2.62 (t, J = 12.0, |
| | 1H), 2.39-2.45 (m, 2H), 2.27-2.34 (m, 2H), 1.93-2.00 (m, 1H), 1.73-1.78 (m, |
| | 1H), 1.44-1.56 (m, 1H), 0.70 (d, J = 7.2, 3H). |
| 89 | (400 mHz, DMSO-d6) δ 10.34 (s, 1H), 8.93 (m, 1H), 8.36-8.39 (m, 2H), |
| | 8.25 (dd, J = 8.8, 8.8, 1H), 7.98-8.08 (m, 2H), 7.50 (d, J = 9.2, 1H), 7.31 (d, J = 6.4, |
| | 1H), 6.76 (bs, 1H), 5.68 (bs, 1H), 4.81 (d, J = 6.8, 2H), 4.73 (dd, J = 13.2, 6.4, |
| | 2H), 3.86 (d, J = 9.6, 1H), 3.74 (d, J = 12.4, 1H), 2.99-3.10 (m, 2H), 2.92 (bs, |
| | 1H), 2.61 (t, J = 12.4, 1H), 2.52 (s, 1H), 1.44-1.56 (m, 1H), 0.70 (d, J = 6.4, 3H). |
| 222 | (400 mHz, CDCl₃) δ 9.91 (s, 1H), 9.27 (s, 1H), 8.36 (d, J = 5.2, 1H), 7.16 (d, |
| | J = 5.6, 1H), 6.83-7.00 (m, 3H), 6.29 (bs, 2H), 2.78-2.81 (m 2H), 2.42 (s, 3H), |
| | 1.96-1.99 (m, 1H), 1.84-1.87 (m, 1H), 1.75-1.79 (m, 1H), 1.24-1.30 (m, 3H), |
| | 0.99 (q, J = 12.4, 1H), 0.87 (d, J = 8.0, 3H), 0.81 (q, J = 12.0, 1H). |
| 48 | (400 MHz, CD₃OD) δ 0.98 (d, H) 1.15 (qd, 2 H) 1.54-1.70 (m, 2 H) 1.93 (d, 1 |
| | H) 2.04 (d, 1 H) 2.17 (d, 1 H) 3.03-3.17 (m, 1 H) 3.19-3.26 (m, 1 H) |
| | 4.64-4.73 (m, 2 H) 5.04 (t, 2 H) 5.32-5.44 (m, 1 H) 6.68 (d, 2 H) 7.74 (d, 1 H) |
| | 7.99 (t, 1 H) 8.37 (dd, 1 H) 8.53 (d, 1 H) 9.14 (s, 1 H) |
| 63 | (400 MHz, CD₃OD) δ 0.98 (d, 3 H) 1.13 (quin, 2 H) 1.51-1.81 (m, 4 H) |
| | 1.85-2.25 (m, 4 H) 3.02-3.13 (m, 1 H) 3.22 (d, 1 H) 3.61 (ddd, 2 H) 3.90-4.00 (m, |
| | 2 H) 4.62-4.75 (m, 1 H) 6.83 (d, 2 H) 7.62 (d, 1 H) 7.97 (t, 1 H) 8.35 (dd, 1 H) |
| | 8.49 (d, 1 H) 9.01 (s, 1 H) |
| 68 | (400 MHz, CD₃OD) δ 9.03 (s, 1H), 8.42 (dd, 1H), 8.37 (d, 1H), 8.03 (t, 1H), |
| | 7.44 (d, 1H), 7.32 (d, 2H), 5.16 (dd, 2H), 4.78 (dt, 2H), 4.37-4.46 (m, 1H), |
| | 2.96 (tt, 1H), 2.81 (tt, 1H), 2.02 (d, 1H), 1.80-1.94 (m, 3H), 1.54 (ddd, 1H), |
| | 1.36 (q, 2H), 1.08 (q,), 0.84-0.95 (m, 4H) |
| 6 | (400 MHz, CD3OD) δ 0.72-0.83 (m, 1 H) 0.89 (d,, 3 H) 1.19 (s, 2 H) |
| | 1.43-1.52 (m, 2 H) 1.52-1.62 (m, 1 H) 1.70-1.81 (m, 1 H) 1.89 (q, 1 H) 2.37 (s, 3 |
| | H) 2.91-3.04 (m, 1 H) 3.68 (s, 1 H) 6.95 (d, 2 H) 7.52 (d, 1 H) 7.90 (t, 1 H) |
| | 8.29 (dd, 1 H) 8.39 (d, 1 H) 8.87 (s, 1 H) |
| 5 | (400 MHz, CD3OD) δ 0.99 (d, 1 H) 1.22-1.39 (m, 1 H) 1.43-1.61 (m, 2 H) |
| | 1.75-1.90 (m, 1 H) 1.93-2.06 (m, 1 H) 2.58-2.74 (m, 1 H) 2.84 (t, 1 H) |
| | 2.94-3.09 (m, 1 H) 3.89 (s, 3 H) 6.79 (d, 2 H) 7.41 (d, 1 H) 7.94 (t, 1 H) |
| | 8.25-8.41 (m, 2 H) 8.98 (s, 1 H) |
| 11 | (400 MHz, CDCl3) δ 9.92 (s, 1H), 9.37 (s, 1H), 8.36-8.42 (m, 2H), 7.74 (t, 1H), |
| | 7.19 (d, 1H), 6.63 (d, 2H), 3.89 (s, 3H), 2.76-2.89 (m, 2H), 1.96-2.03 (m, |
| | 1H), 1.86-1.93 (m, 1H), 1.81 (d, 1H), 1.58 (br. s., 3H), 1.27 (q, 1H), 1.03 (q, |
| | 1H), 0.91 (d, 3H), 0.82 (q, 1H) |
| 4 | (400 MHz, CD3OD) δ ppm 1.01 (d, 3 H) 1.33 (q, 1 H) 1.43-1.65 (m, 2 H) |
| | 1.86 (dd, 1 H) 2.02 (dd, 1 H) 2.48 (s, 3 H) 2.59-2.75 (m, 1 H) 2.86 (t, 1 H) |
| | 2.96-3.13 (m, 1 H) 7.07 (d, 2 H) 7.45 (d, 1 H) 8.00 (t, 1 H) 8.30-8.48 (m, 2 H) |
| | 8.98 (s, 1 H) |
| 8 | (400 MHz, CD3OD) δ 8.84 (s, 1H), 8.43 (d, 1H), 8.39 (dd, 1H), 7.97-8.04 (m, |
| | 1H), 7.46 (d, 1H), 7.06 (d, 2H), 3.21 (m, 1H), 3.04 (m, 1H), 2.49 (s, 3H), |
| | 2.12-2.19 (m, 1H), 2.02-2.09 (m, 1H), 1.92 (d, 1H), 1.64 m, 1H), 1.56 (q, 1H), 1.43 (d, |
| | 1H), 1.14 (t, 1H), 1.01 (d, 3H) |
| 224 | (400 MHz, CDCl3) δ 10.04 (s, 1H), 9.47 (s, 1H), 8.40 (dd, 1H), 8.36 (d, 1H), |
| | 7.77 (dd, 1H), 7.16 (d, 1H), 7.13 (d, 2H), 4.75 (s, 2H), 2.76-2.82 (m, 1H), |
| | 2.52-2.57 (m, 1H), 1.84-1.96 (m, 4H), 1.76-1.80 (m, 1H), 1.50-1.60 (m, 1H), |
| | 1.22-1.25 (m, 1H), 1.08-1.18 (m, 2H), 0.94 (d, 3H), 0.71-0.79 (m, 1H). |
| 212 | (400 MHz, CDCl3) δ 9.92 (s, 1H), 9.27 (s, 1H), 8.42 (dd, 1H), 8.39 (d, 1H), |
| | 7.77 (dd, 1H), 7.18 (d, 1H), 7.07-7.13 (m, 1H), 7.00-7.03 (m, 1H), 3.95 (s, |
| | 3H), 3.39 (s, 1H), 2.75-2.86 (m, 2H), 1.98 (d, 1H), 1.78-1.87 (m, 2H), |
| | 1.19-1.28 (m, 2 H), 0.98 (m, 1H), 0.88 (d, 3H), 0.75-0.84 (m, 1H). |
| 211 | (400 MHz, CDCl3) δ 9.99 (s, 1H), 9.60 (s, 1H), 8.44 (dd, 1H), 8.38 (d, 1H), |
| | 7.77 (dd, 1H), 7.62-7.68 (m, 1H), 7.15 (d, 1H), 7.07-7.11 (m, 1H), 4.70-4.80 (m, |
| | 1H), 4.55-4.60 (m, 1H), 2.75-2.81 (m, 1H), 2.56-2.64 (m, 1H), 1.81-2.09 (m, |
| | 5H), 1.52-1.58 (m, 1H), 1.22-1.31 (m, 2H), 1.01-1.07 (m, 1H), 0.97 (d, 3H), |
| | 0.68-0.75 (m, 1H). |
| 215 | (400 MHz, CD3OD) δ 0.80 (d, 3 H) 1.68 (d, 1 H) 2.52 (t, 1 H) 2.92-3.16 (m, 5 |
| | H) 3.54-3.67 (m, 2 H) 3.98 (s, 4 H) 7.30 (d, 1 H) 7.84 (d, 3 H) 8.09 (t, 1 H) |
| | 8.32 (d, 1 H) 8.50 (dd, 1 H) 9.43 (s, 1 H) |
| 12 | (400 MHz, CDCl3) δ 0.82-0.89 (m, 1 H) 0.92 (d, 3 H) 1.04 (q, 2 H) |
| | 1.21-1.36 (m, 2 H) 1.77-1.86 (m, 1 H) 1.87-1.96 (m, 1 H) 1.97-2.06 (m, 1 H) |
| | 2.75-2.92 (m, 1 H) 3.47 (s, 3 H) 3.76-3.82 (m, 2 H) 4.14-4.20 (m, 2 H) 6.66 (d, 2 |
| | H) 7.18 (d, 1 H) 7.73 (t, 1 H) 8.35-8.39 (m, 1 H) 8.40 (d, 2 H) 9.30-9.36 (m, 1 |
| | H) 9.88 (s, 1 H) |
| 214 | (400 MHz, CD3OD) δ 0.85 (d, 3 H) 1.70 (br. s., 1 H) 2.53 (t, 1 H) |
| | 2.89-3.00 (m, 1 H) 3.10 (d, 2 H) 3.58 (d, 1 H) 3.95 (s, 3 H) 7.13 (t, 1 H) 7.25-7.37 (m, 2 |
| | H) 8.04 (t, 1 H) 8.31 (d, 1 H) 8.45 (dd, 1 H) 9.43 (s, 1 H) |
| 22 | (400 MHz, CD3OD) δ 0.87 (d, 3 H) 1.70-1.87 (m, 1 H) 2.70 (dd, 1 H) |
| | 3.10-3.23 (m, 3 H) 3.46 (s, 2 H) 3.70-3.77 (m, 1 H) 3.78-3.83 (m, 2 H) |
| | 3.89-3.95 (m, 1 H) 4.22-4.27 (m, 2 H) 6.80-6.88 (m, 2 H) 7.48 (d, 1 H) 8.03 (t, 1 H) |
| | 8.39 (d, 1 H) 8.44 (dd, 1 H) 9.30 (s, 1 H) |
| 23 | (400 MHz, CD3OD) δ 0.87 (d, 3 H) 1.78 (br. s., 1 H) 2.61 (s, 3 H) 2.70 (m, 1 H) |
| | 3.17 (m, 3 H) 3.70-3.79 (m, 1 H) 3.93 (d, 2 H) 7.12 (d, 2 H) 7.47 (d, 1 H) |
| | 8.05 (t, 1 H) 8.39 (d, 1 H) 8.46 (m, 1 H) 9.29 (s, 1 H) |
| 24 | (400 MHz, CD3OD) δ 1.08 (d, 3 H) 1.43 (m, 1 H) 1.53-1.68 (m, 1 H) |
| | 1.70-1.86 (m, 1 H) 1.92-2.01 (m, 1 H) 2.14-2.27 (m, 1 H) 3.06 (m, 1 H) |
| | 3.13-3.24 (m, 1 H) 3.46 (s, 3 H) 3.77-3.83 (m, 2 H) 4.12 (q, 1 H) 4.23-4.27 (m, 2 |
| | H) 6.86 (d, 2 H) 7.64 (d, 1 H) 8.01 (t, 1 H) 8.39 (dd, 1 H) 8.50 (d, 1 H) 9.11 (s, |
| | 1 H) |
| 235 | (400 MHz, CD3OD) δ 9.21 (s, 1H), 8.45 (dd, 1H), 8.39 (d, 1H), 8.04 (t, 1H), |
| | 7.49 (d, 2H), 4.00 (dd, 1H), 3.82 (m, 1H), 3.13-3.24 (m, 3H), 2.63-2.85 (m, |
| | 4H), 1.67-1.81 (m, 1H), 1.33 (t, 3H), 0.84 (d, 3H) |
| 36 | (400 MHz, CD3OD) δ 9.19 (s, 1 H) 8.54 (d, 1 H) 8.42 (dd, 1 H) 8.04 (t, 1 H) |
| | 7.73 (d, 1 H) 7.13 (d, 2 H) 3.13-3.29 (m, 2 H) 3.01-3.13 (m, 1 H) |
| | 2.16-2.30 (m, 1 H) 1.92-2.04 (m, 1 H) 1.83 (q, 1 H) 1.51-1.70 (m, 1 H) 1.42 (q, 1 H) |
| | 1.06 (d, 3 H) |
| 217 | (400 MHz, CD3OD) δ 9.33 (s, 1 H) 8.50 (dd, 1 H) 8.37 (d, 1 H) 8.09 (t, 1 H) |
| | 7.32-7.45 (m, 3 H) 3.73-3.85 (m, 1 H) 3.56-3.66 (m, 1 H) 3.02-3.23 (m, 3 |
| | H) 2.56-2.72 (m, 1 H) 1.66-1.84 (m, 1 H) 0.87 (d, 3 H) |
| 218 | (400 MHz, CD3OD) δ 9.28 (s, 1 H) 8.49 (dd, 1 H) 8.39 (d, 1 H) 8.08 (t, 1 H) |
| | 7.47 (d, 1 H) 7.15 (t, 2 H) 3.84-3.97 (m, 1 H) 3.64-3.80 (m, 1 H) |
| | 3.09-3.25 (m, 3 H) 2.60-2.76 (m, 1 H) 1.63-1.86 (m, 1 H) 0.86 (d, 3 H) |
| 27 | (400 MHz, CD3OD) δ 8.98 (s, 1 H) 8.46 (dd, 1 H) 8.38 (d, 1 H) 8.08 (t, H) |
| | 7.64 (d, 2 H) 7.45 (d, 1 H) 3.00-3.11 (m, 1 H) 2.95 (s, 3 H) 2.88 (t, H) |
| | 2.61-2.73 (m, 1 H) 1.98-2.10 (m, 1 H) 1.80-1.94 (m, 1 H) 1.56 (q, 1 H) |
| | 1.41-1.50 (m, 1 H) 1.34 (m, 1 H) 1.02 (d, 3 H) |
| 28 | (400 MHz, CD3OD) δ 8.98 (s, 1 H) 8.46 (dd, 1 H) 8.38 (d, 1 H) 8.08 (t, 1 H) |
| | 7.64 (d, 2 H) 7.45 (d, 1 H) 3.00-3.11 (m, 1 H) 2.96 (s, 3 H) 2.88 (t, 1 H) |
| | 2.61-2.74 (m, 1 H) 1.98-2.11 (m, 1 H) 1.81-1.94 (m, 1 H) 1.42-1.66 (m, 2 H) |
| | 1.26-1.40 (m, 1 H) 1.02 (d, 3 H) |
| 14 | (400 MHz, CD3OD) δ 8.94 (s, 1 H) 8.43-8.55 (m, 2 H) 8.12 (t, 1 H) 7.90 (d, 2 |
| | H) 7.57 (d, 1 H) 3.28 (s, 3 H) 3.10-3.22 (m, 2 H) 2.98-3.09 (m, 1 H) |
| | 2.13-2.27 (m, 1 H) 1.89-2.02 (m, 1 H) 1.78 (q, 1 H) 1.48-1.63 (m, 1 H) 1.39 (q, 1 |
| | H) 1.06 (d, 3 H) |
| 37 | 400 MHz, CD3OD) δ 9.24 (s, 1 H), 8.48-8.43 (m, 1 H), 8.42-8.37 (m, 1 H), |
| | 8.08-8.01 (m, 1 H), 7.51-7.46 (m, 1 H), 7.06 (d, 2 H), 4.04-3.91 (m, 1 H), |
| | 3.85-3.73 (m, 1 H), 3.18 (s, 3 H), 2.77-2.64 (m, 1 H), 2.48 (s, 3 H), |
| | 1.82-1.67 (m, 1 H), 0.85 (d, 3 H) |
| 15 | 400 mHz, DMSOd-6) δ 10.30 (s, 1 H), 8.78-9.03 (m, 1 H), 8.26-8.40 (m, 2 |
| | H), 8.17 (t, 1 H), 7.93-8.08 (m, 3 H), 7.27 (d, 1 H), 6.93 (d, 2 H), 5.67 (br s, 1 |
| | H), 3.85 (s, 3 H), 3.76 (br s, 2 H), 2.82-3.12 (m, 4 H), 1.44-1.58 (m, 1 H), |
| | 0.70 (d, 3 H). |
| 229 | (400 MHz, CDCl3) δ 9.98 (s, 1H), 9.41 (s, 1H), 8.38 (d, 1H), 8.36 (d, 1H), |
| | 7.73 (dd, 1H), 7.17 (d, 1H), 6.69 (d, 2H), 4.18-4.21 (m, 2H), 3.98-34.00 (m, 2H), |
| | 2.73-2.85 (m, 2H), 1.91-2.17 (m, 7 H), 1.79 (d, 1H), 1.52-1.59 (m, 1H), |
| | 1.19-1.28 (m, 1H), 1.04-1.13 (m, 1H), 0.93 (d, 3H), 0.77-0.86 (m, 1H). |
| 21 | (400 mHz, DMSOd-6) δ 10.35 (s, 1H), 8.93 (s, 1H), 8.39 (d, 1H), 8.33 (dd, |
| | 1H), 8.20 (dd, 1H), 8.09 (m, 3H), 7.32 (d, 1H), 6.95 (d, 2H), 5.72 (bs, 1H), |
| | 4.13 (t, 2H), 3.99 (d, 1H), 3.75 (d, 2H), 3.56 (bs, 4H), 3.02-3.13 (m, 2H), 2.95 (bs, |
| | 1H), 2.59-2.65 (m, 1H), 1.50-1.60 (m, 1H), 0.74 (d, 3H). |
| 26 | (400 mHz, DMSOd-6) δ 10.21 (s, 1H), 9.42 (s, 1H), 8.44 (dd, 1H), |
| | 8.26-8.30 (m, 2H), 7.67 (d, 1H), 7.12 (d, 1H), 4.83 (bs, 1H), 3.13 (m, 1H), 3.05 (m, 1H), |
| | 2.93 (s, 3H), 2.60-2.71 (m, 2H), 2.53-2.59 (m, 1H), 2.46-2.53 (m, 2H), |
| | 2.33 (m, 1H), 1.44 (m, 1H), 0.69 (d, 3H). |
| 31 | (400 mHz, DMSOd-6) δ 10.39 (s, 1H), 8.69 (s, 1H), 8.36-8.40 (m, 2H), |
| | 8.24 (dd, 1H), 8.04 (m, 2H), 7.40-7.46 (m, 1H), 7.30 (d, 1H), 6.95 (td, 1H), 5.67 (bs, |
| | 1H), 4.14 (t, 2H), 3.88 (d, 1H), 3.75 (t, 2H), 3.44 (bs, 1H), 2.99-3.11 (m, 2H), |
| | 2.90 (bs, 1H), 2.52-2.67 (M, 1H), 1.46-1.54 (m, 1H), 0.73 (d, 3H). |
| 25 | (400 mHz, DMSOd-6) δ 10.19 (s, 1H), 9.41 (s, 1H), 8.44 (dd, 1H), |
| | 8.26-8.30 (m, 2H), 7.68 (d, 2H), 7.13 (d, 1H), 4.97 (bs, 1H), 3.15 (m, 1H), 3.07 (m, 1H), |
| | 2.93 (s, 3H), 2.60-2.71 (m, 2H), 2.53-2.59 (m, 2H), 2.45 (m, 1H), 2.32 (m, 1H), |
| | 1.44 (m, 1H), 0.67 (d, 3H). |
| 16 | (400 MHz, CD3OD) δ 0.71-0.89 (m, 3 H) 1.60-1.78 (m, 1 H) 2.57-2.74 (m, |
| | 1 H) 3.05-3.23 (m, 3 H) 3.65-3.81 (m, 1 H) 3.87-4.00 (m, 1 H) |
| | 7.42-7.55 (m, 1 H) 7.77-7.94 (m, 2 H) 8.07-8.19 (m, 1 H) 8.40 (d, 1 H) 8.54 (dd, 1 H) |
| | 9.29 (s, 1 H) |
| 40 | (400 MHz, CD3OD) δ 1.05 (d, 3 H) 1.40 (q, 1 H) 1.55 (m, 1 H) 1.79 (q, 1 H) |
| | 1.96 (dd, 1 H) 2.21 (dd, 1 H) 3.03 (td, 1 H) 3.10-3.25 (m, 2 H) 3.47 (s, 3 H) |
| | 4.57 (s, 2 H) 7.21 (d, 2 H) 7.71 (d, 1 H) 8.04 (t, 1 H) 8.42 (dd, 1 H) 8.53 (d, 1 |
| | H) 9.14 (s, 1 H) |
| 41 | (400 MHz, CD3OD) δ 0.99 (d, 3 H) 1.16 (qd, 2 H) 1.57-1.73 (m, 2 H) 1.94 (d, |
| | 1 H) 2.06 (d, 1 H) 2.19 (d, 1 H) 3.07-3.28 (m, 2 H) 3.47 (s, 3 H) 4.57 (s, 2 H) |
| | 7.20 (d, 2 H) 7.78 (d, 1 H) 8.04 (t, 1 H) 8.43 (dd, 1 H) 8.56 (d, 1 H) 9.19 (s, 1 |
| | H) |
| 19 | (400 MHz, CD3OD) δ 9.03 (s, 1 H) 8.52 (d, 1 H) 8.41 (dd, 1 H) 8.03 (t, H) |
| | 7.67 (d, 1 H) 7.11 (d, 2 H) 3.76-3.86 (m, 1 H) 3.05-3.14 (m, 1 H) 2.60 (s, 3 H) |
| | 2.03 (q, 1 H) 1.83-1.94 (m, 1 H) 1.55-1.74 (m, 3 H) 1.37-1.51 (m, 1 H) |
| | 1.01 (d, 3 H) |
| 32 | (400 MHz, CD3OD) δ 1.07 (d, 3 H) 1.43 (q, 1 H) 1.54-1.68 (m, 1 H) 1.82 (q, 1 |
| | H) 1.93-2.03 (m, 1 H) 2.18-2.29 (m, 1 H) 3.01-3.12 (m, 1 H) 3.13-3.29 (m, |
| | 2 H) 3.93 (t, 2 H) 4.18 (t, 2 H) 6.87 (d, 2 H) 7.76 (d, 1 H) 8.02 (t, 1 H) 8.40 (dd, |
| | 1 H) 8.54 (d, 1 H) 9.24 (s, 1 H). |
| 35 | (400 MHz, CD3OD) δ 9.27 (s, 1H), 8.46 (dd, 1H), 8.40 (d, 1H), 8.05 (t, 1H), |
| | 7.51 (d, 1H), 7.13 (d, 2H), 3.97 (d, 1H), 3.89 (t, 2H), 3.76-3.83 (m, 1H), |
| | 3.15-3.22 (m, 3H), 2.95 (t, 2H), 2.72 (dd, 1H), 1.76 (d, 1H), 0.85 (d, 3H) |
| 234 | (400 MHz, CD3OD) δ 9.52 (s, 1H), 8.44 (dd, 1H), 8.26 (d, 1H), 8.02 (t, 1H), |
| | 7.26 (d, 2H), 7.21 (d, 1H), 3.72-3.81 (m, 1H), 3.15-3.22 (m, 1H), 3.02 (q, |
| | 2H), 2.81-2.88 (m, 1H), 2.76 (dt, 1H), 2.59-2.66 (m, 1H), 2.46 (t, 1H), |
| | 1.59-1.70 (m, 1H), 1.27-1.33 (m, 1H), 0.85 (d, 3H) |
| 233 | (400 MHz, CD3OD) δ 9.53 (s, 1H), 8.45 (dd, 1H), 8.27 (d, 1H), 8.03 (t, 1H), |
| | 7.27 (d, 2H), 7.24 (d, 1H), 3.76-3.79 (m, 2H), 3.17-3.23 (m, 1H), 3.06 (q, |
| | 1H), 2.93-2.99 (m, 1H), 2.89 (dt, 1H), 2.68-2.76 (m, 1H), 2.50 (t, 1H), |
| | 1.70 (td, 1H), 1.25-1.37 (m, 1H), 0.87 (d, 3H) |
| 38 | (400 MHz, CD3OD) δ 9.20 (d, 1 H), 8.46 (dd, 1 H), 8.39 (dd, 1 H), 8.05 (t, 1 H), |
| | 7.48 (d, 1 H), 7.19 (d, 2 H), 4.57 (s, 2 H), 3.98 (d, 1 H), 3.76-3.85 (m, 1 H), |
| | 3.47 (s, 3 H), 3.13-3.20 (m, 4 H), 2.64-2.77 (m, 2 H), 0.84 (d, 3 H) |
| 39 | (400 MHz, CD3OD) δ 9.03 (s, 1 H), 8.47 (d, 1 H), 8.40 (dd, 1 H), 8.01 (t, 1 H), |
| | 7.59 (d, 1 H), 7.09 (d, 2 H), 3.10-3.21 (m, 2 H), 3.00-3.08 (m, 1 H), 2.78 (q, 2 |
| | H), 2.16-2.23 (m, 1 H), 1.95 (dd, 1 H), 1.76 (q, 1 H), 1.52-1.61 (m, 1 H), |
| | 1.36-1.44 (m, 1 H), 1.31 (t, 3 H), 1.05 (d, 3 H) |
| 20 | (400 MHz, CD3OD) δ 0.87 (d, 3 H) 1.46 (t, 3 H) 1.73-1.86 (m, 1 H) |
| | 2.59-2.76 (m, 1 H) 3.05-3.23 (m, 3 H) 3.67-3.76 (m, 1 H) 3.90 (d, 1 H) |
| | 4.11-4.20 (m, 2 H) 6.76-6.83 (m, 2 H) 7.46 (d, 1 H) 8.02 (t, 1 H) 8.39 (d, 1 H) 8.43 (dd, 1 |
| | H) 9.31 (s, 1 H) |

### Pim1, Pim2, Pim3 AlphaScreen Assays

Pim 1, Pim 2 & Pim 3 AlphaScreen assays using high ATP (11-125×ATP Km) were used to determine the biochemical activity of the inhibitors. The activity of Pim 1, Pim 2, & Pim 3 is measured using a homogeneous bead based system quantifying the amount of phosphorylated peptide substrate resulting from kinase-catalyzed phosphoryl transfer to a peptide substrate. Compounds to be tested are dissolved in 100% DMSO and directly distributed to a white 384-well plate at 0.25 μl per well. To start the reaction, 5 μl of 100 nM Bad peptide (Biotin-AGAGRSRHSSYPAGT-OH) and ATP (concentrations described below) in assay buffer (50 mM Hepes, pH=7.5, 5 mM MgCl₂, 0.05% BSA, 0.01% Tween-20, 1 mM DTT) is added to each well. This is followed by the addition of 5 μl/well of Pim 1, Pim 2 or Pim 3 kinase in assay buffer (concentrations described below). Final assay concentrations (described below) are in 2.5% DMSO. The reactions are performed for ˜2 hours, then stopped by the addition of 10 μl of 0.75 μg/ml anti-phospho Ser/Thr antibody (Cell Signaling), 10 μg/ml Protein A AlphaScreen beads (Perkin Elmer), and 10 μg/ml streptavidin coated AlphaScreen beads in stop/detection buffer (50 mM EDTA, 95 mM Tris, pH=7.5, 0.01% Tween-20). The stopped reactions are incubated overnight in the dark. The phosphorylated peptide is detected via an oxygen anion initiated chemiluminescence/fluorescence cascade using the Envision plate reader (Perkin Elmer).

AlphaScreen Assay Conditions

| | | b-BAD | | ATP Km |
|---|---|---|---|---|
| Enzyme | Enzyme conc. | peptide conc. | ATP conc. | (app) |
| source | (nM) | (nM) | (uM) | (uM) |
| Pim 1 (INV) | 0.0025 | 50 | 2800 | 246 |
| Pim 2 (INV) | 0.01 | 50 | 500 | 4 |
| Pim 3 (NVS) | 0.005 | 50 | 2500 | 50 |

Compounds of the foregoing examples were tested by the Pim 1, Pim 2 & Pim 3 AlphaScreen assays and found to exhibit an IC₅₀ values as shown in Table 4 below. IC₅₀, the half maximal inhibitory concentration, represents the concentration of test compound that is required for 50% inhibition of its target in vitro.

### Cell Proliferation Assay

KMS11 (human myeloma cell line), were cultured in IMDM supplemented with 10% FBS, sodium pyruvate and antibiotics. Cells were plated in the same medium at a density of 2000 cells per well into 96 well tissue culture plates, with outside wells vacant, on the day of assay.

Test compounds supplied in DMSO were diluted into DMSO at 500 times the desired final concentrations before dilution into culture media to 2 times final concentrations. Equal volumes of 2× compounds were added to the cells in 96 well plates and incubated at 37° C. for 3 days.

After 3 days plates were equilibrated to room temperature and equal volume of CellTiter-Glow Reagent (Promega) was added to the culture wells. The plates were agitated briefly and luminescent signal was measured with luminometer. The percent inhibition of the signal seen in cells treated with DMSO alone vs. cells treated with control compound was calculated and used to determine EC₅₀ values (i.e., the concentration of a test compound that is required to obtain 50% of the maximum effect in the cells) for tested compounds, as shown in Table 4.

Using the procedures of the Pim1, Pim2, Pim3 AlphaScreen Assays the IC₅₀ concentrations of compounds of the previous examples were determined as shown in the following 4.

Using the procedures of Cell Proliferation Assay, the EC₅₀ concentrations of compounds of the examples were determined in KMS11 cells as shown in Table 4.

TABLE 4

| | Pim1 | Pim2 | Pim3 | KMS11-luc |
|---|---|---|---|---|
| EX# | IC50 μM | IC50 μM | IC50 μM | EC50 μM |
| 1 | 0.00037 | 0.02857 | 0.04483 | 0.383 |
| 2 | 0.00001 | 0.00299 | 0.00057 | |
| 3 | 0.00003 | 0.00189 | 0.00055 | 0.053 |
| 4 | 0.00004 | 0.00391 | 0.00079 | 0.070 |
| 5 | 0.00006 | 0.00637 | 0.00111 | 0.062 |
| 6 | 0.00003 | 0.00349 | 0.00104 | 0.041 |
| 7 | 0.00006 | 0.00624 | 0.00120 | |
| 8 | 0.00003 | 0.00231 | 0.00071 | 0.046 |
| 9 | 0.00004 | 0.00397 | 0.00109 | 0.072 |
| 10 | 0.00003 | 0.00496 | 0.00095 | 0.126 |
| 11 | 0.00003 | 0.00154 | 0.00088 | 0.071 |
| 12 | 0.00004 | 0.00291 | 0.00139 | 0.120 |
| 13 | 0.00004 | 0.00936 | 0.00127 | 0.187 |
| 14 | 0.00082 | 0.03734 | 0.05404 | 1.437 |
| 15 | 0.00002 | 0.00114 | 0.00035 | 0.021 |
| 16 | 0.00008 | 0.00207 | 0.00266 | 0.037 |
| 17 | 0.00003 | 0.00192 | 0.00035 | 0.013 |
| 18 | 0.00013 | 0.03228 | 0.00293 | 0.201 |
| 19 | 0.00008 | 0.00842 | 0.00323 | 0.074 |
| 20 | 0.00002 | 0.00097 | 0.00029 | 0.007 |
| 21 | 0.00002 | 0.00119 | 0.00034 | 0.013 |
| 22 | 0.00003 | 0.00150 | 0.00045 | 0.010 |
| 23 | 0.00002 | 0.00096 | 0.00032 | 0.029 |
| 24 | 0.00007 | 0.00557 | 0.00246 | 0.072 |
| 25 | 0.00003 | 0.00094 | 0.00037 | 0.011 |
| 26 | 0.00005 | 0.00188 | 0.00221 | 0.042 |
| 27 | 0.00019 | 0.00315 | 0.00375 | 0.090 |
| 28 | 0.00045 | 0.02218 | 0.03488 | 3.876 |
| 29 | 0.00004 | 0.00233 | 0.00108 | 0.064 |
| 30 | 0.00007 | 0.01136 | 0.00225 | 0.186 |
| 31 | | 0.00136 | | 0.012 |
| 32 | 0.00006 | 0.00476 | 0.00190 | 0.052 |
| 33 | 0.00002 | 0.00152 | 0.00100 | 0.013 |
| 34 | 0.00006 | 0.00290 | 0.00163 | 0.029 |
| 35 | 0.00003 | 0.00109 | 0.00036 | 0.007 |
| 36 | 0.00008 | 0.00671 | 0.00281 | 0.107 |
| 37 | 0.00002 | 0.00094 | 0.00028 | 0.013 |
| 38 | 0.00003 | 0.00135 | 0.00047 | 0.010 |
| 39 | 0.00004 | 0.00218 | 0.00118 | 0.055 |
| 40 | 0.00008 | 0.00398 | 0.00263 | 0.068 |
| 41 | 0.00004 | 0.00278 | 0.00214 | 0.022 |
| 42 | 0.00003 | 0.00277 | 0.00080 | 0.139 |
| 43 | 0.00014 | 0.01531 | 0.01145 | 0.043 |
| 44 | 0.00001 | 0.00203 | 0.00077 | 0.018 |
| 45 | 0.00003 | 0.00315 | 0.00113 | 0.049 |
| 46 | 0.00002 | 0.00277 | 0.00085 | 0.084 |
| 47 | 0.00004 | 0.00633 | 0.00132 | 0.097 |
| 48 | 0.00008 | 0.00416 | 0.00024 | 0.084 |
| 49 | 0.00003 | 0.00267 | 0.00186 | 0.053 |
| 50 | 0.00002 | 0.00328 | 0.00072 | 0.013 |
| 51 | 0.00003 | 0.00434 | 0.00102 | 0.030 |
| 52 | 0.00005 | 0.00479 | 0.00286 | 0.055 |
| 53 | 0.00005 | 0.00256 | 0.00405 | 0.032 |
| 54 | 0.00008 | 0.00473 | 0.00319 | 0.095 |
| 55 | 0.00002 | 0.00242 | 0.00143 | 0.024 |
| 56 | 0.00004 | 0.00428 | 0.00175 | 0.031 |
| 57 | 0.00005 | 0.00569 | 0.00361 | 0.042 |
| 58 | 0.00018 | 0.01134 | 0.00712 | 0.042 |
| 59 | 0.00002 | 0.00277 | 0.00045 | 0.028 |
| 60 | 0.00004 | 0.00233 | 0.00169 | 0.015 |
| 61 | 0.00002 | 0.00100 | 0.00052 | 0.047 |
| 62 | 0.00001 | 0.00103 | 0.00035 | 0.052 |
| 63 | 0.00001 | 0.00204 | 0.00032 | 0.076 |
| 64 | 0.00001 | 0.00196 | 0.00051 | 0.037 |
| 65 | 0.00001 | 0.00384 | 0.00101 | 0.083 |
| 66 | 0.00004 | 0.00175 | 0.00181 | 0.011 |
| 67 | 0.00002 | 0.00169 | 0.00094 | 0.023 |
| 68 | 0.00001 | 0.00160 | 0.00113 | 0.025 |
| 69 | 0.00003 | 0.00238 | 0.00089 | 0.060 |
| 70 | 0.00004 | 0.00401 | 0.00115 | 0.068 |
| 71 | 0.00002 | 0.00221 | 0.00097 | 0.048 |
| 72 | 0.00003 | 0.00268 | 0.00128 | 0.139 |
| 73 | 0.00002 | 0.00127 | 0.00079 | 0.021 |
| 74 | 0.00003 | 0.00123 | 0.00092 | 0.015 |
| 75 | 0.00002 | 0.00155 | 0.00062 | 0.044 |
| 76 | 0.00001 | 0.00071 | 0.00022 | 0.009 |
| 77 | 0.00002 | 0.00184 | 0.00112 | 0.048 |
| 78 | 0.00002 | 0.00123 | 0.00056 | 0.022 |
| 79 | 0.00001 | 0.00108 | 0.00024 | 0.012 |
| 80 | 0.00002 | 0.00216 | 0.00058 | 0.024 |
| 81 | 0.00001 | 0.00042 | 0.00021 | 0.014 |
| 82 | 0.00001 | 0.00105 | 0.00052 | 0.030 |
| 83 | 0.00004 | 0.00243 | 0.00133 | 0.166 |
| 84 | 0.00002 | 0.00148 | 0.00052 | 0.029 |
| 85 | 0.00005 | 0.00220 | 0.00134 | 0.049 |
| 86 | 0.00001 | 0.00091 | 0.00022 | 0.017 |
| 87 | | 0.00146 | | 0.031 |
| 88 | 0.00002 | 0.00118 | 0.00069 | 0.017 |
| 89 | 0.00001 | 0.00067 | 0.00026 | 0.021 |
| 90 | 0.00001 | 0.00072 | 0.00071 | 0.019 |
| 91 | 0.00002 | 0.00144 | 0.00093 | 0.025 |
| 92 | 0.00001 | 0.00051 | 0.00009 | 0.034 |
| 93 | 0.00001 | 0.00078 | 0.00010 | 0.020 |
| 94 | 0.00001 | 0.00129 | 0.00039 | 0.024 |
| 96 | 0.00001 | 0.00116 | 0.00024 | 0.076 |
| 97 | 0.00002 | 0.00155 | 0.00037 | 0.069 |
| 98 | 0.00003 | 0.00138 | 0.00053 | 0.040 |
| 99 | 0.00001 | 0.00057 | 0.00012 | 0.010 |
| 100 | 0.00001 | 0.00053 | 0.00013 | 0.012 |
| 101 | 0.00002 | 0.00130 | 0.00036 | 0.030 |
| 102 | 0.00001 | 0.00075 | 0.00013 | 0.015 |
| 103 | 0.00002 | 0.00068 | 0.00037 | 0.021 |
| 104 | 0.00002 | 0.00095 | 0.00045 | 0.019 |
| 105 | 0.00001 | 0.00064 | 0.00017 | 0.096 |
| 106 | 0.00001 | 0.00052 | 0.00013 | 0.154 |
| 107 | 0.00002 | 0.00103 | 0.00031 | 0.157 |
| 108 | 0.00004 | 0.00293 | 0.00119 | 0.906 |
| 109 | 0.00001 | 0.00038 | 0.00014 | 0.028 |
| 110 | 0.00001 | 0.00095 | 0.00025 | |
| 111 | 0.00001 | 0.00076 | 0.00061 | |
| 112 | 0.00001 | 0.00048 | 0.00021 | |
| 113 | 0.00002 | 0.00151 | 0.00073 | |
| 114 | 0.00004 | 0.00137 | 0.00129 | |
| 115 | 0.00001 | 0.00066 | 0.00012 | |
| 116 | 0.00001 | 0.00058 | 0.00020 | |
| 117 | 0.00001 | 0.00065 | 0.00016 | |
| 118 | 0.00001 | 0.00108 | 0.00029 | |
| 119 | 0.00001 | 0.00136 | 0.00042 | 0.017 |
| 120 | 0.00003 | 0.00378 | 0.00051 | 0.032 |
| 121 | 0.00001 | 0.00156 | 0.00077 | 0.027 |
| 122 | 0.00002 | 0.00271 | 0.00157 | 0.083 |
| 123 | 0.00002 | 0.00080 | 0.00058 | 0.059 |
| 124 | 0.00006 | 0.00718 | 0.00172 | 0.119 |
| 125 | 0.00003 | 0.00182 | 0.00096 | 0.100 |
| 126 | 0.00002 | 0.00133 | 0.00115 | 0.064 |
| 127 | 0.00004 | 0.00210 | 0.00160 | 0.059 |
| 129 | 0.00003 | 0.00344 | 0.00229 | 0.078 |
| 130 | 0.00002 | 0.00136 | 0.00042 | 0.024 |
| 131 | 0.00001 | 0.00131 | 0.00018 | 0.025 |
| 132 | 0.00002 | 0.00478 | 0.00110 | 0.206 |
| 133 | 0.00002 | 0.00177 | 0.00070 | 0.031 |
| 134 | 0.00013 | 0.00957 | 0.00489 | 0.228 |
| 135 | 0.00005 | 0.00454 | 0.00445 | 0.583 |
| 136 | 0.00008 | 0.00559 | 0.00480 | 0.225 |
| 137 | 0.00008 | 0.01034 | 0.00690 | 0.549 |
| 138 | 0.00008 | 0.00330 | 0.00239 | 0.324 |
| 139 | 0.00001 | 0.00063 | 0.00036 | 0.153 |
| 140 | 0.00004 | 0.00605 | 0.00171 | 0.863 |
| 141 | 0.00045 | 0.03743 | 0.01599 | 2.507 |
| 142 | 0.00012 | 0.02838 | 0.01775 | 1.643 |
| 143 | 0.00005 | 0.00942 | 0.00077 | 0.197 |
| 144 | | 0.00261 | | 0.055 |
| 145 | 0.00003 | 0.00350 | 0.00199 | 0.244 |
| 146 | 0.00009 | 0.00558 | 0.00634 | 0.526 |
| 147 | 0.00002 | 0.00277 | 0.00103 | 0.146 |
| 148 | 0.00005 | 0.00328 | 0.00147 | 0.185 |
| 149 | 0.00003 | 0.00296 | 0.00172 | 0.148 |
| 150 | 0.00004 | 0.02017 | 0.00266 | 0.324 |
| 151 | 0.00007 | 0.02430 | 0.00223 | 0.340 |
| 152 | 0.00004 | 0.01200 | 0.00253 | 0.211 |
| 153 | 0.00006 | 0.01673 | 0.00610 | 0.366 |
| 154 | 0.00010 | 0.03205 | 0.00522 | 0.492 |
| 155 | 0.00009 | 0.01441 | 0.00261 | 0.264 |
| 156 | 0.00005 | 0.01816 | 0.00438 | 0.436 |
| 157 | 0.00011 | 0.00796 | 0.00485 | 0.068 |
| 158 | 0.00003 | 0.00334 | 0.00166 | 0.064 |
| 159 | 0.00006 | 0.00478 | 0.00086 | 0.097 |
| 160 | 0.00002 | 0.00374 | 0.00144 | 0.285 |
| 161 | 0.00002 | 0.00143 | 0.00097 | 0.034 |
| 162 | 0.00002 | 0.00845 | 0.00163 | 0.122 |
| 163 | 0.00001 | 0.00093 | 0.00017 | 0.033 |
| 164 | 0.00002 | 0.00213 | 0.00204 | 0.088 |
| 165 | 0.00004 | 0.00187 | 0.00230 | 0.078 |
| 166 | 0.00002 | 0.01293 | 0.00153 | 0.325 |
| 167 | 0.00012 | 0.02043 | 0.01340 | 0.409 |
| 168 | 0.00002 | 0.00136 | 0.00095 | 0.052 |
| 169 | 0.00001 | 0.00116 | 0.00038 | 0.034 |
| 170 | 0.00003 | 0.00283 | 0.00454 | 0.084 |
| 171 | 0.00004 | 0.00315 | 0.00373 | 0.077 |
| 172 | 0.00008 | 0.02971 | 0.01964 | 0.403 |
| 173 | 0.00026 | 0.05853 | 0.02946 | 2.437 |
| 174 | 0.00008 | 0.03004 | 0.01005 | 1.021 |
| 175 | 0.00001 | 0.00098 | 0.00046 | 0.176 |
| 176 | 0.00001 | 0.00162 | 0.00048 | 0.182 |
| 177 | 0.00002 | 0.00133 | 0.00078 | 0.381 |
| 178 | 0.00003 | 0.00142 | 0.00084 | 0.395 |
| 179 | 0.00001 | 0.00051 | 0.00030 | 0.077 |
| 180 | 0.00002 | 0.00092 | 0.00058 | 0.145 |
| 181 | 0.00045 | 0.00475 | 0.02093 | >10.000 |
| 182 | 0.00154 | 0.00932 | 0.05004 | >10.000 |
| 183 | | 0.00093 | | 0.158 |
| 184 | 0.00003 | 0.00354 | 0.00093 | 0.161 |
| 185 | 0.00002 | 0.00404 | 0.00244 | 0.632 |
| 186 | 0.00004 | 0.00300 | 0.00132 | 0.527 |
| 187 | 0.00003 | 0.00247 | 0.00128 | 0.210 |
| 188 | 0.00007 | 0.00396 | 0.00196 | 0.478 |
| 189 | 0.00004 | 0.00267 | 0.00096 | 0.470 |
| 190 | 0.00005 | 0.00326 | 0.00290 | 0.402 |
| 191 | 0.00002 | 0.00231 | 0.00052 | 0.263 |
| 192 | 0.00002 | 0.00349 | 0.00106 | 0.114 |
| 193 | 0.00047 | 0.03183 | 0.01022 | 1.853 |
| 194 | 0.00005 | 0.01041 | 0.00119 | 1.341 |
| 195 | 0.00001 | 0.00039 | 0.00018 | 0.036 |
| 196 | 0.00002 | 0.00276 | 0.00137 | 0.246 |
| 197 | 0.00005 | 0.00256 | 0.00143 | 0.619 |
| 198 | 0.00001 | 0.00033 | 0.00017 | 0.098 |
| 199 | 0.00002 | 0.00100 | 0.00072 | 0.204 |
| 200 | 0.00001 | 0.00056 | 0.00019 | 0.082 |
| 201 | 0.00001 | 0.00098 | 0.00055 | 0.239 |
| 202 | 0.00001 | 0.00109 | 0.00068 | 0.251 |
| 203 | 0.00002 | 0.00118 | 0.00059 | 1.494 |
| 204 | 0.00001 | 0.00038 | 0.00014 | 0.115 |
| 205 | 0.00002 | 0.00152 | 0.00107 | 1.375 |
| 206 | 0.00001 | 0.00166 | 0.00080 | 0.059 |
| 207 | 0.00001 | 0.00123 | 0.00013 | 0.096 |
| 208 | 0.00007 | 0.01577 | 0.00559 | 0.363 |
| 209 | 0.00011 | 0.01100 | 0.00576 | 0.262 |
| 210 | 0.00023 | 0.00588 | 0.01075 | |
| 211 | 0.00014 | 0.00722 | 0.00519 | |
| 212 | 0.00003 | 0.00377 | 0.00103 | 0.050 |
| 213 | 0.00006 | 0.00892 | 0.00145 | |
| 214 | 0.00003 | 0.00107 | 0.00037 | 0.032 |
| 215 | 0.00006 | 0.00254 | 0.00088 | 0.036 |
| 216 | 0.00002 | 0.00202 | 0.00039 | 0.028 |
| 217 | 0.00004 | 0.00239 | 0.00070 | 0.062 |
| 218 | 0.00005 | 0.00232 | 0.00068 | 0.023 |
| 219 | 0.00003 | 0.00201 | 0.00065 | 0.023 |
| 220 | 0.00001 | 0.00064 | 0.00014 | 0.008 |
| 221 | 0.00001 | 0.00073 | 0.00013 | 0.004 |
| 222 | 0.00001 | 0.00081 | 0.00035 | 0.023 |
| 223 | 0.00003 | 0.00378 | 0.00155 | 0.169 |
| 224 | 0.00003 | 0.00255 | 0.00130 | 0.064 |
| 225 | 0.00002 | 0.00178 | 0.00051 | |
| 226 | 0.00004 | 0.00351 | 0.00075 | |
| 227 | 0.00016 | 0.01726 | 0.02390 | 0.221 |
| 228 | 0.00014 | 0.00626 | 0.00785 | 0.094 |
| 229 | 0.00003 | 0.00258 | 0.00082 | 0.026 |
| 230 | 0.00002 | 0.00152 | 0.00086 | 0.062 |
| 231 | 0.00004 | 0.00260 | 0.00153 | 0.046 |
| 232 | 0.00005 | 0.00301 | 0.00207 | 0.047 |
| 233 | 0.00004 | 0.00220 | 0.00103 | 0.020 |
| 234 | 0.00004 | 0.00177 | 0.00065 | 0.009 |
| 235 | 0.00002 | 0.00087 | 0.00025 | 0.020 |
| 236 | | 0.00141 | | 0.013 |
| 237 | 0.00025 | 0.01422 | 0.01603 | 0.181 |
| 238 | 0.00004 | 0.00315 | 0.00136 | 0.055 |
| 239 | 0.00001 | 0.00172 | 0.00110 | 0.031 |
| 240 | 0.00002 | 0.00167 | 0.00043 | 0.023 |
| 241 | 0.00002 | 0.00124 | 0.00061 | 0.030 |

1. A compound of Formula (I): 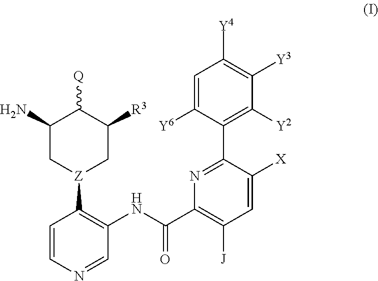
or a pharmaceutically acceptable salt thereof, wherein:
Z is N or CH;
Q is H, Me, or —OH;
R³ is H, Me, or C₂₋₄ alkyl;
X is H or F;
J is H or NH₂;
Y² and Y⁶ are each independently F or Cl, preferably F;
Y³ is H or is selected from the group consisting of CN, OEt, S(O)ₚR, —O(CH₂)_{q}—OH, —O(CH₂)_{q}—OR, —(CH₂)_{q}—OH, —C(CH₃)₂OH, —(CH₂)_{q}—OR, —(CR′₂)₁₋₃—OR′ or —O—(CR′₂)₁₋₃—OR′ where each R′ is independently H or Me, and an optionally substituted member selected from the group consisting of C₁₋₄ alkyl, C₂₋₄ alkyenyl, C₂₋₄ alkynyl, C₁₋₄ alkoxy, C₂₋₄ alkyenyloxy, C₂₋₄ alkynyloxy, C₁₋₄ alkylthio, C₁₋₄ alkylsulfonyl, C₁₋₄ hydroxyalkyl, C₁₋₄ hydroxyalkyloxy, C₃₋₇ cycloalkyl, C₃₋₇ heterocycloalkyl, C₅₋₁₀ heteroaryl, and C₆₋₁₀ aryl, each of which is optionally substituted with up to three groups independently selected from halo, hydroxy, amino, OMe, CN, oxo, R and OR;
when Y³ is H, Y⁴ is selected from the group consisting of CN, R, vinyl, COOH, COOR, S(O)_{q}R, —O(CH₂)_{q}OH, —O(CH₂)ₚOR, —(CH₂)_{q}—OH, —C(CH₃)₂OH, —(CH₂)ₚ—OR, —(CH₂)_{q}—R, —O—(CH₂)_{q}—R, —(CR′₂)₁₋₃—OR′ or —O—(CR′₂)₁₋₃—OR′ where each R′ is independently H or Me, and an optionally substituted member selected from the group consisting of C₁₋₄ alkyl, C₁₋₄ alkoxy, C₁₋₄ alkylthio, C₁₋₄ alkylsulfonyl, C₁₋₄ hydroxyalkyl, C₁₋₄ hydroxyalkyloxy, C₃₋₇ cycloalkyl, C₃₋₇ heterocycloalkyl, C₅₋₁₀ heteroaryl, and C₆₋₁₀ aryl, each of which is optionally substituted with up to two groups independently selected from halo, hydroxy, amino, OMe, CN, oxo, R and OR; and
Y⁴ can be H when Y³ is not H;
or Y³ and Y⁴ taken together form a 5-6 membered ring selected from cycloalkyl, cycloalkenyl, heterocyclyl, heteroaryl and aryl, which ring is optionally substituted with up to two groups independently selected from R, halo, —OH, —OR, —(CH₂)₁₋₃—OR, —O—(CH₂)₁₋₃—OR, —(CH₂)_{q}—OH, and —(CH₂)_{q}—OH;
each R is independently an optionally substituted C₁₋₄ alkyl, C₃₋₇ cycloalkyl, C₅₋₆ cycloalkenyl, C₅₋₆ heterocyclyl, or 3-7 membered cyclic ether, wherein the optional substitutents for R are independently selected from OH, Me, —CH₂OH, COOH, COOMe, CONH₂, CONHMe, CONMe₂, CF₃, OMe, CN, NH₂, halo, oxo, and CN;
each q is independently 1 or 2; and
each p is independently 0, 1 or 2.

2. The compound of claim 1, wherein Z is N.

3. The compound of claim 1, wherein Z is CH.

4. The compound of claim 1, wherein Q is H.

5. The compound of claim 1, wherein Q is —OH.

6. The compound of claim 1, which is a compound of Formula (IIa):

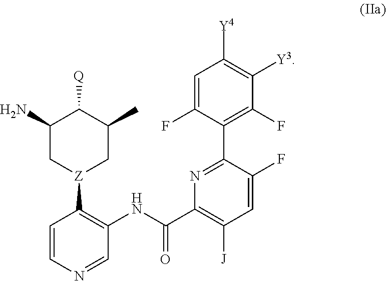

7. The compound of claim 1, which is a compound of Formula (IIb):

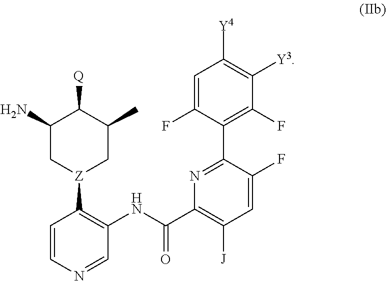

8. The compound of claim 1, wherein X is F.

9. The compound of claim 1, wherein X is H.

10. The compound of claim 1, wherein J is H.

11. The compound of claim 1, wherein J is —NH₂.

12. The compound of claim 1, wherein one of Y³ and Y⁴ is selected from the group consisting of OMe, Me, Et, —CH₂OMe, COOH, COOMe, S(O)ₚMe, —O(CH₂)₂—OH, —(CH₂)₂—OH, —O(CH₂)₂—OMe, —OCH₂—CH(OH)—CH₂OH, —CH(OH)—CH₂OH, —(CH₂)_{q}—OH, —C(CH₃)₂OH, and —(CH₂)_{q}—OR;
where p is 0, 1 or 2,
and each q is 1 or 2.

13. The compound of claim 1, wherein Y³ is H and Y⁴ is selected from the group consisting of CN, OMe, OEt, Me, Et, COOH, COOMe, S(O)_{q}Me, —O(CH₂)₂—OH, —O(CH₂)₂—OMe, —OCH₂—CH(OH)—CH₂OH, —CH(OH)—CH₂OH, —(CH₂)₂—OH, —C(CH₃)₂OH, —CH₂OH, 3-hydroxy-3-oxetanyl, 3-oxetanyloxy, cyclopropyl, 1-hydroxycyclopropyl, 2-hydroxy-2-methylpropoxy, 1-hydroxycyclobutyl, 2-methoxy-2-methylpropoxy, difluoromethyl, isopropoxy, 2-hydroxy-2-methylethyl, 3-tetrahydrofuranyloxy, 1-hydroxyethyl, cyclopropylmethoxy, 4-tetrahydropyranyloxy, difluoromethoxy, and —CH₂OMe.

14. The compound of claim 1, wherein Y⁴ is H and Y³ is selected from the group consisting of CN, Et, COOH, COOMe, S(O)_{q}Me, —O(CH₂)₂—OH, —O(CH₂)₂—OMe, —(CH₂)₂—OH, —OCH₂—CH(OH)—CH₂OH, —CH(OH)—CH₂OH, —CH₂OH, —C(CH₃)₂OH and —CH₂OMe.

15. A compound selected from the compounds in Table 1, 2 and 3;
or a pharmaceutically acceptable salt thereof.

16. A pharmaceutical composition comprising a compound of claim 1 admixed with at least one pharmaceutically acceptable excipient or carrier.

17. The pharmaceutical composition of claim 16, further comprising an additional therapeutic agent.

18. The pharmaceutical composition of claim 17, wherein the additional therapeutic agent is selected from irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib, anthracyclines, rituximab, and trastuzumab.

19. A method to treat a condition associated with excessive levels of PIM Kinase activity, which comprises administering to a subject having the condition an effective amount of a compound or pharmaceutical composition according to any of claim 1.

20. The method of claim 19, wherein the condition is cancer or an autoimmune disorder.

21. The method of claim 20, wherein the cancer is a cancer selected from carcinoma of the lungs, pancreas, thyroid, ovary, bladder, breast, prostate, or colon, melanoma, myeloid leukemia, multiple myeloma, erythroleukemia, villous colon adenoma, and osteosarcoma.

22. The method of claim 21, wherein the autoimmune disorder is selected from Crohn's disease, inflammatory bowel disease, rheumatoid arthritis, and chronic inflammatory diseases.
